(19) United States
(12) Patent Application Publication
(10) Pub. No.: US 2024/0206205 A1
(43) Pub. Date: Jun. 20, 2024

(54) HETEROCYCLIC COMPOUND, ORGANIC LIGHT-EMITTING DEVICE COMPRISING SAME, AND COMPOSITION FOR ORGANIC LAYER

(71) Applicant: LT MATERIALS CO., LTD., Yongin-si, Gyeonggi-do (KR)

(72) Inventors: Su-Yeon KIM, Yongin-si (KR); Young-Seok NO, Yongin-si (KR); Dong-Jun KIM, Yongin-si (KR)

(73) Assignee: LT MATERIALS CO., LTD., Yongin-si, Gyeonggi-do (KR)

(21) Appl. No.: 18/277,132

(22) PCT Filed: Nov. 16, 2021
(86) PCT No.: PCT/KR2021/016789

§ 371 Date: Aug. 14, 2023

(30) Foreign Application Priority Data
Mar. 31, 2021 (KR) 10-2021-0041854

(51) Int. Cl. H10K 50/11; C07D 251/42; C07D 405/14; C07D 409/04; C07D 409/12; C07D 409/14; H10K 50/15; H10K 50/16; H10K 50/17; H10K 50/18; H10K 85/60

(52) U.S. Cl. CPC H10K 50/11; C07D 251/42; C07D 405/14; C07D 409/04; C07D 409/12; C07D 409/14; H10K 50/15; H10K 50/16; H10K 50/171; H10K 50/181; H10K 85/6572

(57) ABSTRACT

The present specification relates to a heterocyclic compound of Chemical Formula 1, an organic light emitting device including same, and a composition for an organic material layer.

### TECHNICAL FIELD

The present specification relates to a heterocyclic compound, an organic light emitting device including same, and a composition for an organic material layer.

The present specification claims priority to and the benefits of Korean Patent Application No. 10-2021-0041854, filed with the Korean Intellectual Property Office on Mar. 31, 2021, the entire contents of which are incorporated herein by reference.

### BACKGROUND ART

An electroluminescent device is one type of self-emissive display devices, and has an advantage of having a wide viewing angle, and a high response speed as well as having an excellent contrast.

An organic light emitting device has a structure disposing an organic thin film between two electrodes. When a voltage is applied to an organic light emitting device having such a structure, electrons and holes injected from the two electrodes bind and pair in the organic thin film, and light emits as these annihilate. The organic thin film may be formed in a single layer or a multilayer as necessary.

A material of the organic thin film may have a light emitting function as necessary. For example, as a material of the organic thin film, compounds capable of forming a light emitting layer themselves alone may be used, or compounds capable of performing a role of a host or a dopant of a host-dopant-based light emitting layer may also be used. In addition thereto, compounds capable of performing roles of hole injection, hole transfer, electron blocking, hole blocking, electron transfer, electron injection and the like may also be used as a material of the organic thin film.

Development of an organic thin film material has been continuously required for enhancing performance, lifetime or efficiency of an organic light emitting device.

### DISCLOSURE

### Technical Problem

The present specification is directed to providing a heterocyclic compound, an organic light emitting device including same, and a composition for an organic material layer.

### Technical Solution

One embodiment of the present specification provides a heterocyclic compound of the following Chemical Formula 1.

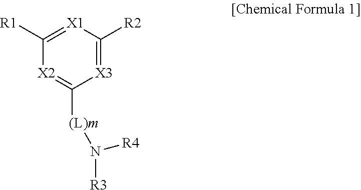

In Chemical Formula 1,
X1 to X3 are each independently N; or CH, and at least two thereof are N,
L is a direct bond; or a substituted or unsubstituted C6 to C60 arylene group,
m is an integer of 1 to 5, and when 2 or greater, Ls are the same as or different from each other,
R1 and R2 are the same as or different from each other and each independently a substituted or unsubstituted C6 to C12 aryl group; a substituted or unsubstituted fluorenyl group; or a substituted or unsubstituted C2 to C60 heteroaryl group, and at least one of R1 and R2 is a substituted or unsubstituted fluorenyl group; represented by the following Chemical Formula A; or represented by the following Chemical Formula B, and
R3 and R4 are the same as or different from each other and each independently a substituted or unsubstituted C6 to C12 aryl group; a substituted or unsubstituted fluorenyl group; or a heteroaryl group substituted or unsubstituted and including O or S, and at least one of R3 and R4 is a substituted or unsubstituted fluorenyl group; or represented by the following Chemical Formula C,

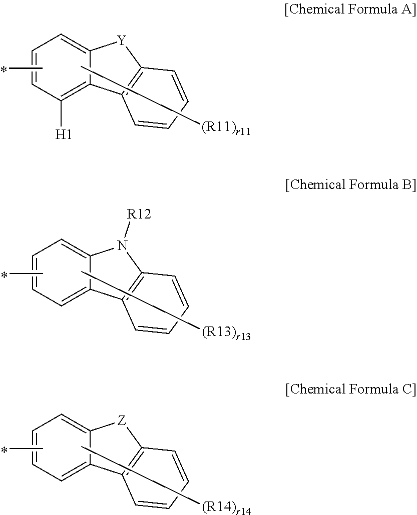

in Chemical Formulae A to C,
Y and Z are each independently O; or S,
H1 is hydrogen; or deuterium,
R11 to R14 are each independently hydrogen; deuterium; a substituted or unsubstituted C1 to C60 alkyl group; a substituted or unsubstituted C6 to C60 aryl group; or a substituted or unsubstituted C2 to C60 heteroaryl group,
r11 is an integer of 0 to 6, and when 2 or greater, R11s are the same as or different from each other,
r13 is an integer of 0 to 7, and when 2 or greater, R13s are the same as or different from each other, and
r14 is an integer of 0 to 7, and when 2 or greater, R14s are the same as or different from each other.

Another embodiment of the present specification provides an organic light emitting device including a first electrode; a second electrode; and one or more organic material layers provided between the first electrode and the second electrode, wherein one or more layers of the organic material layers include the heterocyclic compound of Chemical Formula 1.

Another embodiment of the present specification provides a composition for an organic material layer of an organic light emitting device, the composition including the heterocyclic compound of Chemical Formula 1.

### Advantageous Effects

A compound described in the present specification can be used as a material of an organic material layer of an organic light emitting device. The compound is capable of performing a role of a hole injection material, a hole transfer material, a light emitting material, an electron transfer material, an electron injection material, a charge generation material or the like in an organic light emitting device. Particularly, the compound can be used as a light emitting material of an organic light emitting device.

When the heterocyclic compound of Chemical Formula 1 is used as a light emitting material of an organic light emitting device, that is, included in a light emitting layer and used, an organic light emitting device having superior properties in terms of driving voltage and lifetime can be provided.

Specifically, in the heterocyclic compound of Chemical Formula 1, an amine group bonds to an azine ring through a direct bond or a linker, the azine ring is substituted with at least one of a fluorenyl group, a dibenzofuran group, a dibenzothiophene group or carbazole, and the amine group is substituted with at least one of a fluorenyl group, a dibenzofuran group or a dibenzothiophene group. When the amine group bonds to the azine ring through a direct bond or a linker, hole mobility accelerates helping to enhance light efficiency, and particularly when using the heterocyclic compound as a material of a light emitting layer, a driving voltage of a device can be lowered, light efficiency can be enhanced, and a lifetime of the device can be enhanced by thermal stability of the compound.

### DESCRIPTION OF DRAWINGS

FIG. 1 to FIG. 3 are diagrams each illustrating a lamination structure of an organic light emitting device according to one embodiment of the present specification.

### REFERENCE NUMERAL

100: Substrate
200: Anode
300: Organic Material Layer
301: Hole Injection Layer
302: Hole Transfer Layer
303: Light Emitting Layer
304: Hole Blocking Layer
305: Electron Transfer Layer
306: Electron Injection Layer
400: Cathode

### MODE FOR DISCLOSURE

Hereinafter, the present specification will be described in more detail.

In the present specification, a description of a certain part “including” certain constituents means capable of further including other constituents, and does not exclude other constituents unless particularly stated on the contrary.

A term “substitution” means a hydrogen atom bonding to a carbon atom of a compound being changed to another substituent, and the position of substitution is not limited as long as it is a position at which the hydrogen atom is substituted, that is, a position at which a substituent is capable of substituting, and when two or more substituents substitute, the two or more substituents may be the same as or different from each other.

In the present specification, “substituted or unsubstituted” means being substituted with one or more substituents selected from the group consisting of deuterium; a halogen group; a cyano group; a C1 to C60 alkyl group; a C2 to C60 alkenyl group; a C2 to C60 alkynyl group; a C3 to C60 cycloalkyl group; a C2 to C60 heterocycloalkyl group; a C6 to C60 aryl group; a C2 to C60 heteroaryl group; a silyl group; a phosphine oxide group; and an amine group, or being substituted with a substituent linking two or more substituents selected from among the substituents illustrated above, or being unsubstituted.

In the present specification, a “case of a substituent being not indicated in a chemical formula or compound structure” means that a hydrogen atom bonds to a carbon atom. However, since deuterium (²H) is an isotope of hydrogen, some hydrogen atoms may be deuterium.

In one embodiment of the present application, a “case of a substituent being not indicated in a chemical formula or compound structure” may mean that positions that may come as a substituent may all be hydrogen or deuterium. In other words, since deuterium is an isotope of hydrogen, some hydrogen atoms may be deuterium that is an isotope, and herein, a content of the deuterium may be from 0% to 100%.

In one embodiment of the present application, in a “case of a substituent being not indicated in a chemical formula or compound structure”, hydrogen and deuterium may be mixed in compounds when deuterium is not explicitly excluded such as a deuterium content being 0%, a hydrogen content being 100% or substituents being all hydrogen.

In one embodiment of the present application, deuterium is one of isotopes of hydrogen, is an element having deuteron formed with one proton and one neutron as a nucleus, and may be expressed as hydrogen-2, and the elemental symbol may also be written as D or ²H.

In one embodiment of the present application, an isotope means an atom with the same atomic number (Z) but with a different mass number (A), and may also be interpreted as an element with the same number of protons but with a different number of neutrons.

In one embodiment of the present application, a meaning of a content T % of a specific substituent may be defined as T2/T1×100=T % when the total number of substituents that a basic compound may have is defined as T1, and the number of specific substituents among these is defined as T2.

In other words, in one example, having a deuterium content of 20% in a phenyl group represented by

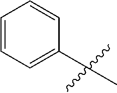 [SMILES: *c1ccccc1]

means that the total number of substituents that the phenyl group may have is 5 (T1 in the formula), and the number of deuterium among these is 1 (T2 in the formula). In other words, having a deuterium content of 20% in a phenyl group may be represented by the following structural formulae.

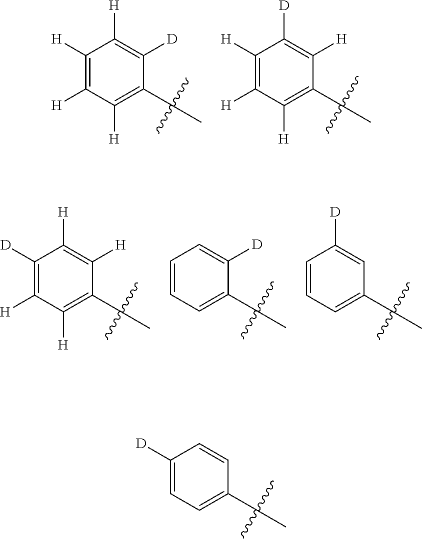 [SMILES: *c1ccccc1[2H] ; *c1cccc([2H])c1 ; *c1ccc([2H])cc1 ; *c1ccccc1[2H] ; *c1cccc([2H])c1 ; *c1ccc([2H])cc1]

In addition, in one embodiment of the present application, “a phenyl group having a deuterium content of 0%” may mean a phenyl group that does not include a deuterium atom, that is, a phenyl group that has 5 hydrogen atoms.

In the present specification, the alkyl group includes linear or branched, and may be further substituted with other substituents. The number of carbon atoms of the alkyl group may be from 1 to 60, specifically from 1 to 40 and more specifically from 1 to 20. Specific examples thereof may include a methyl group, an ethyl group, a propyl group, an n-propyl group, an isopropyl group, a butyl group, an n-butyl group, an isobutyl group, a tert-butyl group, a sec-butyl group, a 1-methyl-butyl group, a 1-ethyl-butyl group, a pentyl group, an n-pentyl group, an isopentyl group, a neopentyl group, a tert-pentyl group, a hexyl group, an n-hexyl group, a 1-methylpentyl group, a 2-methylpentyl group, a 4-methyl-2-pentyl group, a 3,3-dimethylbutyl group, a 2-ethylbutyl group, a heptyl group, an n-heptyl group, a 1-methylhexyl group, an octyl group, an n-octyl group, a tert-octyl group, a 1-methylheptyl group, a 2-ethylhexyl group, a 2-propylpentyl group, an n-nonyl group, a 2,2-dimethylheptyl group, a 1-ethyl-propyl group, a 1,1-dimethyl-propyl group, an isohexyl group, a 2-methylpentyl group, a 4-methylhexyl group, a 5-methylhexyl group and the like, but are not limited thereto.

In the present specification, the alkenyl group includes linear or branched, and may be further substituted with other substituents. The number of carbon atoms of the alkenyl group may be from 2 to 60, specifically from 2 to 40 and more specifically from 2 to 20. Specific examples thereof may include a vinyl group, a 1-propenyl group, an isopropenyl group, a 1-butenyl group, a 2-butenyl group, a 3-butenyl group, a 1-pentenyl group, a 2-pentenyl group, a 3-pentenyl group, a 3-methyl-1-butenyl group, a 1,3-butadienyl group, an allyl group, a 1-phenylvinyl-1-yl group, a 2-phenylvinyl-1-yl group, a 2,2-diphenylvinyl-1-yl group, a 2-phenyl-2-(naphthyl-1-yl)vinyl-1-yl group, a 2,2-bis(diphenyl-1-yl)vinyl-1-yl group, a stilbenyl group, a styrenyl group and the like, but are not limited thereto.

In the present specification, the alkynyl group includes linear or branched, and may be further substituted with other substituents. The number of carbon atoms of the alkynyl group may be from 2 to 60, specifically from 2 to 40 and more specifically from 2 to 20.

In the present specification, the cycloalkyl group includes monocyclic or polycyclic, and may be further substituted with other substituents. Herein, the polycyclic means a group in which the cycloalkyl group is directly linked to or fused with other cyclic groups. Herein, the other cyclic groups may be a cycloalkyl group, but may also be different types of cyclic groups such as a heterocycloalkyl group, an aryl group and a heteroaryl group. The number of carbon groups of the cycloalkyl group may be from 3 to 60, specifically from 3 to 40 and more specifically from 5 to 20. Specific examples thereof may include a cyclopropyl group, a cyclobutyl group, a cyclopentyl group, a 3-methylcyclopentyl group, a 2,3-dimethylcyclopentyl group, a cyclohexyl group, a 3-methylcyclohexyl group, a 4-methylcyclohexyl group, a 2,3-dimethylcyclohexyl group, a 3,4,5-trimethylcyclohexyl group, a 4-tert-butylcyclohexyl group, a cycloheptyl group, a cyclooctyl group and the like, but are not limited thereto.

In the present specification, the heterocycloalkyl group includes O, S, Se, N or Si as a heteroatom, includes monocyclic or polycyclic, and may be further substituted with other substituents. Herein, the polycyclic means a group in which the heterocycloalkyl group is directly linked to or fused with other cyclic groups. Herein, the other cyclic groups may be a heterocycloalkyl group, but may also be different types of cyclic groups such as a cycloalkyl group, an aryl group and a heteroaryl group. The number of carbon atoms of the heterocycloalkyl group may be from 2 to 60, specifically from 2 to 40 and more specifically from 3 to 20.

In the present specification, the aryl group includes monocyclic or polycyclic, and may be further substituted with other substituents. Herein, the polycyclic means a group in which the aryl group is directly linked to or fused with other cyclic groups. Herein, the other cyclic groups may be an aryl group, but may also be different types of cyclic groups such as a cycloalkyl group, a heterocycloalkyl group and a heteroaryl group. The aryl group includes a spiro group. The number of carbon atoms of the aryl group may be from 6 to 60, specifically from 6 to 40 and more specifically from 6 to 25. When the aryl group is dicyclic or higher, the number of carbon atoms may be from 8 to 60, from 8 to 40 or from 8 to 30. Specific examples of the aryl group may include a phenyl group, a biphenyl group, a ter-phenyl group, a naphthyl group, an anthryl group, a chrysenyl group, a phenanthrenyl group, a perylenyl group, a fluoranthenyl group, a triphenylenyl group, a phenalenyl group, a pyrenyl group, a tetracenyl group, a pentacenyl group, a fluorenyl group, an indenyl group, an acenaphthylenyl group, a benzofluorenyl group, a spirobifluorenyl group, a 2,3-dihydro-1H-indenyl group, a fused ring group thereof, and the like, but are not limited thereto.

In the present specification, the fluorenyl group may be substituted, and adjacent substituents may bond to each other to form a ring.

When the fluorenyl group is substituted,

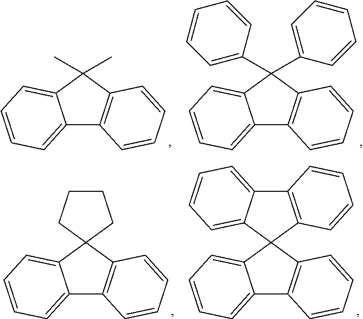

and the like may be included, however, the structure is not limited thereto.

In the present specification, the heteroaryl group includes O, S, SO₂, Se, N or Si as a heteroatom, includes monocyclic or polycyclic, and may be further substituted with other substituents. Herein, the polycyclic means a group in which the heteroaryl group is directly linked to or fused with other cyclic groups. Herein, the other cyclic groups may be a heteroaryl group, but may also be different types of cyclic groups such as a cycloalkyl group, a heterocycloalkyl group and an aryl group. The number of carbon atoms of the heteroaryl group may be from 2 to 60, specifically from 2 to 40 and more specifically from 3 to 25. When the heteroaryl group is dicyclic or higher, the number of carbon atoms may be from 4 to 60, 4 to 40 or 4 to 25. Specific examples of the heteroaryl group may include a pyridyl group, a pyrrolyl group, a pyrimidyl group, a pyridazinyl group, a furanyl group, a thiophene group, an imidazolyl group, a pyrazolyl group, an oxazolyl group, an isoxazolyl group, a thiazolyl group, an isothiazolyl group, a triazolyl group, a furazanyl group, an oxadiazolyl group, a thiadiazolyl group, a dithiazolyl group, a tetrazolyl group, a pyranyl group, a thiopyranyl group, a diazinyl group, an oxazinyl group, a thiazinyl group, a dioxynyl group, a triazinyl group, a tetrazinyl group, a quinolyl group, an isoquinolyl group, a quinazolinyl group, an isoquinazolinyl group, a quinozolinyl group, a naphthyridyl group, an acridinyl group, a phenanthridinyl group, an imidazopyridinyl group, a diazanaphthalenyl group, a triazaindene group, an indolyl group, an indolizinyl group, a benzothiazolyl group, a benzoxazolyl group, a benzimidazolyl group, a benzothiophene group, a benzofuran group, a dibenzothiophene group, a dibenzofuran group, a carbazolyl group, a benzocarbazolyl group, a dibenzocarbazolyl group, a phenazinyl group, a dibenzosilole group, spirobi(dibenzosilole), a dihydrophenazinyl group, a phenoxazinyl group, a phenanthridyl group, a thienyl group, an indolo[2,3-a]carbazolyl group, an indolo[2,3-b]carbazolyl group, an indolinyl group, a 10,11-dihydro-dibenzo[b,f]azepine group, a 9,10-dihydroacridinyl group, a phenanthrazinyl group, a phenothiathiazinyl group, a phthalazinyl group, a naphthylidinyl group, a phenanthrolinyl group, a benzo[c][1,2,5]thiadiazolyl group, 5,10-dihydrobenzo[b,e][1,4]azasilinyl, a pyrazolo[1,5-c]quinazolinyl group, a pyrido[1,2-b]indazolyl group, a pyrido[1,2-a]imidazo[1,2-e]indolinyl group, a benzofuro[2,3-d]pyrimidyl group; a benzothieno[2,3-d]pyrimidyl group; a benzofuro[2,3-a]carbazolyl group, a benzothieno[2,3-a]carbazolyl group, a 1,3-dihydroindolo[2,3-a]carbazolyl group, a benzofuro[3,2-a]carbazolyl group, a benzothieno[3,2-a]carbazolyl group, a 1,3-dihydroindolo[3,2-a]carbazolyl group, a benzofuro[2,3-b]carbazolyl group, a benzothieno[2,3-b]carbazolyl group, a 1,3-dihydroindolo[2,3-b]carbazolyl group, a benzofuro[3,2-b]carbazolyl group, a benzothieno[3,2-b]carbazolyl group, a 1,3-dihydroindolo[3,2-b]carbazolyl group, a benzofuro[2,3-c]carbazolyl group, a benzothieno[2,3-c]carbazolyl group, a 1,3-dihydroindolo[2,3-c]carbazolyl group, a benzofuro[3,2-c]carbazolyl group, a benzothieno[3,2-c]carbazolyl group, a 1,3-dihydroindolo[3,2-c]carbazolyl group, a 1,3-dihydroindeno[2,1-b]carbazolyl group, a 5,11-dihydroindeno[1,2-b]carbazolyl group, a 5,12-dihydroindeno[1,2-c]carbazolyl group, a 5,8-dihydroindeno[2,1-c]carbazolyl group, a 7,12-dihydroindeno[1,2-a]carbazolyl group, a 11,12-dihydroindeno[2,1-a]carbazolyl group and the like, but are not limited thereto.

In the present specification, the silyl group is a substituent including Si and having the Si atom directly linked as a radical, and is represented by —Si(R101) (R102) (R103). R101 to R103 are the same as or different from each other, and may be each independently a substituent formed with at least one of hydrogen; deuterium; a halogen group; an alkyl group; an alkenyl group; an alkoxy group; a cycloalkyl group; an aryl group; and a heteroaryl group. Specific examples of the silyl group may include a trimethylsilyl group

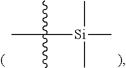 [SMILES: *[Si](C)(C)C]

a triethylsilyl group

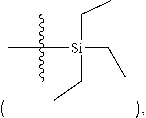

a t-butyldimethylsilyl group

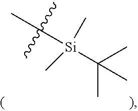

a vinyldimethylsilyl group

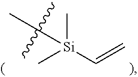 [SMILES: *[Si](C)(C)C=C]

a propyldimethylsilyl group

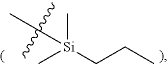 [SMILES: *[Si](C)(C)CCC]

a triphenylsilyl group

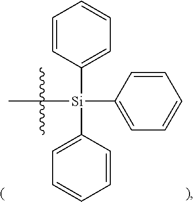 [SMILES: *[Si](c1ccccc1)(c1ccccc1)c1ccccc1]

a diphenylsilyl group

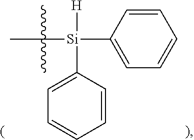 [SMILES: *[SiH](c1ccccc1)c1ccccc1]

a phenylsilyl group

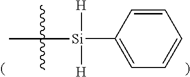 [SMILES: *[SiH2]c1ccccc1]

and the like, but are not limited thereto.

In the present specification, the phosphine oxide group is represented by —P(═O) (R104) (R105), and R104 and R105 are the same as or different from each other and may be each independently a substituent formed with at least one of hydrogen; deuterium; a halogen group; an alkyl group; an alkenyl group; an alkoxy group; a cycloalkyl group; an aryl group; and a heteroaryl group. Specifically, the phosphine oxide group may be substituted with an alkyl group or an aryl group, and as the alkyl group and the aryl group, the examples described above may be used. Examples of the phosphine oxide group may include a dimethylphosphine oxide group, a diphenylphosphine oxide group, a dinaphthylphosphine oxide group and the like, but are not limited thereto.

In the present specification, the amine group is represented by —N(R106) (R107), and R106 and R107 are the same as or different from each other and may be each independently a substituent formed with at least one of hydrogen; deuterium; a halogen group; an alkyl group; an alkenyl group; an alkoxy group; a cycloalkyl group; an aryl group; and a heteroaryl group. The amine group may be selected from the group consisting of —NH₂; a monoalkylamine group; a monoarylamine group; a monoheteroarylamine group; a dialkylamine group; a diarylamine group; a diheteroarylamine group; an alkylarylamine group; an alkylheteroarylamine group; and an arylheteroarylamine group, and although not particularly limited thereto, the number of carbon atoms is preferably from 1 to 30. Specific examples of the amine group may include a methylamine group, a dimethylamine group, an ethylamine group, a diethylamine group, a phenylamine group, a naphthylamine group, a biphenylamine group, a dibiphenylamine group, an anthracenylamine group, a 9-methyl-anthracenylamine group, a diphenylamine group, a phenylnaphthylamine group, a ditolylamine group, a phenyltolylamine group, a triphenylamine group, a biphenylnaphthylamine group, a phenylbiphenylamine group, a biphenylfluorenylamine group, a phenyltriphenylenylamine group, a biphenyltriphenylenylamine group and the like, but are not limited thereto.

In the present specification, the descriptions on the aryl group provided above may be applied to an arylene group except that the arylene group is a divalent group.

In the present specification, a phenylene group may be selected from among the following structures, and may be further substituted with deuterium.

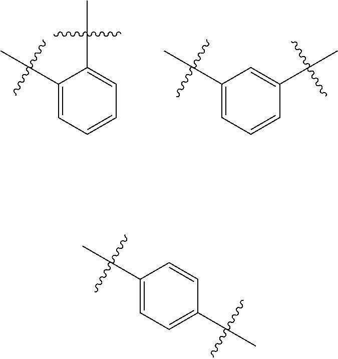 [SMILES: *c1ccccc1* ; *c1cccc(*)c1 ; *c1ccc(*)cc1]

In the present specification,

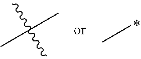

means a substituted position.

In one embodiment of the present specification, X1 to X3 are each independently N; or CH, and at least two thereof are N.

In one embodiment of the present specification, two of X1 to X3 are N, and the other one may be CH.

In one embodiment of the present specification, X1 to X3 may be N.

In one embodiment of the present specification, Chemical Formula 1 may be represented by any one of the following Chemical Formulae 1-A to 1-C.

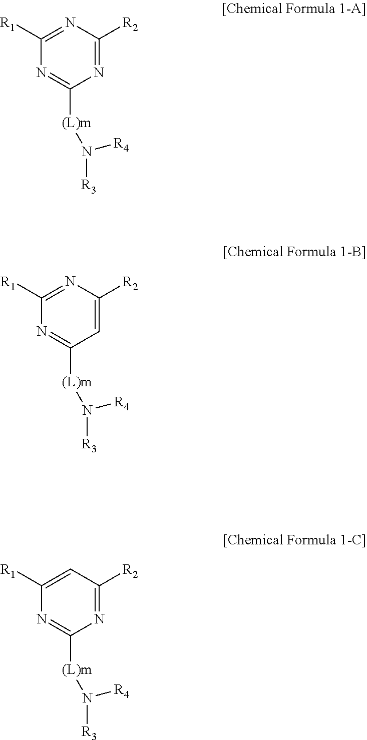

In Chemical Formulae 1-A to 1-C,
each substituent has the same definition as in Chemical Formula 1.

In one embodiment of the present specification, Chemical Formula 1 may be represented by any one of the following Chemical Formulae 1-1 to 1-3.

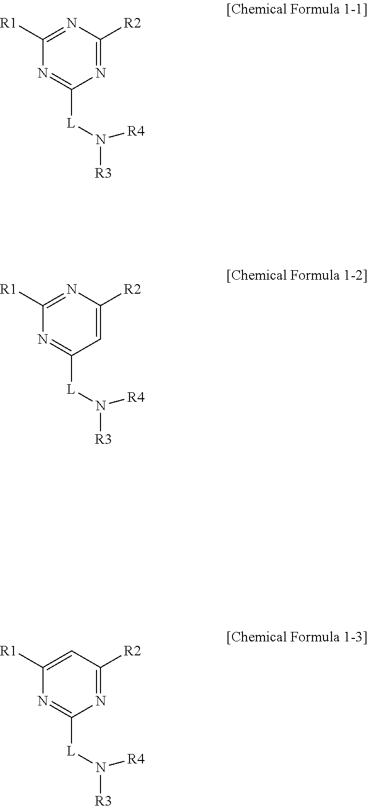

In Chemical Formulae 1-1 to 1-3,
each substituent has the same definition as in Chemical Formula 1.

In one embodiment of the present specification, Chemical Formula 1 may be represented by Chemical Formula 1-1.

In one embodiment of the present specification, Chemical Formula 1 may be represented by Chemical Formula 1-2.

In one embodiment of the present specification, Chemical Formula 1 may be represented by Chemical Formula 1-3.

In one embodiment of the present specification, L is a direct bond; or a substituted or unsubstituted C6 to C60 arylene group.

In one embodiment of the present specification, L is a direct bond; or a substituted or unsubstituted C6 to C30 arylene group.

In one embodiment of the present specification, L is a direct bond; or a substituted or unsubstituted C6 to C15 arylene group.

In one embodiment of the present specification, L may be a direct bond; or a substituted or unsubstituted phenylene group.

In one embodiment of the present specification, L may be a direct bond; or a phenylene group unsubstituted or substituted with deuterium.

In one embodiment of the present specification, m is an integer of 1 to 5.

In one embodiment of the present specification, m may be an integer of 1 to 3.

In one embodiment of the present specification, when m is 1, Chemical Formula 1 may be represented by the following Chemical Formula 1-(1).

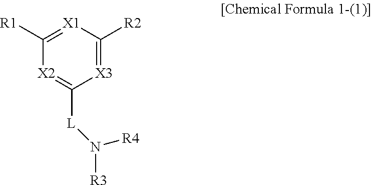

In Chemical Formula 1-(1),
each substituent has the same definition as in Chemical Formula 1.

In one embodiment of the present specification, Chemical Formula 1 may be represented by the following Chemical Formula 1-1-1 or 1-1-2.

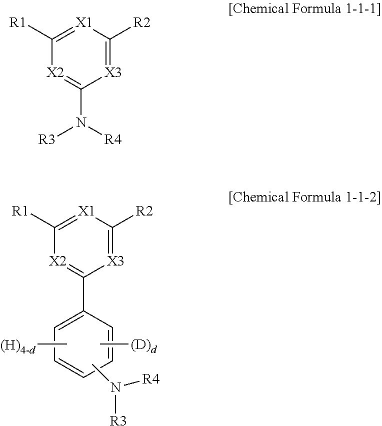

In Chemical Formulae 1-1-1 and 1-1-2,
H is hydrogen, D is deuterium, d is an integer of 0 to 4, and the rest of the substituents have the same definitions as in Chemical Formula 1.

In one embodiment of the present specification, R1 and R2 are the same as or different from each other and each independently a substituted or unsubstituted C6 to C12 aryl group; a substituted or unsubstituted fluorenyl group; or a substituted or unsubstituted C2 to C60 heteroaryl group, and at least one of R1 and R2 is a substituted or unsubstituted fluorenyl group; represented by the following Chemical Formula A; or represented by the following Chemical Formula B.

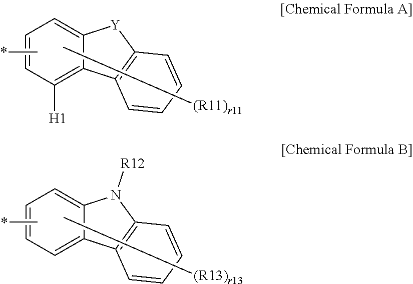

In Chemical Formulae A and B,
Y is O; or S,
H1 is hydrogen; or deuterium,
R11 to R13 are each independently hydrogen; deuterium; a substituted or unsubstituted C1 to C60 alkyl group; a substituted or unsubstituted C6 to C60 aryl group; or a substituted or unsubstituted C2 to C60 heteroaryl group,
r11 is an integer of 0 to 6, and when 2 or greater, R11s are the same as or different from each other, and
r13 is an integer of 0 to 7, and when 2 or greater, R13s are the same as or different from each other.

In one embodiment of the present specification, R1 and R2 are the same as or different from each other and each independently a substituted or unsubstituted C6 to C12 aryl group; a substituted or unsubstituted fluorenyl group; or a substituted or unsubstituted C2 to C30 heteroaryl group, and at least one of R1 and R2 is a substituted or unsubstituted fluorenyl group; represented by Chemical Formula A; or represented by Chemical Formula B.

In one embodiment of the present specification, R1 and R2 are the same as or different from each other and each independently a C6 to C12 aryl group unsubstituted or substituted with deuterium; a fluorenyl group unsubstituted or substituted with an alkyl group; or a substituted or unsubstituted C2 to C30 heteroaryl group, and at least one of R1 and R2 is a substituted or unsubstituted fluorenyl group; represented by Chemical Formula A; or represented by Chemical Formula B.

In one embodiment of the present specification, R1 and R2 are the same as or different from each other and each independently a phenyl group unsubstituted or substituted with deuterium; a biphenyl group unsubstituted or substituted with deuterium; a fluorenyl group unsubstituted or substituted with an alkyl group; or a substituted or unsubstituted C2 to C30 heteroaryl group, and at least one of R1 and R2 is a fluorenyl group unsubstituted or substituted with an alkyl group; represented by Chemical Formula A; or represented by Chemical Formula B.

In one embodiment of the present specification, R1 and R2 are the same as or different from each other and each independently a C6 to C12 aryl group unsubstituted or substituted with deuterium; a fluorenyl group unsubstituted or substituted with one or more substituents of deuterium and an alkyl group; or a substituted or unsubstituted C2 to C30 heteroaryl group, and at least one of R1 and R2 is a substituted or unsubstituted fluorenyl group; represented by Chemical Formula A; or represented by Chemical Formula B.

In one embodiment of the present specification, R1 and R2 are the same as or different from each other and each independently a phenyl group unsubstituted or substituted with deuterium; a biphenyl group unsubstituted or substituted with deuterium; a fluorenyl group unsubstituted or substituted with one or more substituents of deuterium and an alkyl group; or a substituted or unsubstituted C2 to C30 heteroaryl group, and at least one of R1 and R2 is a fluorenyl group unsubstituted or substituted with an alkyl group; represented by Chemical Formula A; or represented by Chemical Formula B.

In one embodiment of the present specification, R1 and R2 are the same as or different from each other and each independently a phenyl group unsubstituted or substituted with deuterium; a biphenyl group unsubstituted or substituted with deuterium; a fluorenyl group unsubstituted or substituted with one or more substituents of deuterium and an alkyl group; a substituted or unsubstituted dibenzofuran group; a substituted or unsubstituted dibenzothiophene group; or a substituted or unsubstituted carbazole group, and at least one of R1 and R2 is a fluorenyl group unsubstituted or substituted with an alkyl group; represented by Chemical Formula A; or represented by Chemical Formula B.

In one embodiment of the present specification, as for R1 and R2, i) any one of R1 and R2 is a substituted or unsubstituted fluorenyl group; represented by Chemical Formula A; or represented by Chemical Formula B, and the other one is a substituted or unsubstituted C6 to C12 aryl group, or ii) R1 and R2 may be each independently a substituted or unsubstituted fluorenyl group; represented by Chemical Formula A; or represented by Chemical Formula B.

In one embodiment of the present specification, as for R1 and R2, i) any one of R1 and R2 is a fluorenyl group unsubstituted or substituted with an alkyl group; represented by Chemical Formula A; or represented by Chemical Formula B, and the other one is a C6 to C12 aryl group unsubstituted or substituted with deuterium, or ii) R1 and R2 may be each independently a fluorenyl group unsubstituted or substituted with an alkyl group; represented by Chemical Formula A; or represented by Chemical Formula B.

In one embodiment of the present specification, Y of Chemical Formula A is O; or S.

In one embodiment of the present specification, R11 of Chemical Formula A may be hydrogen; deuterium; or a substituted or unsubstituted C6 to C60 aryl group.

In one embodiment of the present specification, R11 of Chemical Formula A may be hydrogen; deuterium; or a substituted or unsubstituted C6 to C30 aryl group.

In one embodiment of the present specification, R11 of Chemical Formula A may be hydrogen; deuterium; or a substituted or unsubstituted C6 to C15 aryl group.

In one embodiment of the present specification, R11 of Chemical Formula A may be hydrogen; deuterium; or a substituted or unsubstituted phenyl group.

In one embodiment of the present specification, R11 of Chemical Formula A may be hydrogen; deuterium; or a phenyl group.

In one embodiment of the present specification, R12 of Chemical Formula B may be a substituted or unsubstituted C6 to C60 aryl group.

In one embodiment of the present specification, R12 of Chemical Formula B may be a substituted or unsubstituted C6 to C30 aryl group.

In one embodiment of the present specification, R12 of Chemical Formula B may be a substituted or unsubstituted C6 to C15 aryl group.

In one embodiment of the present specification, R12 of Chemical Formula B may be a substituted or unsubstituted phenyl group.

In one embodiment of the present specification, R12 of Chemical Formula B may be a phenyl group unsubstituted or substituted with deuterium.

In one embodiment of the present specification, R13 of Chemical Formula B may be hydrogen; deuterium; or a substituted or unsubstituted C6 to C60 aryl group.

In one embodiment of the present specification, R13 of Chemical Formula B may be hydrogen; deuterium; or a substituted or unsubstituted C6 to C30 aryl group.

In one embodiment of the present specification, R13 of Chemical Formula B may be hydrogen; deuterium; or a substituted or unsubstituted C6 to C15 aryl group.

In one embodiment of the present specification, R13 of Chemical Formula B may be hydrogen; or deuterium.

In one embodiment of the present specification, R3 and R4 are the same as or different from each other and each independently a substituted or unsubstituted C6 to C12 aryl group; a substituted or unsubstituted fluorenyl group; or a heteroaryl group substituted or unsubstituted and including O or S, and at least one of R3 and R4 is a substituted or unsubstituted fluorenyl group; or represented by the following Chemical Formula C.

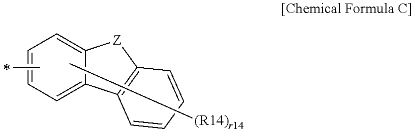

In Chemical Formula C,
Z is O; or S,
R14 is hydrogen; deuterium; a substituted or unsubstituted C1 to C60 alkyl group; a substituted or unsubstituted C6 to C60 aryl group; or a substituted or unsubstituted C2 to C60 heteroaryl group, and
r14 is an integer of 0 to 7, and when 2 or greater, R14s are the same as or different from each other.

In one embodiment of the present specification, R3 and R4 do not include carbazole.

In one embodiment of the present specification, R3 and R4 are the same as or different from each other and each independently a substituted or unsubstituted C6 to C12 aryl group; a substituted or unsubstituted fluorenyl group; a substituted or unsubstituted dibenzofuran group; or a substituted or unsubstituted dibenzothiophene group, and at least one of R3 and R4 is a substituted or unsubstituted fluorenyl group; or represented by Chemical Formula C.

In one embodiment of the present specification, R3 and R4 are the same as or different from each other and each independently a substituted or unsubstituted phenyl group; a substituted or unsubstituted biphenyl group; a substituted or unsubstituted fluorenyl group; a substituted or unsubstituted dibenzofuran group; or a substituted or unsubstituted dibenzothiophene group, and at least one of R3 and R4 is a substituted or unsubstituted fluorenyl group; or represented by Chemical Formula C.

In one embodiment of the present specification, R3 and R4 are the same as or different from each other and each independently a phenyl group unsubstituted or substituted with deuterium; a biphenyl group unsubstituted or substituted with deuterium; a fluorenyl group unsubstituted or substituted with one or more substituents of deuterium and an alkyl group; a dibenzofuran group unsubstituted or substituted with deuterium; or a dibenzothiophene group unsubstituted or substituted with deuterium, and at least one of R3 and R4 is a fluorenyl group unsubstituted or substituted with one or more substituents of deuterium and an alkyl group; or represented by Chemical Formula C.

In one embodiment of the present specification, as for R3 and R4, i) any one of R3 and R4 is a substituted or unsubstituted fluorenyl group; or represented by Chemical Formula C, and the other one is a substituted or unsubstituted C6 to C12 aryl group, or ii) R3 and R4 may be each independently a substituted or unsubstituted fluorenyl group; or represented by Chemical Formula C.

In one embodiment of the present specification, as for R3 and R4, i) any one of R3 and R4 is a fluorenyl group unsubstituted or substituted with one or more substituents of deuterium and an alkyl group; or represented by Chemical Formula C, and the other one is a C6 to C12 aryl group unsubstituted or substituted with deuterium, or ii) R3 and R4 may be each independently a fluorenyl group unsubstituted or substituted with one or more substituents of deuterium and an alkyl group; or represented by Chemical Formula C.

In one embodiment of the present specification, as for R3 and R4, i) any one of R3 and R4 is a fluorenyl group unsubstituted or substituted with one or more substituents of deuterium and an alkyl group; or represented by Chemical Formula C, and the other one is a phenyl group unsubstituted or substituted with deuterium; or a biphenyl group unsubstituted or substituted with deuterium, or ii) R3 and R4 may be each independently a fluorenyl group unsubstituted or substituted with one or more substituents of deuterium and an alkyl group; or represented by Chemical Formula C.

In one embodiment of the present specification, Z of Chemical Formula C is O; or S.

In one embodiment of the present specification, R14 of Chemical Formula C may be hydrogen; deuterium; or a substituted or unsubstituted C6 to C60 aryl group.

In one embodiment of the present specification, R14 of Chemical Formula C may be hydrogen; deuterium; or a substituted or unsubstituted C6 to C30 aryl group.

In one embodiment of the present specification, R14 of Chemical Formula C may be hydrogen; deuterium; or a substituted or unsubstituted C6 to C15 aryl group.

In one embodiment of the present specification, R14 of Chemical Formula C may be hydrogen; deuterium; or a substituted or unsubstituted phenyl group.

In one embodiment of the present specification, R14 of Chemical Formula C may be hydrogen; deuterium; or a C6 to C30 aryl group.

In one embodiment of the present specification, R14 of Chemical Formula C may be hydrogen; deuterium; or a C6 to C15 aryl group.

In one embodiment of the present specification, R14 of Chemical Formula C may be hydrogen; deuterium; or a phenyl group.

In one embodiment of the present specification, Chemical Formula 1 may have a deuterium content of 0% to 100%.

In one embodiment of the present specification, Chemical Formula 1 may have a deuterium content of either 0%, or 30% to 100%.

In one embodiment of the present specification, Chemical Formula 1 may have a deuterium content of either 0%, or 45% to 100%.

In one embodiment of the present specification, when Chemical Formula 1 includes deuterium, superior effects may be provided in terms of driving voltage, light emission efficiency and lifetime.

In one embodiment of the present application, the heterocyclic compound of Chemical Formula 1 satisfies the above-mentioned deuterium content range, and although the compound not including deuterium and the compound including deuterium have almost similar photochemical properties, the material including deuterium tends to be packed with narrower intermolecular distances when deposited on a thin film.

Accordingly, when manufacturing an EOD (electron only device) and a HOD (hole only device) and checking voltage-dependent current density, it is identified that, among the compounds of Chemical Formula 1, the compound including deuterium exhibits far more balanced charge transfer properties compared to the compound not including deuterium.

In addition, when examining the thin film surface using an atomic force microscope (AFM), it is identified that the thin film manufactured using the compound including deuterium is deposited as a more uniform surface with no aggregated places.

In one embodiment of the present specification, Chemical Formula 1 may be represented by any one of the following compounds, but is not limited thereto.

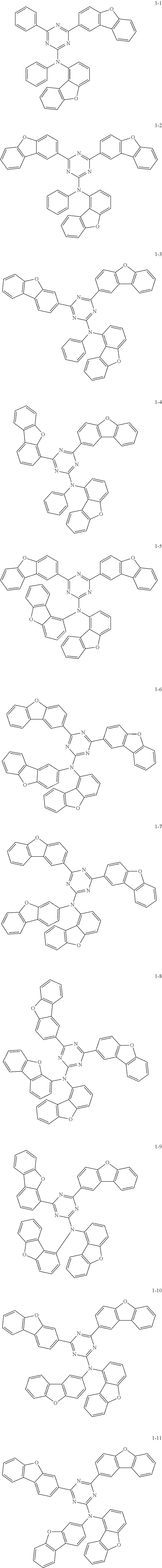

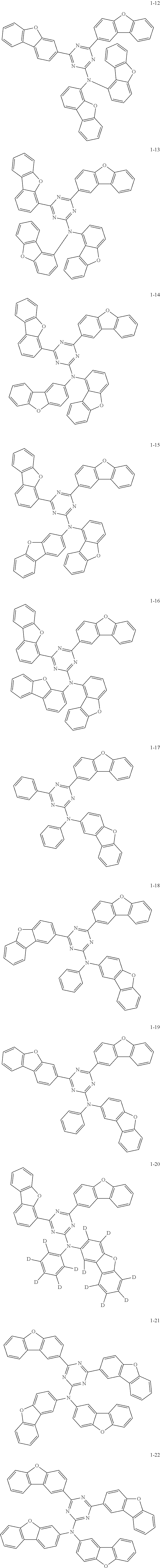

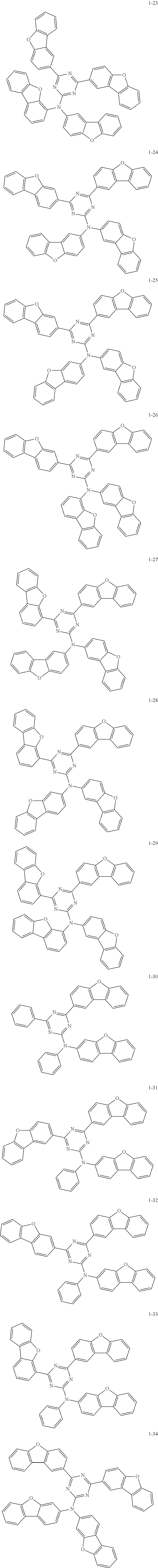

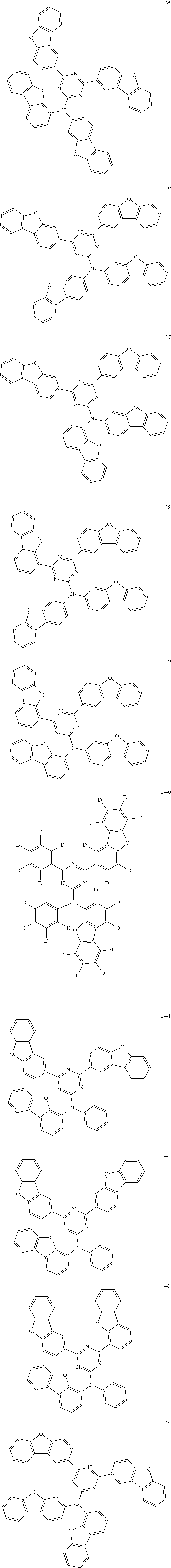

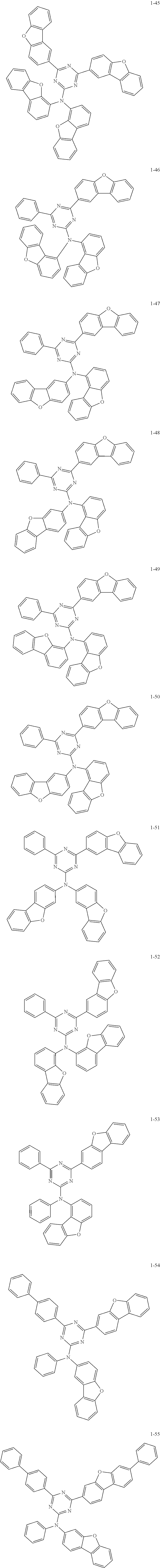

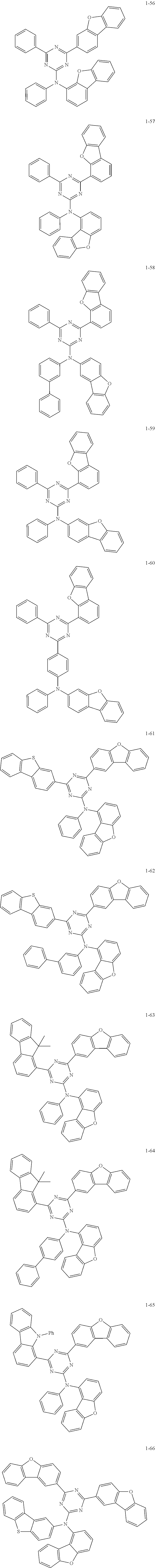

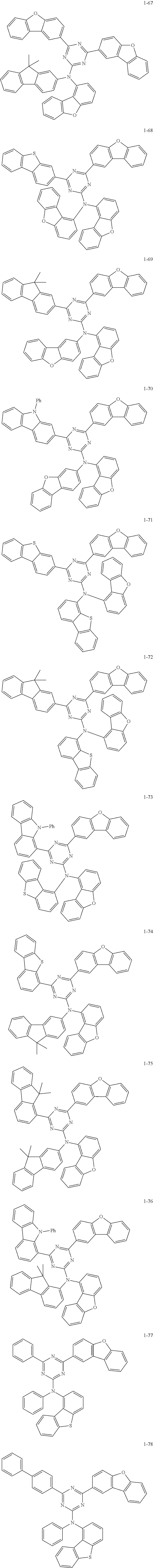

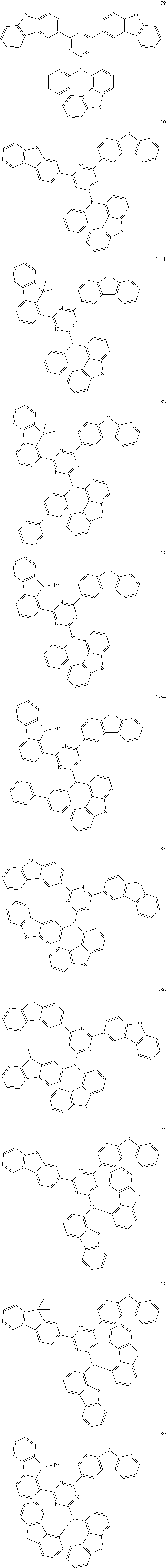

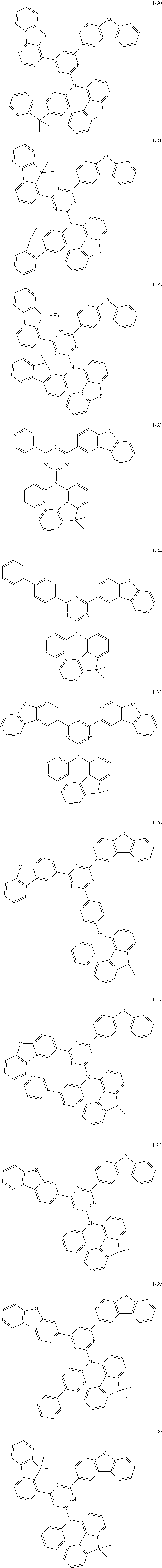

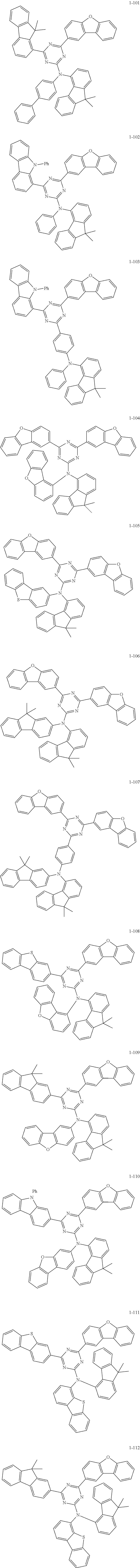

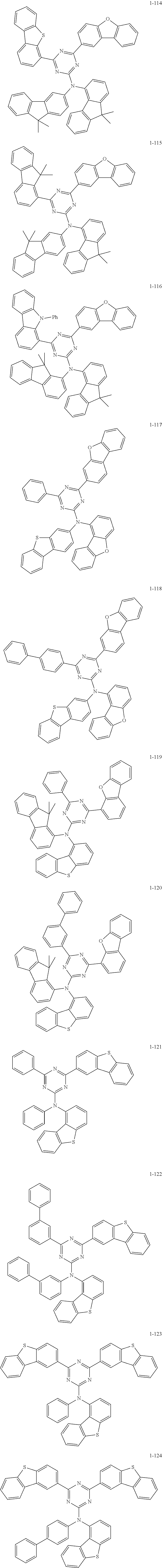

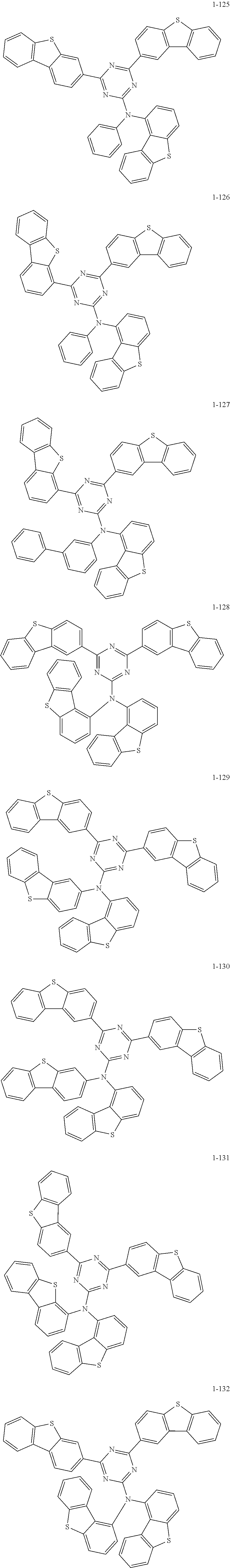

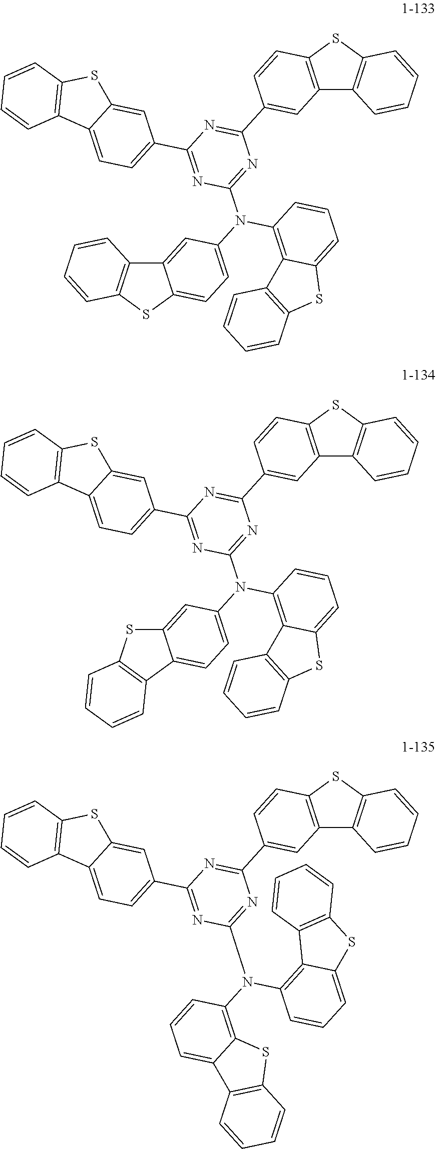 [SMILES: c1ccc2c(c1)sc1cc(-c3nc(-c4ccc5sc6ccccc6c5c4)nc(N(c4ccc5sc6ccccc6c5c4)c4cccc5sc6ccccc6c45)n3)ccc12 ; c1ccc2c(c1)sc1cc(-c3nc(-c4ccc5sc6ccccc6c5c4)nc(N(c4ccc5c(c4)sc4ccccc45)c4cccc5sc6ccccc6c45)n3)ccc12 ; c1ccc2c(c1)sc1cc(-c3nc(-c4ccc5sc6ccccc6c5c4)nc(N(c4cccc5c4sc4ccccc45)c4cccc5sc6ccccc6c45)n3)ccc12]

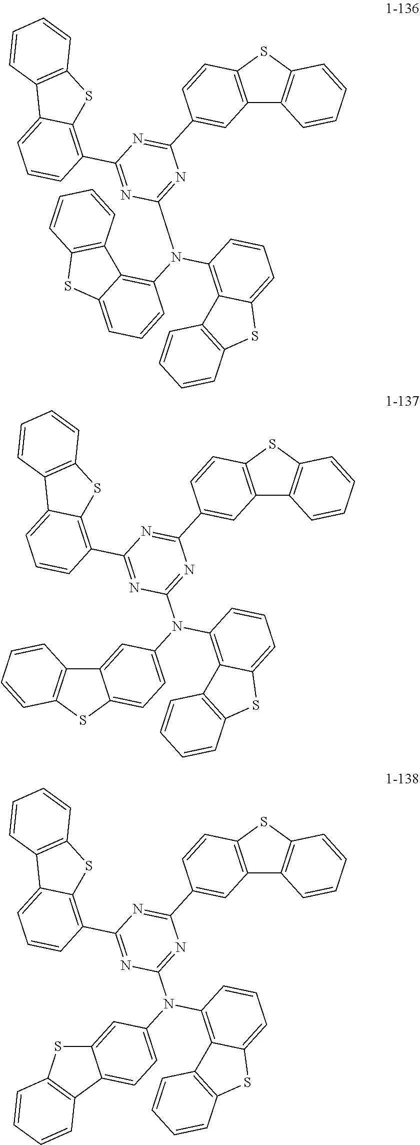 [SMILES: c1ccc2c(c1)sc1ccc(-c3nc(-c4cccc5c4sc4ccccc45)nc(N(c4cccc5sc6ccccc6c45)c4cccc5sc6ccccc6c45)n3)cc12 ; c1ccc2c(c1)sc1ccc(-c3nc(-c4cccc5c4sc4ccccc45)nc(N(c4ccc5sc6ccccc6c5c4)c4cccc5sc6ccccc6c45)n3)cc12 ; c1ccc2c(c1)sc1cc(N(c3nc(-c4ccc5sc6ccccc6c5c4)nc(-c4cccc5c4sc4ccccc45)n3)c3cccc4sc5ccccc5c34)ccc12]

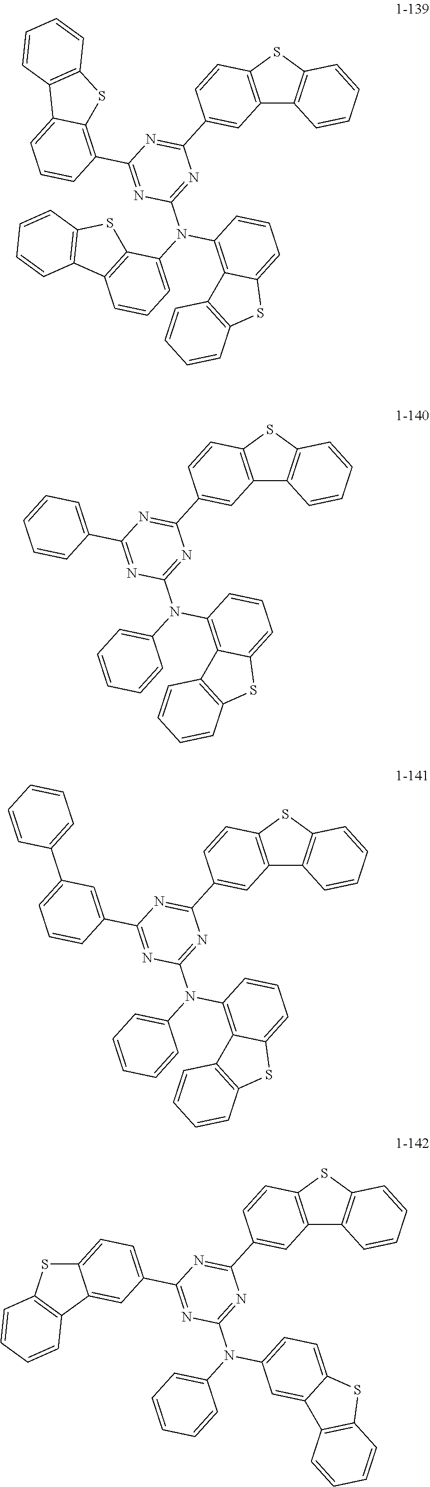 [SMILES: c1ccc2c(c1)sc1ccc(-c3nc(-c4cccc5c4sc4ccccc45)nc(N(c4cccc5c4sc4ccccc45)c4cccc5sc6ccccc6c45)n3)cc12 ; c1ccc(-c2nc(-c3ccc4sc5ccccc5c4c3)nc(N(c3ccccc3)c3cccc4sc5ccccc5c34)n2)cc1 ; c1ccc(-c2cccc(-c3nc(-c4ccc5sc6ccccc6c5c4)nc(N(c4ccccc4)c4cccc5sc6ccccc6c45)n3)c2)cc1 ; c1ccc(N(c2ccc3sc4ccccc4c3c2)c2nc(-c3ccc4sc5ccccc5c4c3)nc(-c3ccc4sc5ccccc5c4c3)n2)cc1]

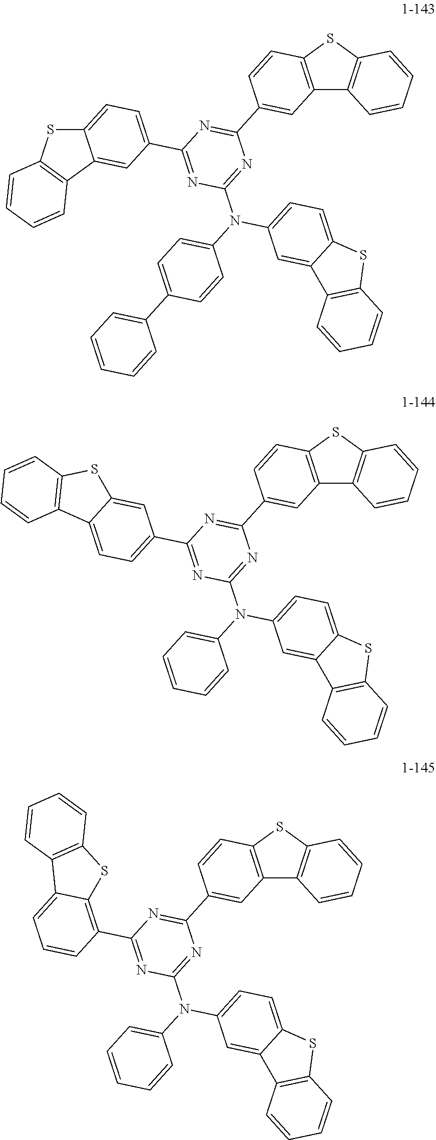 [SMILES: c1ccc(-c2ccc(N(c3ccc4sc5ccccc5c4c3)c3nc(-c4ccc5sc6ccccc6c5c4)nc(-c4ccc5sc6ccccc6c5c4)n3)cc2)cc1 ; c1ccc(N(c2ccc3sc4ccccc4c3c2)c2nc(-c3ccc4c(c3)sc3ccccc34)nc(-c3ccc4sc5ccccc5c4c3)n2)cc1 ; c1ccc(N(c2ccc3sc4ccccc4c3c2)c2nc(-c3ccc4sc5ccccc5c4c3)nc(-c3cccc4c3sc3ccccc34)n2)cc1]

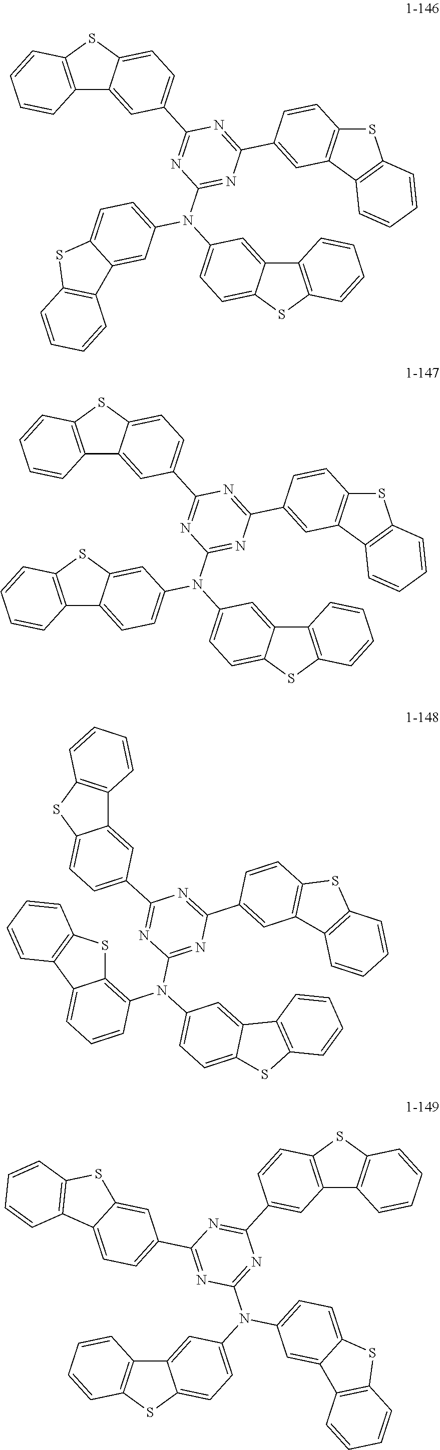 [SMILES: c1ccc2c(c1)sc1ccc(-c3nc(-c4ccc5sc6ccccc6c5c4)nc(N(c4ccc5sc6ccccc6c5c4)c4ccc5sc6ccccc6c5c4)n3)cc12 ; c1ccc2c(c1)sc1cc(N(c3ccc4sc5ccccc5c4c3)c3nc(-c4ccc5sc6ccccc6c5c4)nc(-c4ccc5sc6ccccc6c5c4)n3)ccc12 ; c1ccc2c(c1)sc1ccc(-c3nc(-c4ccc5sc6ccccc6c5c4)nc(N(c4ccc5sc6ccccc6c5c4)c4cccc5c4sc4ccccc45)n3)cc12 ; c1ccc2c(c1)sc1cc(-c3nc(-c4ccc5sc6ccccc6c5c4)nc(N(c4ccc5sc6ccccc6c5c4)c4ccc5sc6ccccc6c5c4)n3)ccc12]

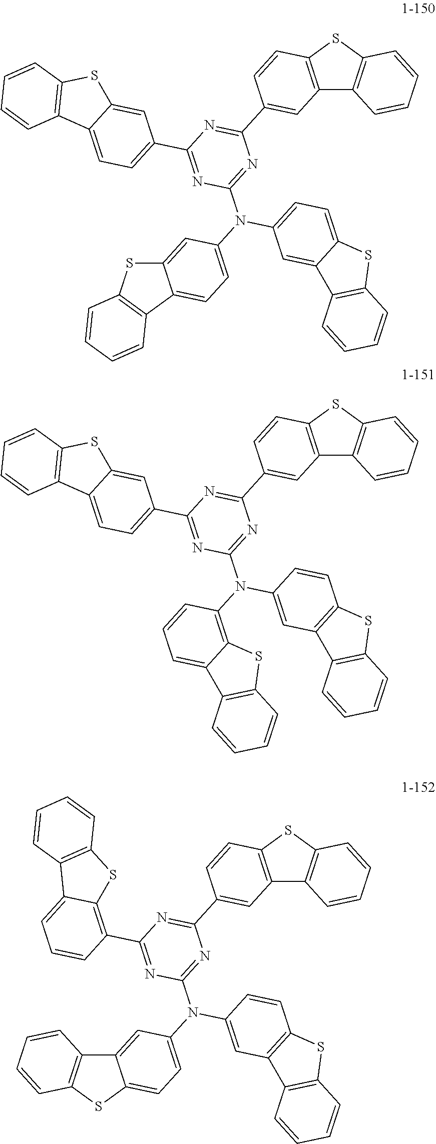 [SMILES: c1ccc2c(c1)sc1cc(-c3nc(-c4ccc5sc6ccccc6c5c4)nc(N(c4ccc5c(c4)sc4ccccc45)c4ccc5sc6ccccc6c5c4)n3)ccc12 ; c1ccc2c(c1)sc1cc(-c3nc(-c4ccc5sc6ccccc6c5c4)nc(N(c4ccc5sc6ccccc6c5c4)c4cccc5c4sc4ccccc45)n3)ccc12 ; c1ccc2c(c1)sc1ccc(-c3nc(-c4cccc5c4sc4ccccc45)nc(N(c4ccc5sc6ccccc6c5c4)c4ccc5sc6ccccc6c5c4)n3)cc12]

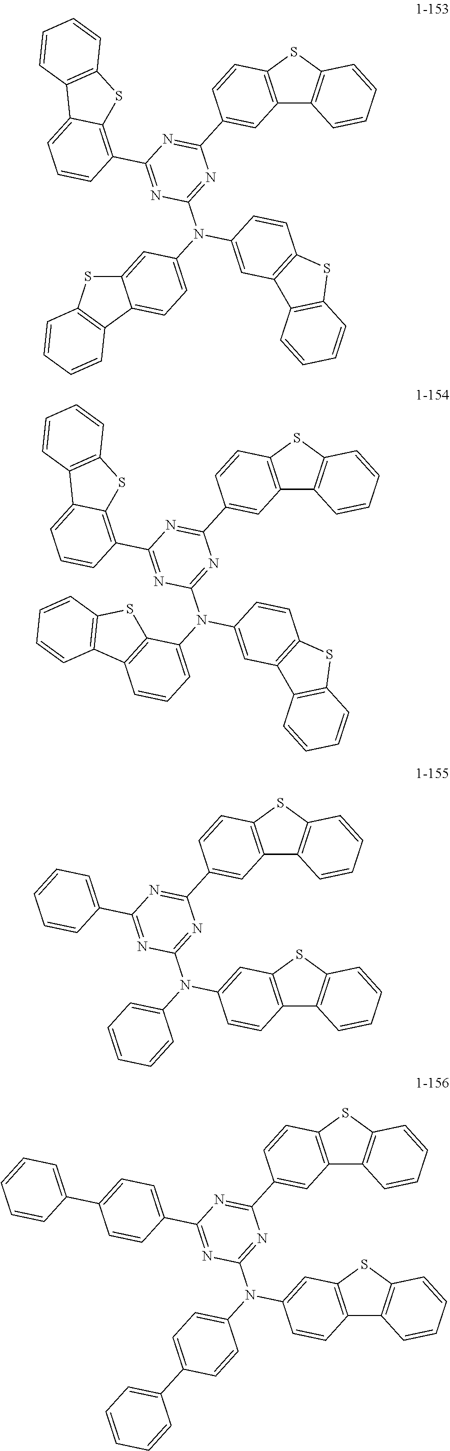

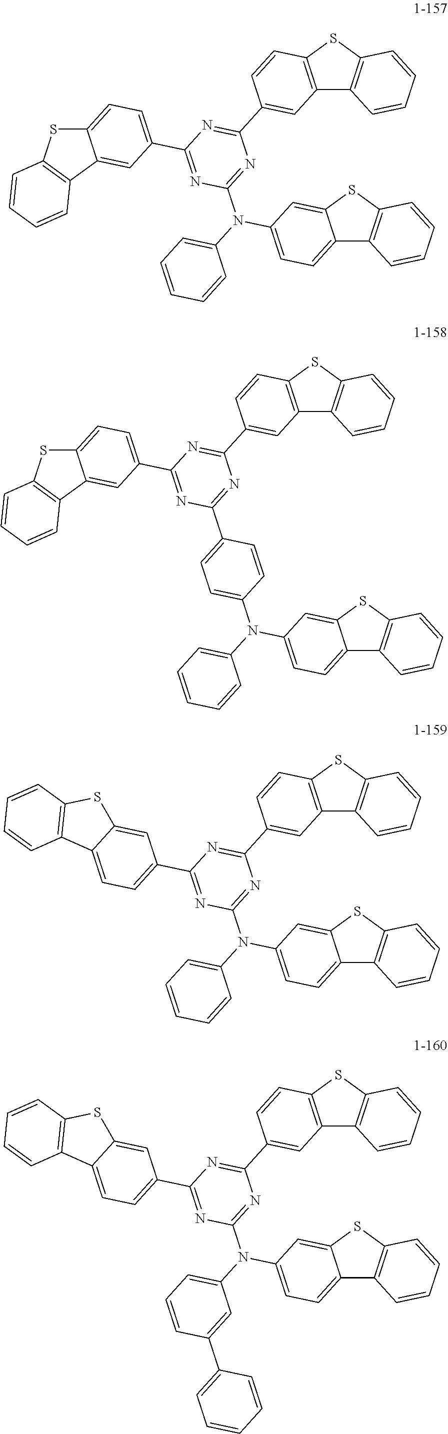 [SMILES: c1ccc(N(c2ccc3c(c2)sc2ccccc23)c2nc(-c3ccc4sc5ccccc5c4c3)nc(-c3ccc4sc5ccccc5c4c3)n2)cc1 ; c1ccc(N(c2ccc(-c3nc(-c4ccc5sc6ccccc6c5c4)nc(-c4ccc5sc6ccccc6c5c4)n3)cc2)c2ccc3c(c2)sc2ccccc23)cc1 ; c1ccc(N(c2ccc3c(c2)sc2ccccc23)c2nc(-c3ccc4c(c3)sc3ccccc34)nc(-c3ccc4sc5ccccc5c4c3)n2)cc1 ; c1ccc(-c2cccc(N(c3ccc4c(c3)sc3ccccc34)c3nc(-c4ccc5c(c4)sc4ccccc45)nc(-c4ccc5sc6ccccc6c5c4)n3)c2)cc1]

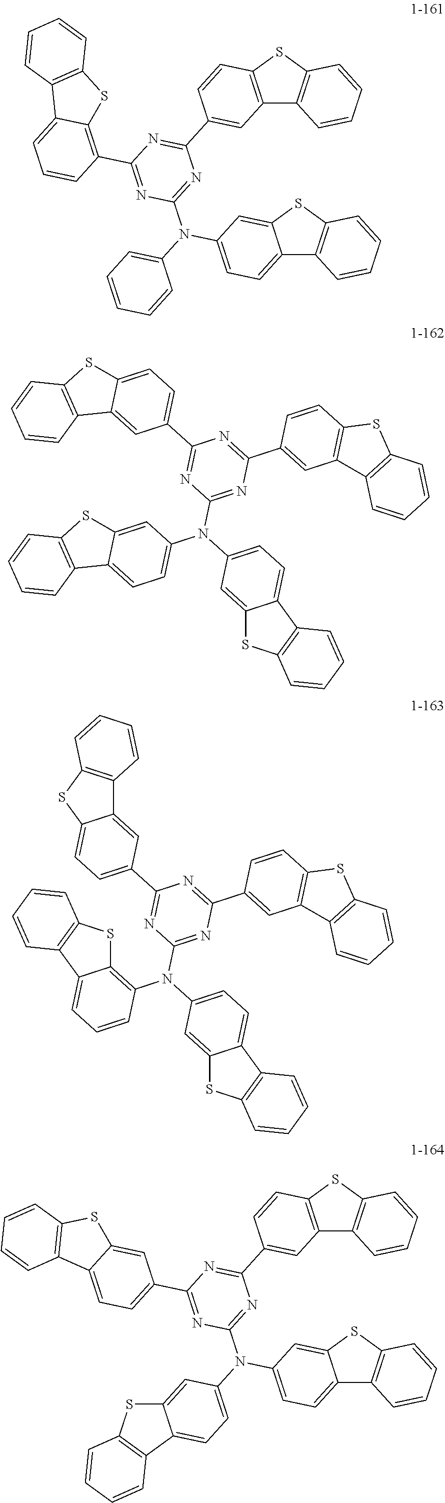 [SMILES: c1ccc(N(c2ccc3c(c2)sc2ccccc23)c2nc(-c3ccc4sc5ccccc5c4c3)nc(-c3cccc4c3sc3ccccc34)n2)cc1 ; c1ccc2c(c1)sc1cc(N(c3ccc4c(c3)sc3ccccc34)c3nc(-c4ccc5sc6ccccc6c5c4)nc(-c4ccc5sc6ccccc6c5c4)n3)ccc12 ; c1ccc2c(c1)sc1cc(N(c3nc(-c4ccc5sc6ccccc6c5c4)nc(-c4ccc5sc6ccccc6c5c4)n3)c3cccc4c3sc3ccccc34)ccc12 ; c1ccc2c(c1)sc1cc(-c3nc(-c4ccc5sc6ccccc6c5c4)nc(N(c4ccc5c(c4)sc4ccccc45)c4ccc5c(c4)sc4ccccc45)n3)ccc12]

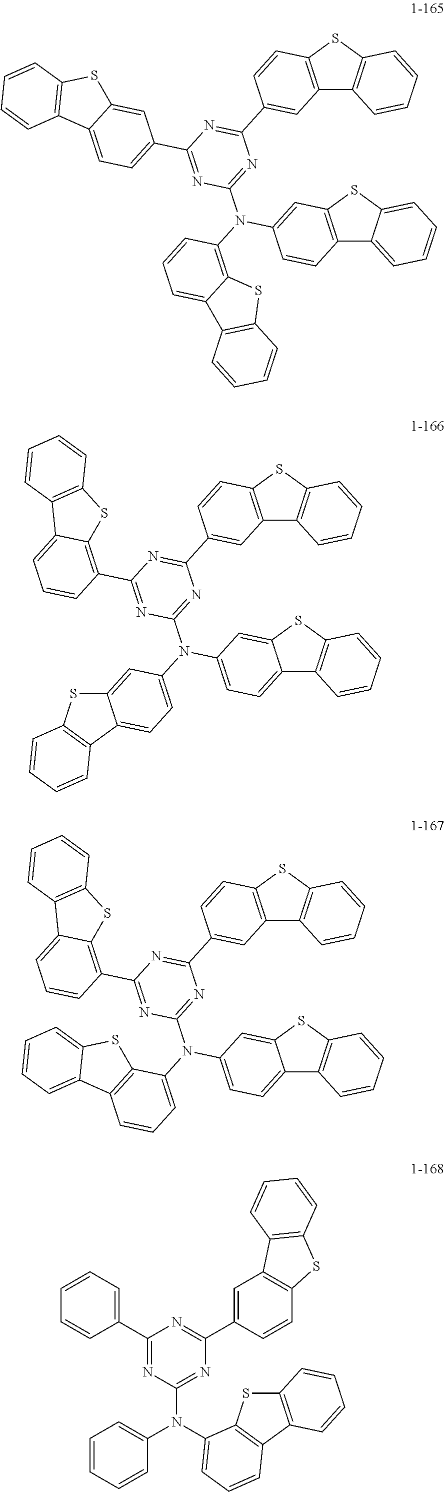

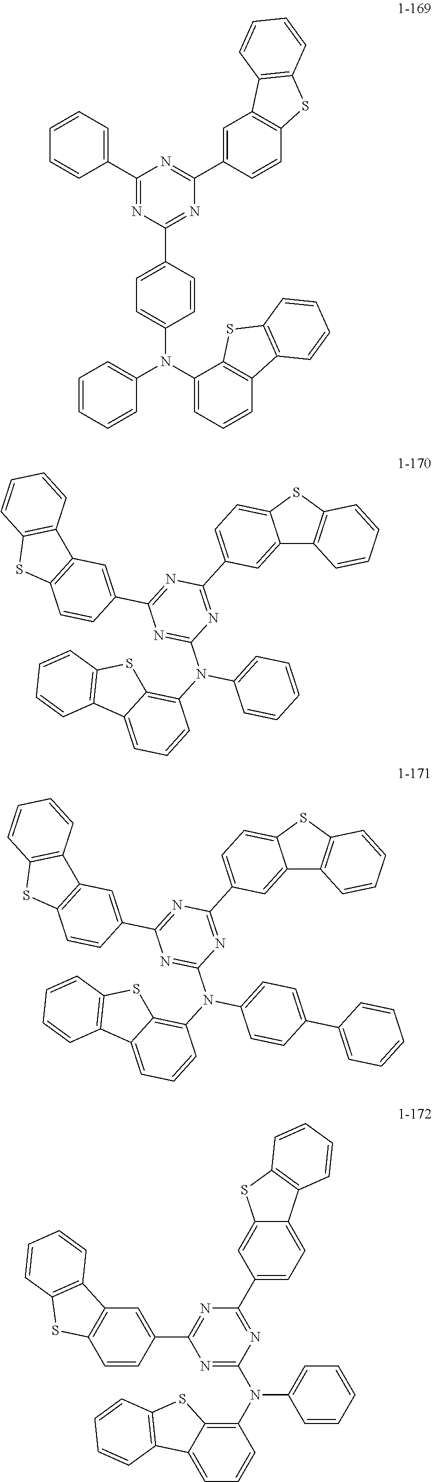 [SMILES: c1ccc(-c2nc(-c3ccc(N(c4ccccc4)c4cccc5c4sc4ccccc45)cc3)nc(-c3ccc4sc5ccccc5c4c3)n2)cc1 ; c1ccc(N(c2nc(-c3ccc4sc5ccccc5c4c3)nc(-c3ccc4sc5ccccc5c4c3)n2)c2cccc3c2sc2ccccc23)cc1 ; c1ccc(-c2ccc(N(c3nc(-c4ccc5sc6ccccc6c5c4)nc(-c4ccc5sc6ccccc6c5c4)n3)c3cccc4c3sc3ccccc34)cc2)cc1 ; c1ccc(N(c2nc(-c3ccc4c(c3)sc3ccccc34)nc(-c3ccc4sc5ccccc5c4c3)n2)c2cccc3c2sc2ccccc23)cc1]

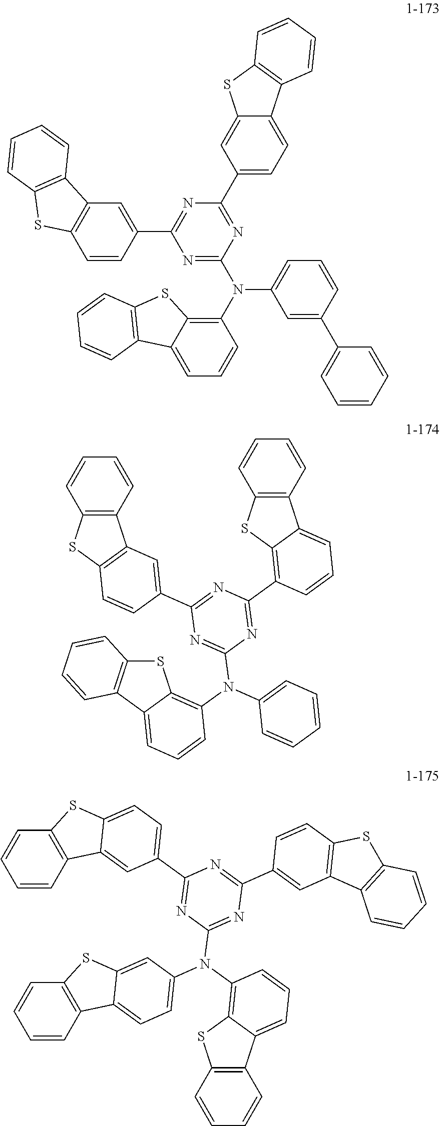 [SMILES: c1ccc(-c2cccc(N(c3nc(-c4ccc5c(c4)sc4ccccc45)nc(-c4ccc5sc6ccccc6c5c4)n3)c3cccc4c3sc3ccccc34)c2)cc1 ; c1ccc(N(c2nc(-c3ccc4sc5ccccc5c4c3)nc(-c3cccc4c3sc3ccccc34)n2)c2cccc3c2sc2ccccc23)cc1 ; c1ccc2c(c1)sc1cc(N(c3nc(-c4ccc5sc6ccccc6c5c4)nc(-c4ccc5sc6ccccc6c5c4)n3)c3cccc4c3sc3ccccc34)ccc12]

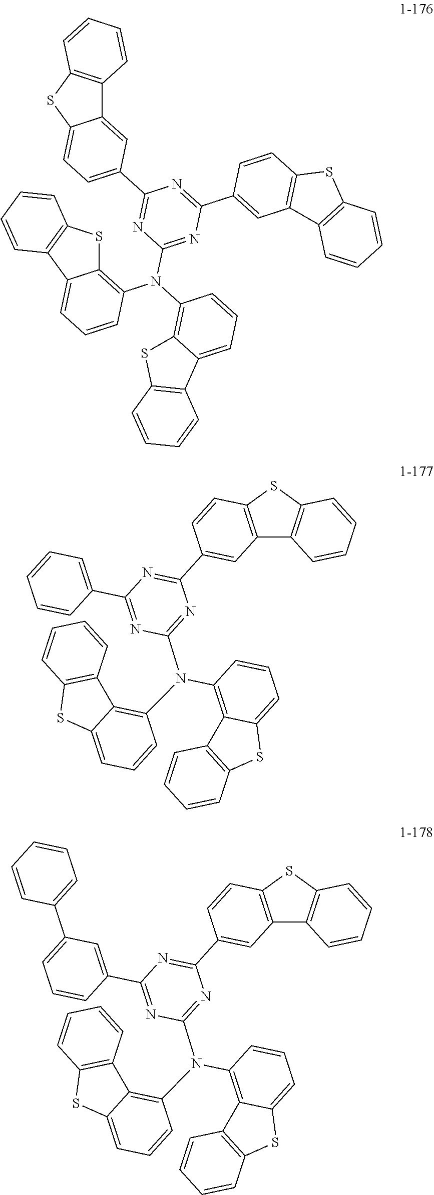 [SMILES: c1ccc2c(c1)sc1ccc(-c3nc(-c4ccc5sc6ccccc6c5c4)nc(N(c4cccc5c4sc4ccccc45)c4cccc5c4sc4ccccc45)n3)cc12 ; c1ccc(-c2nc(-c3ccc4sc5ccccc5c4c3)nc(N(c3cccc4sc5ccccc5c34)c3cccc4sc5ccccc5c34)n2)cc1 ; c1ccc(-c2cccc(-c3nc(-c4ccc5sc6ccccc6c5c4)nc(N(c4cccc5sc6ccccc6c45)c4cccc5sc6ccccc6c45)n3)c2)cc1]

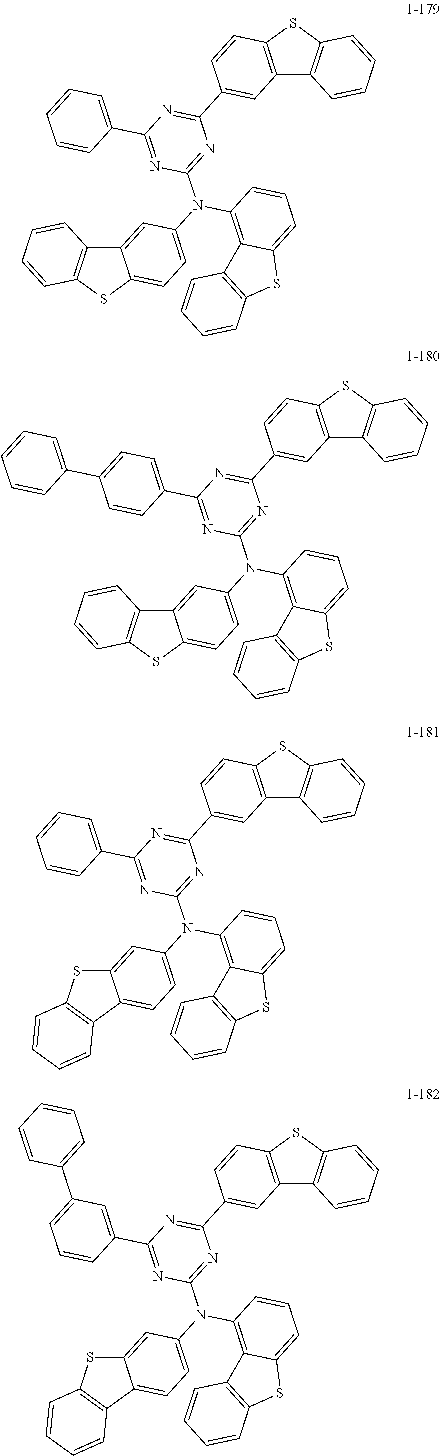

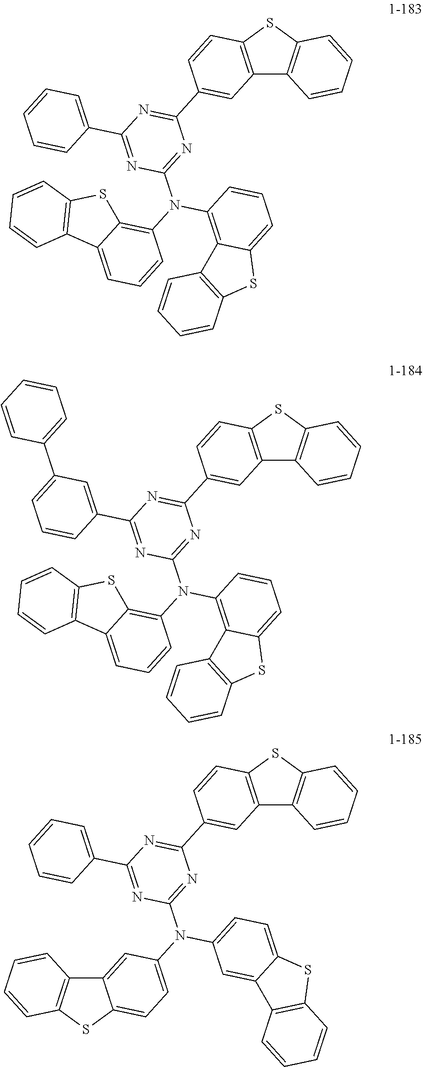 [SMILES: c1ccc(-c2nc(-c3ccc4sc5ccccc5c4c3)nc(N(c3cccc4c3sc3ccccc34)c3cccc4sc5ccccc5c34)n2)cc1 ; c1ccc(-c2cccc(-c3nc(-c4ccc5sc6ccccc6c5c4)nc(N(c4cccc5c4sc4ccccc45)c4cccc5sc6ccccc6c45)n3)c2)cc1 ; c1ccc(-c2nc(-c3ccc4sc5ccccc5c4c3)nc(N(c3ccc4sc5ccccc5c4c3)c3ccc4sc5ccccc5c4c3)n2)cc1]

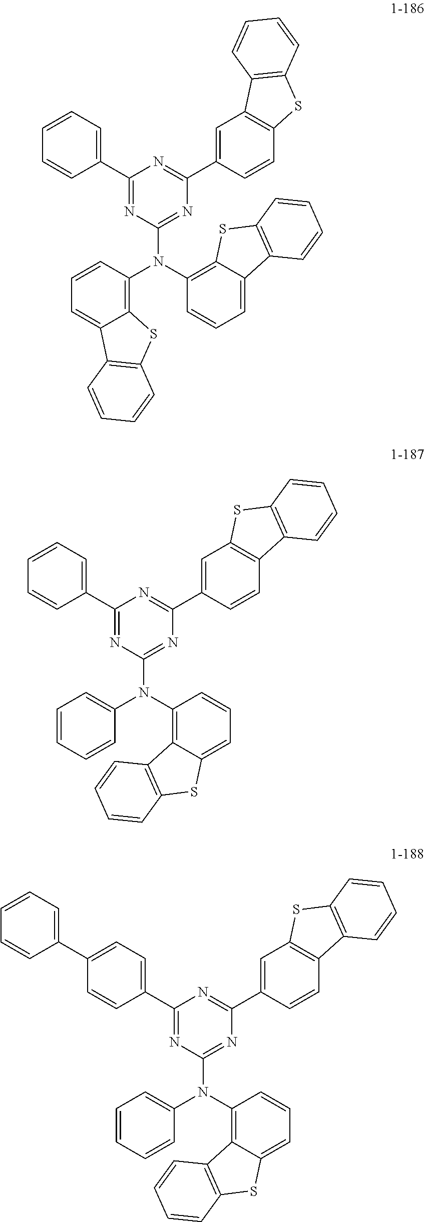 [SMILES: c1ccc(-c2nc(-c3ccc4sc5ccccc5c4c3)nc(N(c3cccc4c3sc3ccccc34)c3cccc4c3sc3ccccc34)n2)cc1 ; c1ccc(-c2nc(-c3ccc4c(c3)sc3ccccc34)nc(N(c3ccccc3)c3cccc4sc5ccccc5c34)n2)cc1 ; c1ccc(-c2ccc(-c3nc(-c4ccc5c(c4)sc4ccccc45)nc(N(c4ccccc4)c4cccc5sc6ccccc6c45)n3)cc2)cc1]

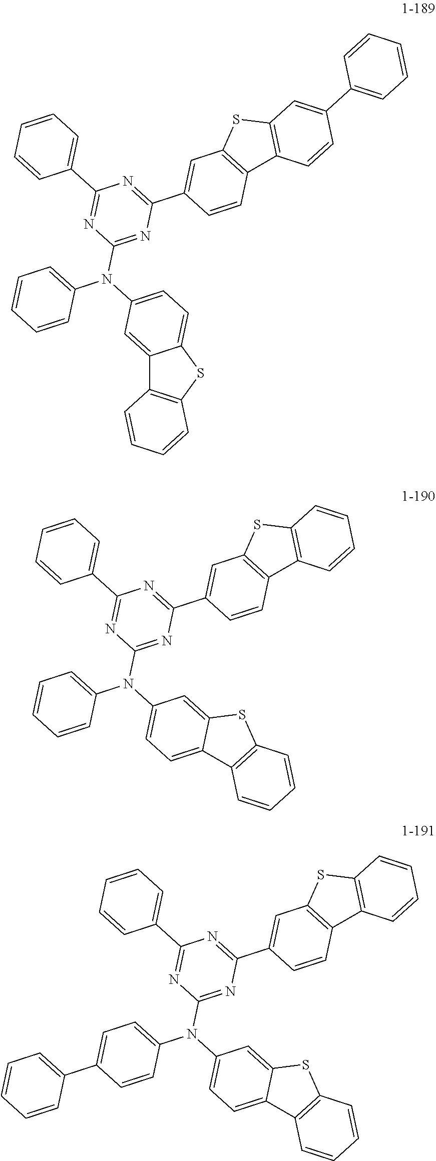 [SMILES: c1ccc(-c2ccc3c(c2)sc2cc(-c4nc(-c5ccccc5)nc(N(c5ccccc5)c5ccc6sc7ccccc7c6c5)n4)ccc23)cc1 ; c1ccc(-c2nc(-c3ccc4c(c3)sc3ccccc34)nc(N(c3ccccc3)c3ccc4c(c3)sc3ccccc34)n2)cc1 ; c1ccc(-c2ccc(N(c3ccc4c(c3)sc3ccccc34)c3nc(-c4ccccc4)nc(-c4ccc5c(c4)sc4ccccc45)n3)cc2)cc1]

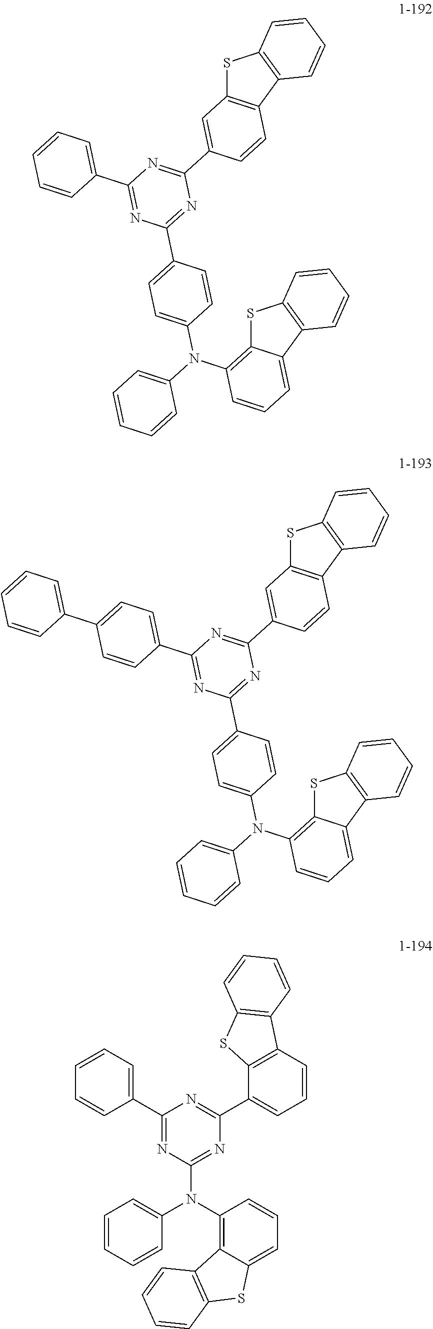 [SMILES: c1ccc(-c2nc(-c3ccc(N(c4ccccc4)c4cccc5c4sc4ccccc45)cc3)nc(-c3ccc4c(c3)sc3ccccc34)n2)cc1 ; c1ccc(-c2ccc(-c3nc(-c4ccc(N(c5ccccc5)c5cccc6c5sc5ccccc56)cc4)nc(-c4ccc5c(c4)sc4ccccc45)n3)cc2)cc1 ; c1ccc(-c2nc(-c3cccc4c3sc3ccccc34)nc(N(c3ccccc3)c3cccc4sc5ccccc5c34)n2)cc1]

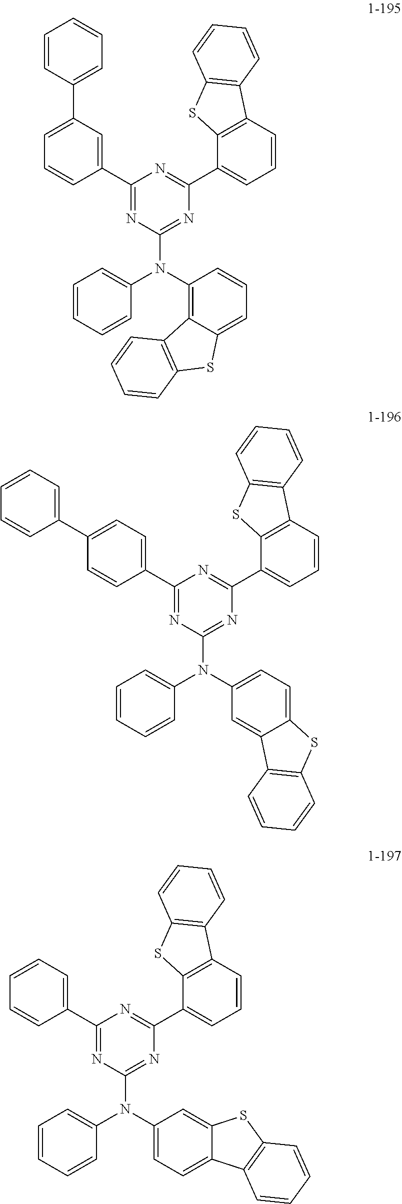 [SMILES: c1ccc(-c2cccc(-c3nc(-c4cccc5c4sc4ccccc45)nc(N(c4ccccc4)c4cccc5sc6ccccc6c45)n3)c2)cc1 ; c1ccc(-c2ccc(-c3nc(-c4cccc5c4sc4ccccc45)nc(N(c4ccccc4)c4ccc5sc6ccccc6c5c4)n3)cc2)cc1 ; c1ccc(-c2nc(-c3cccc4c3sc3ccccc34)nc(N(c3ccccc3)c3ccc4c(c3)sc3ccccc34)n2)cc1]

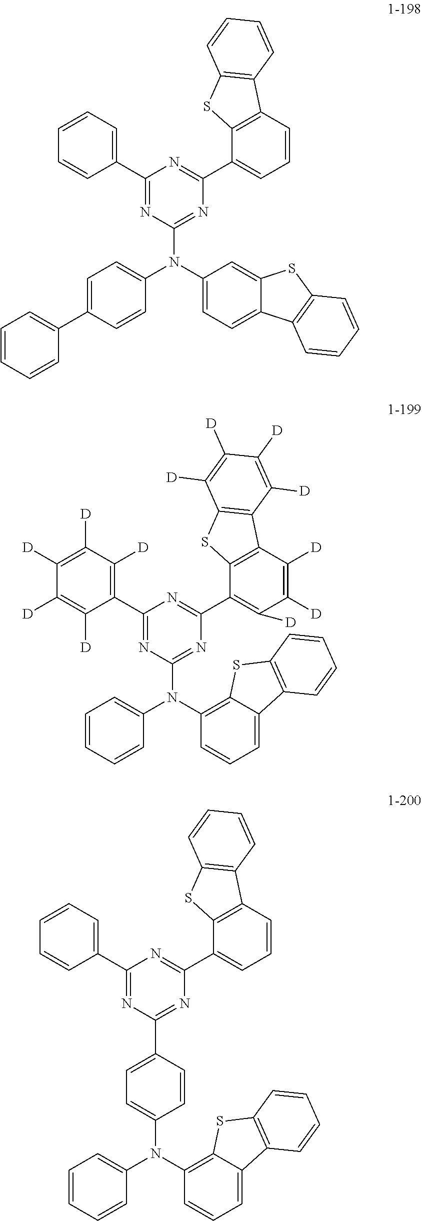 [SMILES: c1ccc(-c2ccc(N(c3ccc4c(c3)sc3ccccc34)c3nc(-c4ccccc4)nc(-c4cccc5c4sc4ccccc45)n3)cc2)cc1 ; [2H]c1c([2H])c([2H])c(-c2nc(-c3c([2H])c([2H])c([2H])c4c3sc3c([2H])c([2H])c([2H])c([2H])c34)nc(N(c3ccccc3)c3cccc4c3sc3ccccc34)n2)c([2H])c1[2H] ; c1ccc(-c2nc(-c3ccc(N(c4ccccc4)c4cccc5c4sc4ccccc45)cc3)nc(-c3cccc4c3sc3ccccc34)n2)cc1]

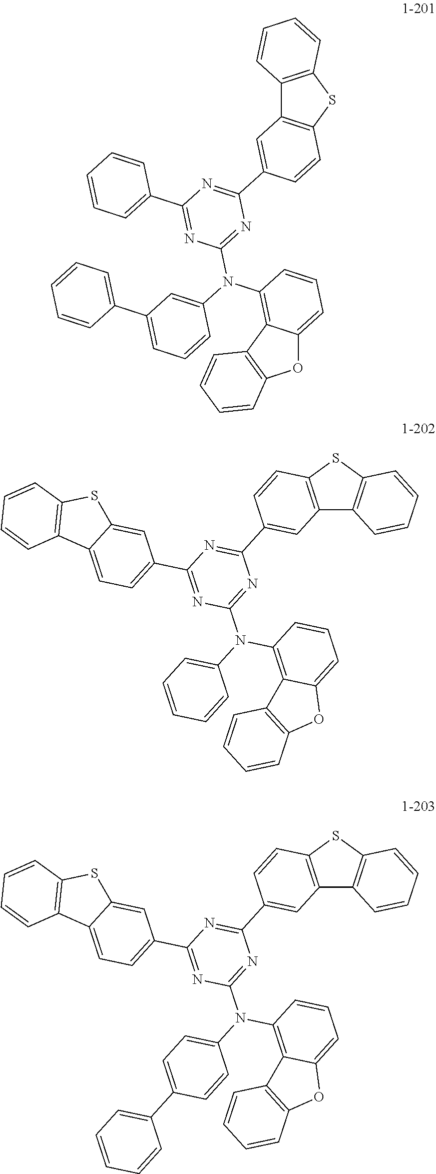 [SMILES: c1ccc(-c2cccc(N(c3nc(-c4ccccc4)nc(-c4ccc5sc6ccccc6c5c4)n3)c3cccc4oc5ccccc5c34)c2)cc1 ; c1ccc(N(c2nc(-c3ccc4c(c3)sc3ccccc34)nc(-c3ccc4sc5ccccc5c4c3)n2)c2cccc3oc4ccccc4c23)cc1 ; c1ccc(-c2ccc(N(c3nc(-c4ccc5c(c4)sc4ccccc45)nc(-c4ccc5sc6ccccc6c5c4)n3)c3cccc4oc5ccccc5c34)cc2)cc1]

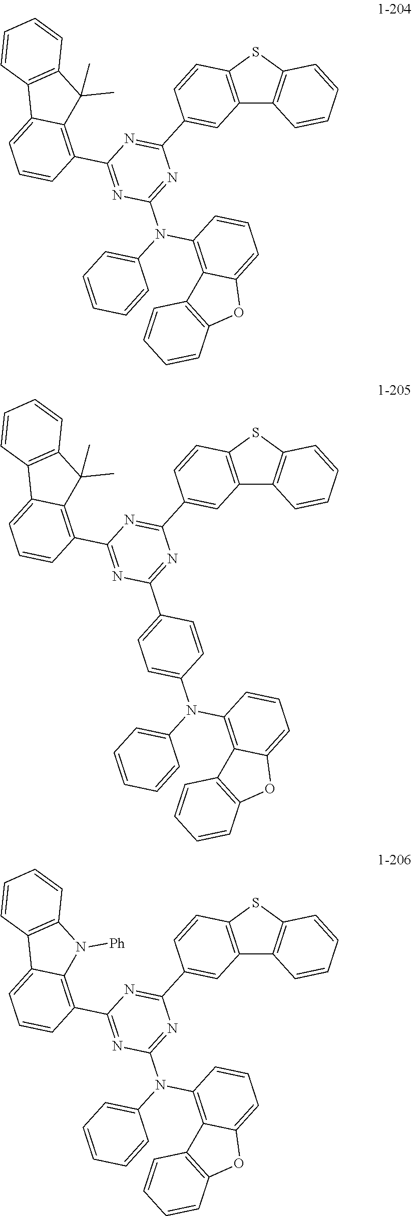 [SMILES: CC1(C)c2ccccc2-c2cccc(-c3nc(-c4ccc5sc6ccccc6c5c4)nc(N(c4ccccc4)c4cccc5oc6ccccc6c45)n3)c21 ; CC1(C)c2ccccc2-c2cccc(-c3nc(-c4ccc(N(c5ccccc5)c5cccc6oc7ccccc7c56)cc4)nc(-c4ccc5sc6ccccc6c5c4)n3)c21 ; c1ccc(N(c2nc(-c3ccc4sc5ccccc5c4c3)nc(-c3cccc4c5ccccc5n(-c5ccccc5)c34)n2)c2cccc3oc4ccccc4c23)cc1]

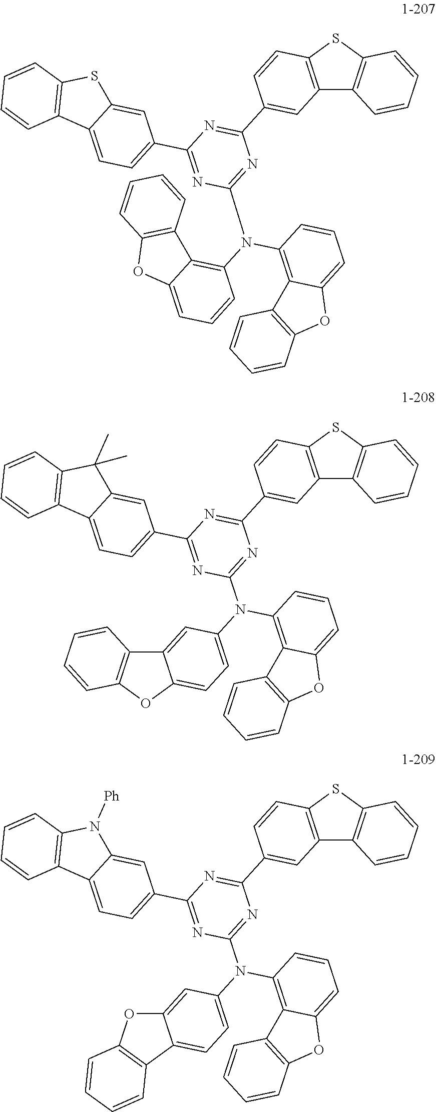 [SMILES: c1ccc2c(c1)oc1cccc(N(c3nc(-c4ccc5c(c4)sc4ccccc45)nc(-c4ccc5sc6ccccc6c5c4)n3)c3cccc4oc5ccccc5c34)c12 ; CC1(C)c2ccccc2-c2ccc(-c3nc(-c4ccc5sc6ccccc6c5c4)nc(N(c4ccc5oc6ccccc6c5c4)c4cccc5oc6ccccc6c45)n3)cc21 ; c1ccc(-n2c3ccccc3c3ccc(-c4nc(-c5ccc6sc7ccccc7c6c5)nc(N(c5ccc6c(c5)oc5ccccc56)c5cccc6oc7ccccc7c56)n4)cc32)cc1]

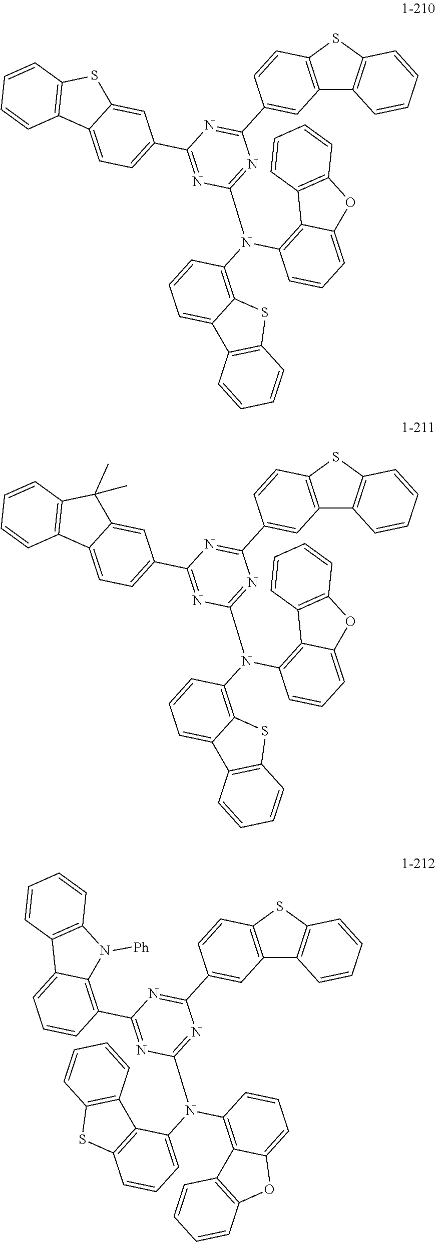 [SMILES: c1ccc2c(c1)oc1cccc(N(c3nc(-c4ccc5c(c4)sc4ccccc45)nc(-c4ccc5sc6ccccc6c5c4)n3)c3cccc4c3sc3ccccc34)c12 ; CC1(C)c2ccccc2-c2ccc(-c3nc(-c4ccc5sc6ccccc6c5c4)nc(N(c4cccc5c4sc4ccccc45)c4cccc5oc6ccccc6c45)n3)cc21 ; c1ccc(-n2c3ccccc3c3cccc(-c4nc(-c5ccc6sc7ccccc7c6c5)nc(N(c5cccc6oc7ccccc7c56)c5cccc6sc7ccccc7c56)n4)c32)cc1]

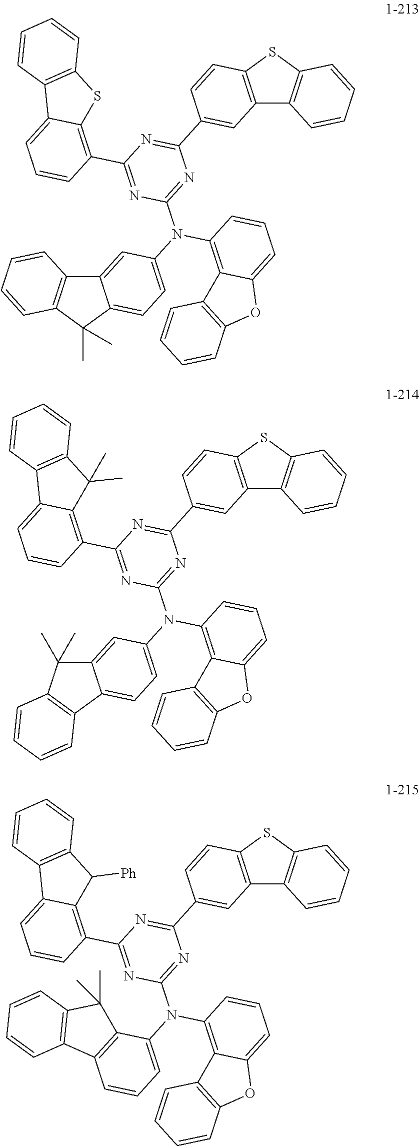

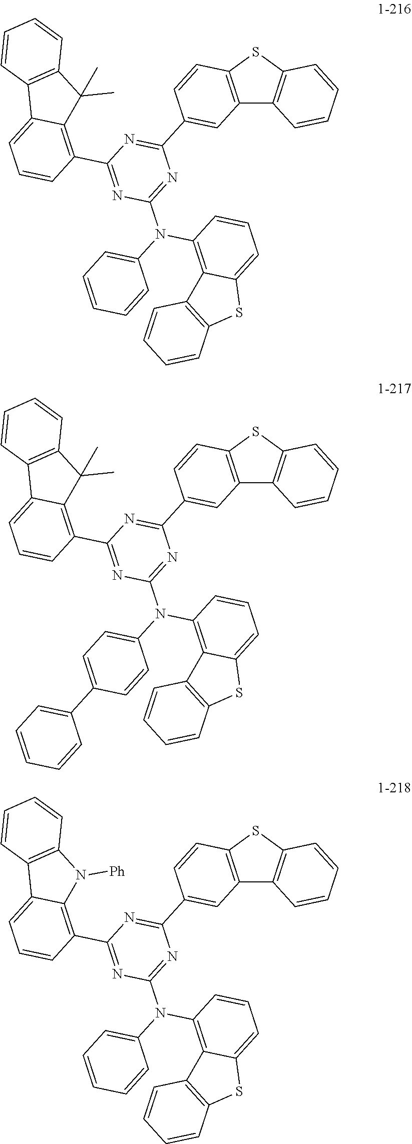

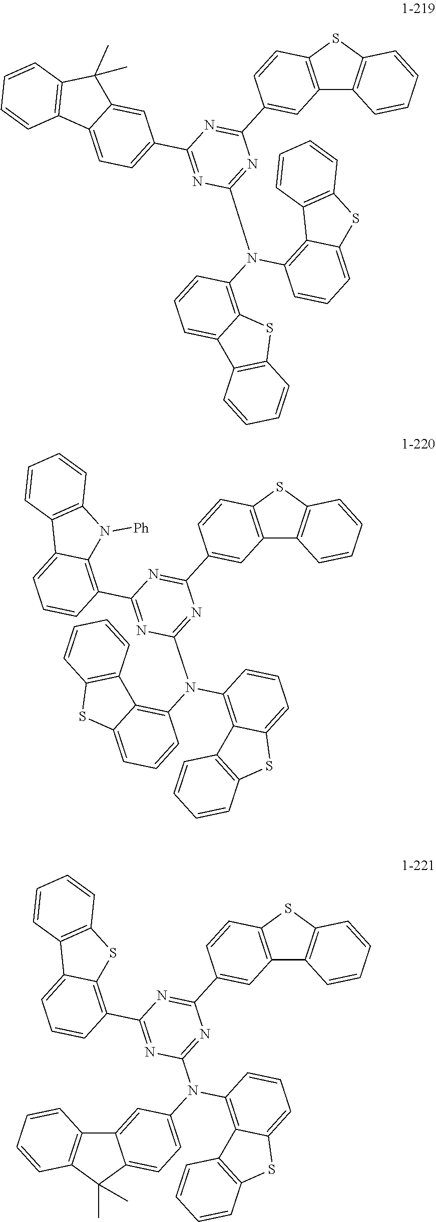 [SMILES: CC1(C)c2ccccc2-c2ccc(-c3nc(-c4ccc5sc6ccccc6c5c4)nc(N(c4cccc5c4sc4ccccc45)c4cccc5sc6ccccc6c45)n3)cc21 ; c1ccc(-n2c3ccccc3c3cccc(-c4nc(-c5ccc6sc7ccccc7c6c5)nc(N(c5cccc6sc7ccccc7c56)c5cccc6sc7ccccc7c56)n4)c32)cc1 ; CC1(C)c2ccccc2-c2cc(N(c3nc(-c4ccc5sc6ccccc6c5c4)nc(-c4cccc5c4sc4ccccc45)n3)c3cccc4sc5ccccc5c34)ccc21]

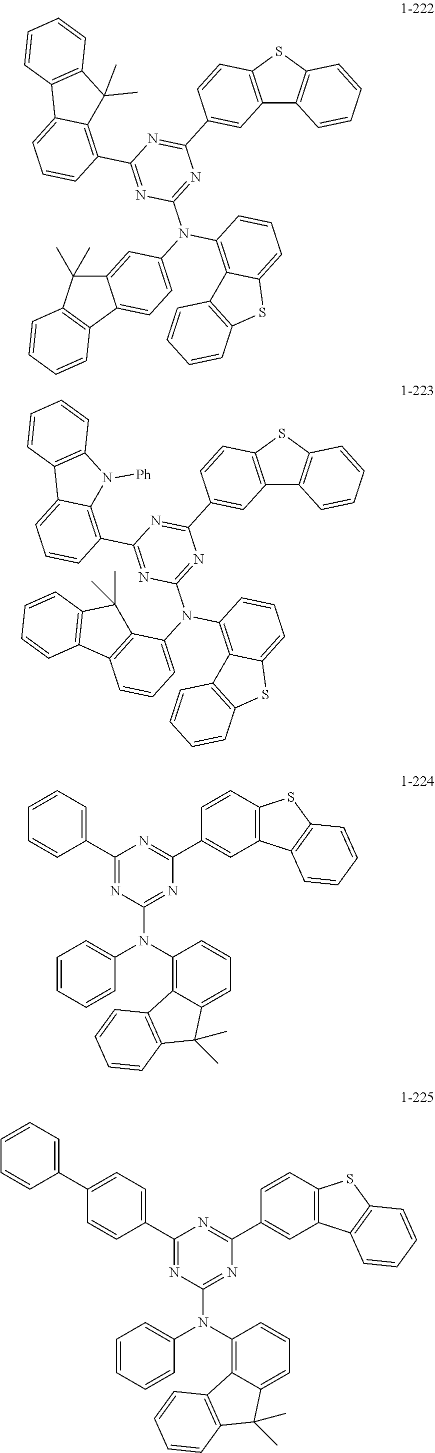

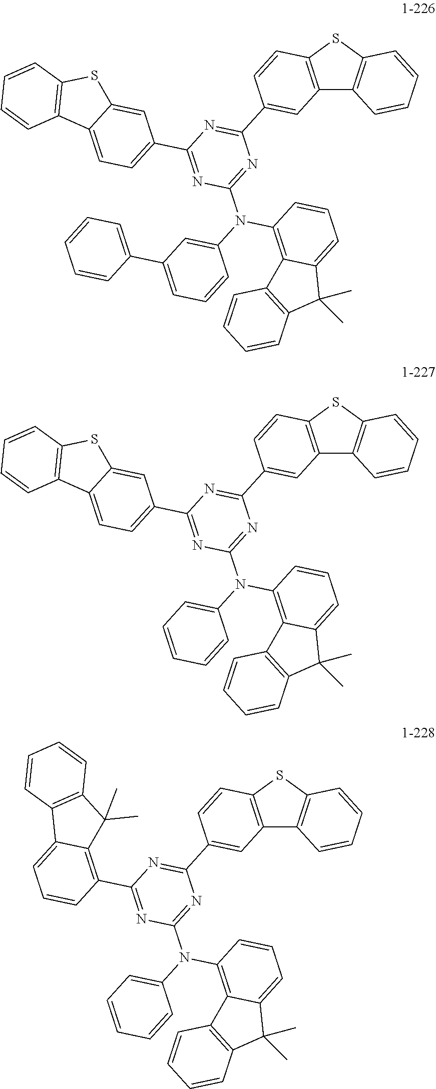 [SMILES: CC1(C)c2ccccc2-c2c(N(c3cccc(-c4ccccc4)c3)c3nc(-c4ccc5c(c4)sc4ccccc45)nc(-c4ccc5sc6ccccc6c5c4)n3)cccc21 ; CC1(C)c2ccccc2-c2c(N(c3ccccc3)c3nc(-c4ccc5c(c4)sc4ccccc45)nc(-c4ccc5sc6ccccc6c5c4)n3)cccc21 ; CC1(C)c2ccccc2-c2c(N(c3ccccc3)c3nc(-c4ccc5sc6ccccc6c5c4)nc(-c4cccc5c4C(C)(C)c4ccccc4-5)n3)cccc21]

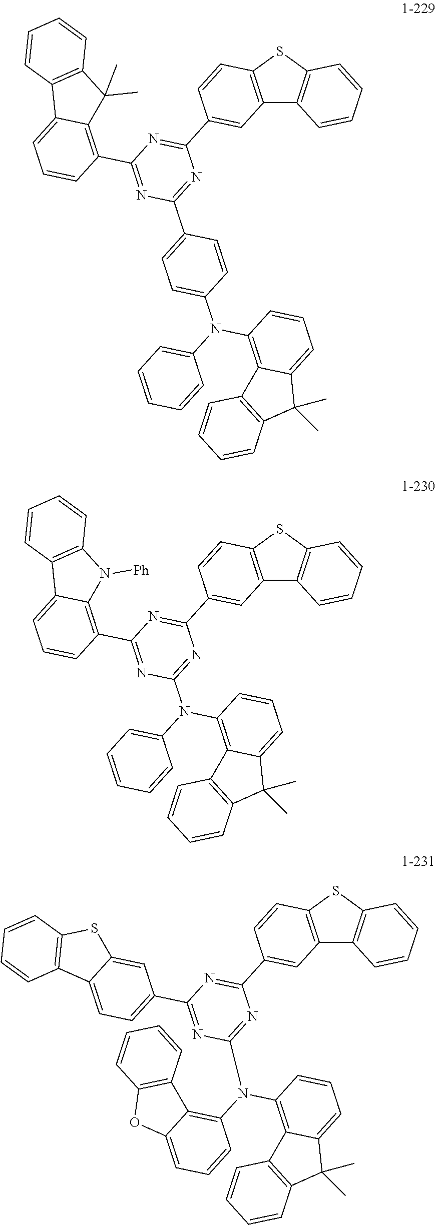 [SMILES: CC1(C)c2ccccc2-c2c(N(c3ccccc3)c3ccc(-c4nc(-c5ccc6sc7ccccc7c6c5)nc(-c5cccc6c5C(C)(C)c5ccccc5-6)n4)cc3)cccc21 ; CC1(C)c2ccccc2-c2c(N(c3ccccc3)c3nc(-c4ccc5sc6ccccc6c5c4)nc(-c4cccc5c6ccccc6n(-c6ccccc6)c45)n3)cccc21 ; CC1(C)c2ccccc2-c2c(N(c3nc(-c4ccc5c(c4)sc4ccccc45)nc(-c4ccc5sc6ccccc6c5c4)n3)c3cccc4oc5ccccc5c34)cccc21]

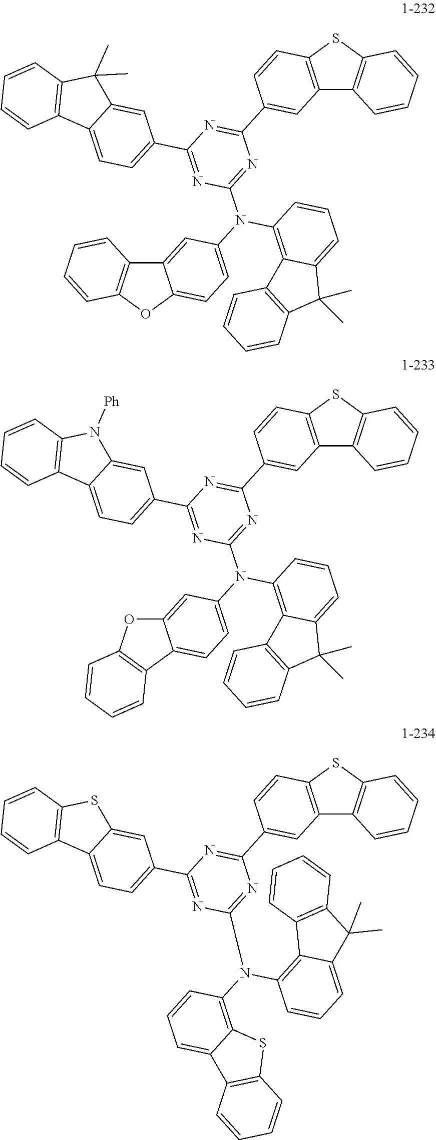 [SMILES: CC1(C)c2ccccc2-c2ccc(-c3nc(-c4ccc5sc6ccccc6c5c4)nc(N(c4ccc5oc6ccccc6c5c4)c4cccc5c4-c4ccccc4C5(C)C)n3)cc21 ; CC1(C)c2ccccc2-c2c(N(c3ccc4c(c3)oc3ccccc34)c3nc(-c4ccc5sc6ccccc6c5c4)nc(-c4ccc5c6ccccc6n(-c6ccccc6)c5c4)n3)cccc21 ; CC1(C)c2ccccc2-c2c(N(c3nc(-c4ccc5c(c4)sc4ccccc45)nc(-c4ccc5sc6ccccc6c5c4)n3)c3cccc4c3sc3ccccc34)cccc21]

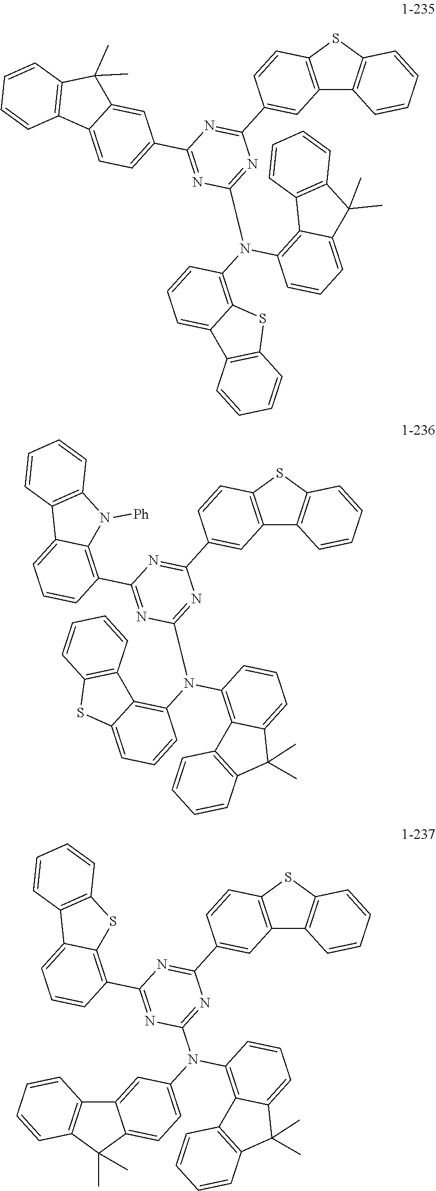 [SMILES: CC1(C)c2ccccc2-c2ccc(-c3nc(-c4ccc5sc6ccccc6c5c4)nc(N(c4cccc5c4-c4ccccc4C5(C)C)c4cccc5c4sc4ccccc45)n3)cc21 ; CC1(C)c2ccccc2-c2c(N(c3nc(-c4ccc5sc6ccccc6c5c4)nc(-c4cccc5c6ccccc6n(-c6ccccc6)c45)n3)c3cccc4sc5ccccc5c34)cccc21 ; CC1(C)c2ccccc2-c2cc(N(c3nc(-c4ccc5sc6ccccc6c5c4)nc(-c4cccc5c4sc4ccccc45)n3)c3cccc4c3-c3ccccc3C4(C)C)ccc21]

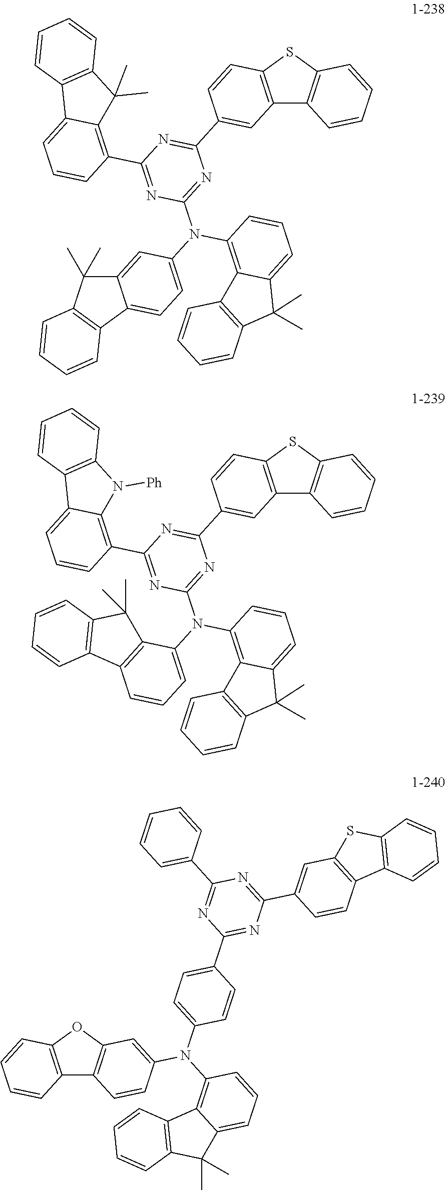

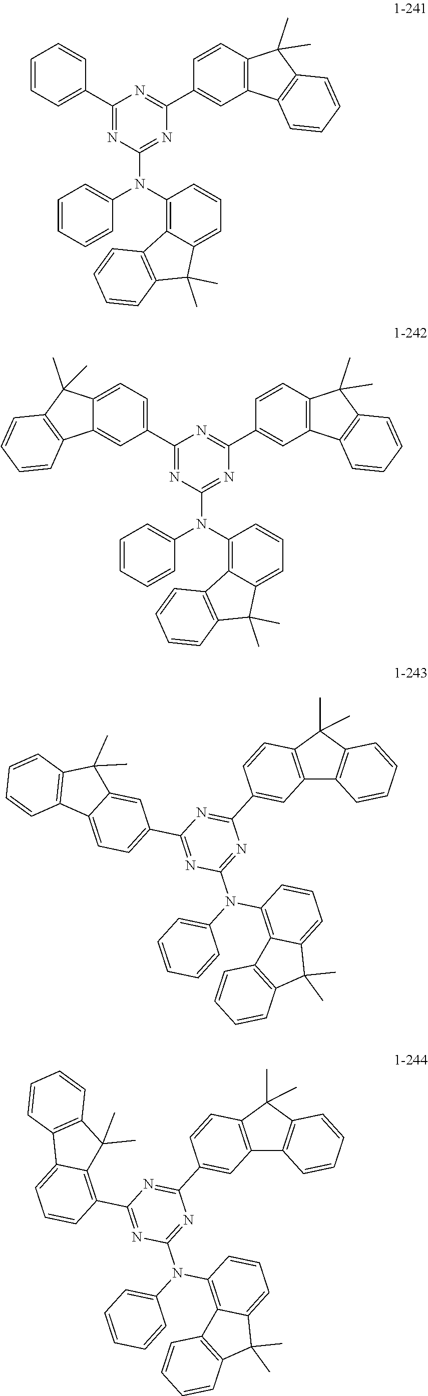 [SMILES: CC1(C)c2ccccc2-c2cc(-c3nc(-c4ccccc4)nc(N(c4ccccc4)c4cccc5c4-c4ccccc4C5(C)C)n3)ccc21 ; CC1(C)c2ccccc2-c2cc(-c3nc(-c4ccc5c(c4)-c4ccccc4C5(C)C)nc(N(c4ccccc4)c4cccc5c4-c4ccccc4C5(C)C)n3)ccc21 ; CC1(C)c2ccccc2-c2cc(-c3nc(-c4ccc5c(c4)C(C)(C)c4ccccc4-5)nc(N(c4ccccc4)c4cccc5c4-c4ccccc4C5(C)C)n3)ccc21 ; CC1(C)c2ccccc2-c2cc(-c3nc(-c4cccc5c4C(C)(C)c4ccccc4-5)nc(N(c4ccccc4)c4cccc5c4-c4ccccc4C5(C)C)n3)ccc21]

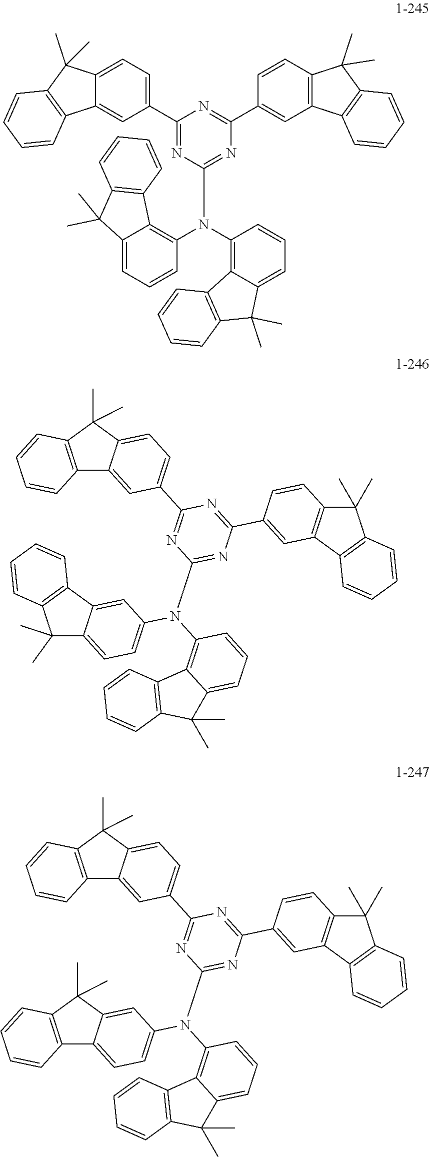 [SMILES: CC1(C)c2ccccc2-c2cc(-c3nc(-c4ccc5c(c4)-c4ccccc4C5(C)C)nc(N(c4cccc5c4-c4ccccc4C5(C)C)c4cccc5c4-c4ccccc4C5(C)C)n3)ccc21 ; CC1(C)c2ccccc2-c2cc(-c3nc(-c4ccc5c(c4)-c4ccccc4C5(C)C)nc(N(c4ccc5c(c4)-c4ccccc4C5(C)C)c4cccc5c4-c4ccccc4C5(C)C)n3)ccc21 ; CC1(C)c2ccccc2-c2cc(-c3nc(-c4ccc5c(c4)-c4ccccc4C5(C)C)nc(N(c4ccc5c(c4)C(C)(C)c4ccccc4-5)c4cccc5c4-c4ccccc4C5(C)C)n3)ccc21]

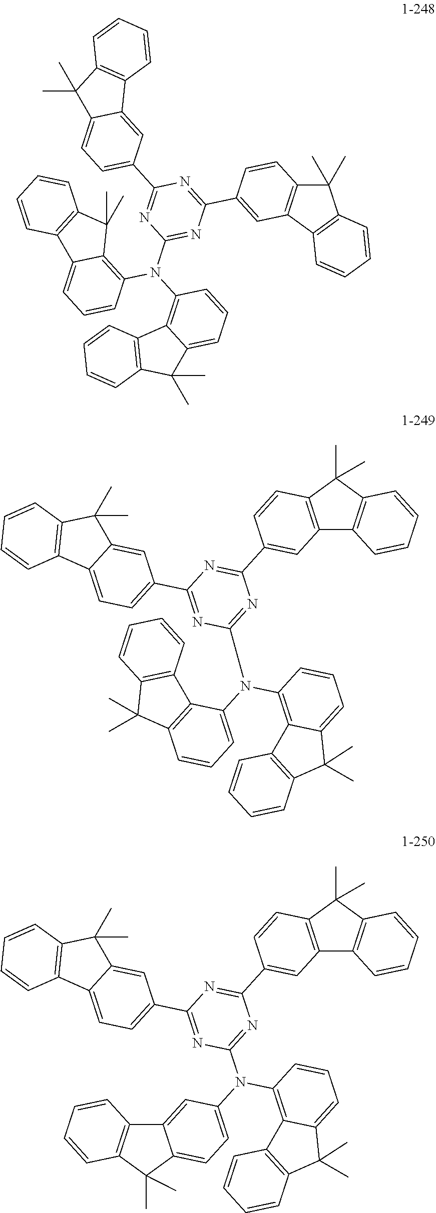 [SMILES: CC1(C)c2ccccc2-c2cc(-c3nc(-c4ccc5c(c4)-c4ccccc4C5(C)C)nc(N(c4cccc5c4-c4ccccc4C5(C)C)c4cccc5c4C(C)(C)c4ccccc4-5)n3)ccc21 ; CC1(C)c2ccccc2-c2cc(-c3nc(-c4ccc5c(c4)C(C)(C)c4ccccc4-5)nc(N(c4cccc5c4-c4ccccc4C5(C)C)c4cccc5c4-c4ccccc4C5(C)C)n3)ccc21 ; CC1(C)c2ccccc2-c2cc(-c3nc(-c4ccc5c(c4)C(C)(C)c4ccccc4-5)nc(N(c4ccc5c(c4)-c4ccccc4C5(C)C)c4cccc5c4-c4ccccc4C5(C)C)n3)ccc21]

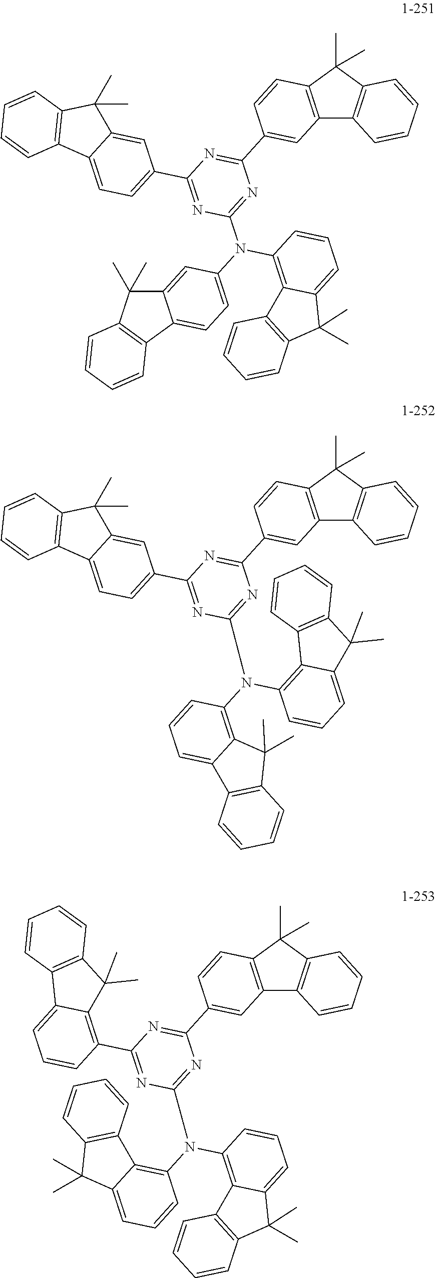 [SMILES: CC1(C)c2ccccc2-c2cc(-c3nc(-c4ccc5c(c4)C(C)(C)c4ccccc4-5)nc(N(c4ccc5c(c4)C(C)(C)c4ccccc4-5)c4cccc5c4-c4ccccc4C5(C)C)n3)ccc21 ; CC1(C)c2ccccc2-c2cc(-c3nc(-c4ccc5c(c4)C(C)(C)c4ccccc4-5)nc(N(c4cccc5c4-c4ccccc4C5(C)C)c4cccc5c4C(C)(C)c4ccccc4-5)n3)ccc21 ; CC1(C)c2ccccc2-c2cc(-c3nc(-c4cccc5c4C(C)(C)c4ccccc4-5)nc(N(c4cccc5c4-c4ccccc4C5(C)C)c4cccc5c4-c4ccccc4C5(C)C)n3)ccc21]

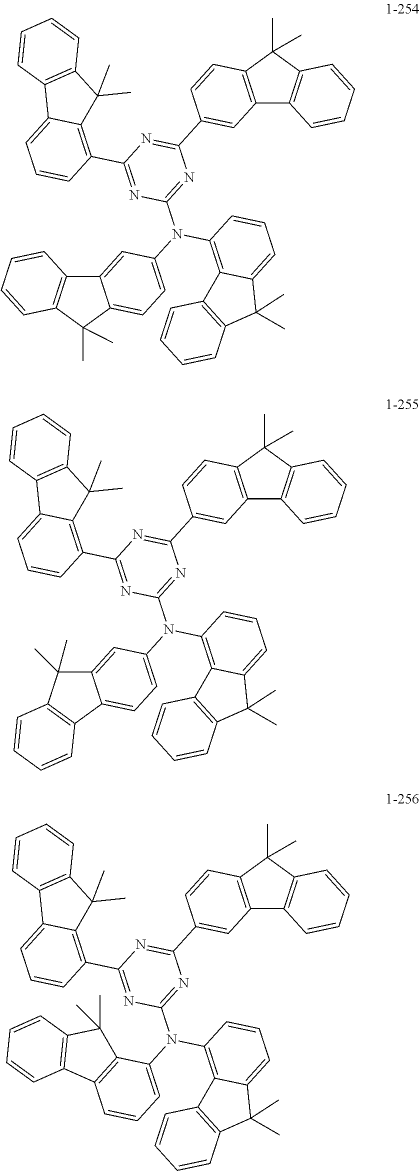 [SMILES: CC1(C)c2ccccc2-c2cc(-c3nc(-c4cccc5c4C(C)(C)c4ccccc4-5)nc(N(c4ccc5c(c4)-c4ccccc4C5(C)C)c4cccc5c4-c4ccccc4C5(C)C)n3)ccc21 ; CC1(C)c2ccccc2-c2cc(-c3nc(-c4cccc5c4C(C)(C)c4ccccc4-5)nc(N(c4ccc5c(c4)C(C)(C)c4ccccc4-5)c4cccc5c4-c4ccccc4C5(C)C)n3)ccc21 ; CC1(C)c2ccccc2-c2cc(-c3nc(-c4cccc5c4C(C)(C)c4ccccc4-5)nc(N(c4cccc5c4-c4ccccc4C5(C)C)c4cccc5c4C(C)(C)c4ccccc4-5)n3)ccc21]

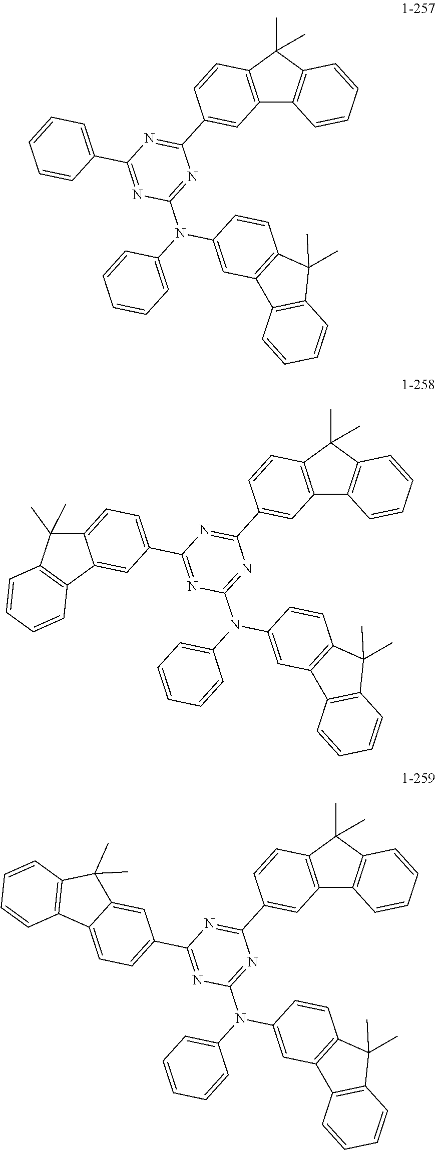 [SMILES: CC1(C)c2ccccc2-c2cc(-c3nc(-c4ccccc4)nc(N(c4ccccc4)c4ccc5c(c4)-c4ccccc4C5(C)C)n3)ccc21 ; CC1(C)c2ccccc2-c2cc(-c3nc(-c4ccc5c(c4)-c4ccccc4C5(C)C)nc(N(c4ccccc4)c4ccc5c(c4)-c4ccccc4C5(C)C)n3)ccc21 ; CC1(C)c2ccccc2-c2cc(-c3nc(-c4ccc5c(c4)C(C)(C)c4ccccc4-5)nc(N(c4ccccc4)c4ccc5c(c4)-c4ccccc4C5(C)C)n3)ccc21]

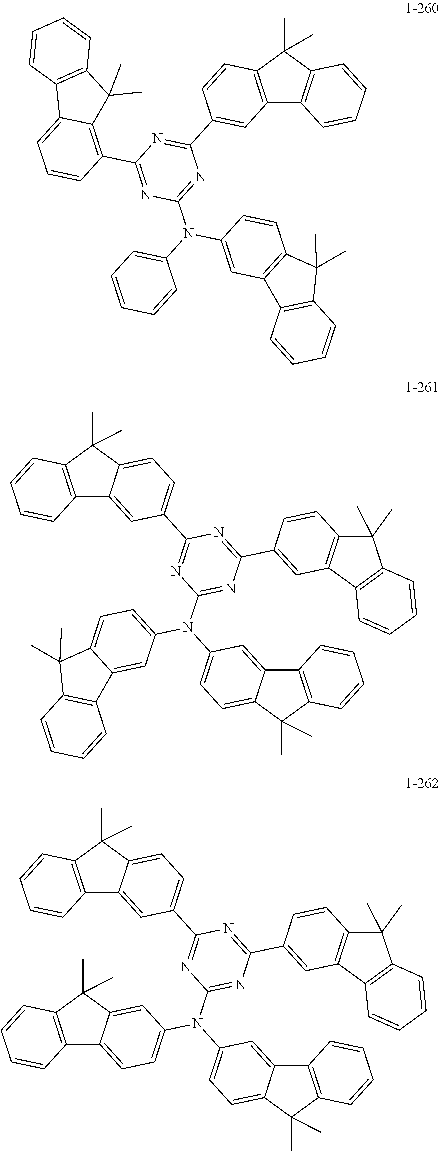 [SMILES: CC1(C)c2ccccc2-c2cc(-c3nc(-c4cccc5c4C(C)(C)c4ccccc4-5)nc(N(c4ccccc4)c4ccc5c(c4)-c4ccccc4C5(C)C)n3)ccc21 ; CC1(C)c2ccccc2-c2cc(-c3nc(-c4ccc5c(c4)-c4ccccc4C5(C)C)nc(N(c4ccc5c(c4)-c4ccccc4C5(C)C)c4ccc5c(c4)-c4ccccc4C5(C)C)n3)ccc21 ; CC1(C)c2ccccc2-c2cc(-c3nc(-c4ccc5c(c4)-c4ccccc4C5(C)C)nc(N(c4ccc5c(c4)-c4ccccc4C5(C)C)c4ccc5c(c4)C(C)(C)c4ccccc4-5)n3)ccc21]

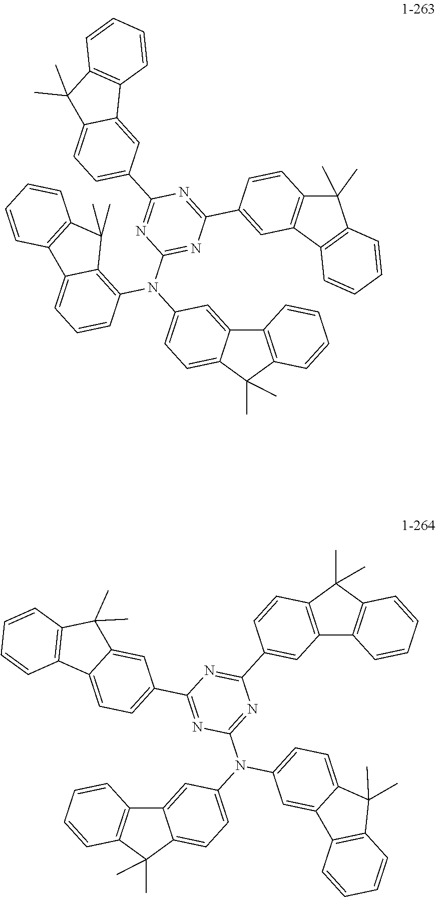 [SMILES: CC1(C)c2ccccc2-c2cc(-c3nc(-c4ccc5c(c4)-c4ccccc4C5(C)C)nc(N(c4ccc5c(c4)-c4ccccc4C5(C)C)c4cccc5c4C(C)(C)c4ccccc4-5)n3)ccc21 ; CC1(C)c2ccccc2-c2cc(-c3nc(-c4ccc5c(c4)C(C)(C)c4ccccc4-5)nc(N(c4ccc5c(c4)-c4ccccc4C5(C)C)c4ccc5c(c4)-c4ccccc4C5(C)C)n3)ccc21]

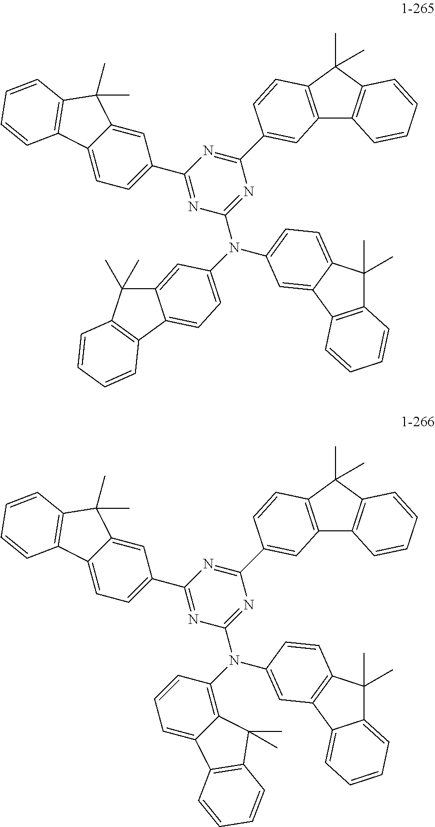 [SMILES: CC1(C)c2ccccc2-c2cc(-c3nc(-c4ccc5c(c4)C(C)(C)c4ccccc4-5)nc(N(c4ccc5c(c4)-c4ccccc4C5(C)C)c4ccc5c(c4)C(C)(C)c4ccccc4-5)n3)ccc21 ; CC1(C)c2ccccc2-c2cc(-c3nc(-c4ccc5c(c4)C(C)(C)c4ccccc4-5)nc(N(c4ccc5c(c4)-c4ccccc4C5(C)C)c4cccc5c4C(C)(C)c4ccccc4-5)n3)ccc21]

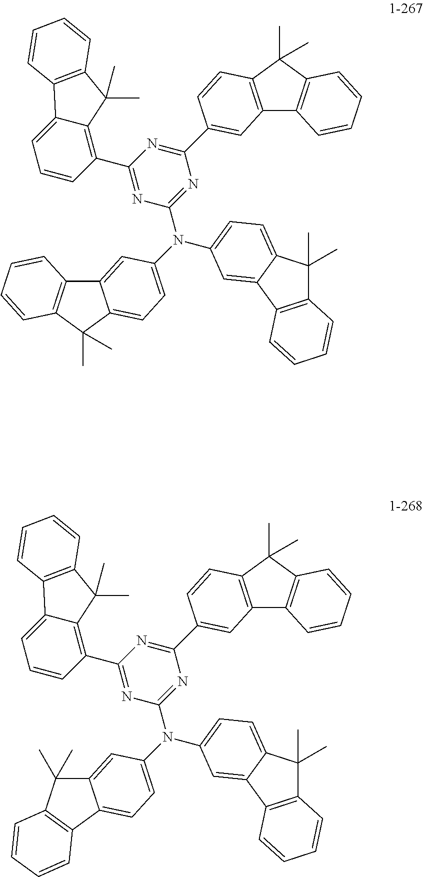 [SMILES: CC1(C)c2ccccc2-c2cc(-c3nc(-c4cccc5c4C(C)(C)c4ccccc4-5)nc(N(c4ccc5c(c4)-c4ccccc4C5(C)C)c4ccc5c(c4)-c4ccccc4C5(C)C)n3)ccc21 ; CC1(C)c2ccccc2-c2cc(-c3nc(-c4cccc5c4C(C)(C)c4ccccc4-5)nc(N(c4ccc5c(c4)-c4ccccc4C5(C)C)c4ccc5c(c4)C(C)(C)c4ccccc4-5)n3)ccc21]

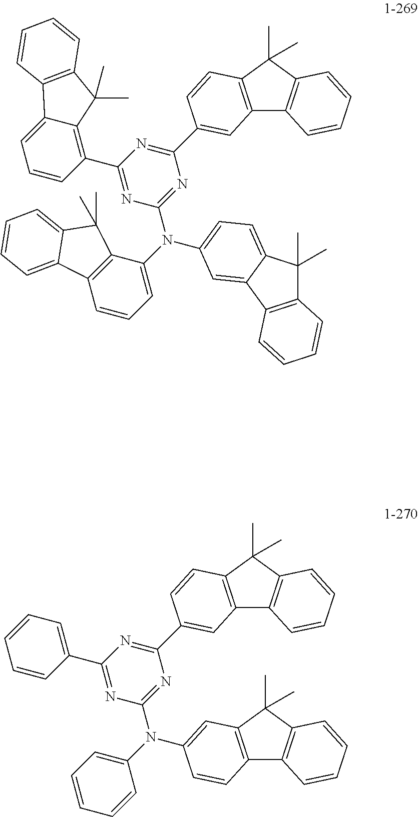 [SMILES: CC1(C)c2ccccc2-c2cc(-c3nc(-c4cccc5c4C(C)(C)c4ccccc4-5)nc(N(c4ccc5c(c4)-c4ccccc4C5(C)C)c4cccc5c4C(C)(C)c4ccccc4-5)n3)ccc21 ; CC1(C)c2ccccc2-c2cc(-c3nc(-c4ccccc4)nc(N(c4ccccc4)c4ccc5c(c4)C(C)(C)c4ccccc4-5)n3)ccc21]

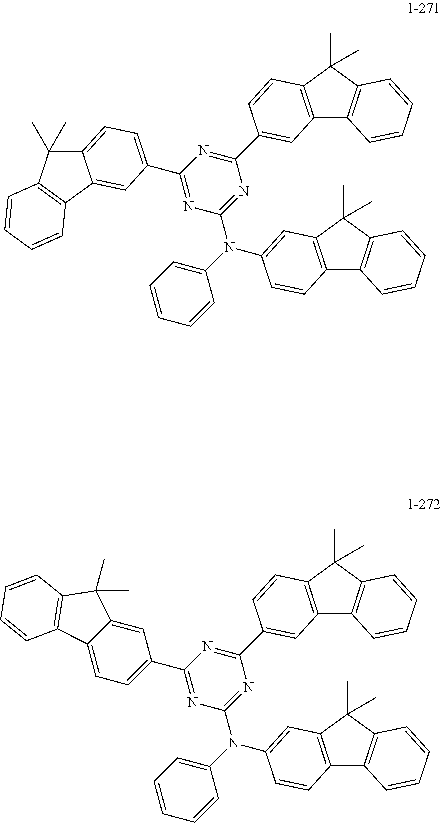 [SMILES: CC1(C)c2ccccc2-c2cc(-c3nc(-c4ccc5c(c4)-c4ccccc4C5(C)C)nc(N(c4ccccc4)c4ccc5c(c4)C(C)(C)c4ccccc4-5)n3)ccc21 ; CC1(C)c2ccccc2-c2cc(-c3nc(-c4ccc5c(c4)C(C)(C)c4ccccc4-5)nc(N(c4ccccc4)c4ccc5c(c4)C(C)(C)c4ccccc4-5)n3)ccc21]

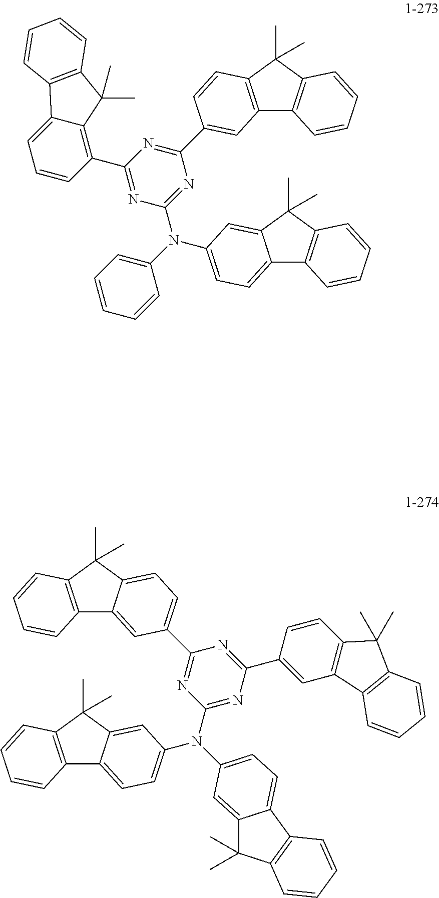 [SMILES: CC1(C)c2ccccc2-c2cc(-c3nc(-c4cccc5c4C(C)(C)c4ccccc4-5)nc(N(c4ccccc4)c4ccc5c(c4)C(C)(C)c4ccccc4-5)n3)ccc21 ; CC1(C)c2ccccc2-c2cc(-c3nc(-c4ccc5c(c4)-c4ccccc4C5(C)C)nc(N(c4ccc5c(c4)C(C)(C)c4ccccc4-5)c4ccc5c(c4)C(C)(C)c4ccccc4-5)n3)ccc21]

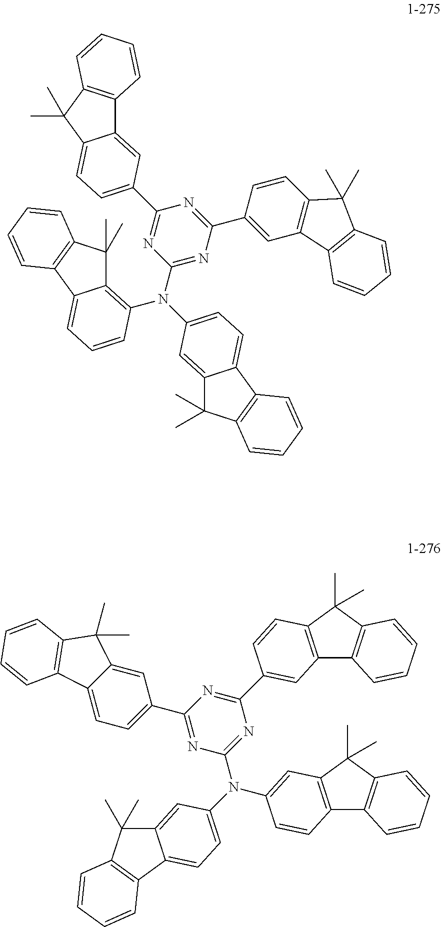 [SMILES: CC1(C)c2ccccc2-c2cc(-c3nc(-c4ccc5c(c4)-c4ccccc4C5(C)C)nc(N(c4ccc5c(c4)C(C)(C)c4ccccc4-5)c4cccc5c4C(C)(C)c4ccccc4-5)n3)ccc21 ; CC1(C)c2ccccc2-c2cc(-c3nc(-c4ccc5c(c4)C(C)(C)c4ccccc4-5)nc(N(c4ccc5c(c4)C(C)(C)c4ccccc4-5)c4ccc5c(c4)C(C)(C)c4ccccc4-5)n3)ccc21]

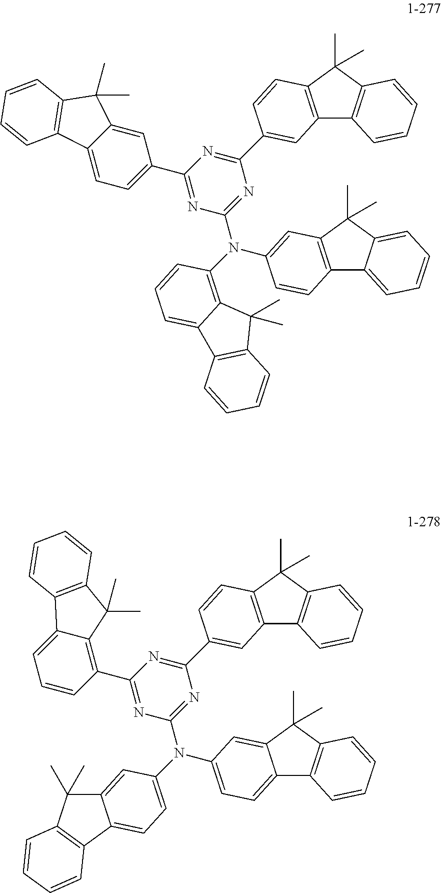 [SMILES: CC1(C)c2ccccc2-c2cc(-c3nc(-c4ccc5c(c4)C(C)(C)c4ccccc4-5)nc(N(c4ccc5c(c4)C(C)(C)c4ccccc4-5)c4cccc5c4C(C)(C)c4ccccc4-5)n3)ccc21 ; CC1(C)c2ccccc2-c2cc(-c3nc(-c4cccc5c4C(C)(C)c4ccccc4-5)nc(N(c4ccc5c(c4)C(C)(C)c4ccccc4-5)c4ccc5c(c4)C(C)(C)c4ccccc4-5)n3)ccc21]

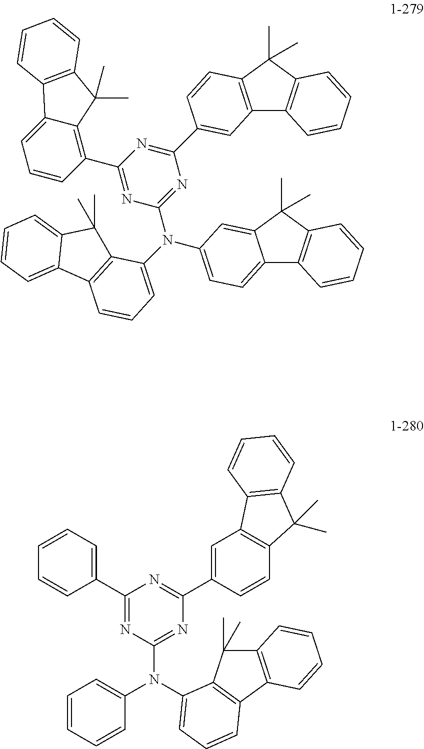 [SMILES: CC1(C)c2ccccc2-c2cc(-c3nc(-c4cccc5c4C(C)(C)c4ccccc4-5)nc(N(c4ccc5c(c4)C(C)(C)c4ccccc4-5)c4cccc5c4C(C)(C)c4ccccc4-5)n3)ccc21 ; CC1(C)c2ccccc2-c2cc(-c3nc(-c4ccccc4)nc(N(c4ccccc4)c4cccc5c4C(C)(C)c4ccccc4-5)n3)ccc21]

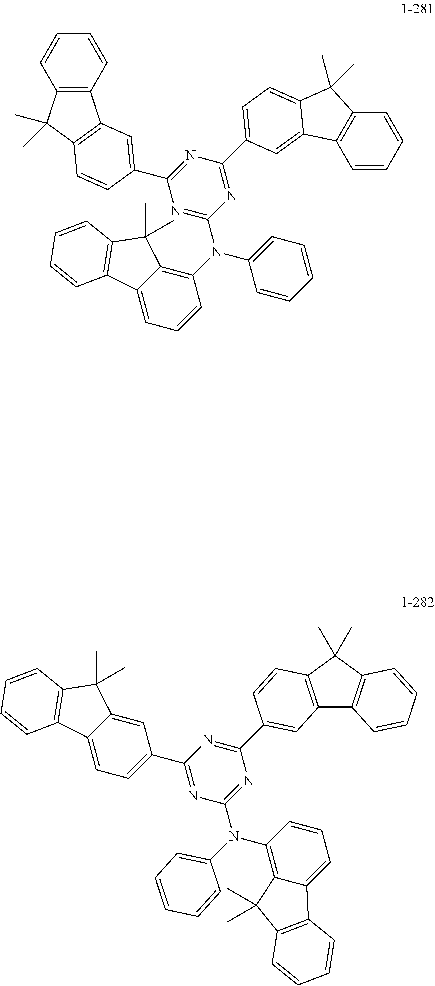 [SMILES: CC1(C)c2ccccc2-c2cc(-c3nc(-c4ccc5c(c4)-c4ccccc4C5(C)C)nc(N(c4ccccc4)c4cccc5c4C(C)(C)c4ccccc4-5)n3)ccc21 ; CC1(C)c2ccccc2-c2cc(-c3nc(-c4ccc5c(c4)C(C)(C)c4ccccc4-5)nc(N(c4ccccc4)c4cccc5c4C(C)(C)c4ccccc4-5)n3)ccc21]

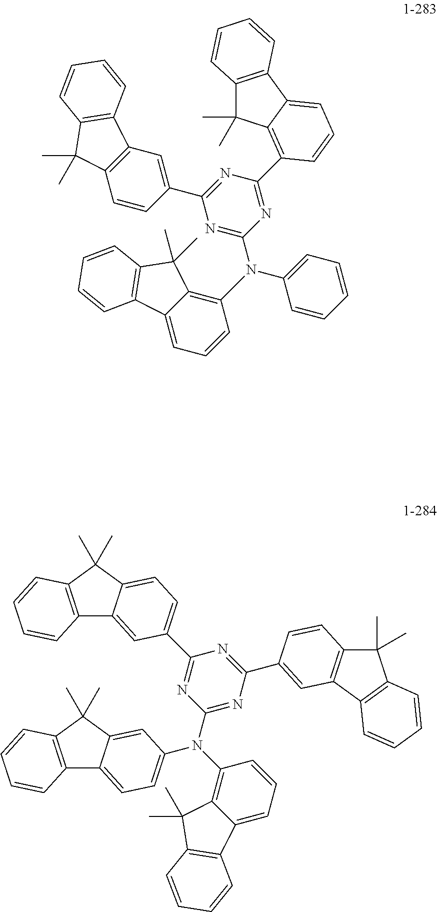 [SMILES: CC1(C)c2ccccc2-c2cc(-c3nc(-c4cccc5c4C(C)(C)c4ccccc4-5)nc(N(c4ccccc4)c4cccc5c4C(C)(C)c4ccccc4-5)n3)ccc21 ; CC1(C)c2ccccc2-c2cc(-c3nc(-c4ccc5c(c4)-c4ccccc4C5(C)C)nc(N(c4ccc5c(c4)C(C)(C)c4ccccc4-5)c4cccc5c4C(C)(C)c4ccccc4-5)n3)ccc21]

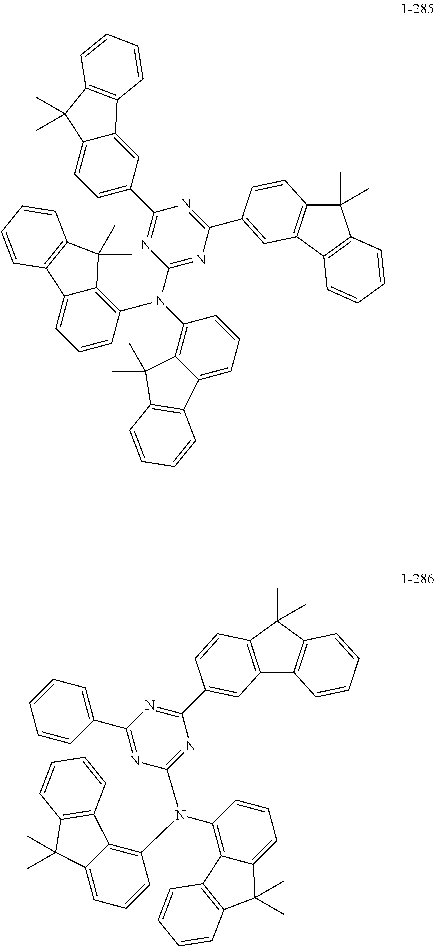 [SMILES: CC1(C)c2ccccc2-c2cc(-c3nc(-c4ccc5c(c4)-c4ccccc4C5(C)C)nc(N(c4cccc5c4C(C)(C)c4ccccc4-5)c4cccc5c4C(C)(C)c4ccccc4-5)n3)ccc21 ; CC1(C)c2ccccc2-c2cc(-c3nc(-c4ccccc4)nc(N(c4cccc5c4-c4ccccc4C5(C)C)c4cccc5c4-c4ccccc4C5(C)C)n3)ccc21]

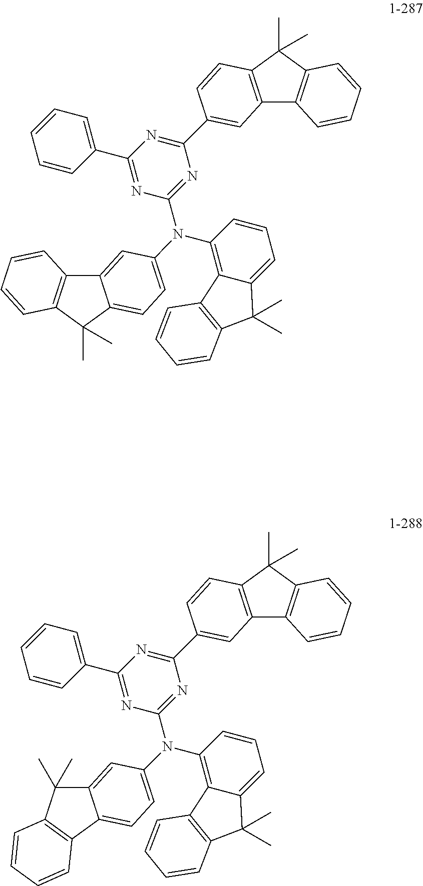 [SMILES: CC1(C)c2ccccc2-c2cc(-c3nc(-c4ccccc4)nc(N(c4ccc5c(c4)-c4ccccc4C5(C)C)c4cccc5c4-c4ccccc4C5(C)C)n3)ccc21 ; CC1(C)c2ccccc2-c2cc(-c3nc(-c4ccccc4)nc(N(c4ccc5c(c4)C(C)(C)c4ccccc4-5)c4cccc5c4-c4ccccc4C5(C)C)n3)ccc21]

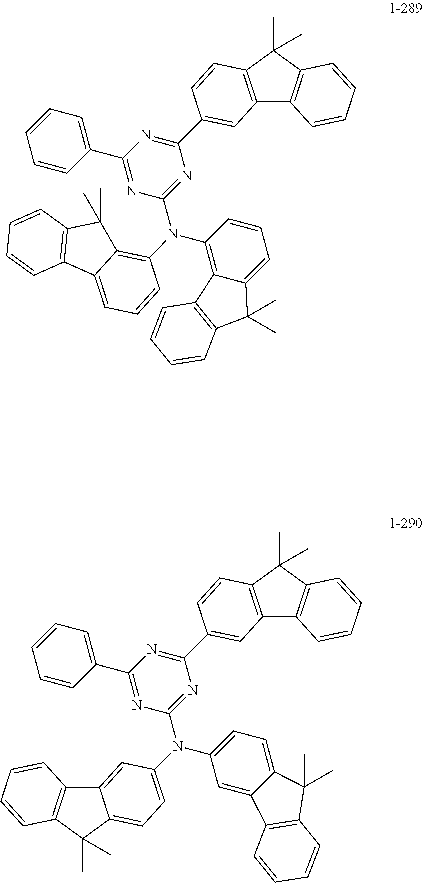 [SMILES: CC1(C)c2ccccc2-c2cc(-c3nc(-c4ccccc4)nc(N(c4cccc5c4-c4ccccc4C5(C)C)c4cccc5c4C(C)(C)c4ccccc4-5)n3)ccc21 ; CC1(C)c2ccccc2-c2cc(-c3nc(-c4ccccc4)nc(N(c4ccc5c(c4)-c4ccccc4C5(C)C)c4ccc5c(c4)-c4ccccc4C5(C)C)n3)ccc21]

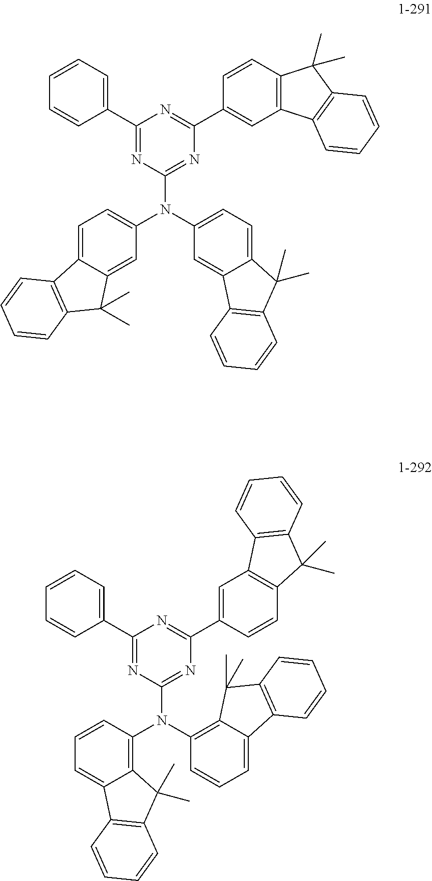 [SMILES: CC1(C)c2ccccc2-c2cc(-c3nc(-c4ccccc4)nc(N(c4ccc5c(c4)-c4ccccc4C5(C)C)c4ccc5c(c4)C(C)(C)c4ccccc4-5)n3)ccc21 ; CC1(C)c2ccccc2-c2cc(-c3nc(-c4ccccc4)nc(N(c4cccc5c4C(C)(C)c4ccccc4-5)c4cccc5c4C(C)(C)c4ccccc4-5)n3)ccc21]

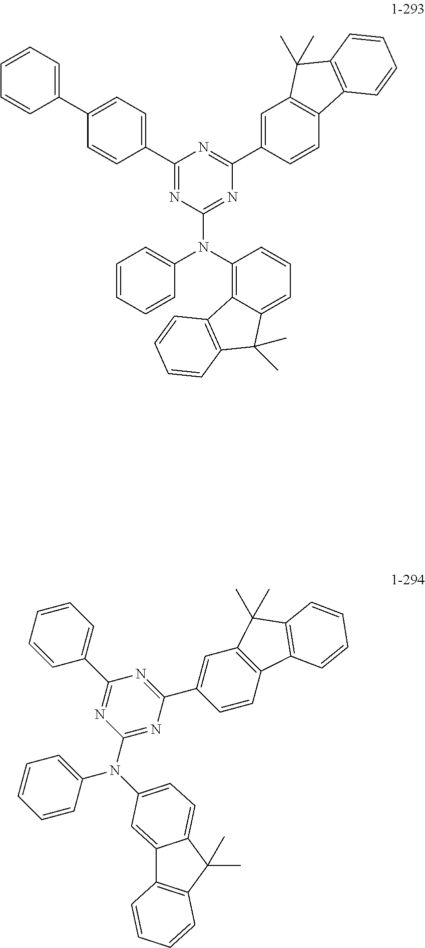 [SMILES: CC1(C)c2ccccc2-c2ccc(-c3nc(-c4ccc(-c5ccccc5)cc4)nc(N(c4ccccc4)c4cccc5c4-c4ccccc4C5(C)C)n3)cc21 ; CC1(C)c2ccccc2-c2cc(N(c3ccccc3)c3nc(-c4ccccc4)nc(-c4ccc5c(c4)C(C)(C)c4ccccc4-5)n3)ccc21]

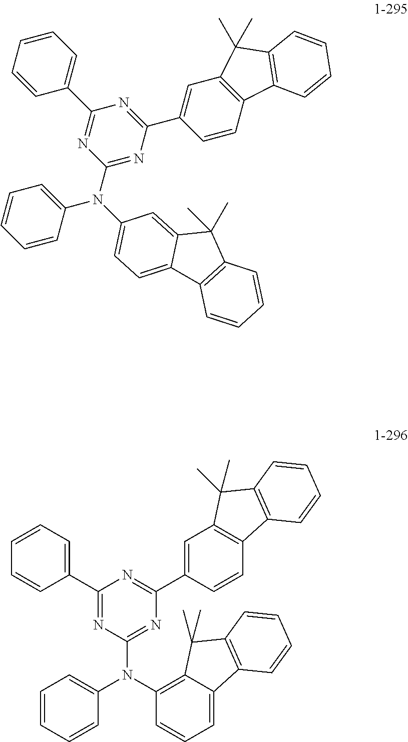 [SMILES: CC1(C)c2ccccc2-c2ccc(-c3nc(-c4ccccc4)nc(N(c4ccccc4)c4ccc5c(c4)C(C)(C)c4ccccc4-5)n3)cc21 ; CC1(C)c2ccccc2-c2ccc(-c3nc(-c4ccccc4)nc(N(c4ccccc4)c4cccc5c4C(C)(C)c4ccccc4-5)n3)cc21]

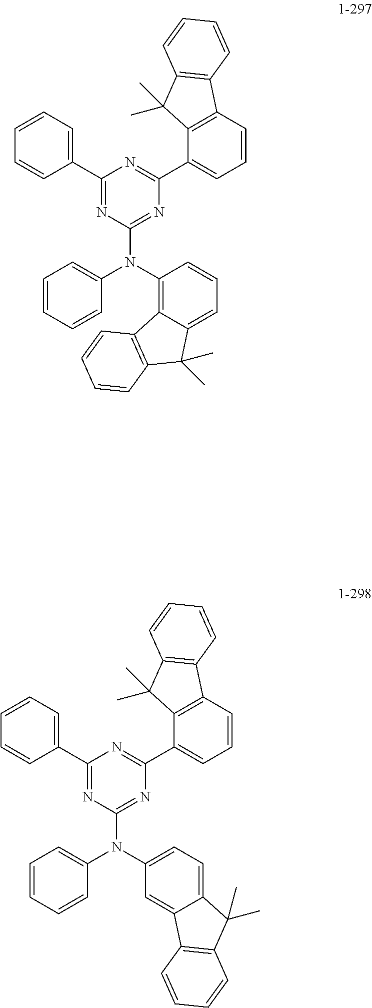 [SMILES: CC1(C)c2ccccc2-c2c(N(c3ccccc3)c3nc(-c4ccccc4)nc(-c4cccc5c4C(C)(C)c4ccccc4-5)n3)cccc21 ; CC1(C)c2ccccc2-c2cc(N(c3ccccc3)c3nc(-c4ccccc4)nc(-c4cccc5c4C(C)(C)c4ccccc4-5)n3)ccc21]

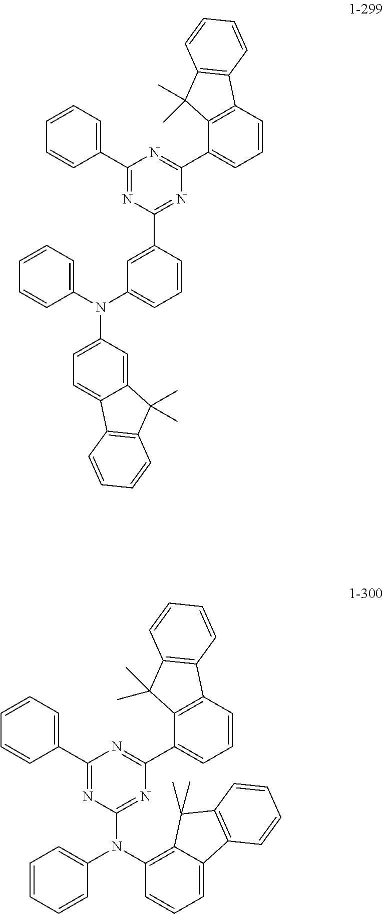 [SMILES: CC1(C)c2ccccc2-c2ccc(N(c3ccccc3)c3cccc(-c4nc(-c5ccccc5)nc(-c5cccc6c5C(C)(C)c5ccccc5-6)n4)c3)cc21 ; CC1(C)c2ccccc2-c2cccc(-c3nc(-c4ccccc4)nc(N(c4ccccc4)c4cccc5c4C(C)(C)c4ccccc4-5)n3)c21]

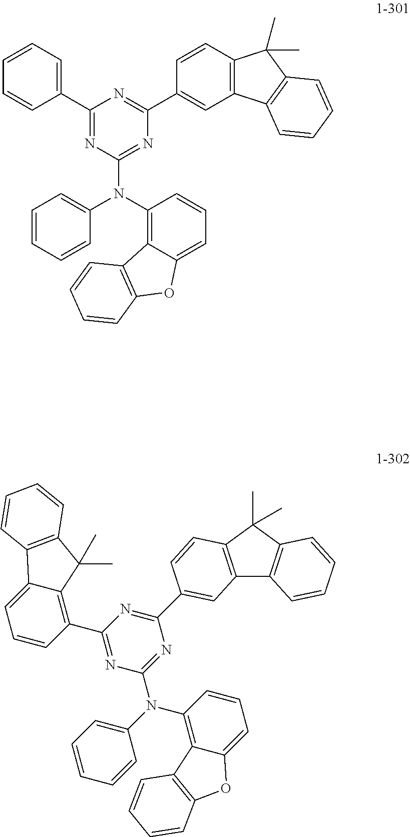 [SMILES: CC1(C)c2ccccc2-c2cc(-c3nc(-c4ccccc4)nc(N(c4ccccc4)c4cccc5oc6ccccc6c45)n3)ccc21 ; CC1(C)c2ccccc2-c2cc(-c3nc(-c4cccc5c4C(C)(C)c4ccccc4-5)nc(N(c4ccccc4)c4cccc5oc6ccccc6c45)n3)ccc21]

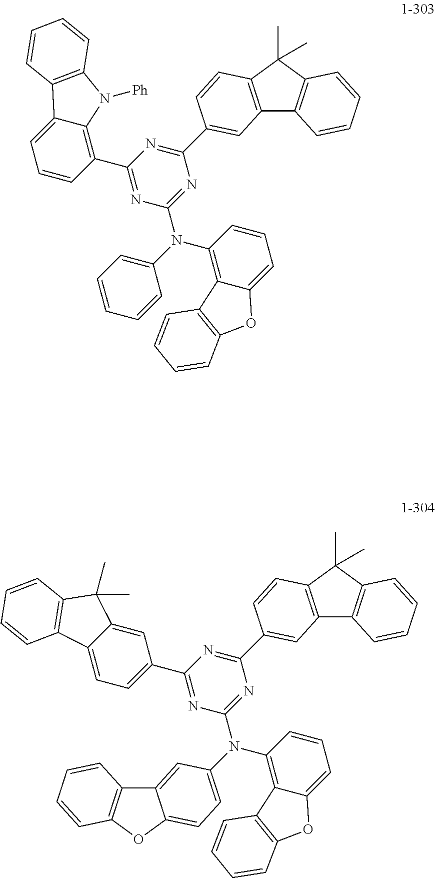 [SMILES: CC1(C)c2ccccc2-c2cc(-c3nc(-c4cccc5c6ccccc6n(-c6ccccc6)c45)nc(N(c4ccccc4)c4cccc5oc6ccccc6c45)n3)ccc21 ; CC1(C)c2ccccc2-c2cc(-c3nc(-c4ccc5c(c4)C(C)(C)c4ccccc4-5)nc(N(c4ccc5oc6ccccc6c5c4)c4cccc5oc6ccccc6c45)n3)ccc21]

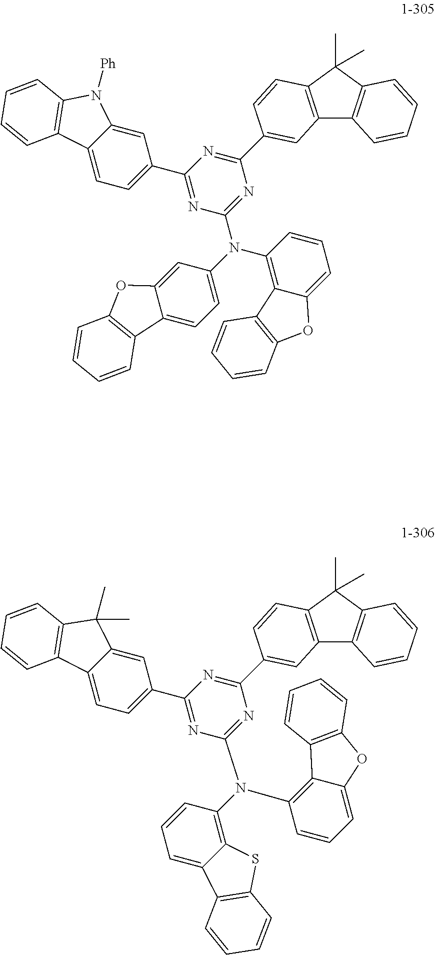 [SMILES: CC1(C)c2ccccc2-c2cc(-c3nc(-c4ccc5c6ccccc6n(-c6ccccc6)c5c4)nc(N(c4ccc5c(c4)oc4ccccc45)c4cccc5oc6ccccc6c45)n3)ccc21 ; CC1(C)c2ccccc2-c2cc(-c3nc(-c4ccc5c(c4)C(C)(C)c4ccccc4-5)nc(N(c4cccc5c4sc4ccccc45)c4cccc5oc6ccccc6c45)n3)ccc21]

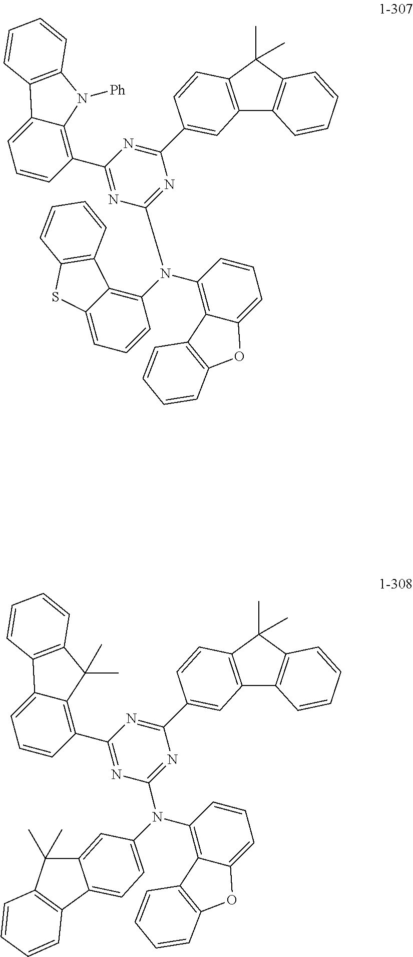 [SMILES: CC1(C)c2ccccc2-c2cc(-c3nc(-c4cccc5c6ccccc6n(-c6ccccc6)c45)nc(N(c4cccc5oc6ccccc6c45)c4cccc5sc6ccccc6c45)n3)ccc21 ; CC1(C)c2ccccc2-c2cc(-c3nc(-c4cccc5c4C(C)(C)c4ccccc4-5)nc(N(c4ccc5c(c4)C(C)(C)c4ccccc4-5)c4cccc5oc6ccccc6c45)n3)ccc21]

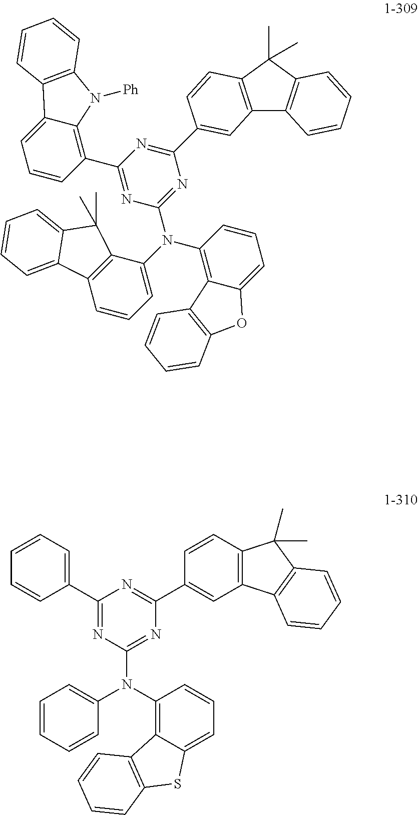 [SMILES: CC1(C)c2ccccc2-c2cc(-c3nc(-c4cccc5c6ccccc6n(-c6ccccc6)c45)nc(N(c4cccc5c4C(C)(C)c4ccccc4-5)c4cccc5oc6ccccc6c45)n3)ccc21 ; CC1(C)c2ccccc2-c2cc(-c3nc(-c4ccccc4)nc(N(c4ccccc4)c4cccc5sc6ccccc6c45)n3)ccc21]

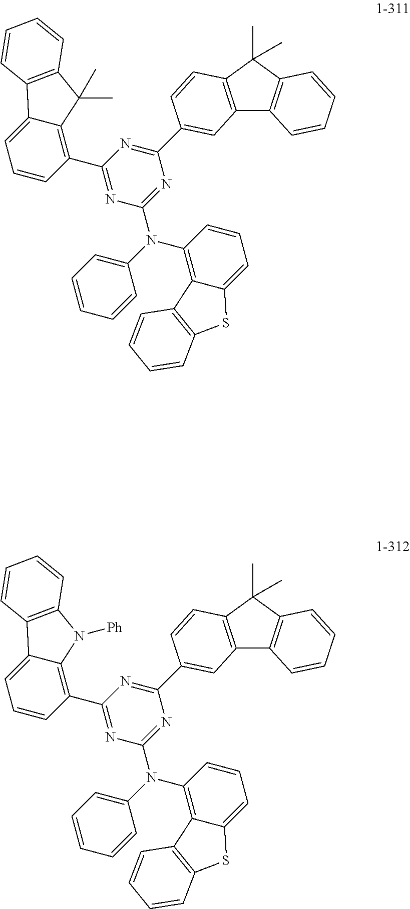 [SMILES: CC1(C)c2ccccc2-c2cc(-c3nc(-c4cccc5c4C(C)(C)c4ccccc4-5)nc(N(c4ccccc4)c4cccc5sc6ccccc6c45)n3)ccc21 ; CC1(C)c2ccccc2-c2cc(-c3nc(-c4cccc5c6ccccc6n(-c6ccccc6)c45)nc(N(c4ccccc4)c4cccc5sc6ccccc6c45)n3)ccc21]

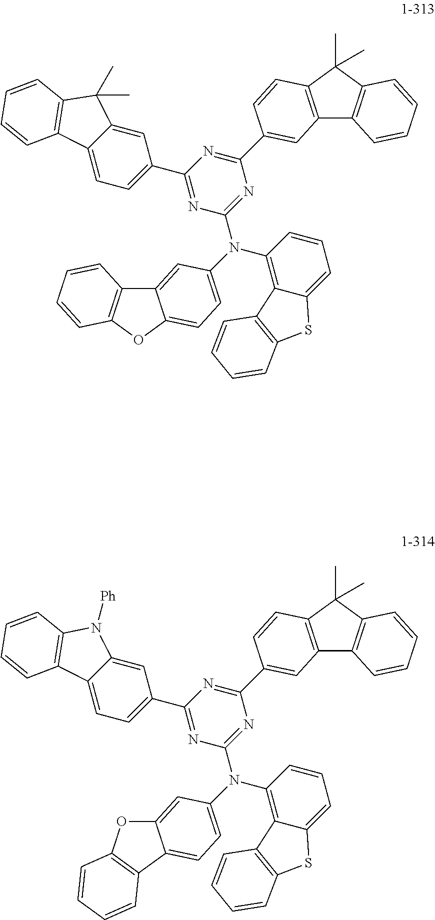 [SMILES: CC1(C)c2ccccc2-c2cc(-c3nc(-c4ccc5c(c4)C(C)(C)c4ccccc4-5)nc(N(c4ccc5oc6ccccc6c5c4)c4cccc5sc6ccccc6c45)n3)ccc21 ; CC1(C)c2ccccc2-c2cc(-c3nc(-c4ccc5c6ccccc6n(-c6ccccc6)c5c4)nc(N(c4ccc5c(c4)oc4ccccc45)c4cccc5sc6ccccc6c45)n3)ccc21]

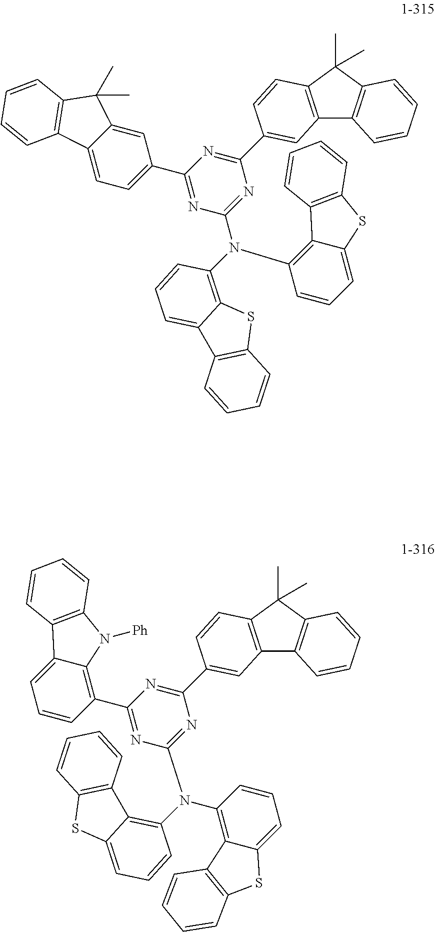 [SMILES: CC1(C)c2ccccc2-c2cc(-c3nc(-c4ccc5c(c4)C(C)(C)c4ccccc4-5)nc(N(c4cccc5c4sc4ccccc45)c4cccc5sc6ccccc6c45)n3)ccc21 ; CC1(C)c2ccccc2-c2cc(-c3nc(-c4cccc5c6ccccc6n(-c6ccccc6)c45)nc(N(c4cccc5sc6ccccc6c45)c4cccc5sc6ccccc6c45)n3)ccc21]

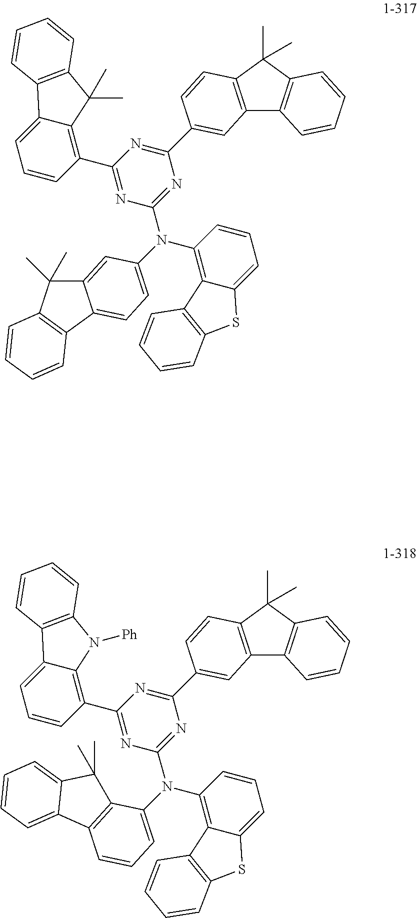 [SMILES: CC1(C)c2ccccc2-c2cc(-c3nc(-c4cccc5c4C(C)(C)c4ccccc4-5)nc(N(c4ccc5c(c4)C(C)(C)c4ccccc4-5)c4cccc5sc6ccccc6c45)n3)ccc21 ; CC1(C)c2ccccc2-c2cc(-c3nc(-c4cccc5c6ccccc6n(-c6ccccc6)c45)nc(N(c4cccc5c4C(C)(C)c4ccccc4-5)c4cccc5sc6ccccc6c45)n3)ccc21]

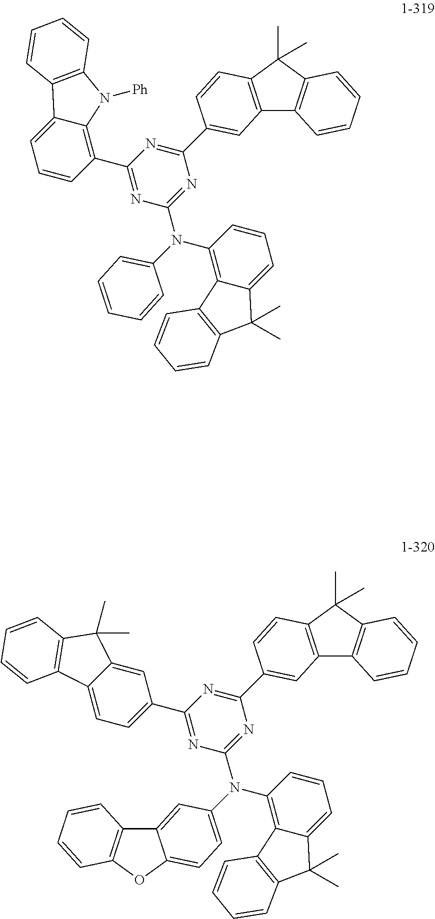 [SMILES: CC1(C)c2ccccc2-c2cc(-c3nc(-c4cccc5c6ccccc6n(-c6ccccc6)c45)nc(N(c4ccccc4)c4cccc5c4-c4ccccc4C5(C)C)n3)ccc21 ; CC1(C)c2ccccc2-c2cc(-c3nc(-c4ccc5c(c4)C(C)(C)c4ccccc4-5)nc(N(c4ccc5oc6ccccc6c5c4)c4cccc5c4-c4ccccc4C5(C)C)n3)ccc21]

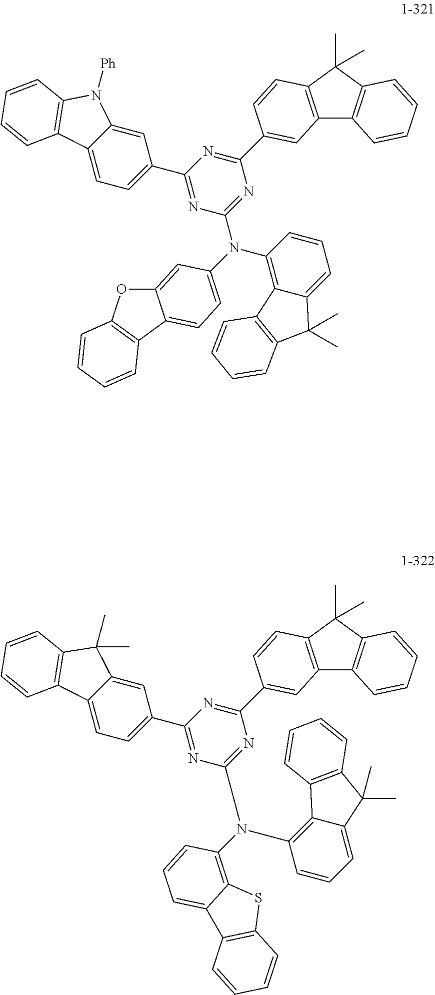 [SMILES: CC1(C)c2ccccc2-c2cc(-c3nc(-c4ccc5c6ccccc6n(-c6ccccc6)c5c4)nc(N(c4ccc5c(c4)oc4ccccc45)c4cccc5c4-c4ccccc4C5(C)C)n3)ccc21 ; CC1(C)c2ccccc2-c2cc(-c3nc(-c4ccc5c(c4)C(C)(C)c4ccccc4-5)nc(N(c4cccc5c4-c4ccccc4C5(C)C)c4cccc5c4sc4ccccc45)n3)ccc21]

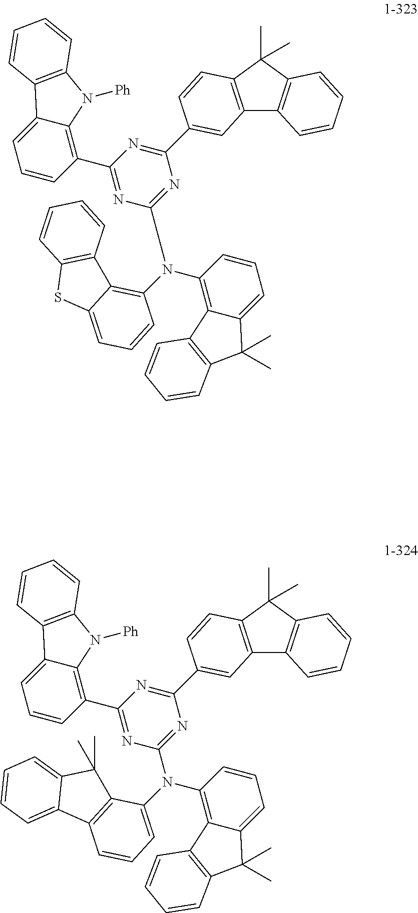 [SMILES: CC1(C)c2ccccc2-c2cc(-c3nc(-c4cccc5c6ccccc6n(-c6ccccc6)c45)nc(N(c4cccc5c4-c4ccccc4C5(C)C)c4cccc5sc6ccccc6c45)n3)ccc21 ; CC1(C)c2ccccc2-c2cc(-c3nc(-c4cccc5c6ccccc6n(-c6ccccc6)c45)nc(N(c4cccc5c4-c4ccccc4C5(C)C)c4cccc5c4C(C)(C)c4ccccc4-5)n3)ccc21]

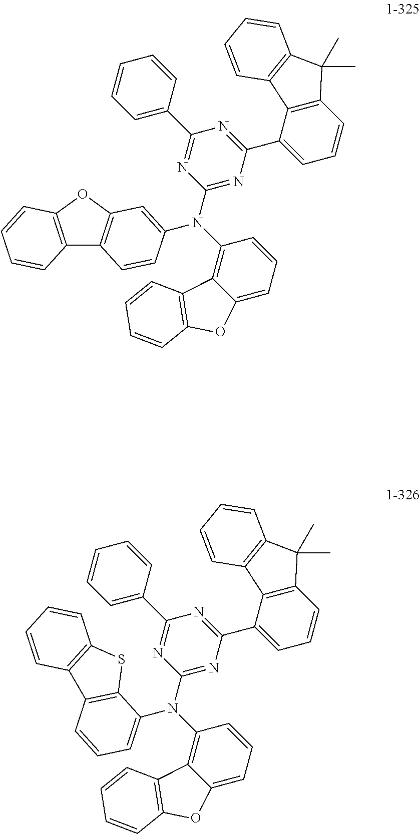 [SMILES: CC1(C)c2ccccc2-c2c(-c3nc(-c4ccccc4)nc(N(c4ccc5c(c4)oc4ccccc45)c4cccc5oc6ccccc6c45)n3)cccc21 ; CC1(C)c2ccccc2-c2c(-c3nc(-c4ccccc4)nc(N(c4cccc5c4sc4ccccc45)c4cccc5oc6ccccc6c45)n3)cccc21]

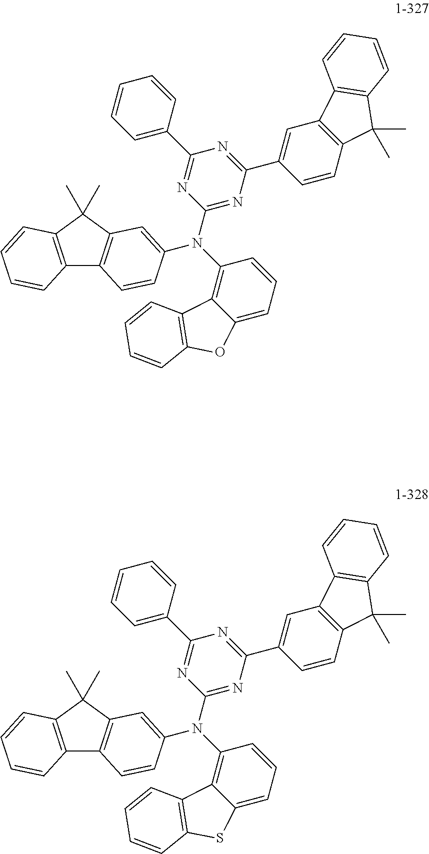 [SMILES: CC1(C)c2ccccc2-c2cc(-c3nc(-c4ccccc4)nc(N(c4ccc5c(c4)C(C)(C)c4ccccc4-5)c4cccc5oc6ccccc6c45)n3)ccc21 ; CC1(C)c2ccccc2-c2cc(-c3nc(-c4ccccc4)nc(N(c4ccc5c(c4)C(C)(C)c4ccccc4-5)c4cccc5sc6ccccc6c45)n3)ccc21]

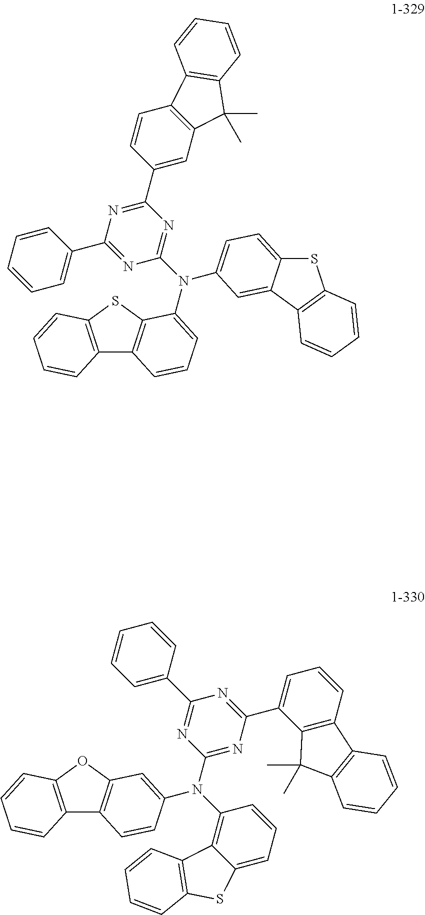 [SMILES: CC1(C)c2ccccc2-c2ccc(-c3nc(-c4ccccc4)nc(N(c4ccc5sc6ccccc6c5c4)c4cccc5c4sc4ccccc45)n3)cc21 ; CC1(C)c2ccccc2-c2cccc(-c3nc(-c4ccccc4)nc(N(c4ccc5c(c4)oc4ccccc45)c4cccc5sc6ccccc6c45)n3)c21]

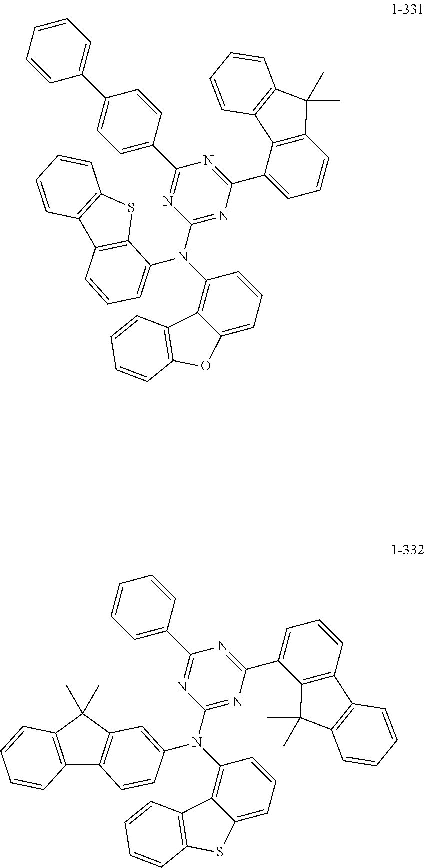 [SMILES: CC1(C)c2ccccc2-c2c(-c3nc(-c4ccc(-c5ccccc5)cc4)nc(N(c4cccc5c4sc4ccccc45)c4cccc5oc6ccccc6c45)n3)cccc21 ; CC1(C)c2ccccc2-c2ccc(N(c3nc(-c4ccccc4)nc(-c4cccc5c4C(C)(C)c4ccccc4-5)n3)c3cccc4sc5ccccc5c34)cc21]

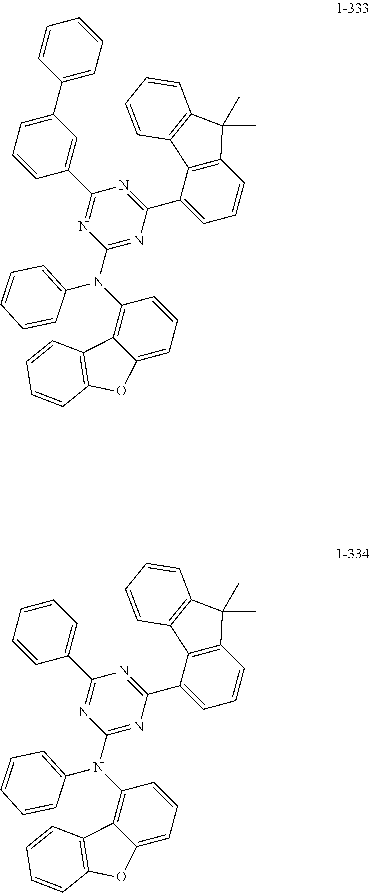 [SMILES: CC1(C)c2ccccc2-c2c(-c3nc(-c4cccc(-c5ccccc5)c4)nc(N(c4ccccc4)c4cccc5oc6ccccc6c45)n3)cccc21 ; CC1(C)c2ccccc2-c2c(-c3nc(-c4ccccc4)nc(N(c4ccccc4)c4cccc5oc6ccccc6c45)n3)cccc21]

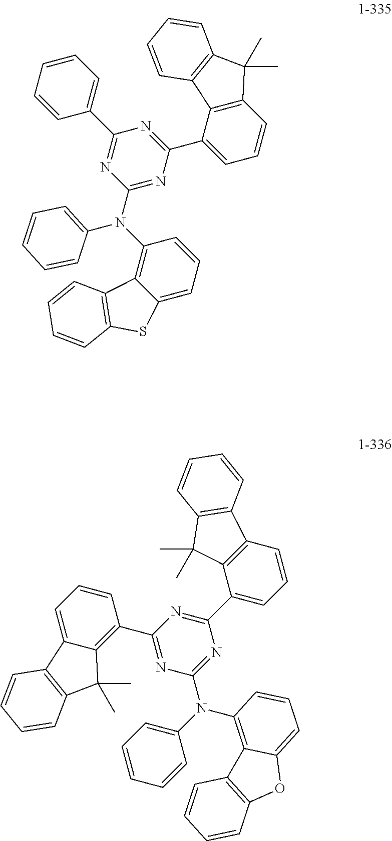 [SMILES: CC1(C)c2ccccc2-c2c(-c3nc(-c4ccccc4)nc(N(c4ccccc4)c4cccc5sc6ccccc6c45)n3)cccc21 ; CC1(C)c2ccccc2-c2cccc(-c3nc(-c4cccc5c4C(C)(C)c4ccccc4-5)nc(N(c4ccccc4)c4cccc5oc6ccccc6c45)n3)c21]

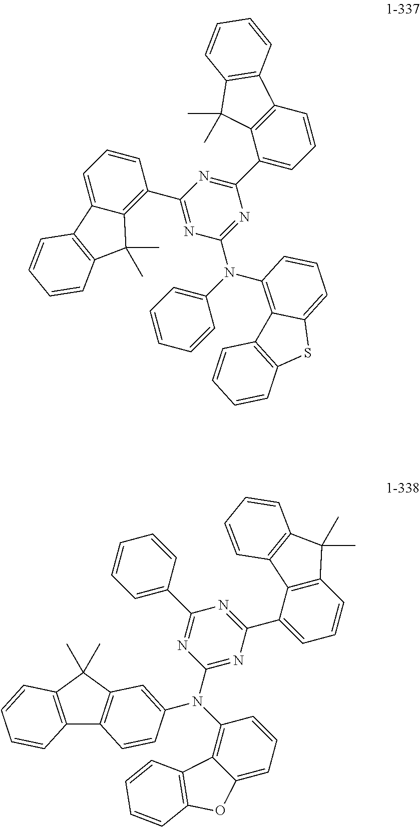 [SMILES: CC1(C)c2ccccc2-c2cccc(-c3nc(-c4cccc5c4C(C)(C)c4ccccc4-5)nc(N(c4ccccc4)c4cccc5sc6ccccc6c45)n3)c21 ; CC1(C)c2ccccc2-c2ccc(N(c3nc(-c4ccccc4)nc(-c4cccc5c4-c4ccccc4C5(C)C)n3)c3cccc4oc5ccccc5c34)cc21]

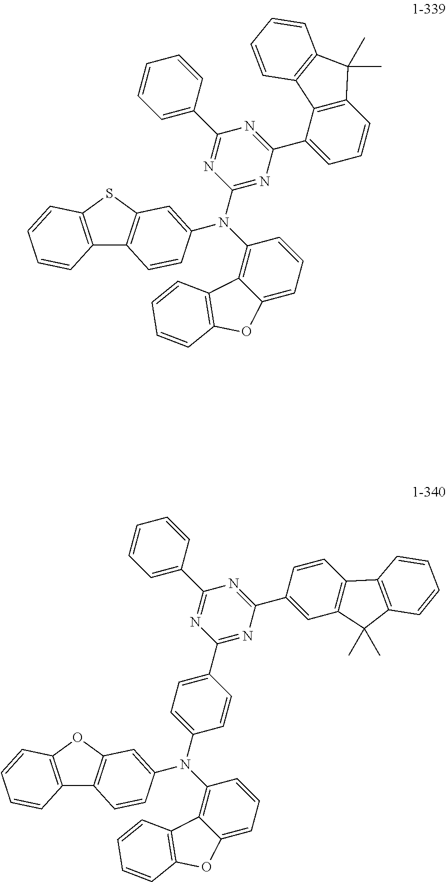 [SMILES: CC1(C)c2ccccc2-c2c(-c3nc(-c4ccccc4)nc(N(c4ccc5c(c4)sc4ccccc45)c4cccc5oc6ccccc6c45)n3)cccc21 ; CC1(C)c2ccccc2-c2ccc(-c3nc(-c4ccccc4)nc(-c4ccc(N(c5ccc6c(c5)oc5ccccc56)c5cccc6oc7ccccc7c56)cc4)n3)cc21]

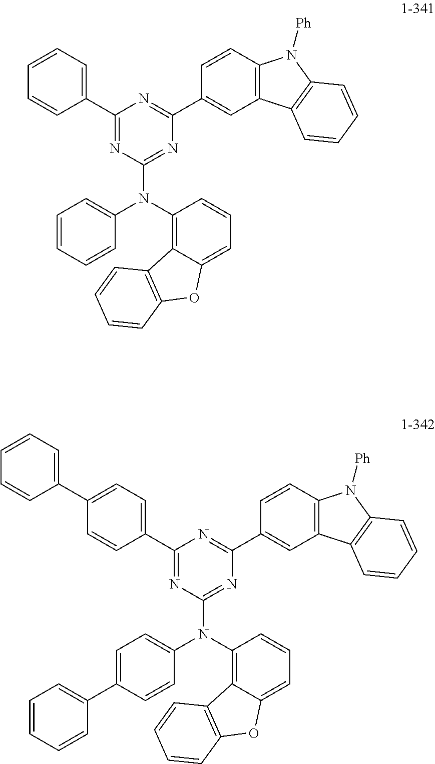 [SMILES: c1ccc(-c2nc(-c3ccc4c(c3)c3ccccc3n4-c3ccccc3)nc(N(c3ccccc3)c3cccc4oc5ccccc5c34)n2)cc1 ; c1ccc(-c2ccc(-c3nc(-c4ccc5c(c4)c4ccccc4n5-c4ccccc4)nc(N(c4ccc(-c5ccccc5)cc4)c4cccc5oc6ccccc6c45)n3)cc2)cc1]

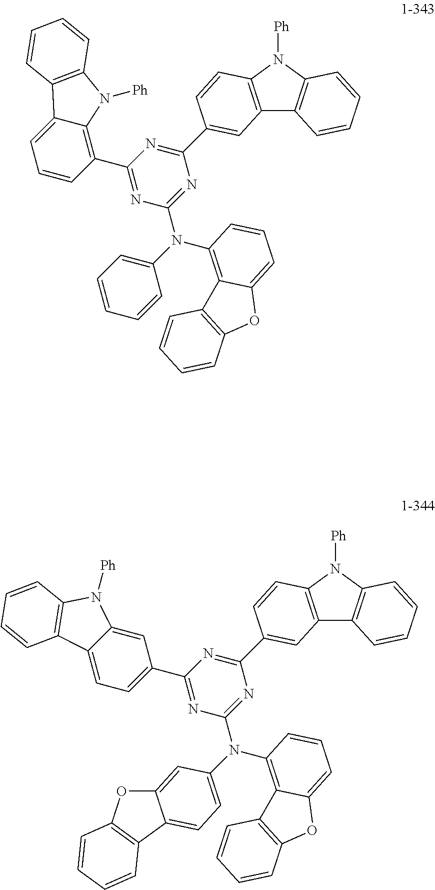 [SMILES: c1ccc(N(c2nc(-c3ccc4c(c3)c3ccccc3n4-c3ccccc3)nc(-c3cccc4c5ccccc5n(-c5ccccc5)c34)n2)c2cccc3oc4ccccc4c23)cc1 ; c1ccc(-n2c3ccccc3c3cc(-c4nc(-c5ccc6c7ccccc7n(-c7ccccc7)c6c5)nc(N(c5ccc6c(c5)oc5ccccc56)c5cccc6oc7ccccc7c56)n4)ccc32)cc1]

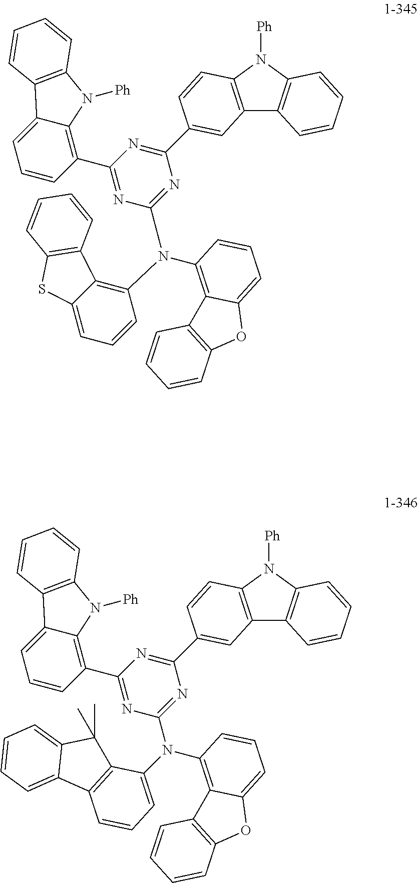 [SMILES: c1ccc(-n2c3ccccc3c3cc(-c4nc(-c5cccc6c7ccccc7n(-c7ccccc7)c56)nc(N(c5cccc6oc7ccccc7c56)c5cccc6sc7ccccc7c56)n4)ccc32)cc1 ; CC1(C)c2ccccc2-c2cccc(N(c3nc(-c4ccc5c(c4)c4ccccc4n5-c4ccccc4)nc(-c4cccc5c6ccccc6n(-c6ccccc6)c45)n3)c3cccc4oc5ccccc5c34)c21]

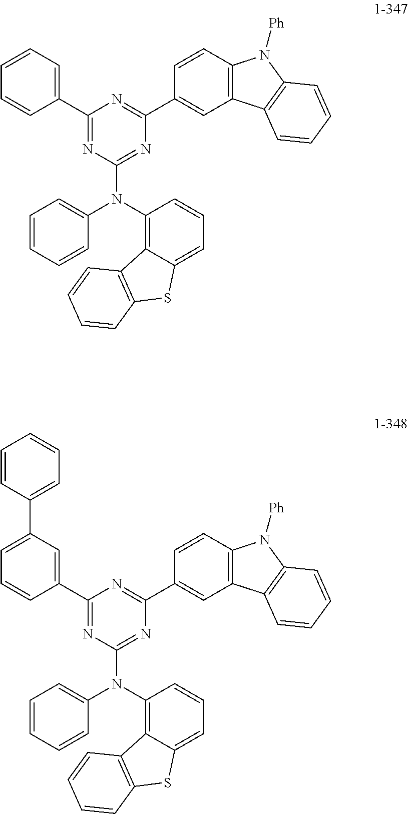 [SMILES: c1ccc(-c2nc(-c3ccc4c(c3)c3ccccc3n4-c3ccccc3)nc(N(c3ccccc3)c3cccc4sc5ccccc5c34)n2)cc1 ; c1ccc(-c2cccc(-c3nc(-c4ccc5c(c4)c4ccccc4n5-c4ccccc4)nc(N(c4ccccc4)c4cccc5sc6ccccc6c45)n3)c2)cc1]

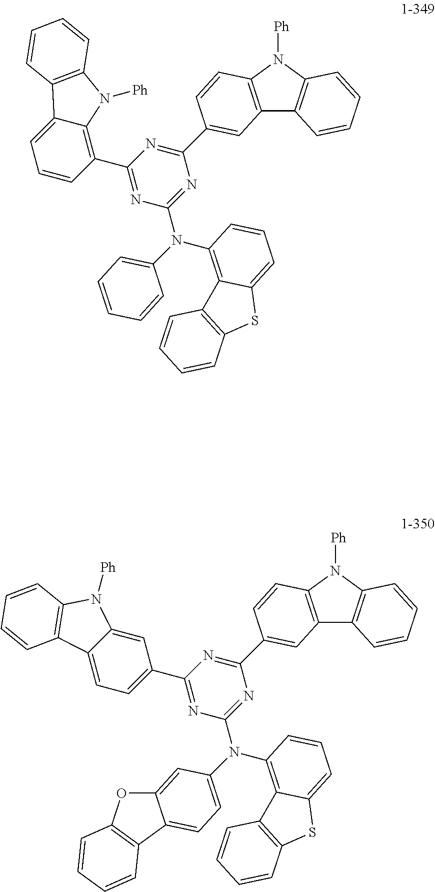 [SMILES: c1ccc(N(c2nc(-c3ccc4c(c3)c3ccccc3n4-c3ccccc3)nc(-c3cccc4c5ccccc5n(-c5ccccc5)c34)n2)c2cccc3sc4ccccc4c23)cc1 ; c1ccc(-n2c3ccccc3c3cc(-c4nc(-c5ccc6c7ccccc7n(-c7ccccc7)c6c5)nc(N(c5ccc6c(c5)oc5ccccc56)c5cccc6sc7ccccc7c56)n4)ccc32)cc1]

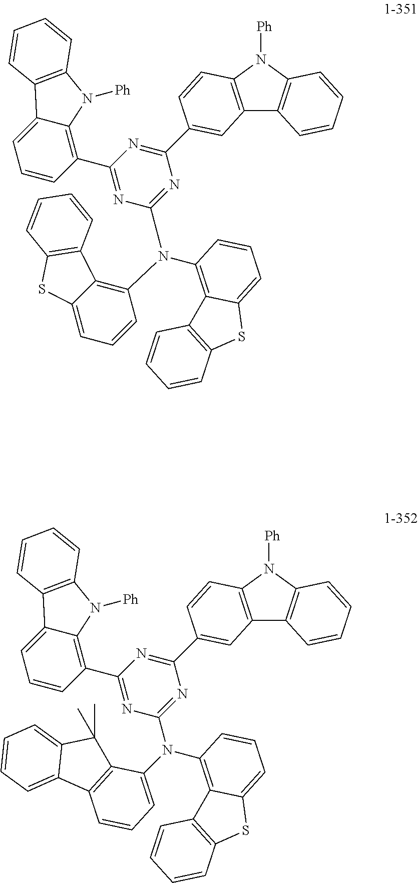 [SMILES: c1ccc(-n2c3ccccc3c3cc(-c4nc(-c5cccc6c7ccccc7n(-c7ccccc7)c56)nc(N(c5cccc6sc7ccccc7c56)c5cccc6sc7ccccc7c56)n4)ccc32)cc1 ; CC1(C)c2ccccc2-c2cccc(N(c3nc(-c4ccc5c(c4)c4ccccc4n5-c4ccccc4)nc(-c4cccc5c6ccccc6n(-c6ccccc6)c45)n3)c3cccc4sc5ccccc5c34)c21]

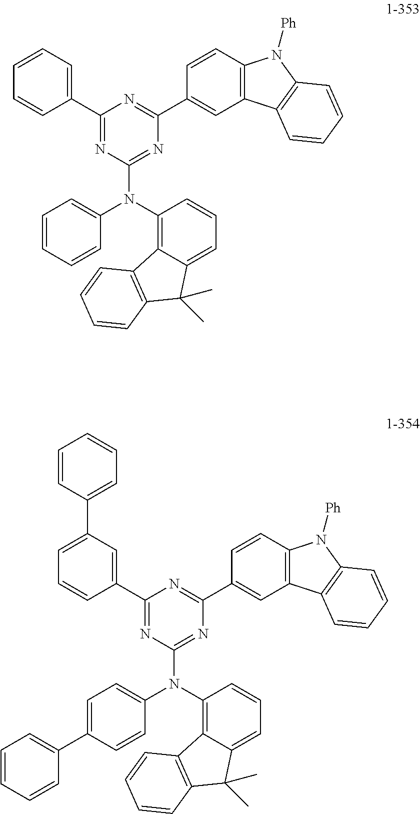 [SMILES: CC1(C)c2ccccc2-c2c(N(c3ccccc3)c3nc(-c4ccccc4)nc(-c4ccc5c(c4)c4ccccc4n5-c4ccccc4)n3)cccc21 ; CC1(C)c2ccccc2-c2c(N(c3ccc(-c4ccccc4)cc3)c3nc(-c4cccc(-c5ccccc5)c4)nc(-c4ccc5c(c4)c4ccccc4n5-c4ccccc4)n3)cccc21]

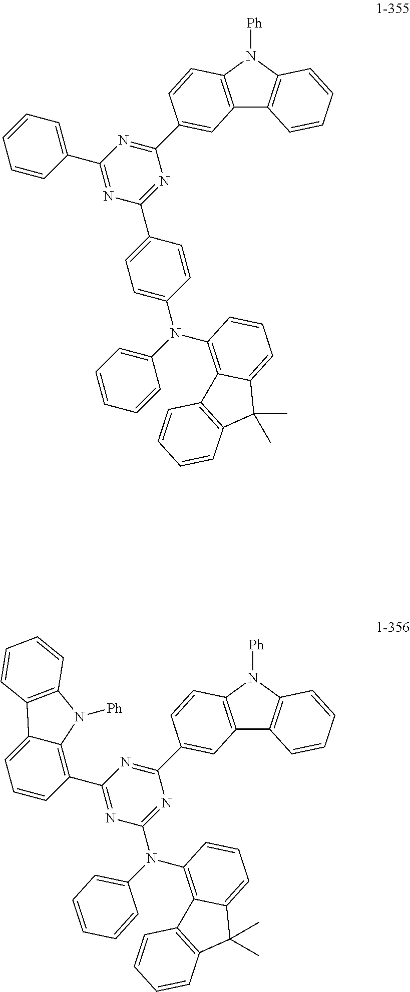 [SMILES: CC1(C)c2ccccc2-c2c(N(c3ccccc3)c3ccc(-c4nc(-c5ccccc5)nc(-c5ccc6c(c5)c5ccccc5n6-c5ccccc5)n4)cc3)cccc21 ; CC1(C)c2ccccc2-c2c(N(c3ccccc3)c3nc(-c4ccc5c(c4)c4ccccc4n5-c4ccccc4)nc(-c4cccc5c6ccccc6n(-c6ccccc6)c45)n3)cccc21]

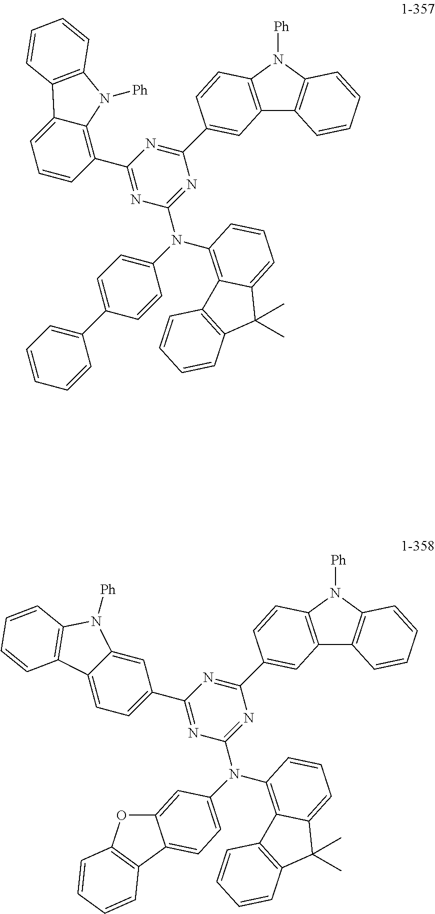 [SMILES: CC1(C)c2ccccc2-c2c(N(c3ccc(-c4ccccc4)cc3)c3nc(-c4ccc5c(c4)c4ccccc4n5-c4ccccc4)nc(-c4cccc5c6ccccc6n(-c6ccccc6)c45)n3)cccc21 ; CC1(C)c2ccccc2-c2c(N(c3ccc4c(c3)oc3ccccc34)c3nc(-c4ccc5c(c4)c4ccccc4n5-c4ccccc4)nc(-c4ccc5c6ccccc6n(-c6ccccc6)c5c4)n3)cccc21]

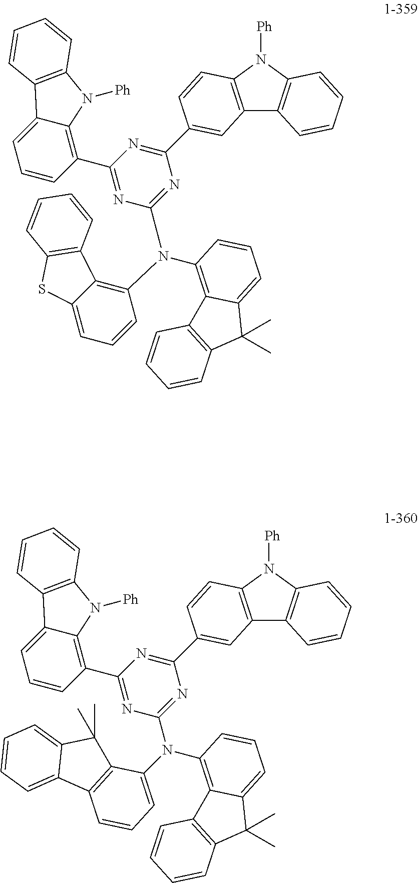 [SMILES: CC1(C)c2ccccc2-c2c(N(c3nc(-c4ccc5c(c4)c4ccccc4n5-c4ccccc4)nc(-c4cccc5c6ccccc6n(-c6ccccc6)c45)n3)c3cccc4sc5ccccc5c34)cccc21 ; CC1(C)c2ccccc2-c2c(N(c3nc(-c4ccc5c(c4)c4ccccc4n5-c4ccccc4)nc(-c4cccc5c6ccccc6n(-c6ccccc6)c45)n3)c3cccc4c3C(C)(C)c3ccccc3-4)cccc21]

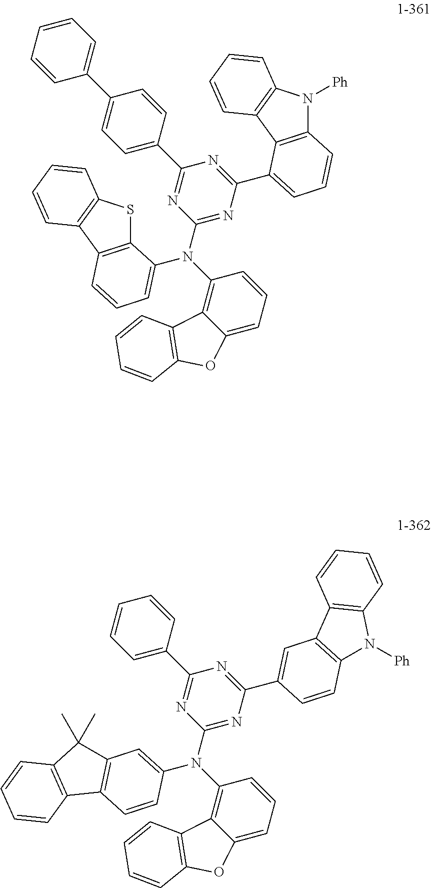 [SMILES: c1ccc(-c2ccc(-c3nc(-c4cccc5c4c4ccccc4n5-c4ccccc4)nc(N(c4cccc5c4sc4ccccc45)c4cccc5oc6ccccc6c45)n3)cc2)cc1 ; CC1(C)c2ccccc2-c2ccc(N(c3nc(-c4ccccc4)nc(-c4ccc5c(c4)c4ccccc4n5-c4ccccc4)n3)c3cccc4oc5ccccc5c34)cc21]

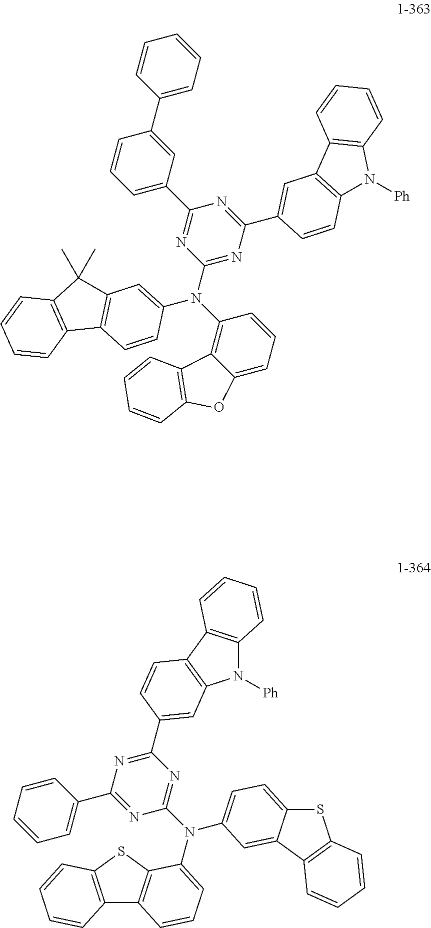 [SMILES: CC1(C)c2ccccc2-c2ccc(N(c3nc(-c4cccc(-c5ccccc5)c4)nc(-c4ccc5c(c4)c4ccccc4n5-c4ccccc4)n3)c3cccc4oc5ccccc5c34)cc21 ; c1ccc(-c2nc(-c3ccc4c5ccccc5n(-c5ccccc5)c4c3)nc(N(c3ccc4sc5ccccc5c4c3)c3cccc4c3sc3ccccc34)n2)cc1]

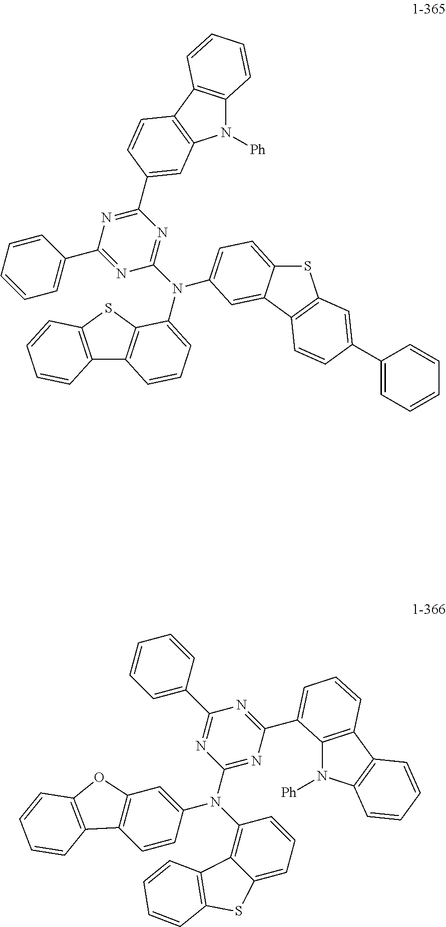 [SMILES: c1ccc(-c2ccc3c(c2)sc2ccc(N(c4nc(-c5ccccc5)nc(-c5ccc6c7ccccc7n(-c7ccccc7)c6c5)n4)c4cccc5c4sc4ccccc45)cc23)cc1 ; c1ccc(-c2nc(-c3cccc4c5ccccc5n(-c5ccccc5)c34)nc(N(c3ccc4c(c3)oc3ccccc34)c3cccc4sc5ccccc5c34)n2)cc1]

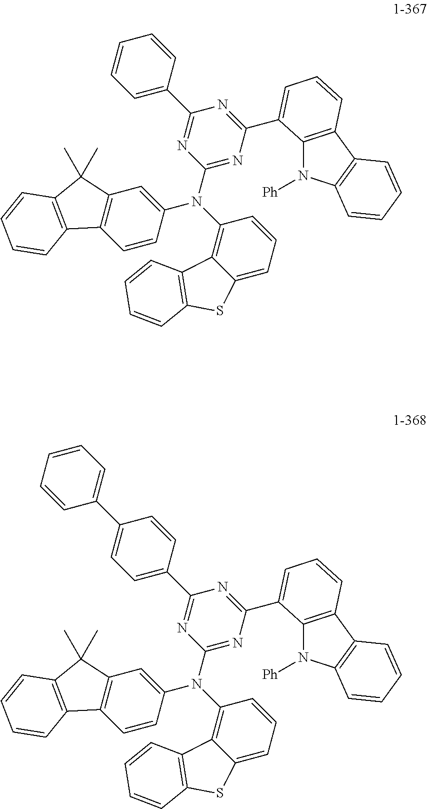 [SMILES: CC1(C)c2ccccc2-c2ccc(N(c3nc(-c4ccccc4)nc(-c4cccc5c6ccccc6n(-c6ccccc6)c45)n3)c3cccc4sc5ccccc5c34)cc21 ; CC1(C)c2ccccc2-c2ccc(N(c3nc(-c4ccc(-c5ccccc5)cc4)nc(-c4cccc5c6ccccc6n(-c6ccccc6)c45)n3)c3cccc4sc5ccccc5c34)cc21]

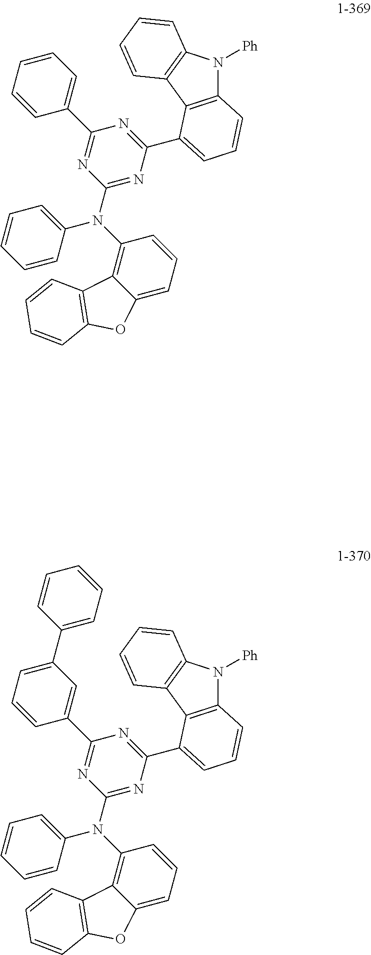 [SMILES: c1ccc(-c2nc(-c3cccc4c3c3ccccc3n4-c3ccccc3)nc(N(c3ccccc3)c3cccc4oc5ccccc5c34)n2)cc1 ; c1ccc(-c2cccc(-c3nc(-c4cccc5c4c4ccccc4n5-c4ccccc4)nc(N(c4ccccc4)c4cccc5oc6ccccc6c45)n3)c2)cc1]

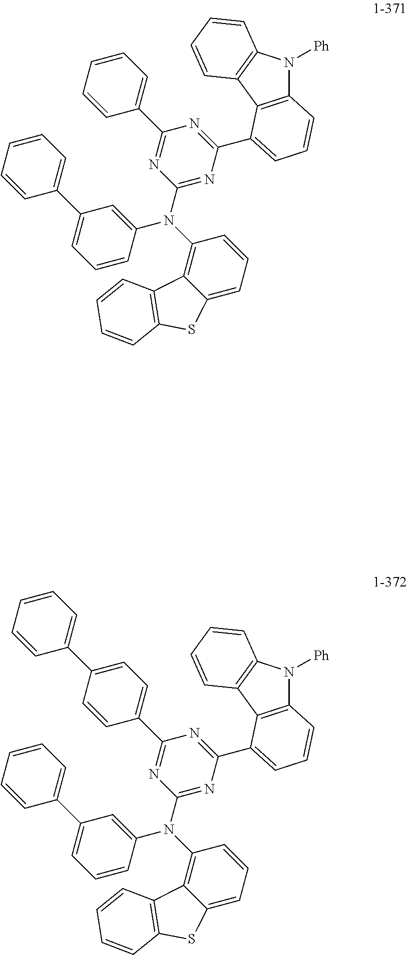 [SMILES: c1ccc(-c2cccc(N(c3nc(-c4ccccc4)nc(-c4cccc5c4c4ccccc4n5-c4ccccc4)n3)c3cccc4sc5ccccc5c34)c2)cc1 ; c1ccc(-c2ccc(-c3nc(-c4cccc5c4c4ccccc4n5-c4ccccc4)nc(N(c4cccc(-c5ccccc5)c4)c4cccc5sc6ccccc6c45)n3)cc2)cc1]

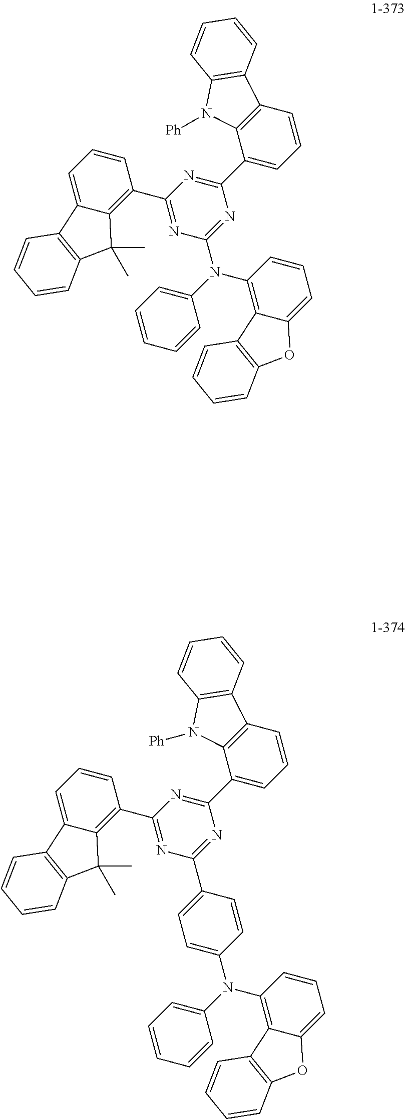 [SMILES: CC1(C)c2ccccc2-c2cccc(-c3nc(-c4cccc5c6ccccc6n(-c6ccccc6)c45)nc(N(c4ccccc4)c4cccc5oc6ccccc6c45)n3)c21 ; CC1(C)c2ccccc2-c2cccc(-c3nc(-c4ccc(N(c5ccccc5)c5cccc6oc7ccccc7c56)cc4)nc(-c4cccc5c6ccccc6n(-c6ccccc6)c45)n3)c21]

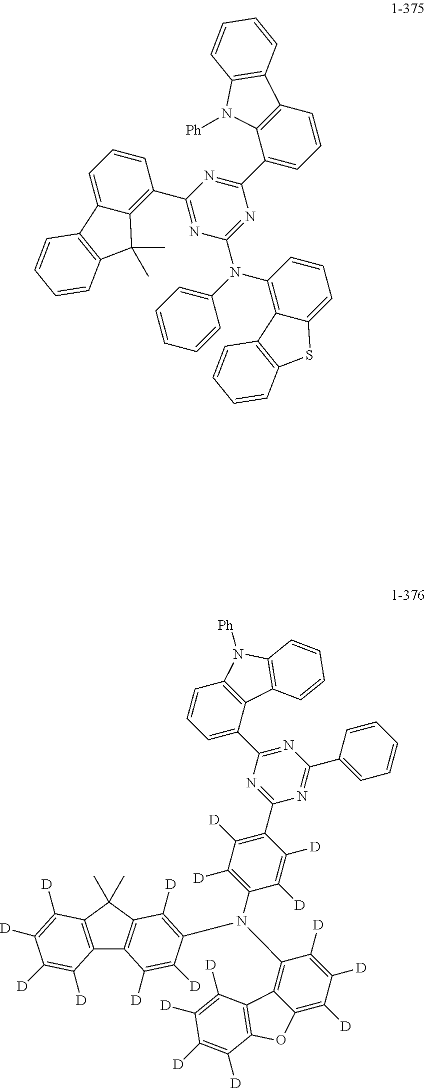 [SMILES: CC1(C)c2ccccc2-c2cccc(-c3nc(-c4cccc5c6ccccc6n(-c6ccccc6)c45)nc(N(c4ccccc4)c4cccc5sc6ccccc6c45)n3)c21 ; [2H]c1c([2H])c([2H])c2c(c1[2H])-c1c([2H])c([2H])c(N(c3c([2H])c([2H])c(-c4nc(-c5ccccc5)nc(-c5cccc6c5c5ccccc5n6-c5ccccc5)n4)c([2H])c3[2H])c3c([2H])c([2H])c([2H])c4oc5c([2H])c([2H])c([2H])c([2H])c5c34)c([2H])c1C2(C)C]

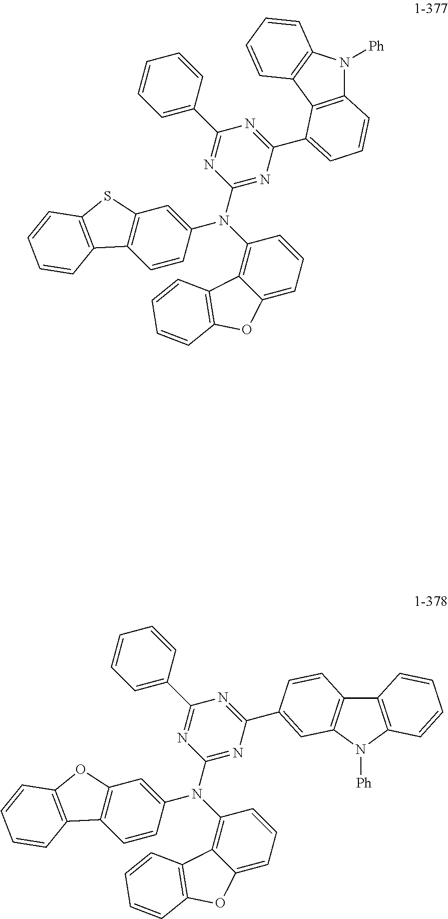 [SMILES: c1ccc(-c2nc(-c3cccc4c3c3ccccc3n4-c3ccccc3)nc(N(c3ccc4c(c3)sc3ccccc34)c3cccc4oc5ccccc5c34)n2)cc1 ; c1ccc(-c2nc(-c3ccc4c5ccccc5n(-c5ccccc5)c4c3)nc(N(c3ccc4c(c3)oc3ccccc34)c3cccc4oc5ccccc5c34)n2)cc1]

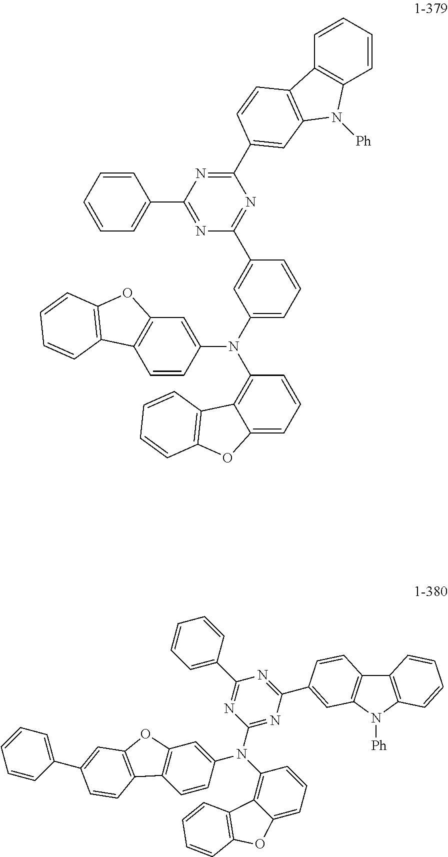 [SMILES: c1ccc(-c2nc(-c3cccc(N(c4ccc5c(c4)oc4ccccc45)c4cccc5oc6ccccc6c45)c3)nc(-c3ccc4c5ccccc5n(-c5ccccc5)c4c3)n2)cc1 ; c1ccc(-c2ccc3c(c2)oc2cc(N(c4nc(-c5ccccc5)nc(-c5ccc6c7ccccc7n(-c7ccccc7)c6c5)n4)c4cccc5oc6ccccc6c45)ccc23)cc1]

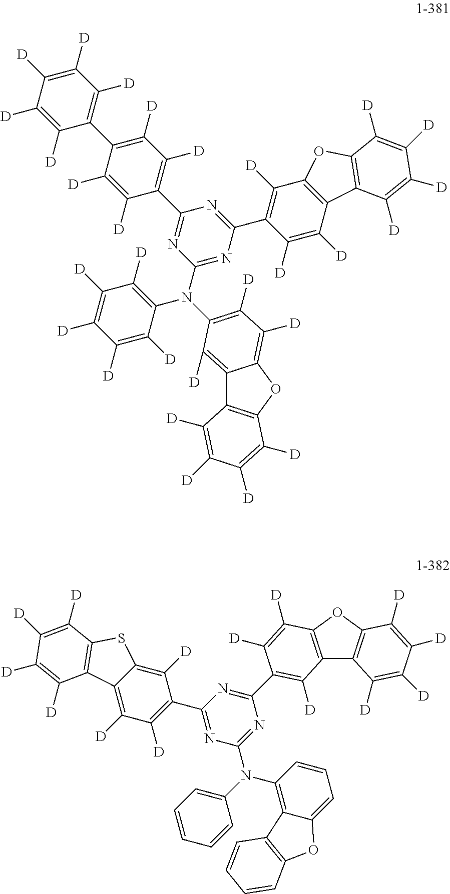 [SMILES: [2H]c1c([2H])c([2H])c(-c2c([2H])c([2H])c(-c3nc(-c4c([2H])c([2H])c5c(oc6c([2H])c([2H])c([2H])c([2H])c65)c4[2H])nc(N(c4c([2H])c([2H])c([2H])c([2H])c4[2H])c4c([2H])c([2H])c5oc6c([2H])c([2H])c([2H])c([2H])c6c5c4[2H])n3)c([2H])c2[2H])c([2H])c1[2H] ; [2H]c1c([2H])c([2H])c2c(oc3c([2H])c([2H])c(-c4nc(-c5c([2H])c([2H])c6c(sc7c([2H])c([2H])c([2H])c([2H])c76)c5[2H])nc(N(c5ccccc5)c5cccc6oc7ccccc7c56)n4)c([2H])c32)c1[2H]]

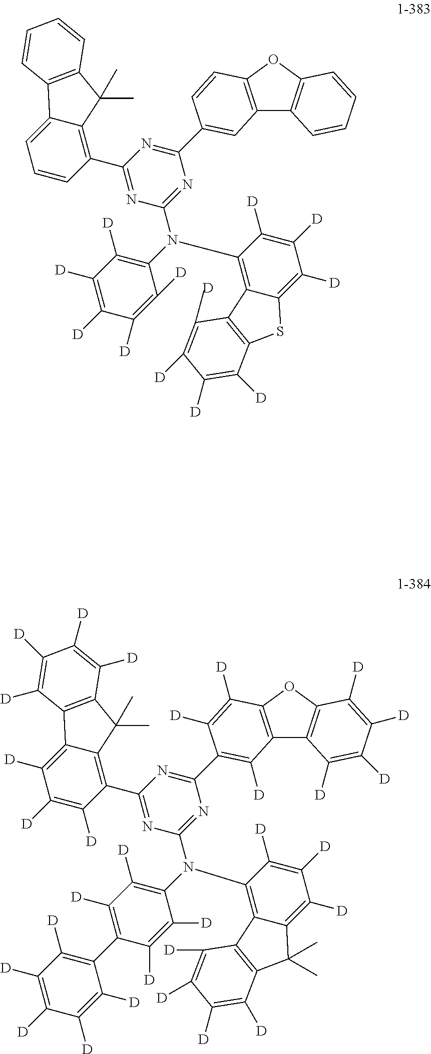

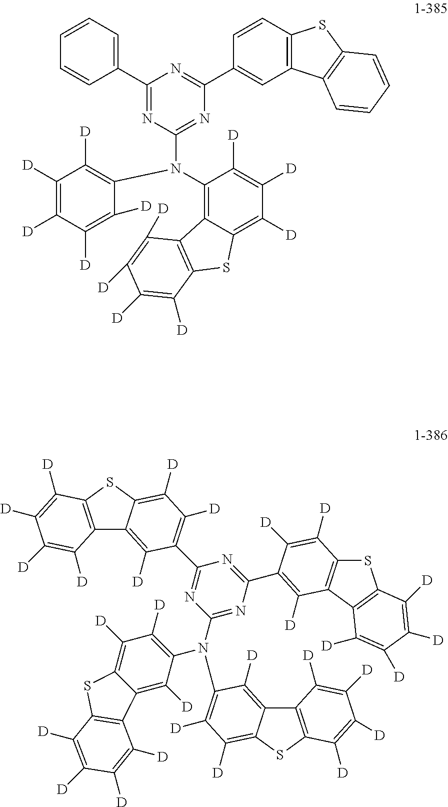 [SMILES: [2H]c1c([2H])c([2H])c(N(c2nc(-c3ccccc3)nc(-c3ccc4sc5ccccc5c4c3)n2)c2c([2H])c([2H])c([2H])c3sc4c([2H])c([2H])c([2H])c([2H])c4c23)c([2H])c1[2H] ; [2H]c1c([2H])c([2H])c2c(sc3c([2H])c([2H])c(-c4nc(-c5c([2H])c([2H])c6sc7c([2H])c([2H])c([2H])c([2H])c7c6c5[2H])nc(N(c5c([2H])c([2H])c6sc7c([2H])c([2H])c([2H])c([2H])c7c6c5[2H])c5c([2H])c([2H])c6sc7c([2H])c([2H])c([2H])c([2H])c7c6c5[2H])n4)c([2H])c32)c1[2H]]

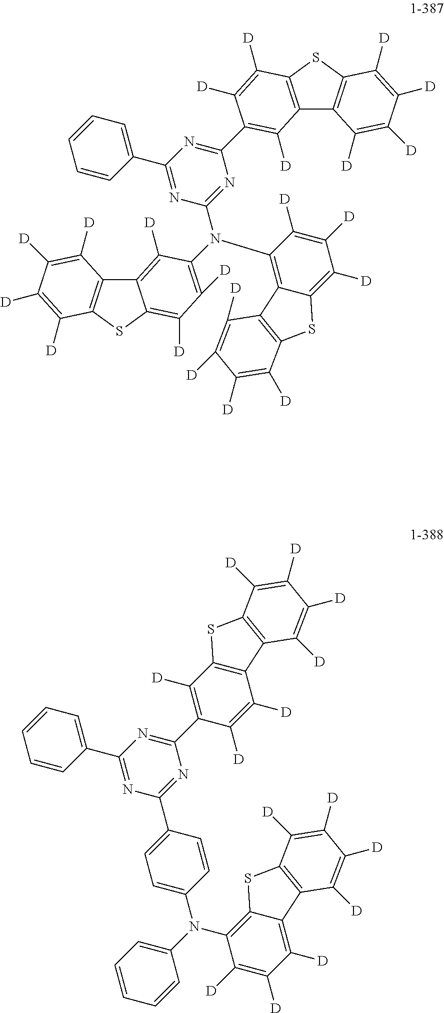 [SMILES: [2H]c1c([2H])c([2H])c2c(sc3c([2H])c([2H])c(-c4nc(-c5ccccc5)nc(N(c5c([2H])c([2H])c6sc7c([2H])c([2H])c([2H])c([2H])c7c6c5[2H])c5c([2H])c([2H])c([2H])c6sc7c([2H])c([2H])c([2H])c([2H])c7c56)n4)c([2H])c32)c1[2H] ; [2H]c1c([2H])c([2H])c2c(sc3c([2H])c(-c4nc(-c5ccccc5)nc(-c5ccc(N(c6ccccc6)c6c([2H])c([2H])c([2H])c7c6sc6c([2H])c([2H])c([2H])c([2H])c67)cc5)n4)c([2H])c([2H])c32)c1[2H]]

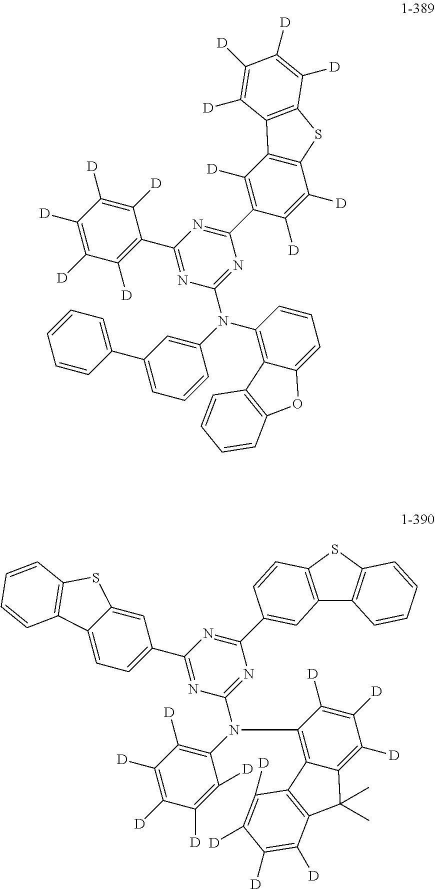 [SMILES: [2H]c1c([2H])c([2H])c(-c2nc(-c3c([2H])c([2H])c4sc5c([2H])c([2H])c([2H])c([2H])c5c4c3[2H])nc(N(c3cccc(-c4ccccc4)c3)c3cccc4oc5ccccc5c34)n2)c([2H])c1[2H] ; [2H]c1c([2H])c([2H])c(N(c2nc(-c3ccc4c(c3)sc3ccccc34)nc(-c3ccc4sc5ccccc5c4c3)n2)c2c([2H])c([2H])c([2H])c3c2-c2c([2H])c([2H])c([2H])c([2H])c2C3(C)C)c([2H])c1[2H]]

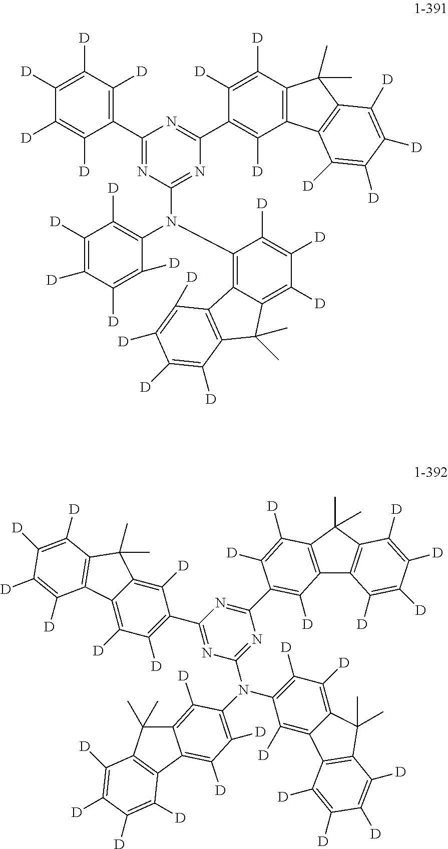

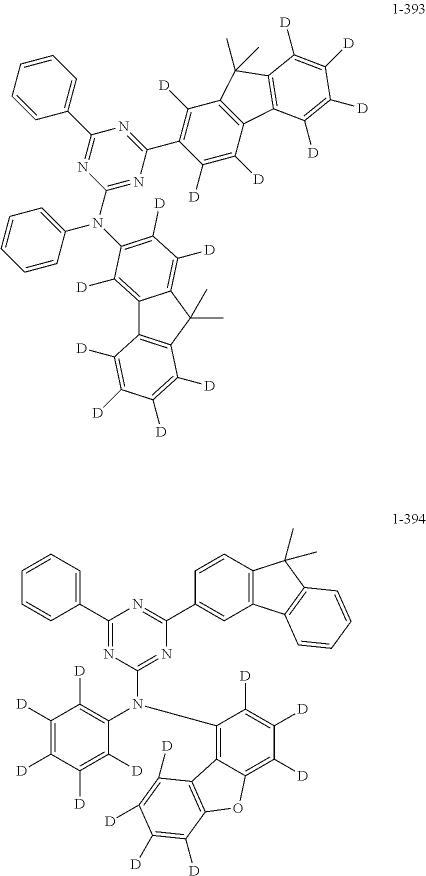 [SMILES: [2H]c1c([2H])c([2H])c2c(c1[2H])-c1c([2H])c(N(c3ccccc3)c3nc(-c4ccccc4)nc(-c4c([2H])c([2H])c5c(c4[2H])C(C)(C)c4c([2H])c([2H])c([2H])c([2H])c4-5)n3)c([2H])c([2H])c1C2(C)C ; [2H]c1c([2H])c([2H])c(N(c2nc(-c3ccccc3)nc(-c3ccc4c(c3)-c3ccccc3C4(C)C)n2)c2c([2H])c([2H])c([2H])c3oc4c([2H])c([2H])c([2H])c([2H])c4c23)c([2H])c1[2H]]

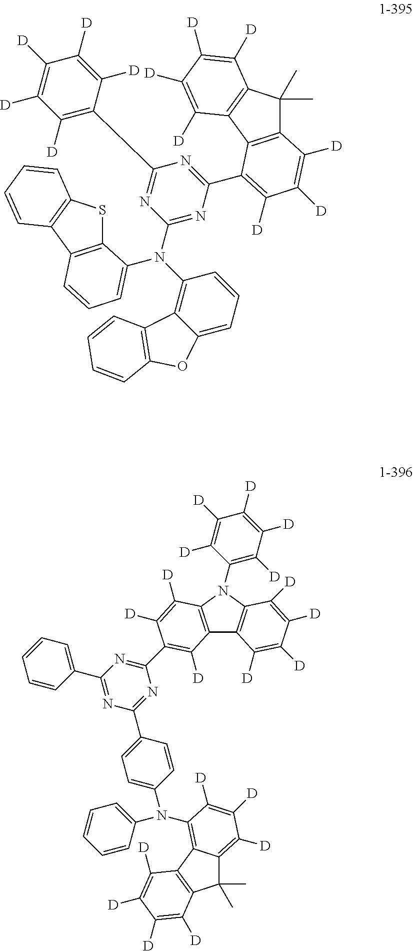 [SMILES: [2H]c1c([2H])c([2H])c(-c2nc(-c3c([2H])c([2H])c([2H])c4c3-c3c([2H])c([2H])c([2H])c([2H])c3C4(C)C)nc(N(c3cccc4c3sc3ccccc34)c3cccc4oc5ccccc5c34)n2)c([2H])c1[2H] ; [2H]c1c([2H])c([2H])c(-n2c3c([2H])c([2H])c([2H])c([2H])c3c3c([2H])c(-c4nc(-c5ccccc5)nc(-c5ccc(N(c6ccccc6)c6c([2H])c([2H])c([2H])c7c6-c6c([2H])c([2H])c([2H])c([2H])c6C7(C)C)cc5)n4)c([2H])c([2H])c32)c([2H])c1[2H]]

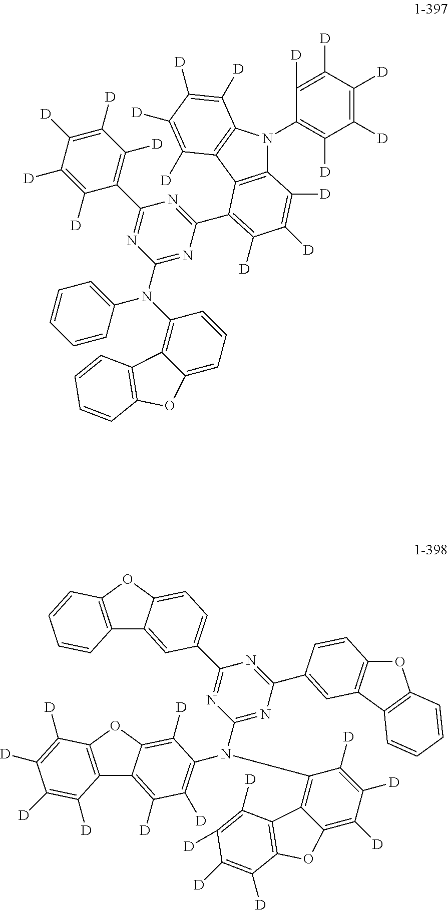 [SMILES: [2H]c1c([2H])c([2H])c(-c2nc(-c3c([2H])c([2H])c([2H])c4c3c3c([2H])c([2H])c([2H])c([2H])c3n4-c3c([2H])c([2H])c([2H])c([2H])c3[2H])nc(N(c3ccccc3)c3cccc4oc5ccccc5c34)n2)c([2H])c1[2H] ; [2H]c1c([2H])c([2H])c2c(oc3c([2H])c(N(c4nc(-c5ccc6oc7ccccc7c6c5)nc(-c5ccc6oc7ccccc7c6c5)n4)c4c([2H])c([2H])c([2H])c5oc6c([2H])c([2H])c([2H])c([2H])c6c45)c([2H])c([2H])c32)c1[2H]]

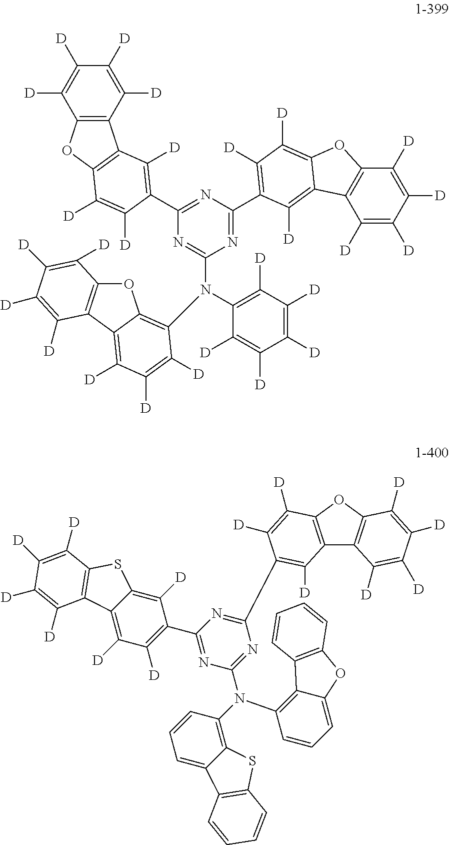 [SMILES: [2H]c1c([2H])c([2H])c(N(c2nc(-c3c([2H])c([2H])c4oc5c([2H])c([2H])c([2H])c([2H])c5c4c3[2H])nc(-c3c([2H])c([2H])c4oc5c([2H])c([2H])c([2H])c([2H])c5c4c3[2H])n2)c2c([2H])c([2H])c([2H])c3c2oc2c([2H])c([2H])c([2H])c([2H])c23)c([2H])c1[2H] ; [2H]c1c([2H])c([2H])c2c(oc3c([2H])c([2H])c(-c4nc(-c5c([2H])c([2H])c6c(sc7c([2H])c([2H])c([2H])c([2H])c76)c5[2H])nc(N(c5cccc6c5sc5ccccc56)c5cccc6oc7ccccc7c56)n4)c([2H])c32)c1[2H]]

In the compounds, Ph is a phenyl group.

In addition, by introducing various substituents to the structure of Chemical Formula 1, compounds having unique properties of the introduced substituents may be synthesized. For example, by introducing substituents normally used as hole injection layer materials, hole transfer layer materials, light emitting layer materials, electron transfer layer materials and charge generation layer materials used for manufacturing an organic light emitting device to the core structure, materials satisfying conditions required for each organic material layer may be synthesized.

In addition, by introducing various substituents to the structure of Chemical Formula 1, the energy band gap may be finely controlled, and meanwhile, properties at interfaces between organic materials are enhanced, and material applications may become diverse.

One embodiment of the present specification provides an organic light emitting device including a first electrode; a second electrode; and one or more organic material layers provided between the first electrode and the second electrode, wherein one or more layers of the organic material layers include the heterocyclic compound of Chemical Formula 1.

In one embodiment of the present specification, the first electrode may be an anode, and the second electrode may be a cathode.

In another embodiment of the present specification, the first electrode may be a cathode, and the second electrode may be an anode.

In one embodiment of the present specification, the organic light emitting device may be a blue organic light emitting device, and the heterocyclic compound of Chemical Formula 1 may be used as a material of the blue organic light emitting device. For example, the heterocyclic compound of Chemical Formula 1 may be included in a light emitting layer of the blue organic light emitting device.

In one embodiment of the present specification, the organic light emitting device may be a green organic light emitting device, and the heterocyclic compound of Chemical Formula 1 may be used as a material of the green organic light emitting device. For example, the heterocyclic compound of Chemical Formula 1 may be included in a light emitting layer of the green organic light emitting device.

In one embodiment of the present specification, the organic light emitting device may be a red organic light emitting device, and the heterocyclic compound of Chemical Formula 1 may be used as a material of the red organic light emitting device. For example, the heterocyclic compound of Chemical Formula 1 may be included in a light emitting layer of the red organic light emitting device.

The organic light emitting device of the present specification may be manufactured using common organic light emitting device manufacturing methods and materials except that one or more organic material layers are formed using the heterocyclic compound described above.

The heterocyclic compound may be formed into an organic material layer using a solution coating method as well as a vacuum deposition method when manufacturing the organic light emitting device. Herein, the solution coating method means spin coating, dip coating, inkjet printing, screen printing, a spray method, roll coating and the like, but is not limited thereto.

The organic material layer of the organic light emitting device of the present specification may be formed in a single layer structure, but may be formed in a multilayer structure in which two or more organic material layers are laminated. For example, the organic light emitting device of the present disclosure may have a structure including a hole injection layer, a hole transfer layer, a light emitting layer, an electron transfer layer, an electron injection layer and the like as the organic material layer. However, the structure of the organic light emitting device is not limited thereto, and may include a smaller number of organic material layers.

In the organic light emitting device of the present specification, the organic material layer includes a light emitting layer, and the light emitting layer may include the heterocyclic compound of Chemical Formula 1.

In the organic light emitting device of the present specification, the organic material layer includes a light emitting layer, the light emitting layer includes a host, and the host may include the heterocyclic compound of Chemical Formula 1.

In the organic light emitting device of the present specification, the organic material layer may further include a compound of the following Chemical Formula 2 together with the heterocyclic compound of Chemical Formula 1.

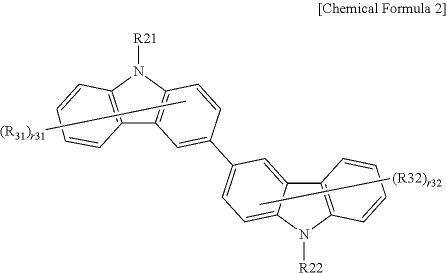

In Chemical Formula 2,
R21 and R22 are the same as or different from each other, and each independently deuterium; a halogen group; a cyano group; a substituted or unsubstituted C1 to C60 alkyl group; a substituted or unsubstituted C3 to C60 cycloalkyl group; or a substituted or unsubstituted C6 to C60 aryl group,
R31 and R32 are the same as or different from each other, and each independently hydrogen; deuterium; a halogen group; a cyano group; a substituted or unsubstituted C1 to C60 alkyl group; a substituted or unsubstituted C3 to C60 cycloalkyl group; a substituted or unsubstituted C6 to C60 aryl group; or a substituted or unsubstituted C2 to C60 heteroaryl group,
r31 is an integer of 0 to 7, and when 2 or greater, R31s are the same as or different from each other, and
r32 is an integer of 0 to 7, and when 2 or greater, R32s are the same as or different from each other.

In the organic light emitting device of the present specification, the organic material layer may include the heterocyclic compound of Chemical Formula 1 and the compound of Chemical Formula 2 at the same time.

In one embodiment of the present specification, R21 and R22 are the same as or different from each other, and may be each independently deuterium; a halogen group; a cyano group; or a substituted or unsubstituted C6 to C60 aryl group.

In one embodiment of the present specification, R21 and R22 are the same as or different from each other, and may be each independently deuterium; a halogen group; a cyano group; or a substituted or unsubstituted C6 to C40 aryl group.

In one embodiment of the present specification, R21 and R22 are the same as or different from each other, and may be each independently deuterium; a halogen group; a cyano group; or a substituted or unsubstituted C6 to C30 aryl group.

In one embodiment of the present specification, R21 and R22 are the same as or different from each other, and may be each independently deuterium; a halogen group; a cyano group; a substituted or unsubstituted phenyl group; a substituted or unsubstituted biphenyl group; a substituted or unsubstituted terphenyl group; a substituted or unsubstituted naphthyl group; a substituted or unsubstituted fluorenyl group; or a substituted or unsubstituted triphenylenyl group.

In one embodiment of the present specification, R21 and R22 are the same as or different from each other, and may be each independently deuterium; a halogen group; a cyano group; a phenyl group unsubstituted or substituted with a cyano group or a silyl group; a biphenyl group; a terphenyl group; a naphthyl group; a fluorenyl group unsubstituted or substituted with an alkyl group or an aryl group; 9,9′-spirobi[fluorene]; or a triphenylenyl group.

In one embodiment of the present specification, R21 and R22 are the same as or different from each other, and may be each independently deuterium; a halogen group; a cyano group; a phenyl group unsubstituted or substituted with a cyano group or a triphenylsilyl group

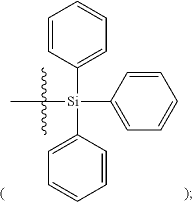 [SMILES: *[Si](c1ccccc1)(c1ccccc1)c1ccccc1]

a biphenyl group; a terphenyl group; a naphthyl group; a fluorenyl group unsubstituted or substituted with a methyl group or a phenyl group; 9,9′-spirobi[fluorene]; or a triphenylenyl group.

In one embodiment of the present specification, R31 and R32 are the same as or different from each other, and may be each independently hydrogen; or deuterium.

In one embodiment of the present specification, Chemical Formula 2 may be represented by any one of the following compounds, but is not limited thereto.

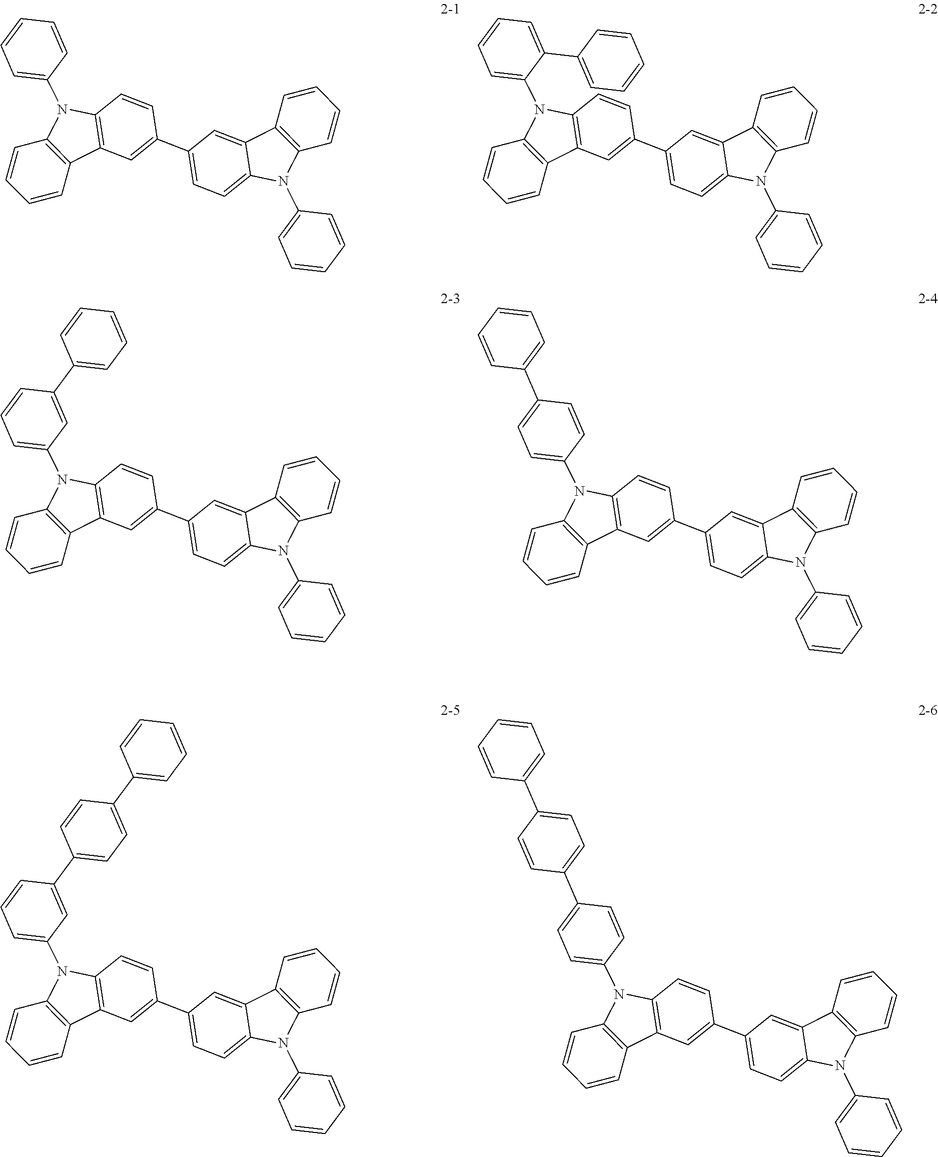 [SMILES: c1ccc(-n2c3ccccc3c3cc(-c4ccc5c(c4)c4ccccc4n5-c4ccccc4)ccc32)cc1 ; c1ccc(-c2ccccc2-n2c3ccccc3c3cc(-c4ccc5c(c4)c4ccccc4n5-c4ccccc4)ccc32)cc1 ; c1ccc(-c2cccc(-n3c4ccccc4c4cc(-c5ccc6c(c5)c5ccccc5n6-c5ccccc5)ccc43)c2)cc1 ; c1ccc(-c2ccc(-n3c4ccccc4c4cc(-c5ccc6c(c5)c5ccccc5n6-c5ccccc5)ccc43)cc2)cc1 ; c1ccc(-c2ccc(-c3cccc(-n4c5ccccc5c5cc(-c6ccc7c(c6)c6ccccc6n7-c6ccccc6)ccc54)c3)cc2)cc1 ; c1ccc(-c2ccc(-c3ccc(-n4c5ccccc5c5cc(-c6ccc7c(c6)c6ccccc6n7-c6ccccc6)ccc54)cc3)cc2)cc1]

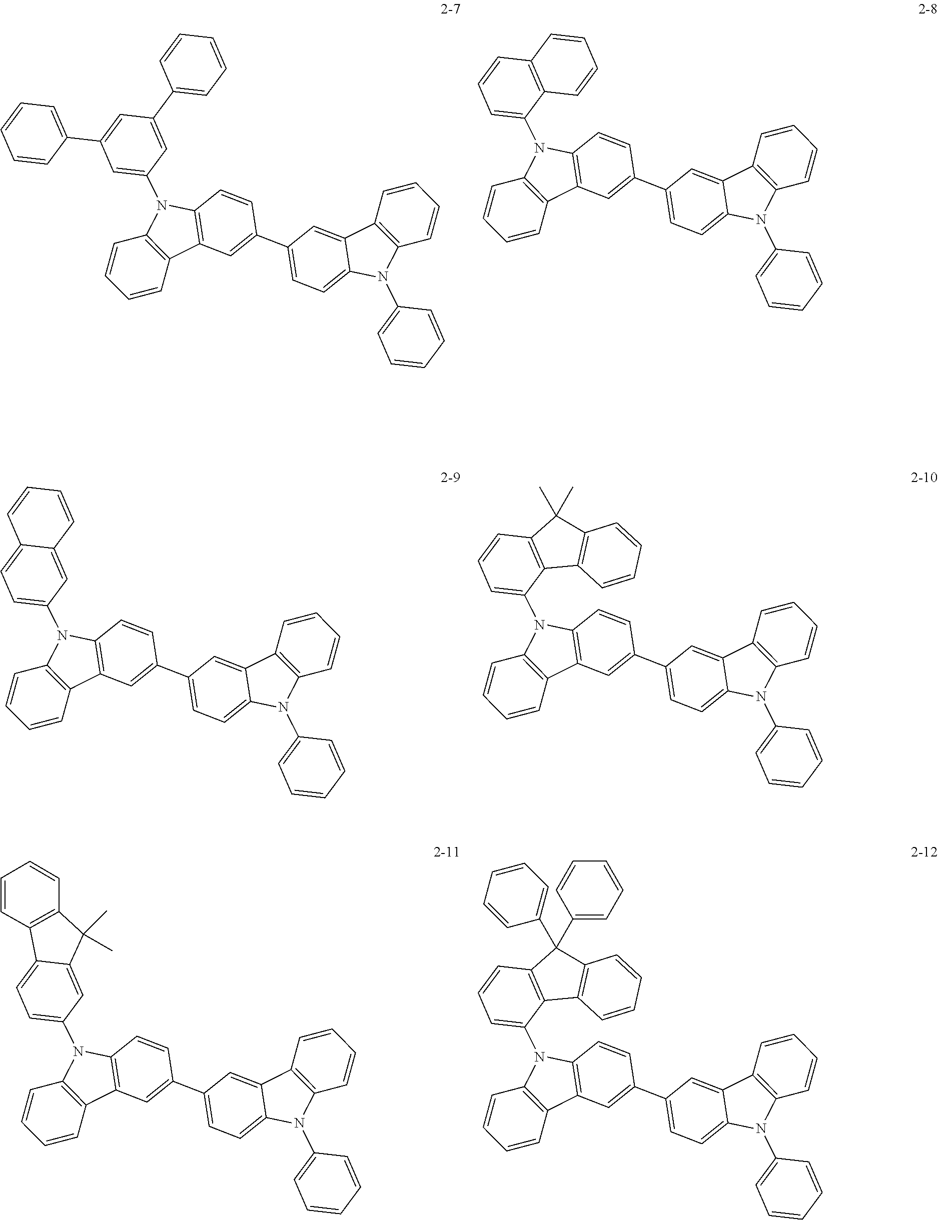

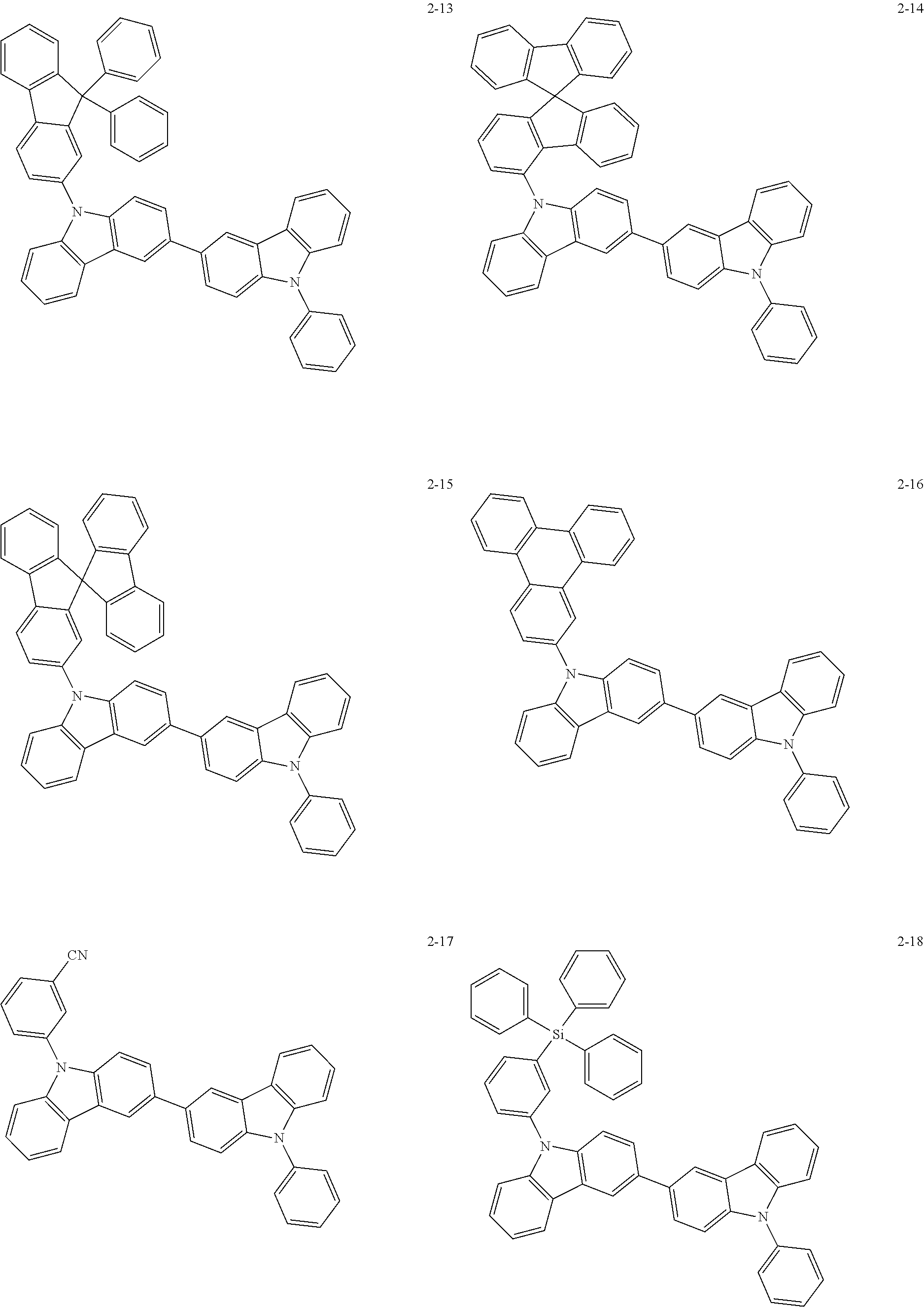

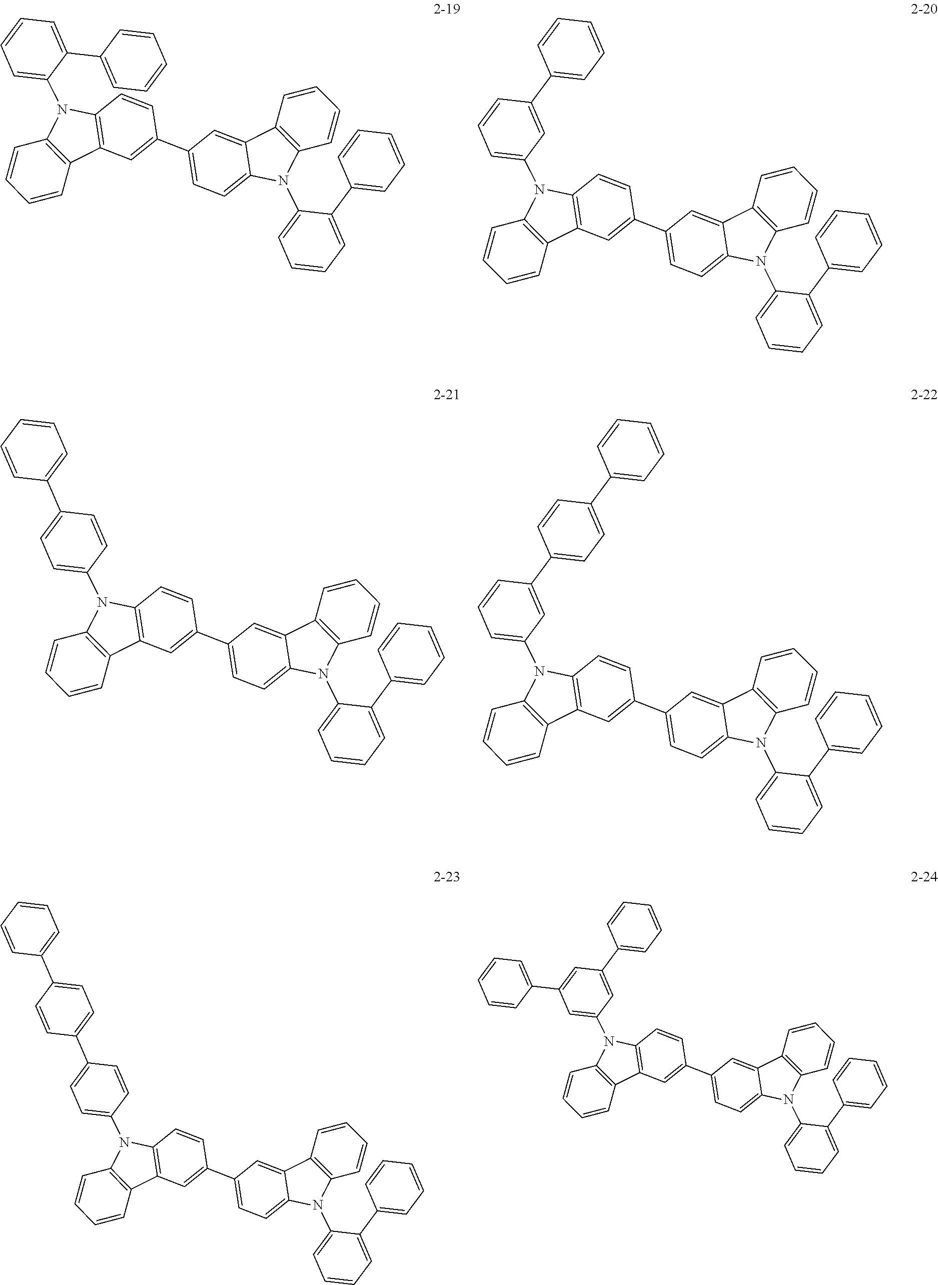

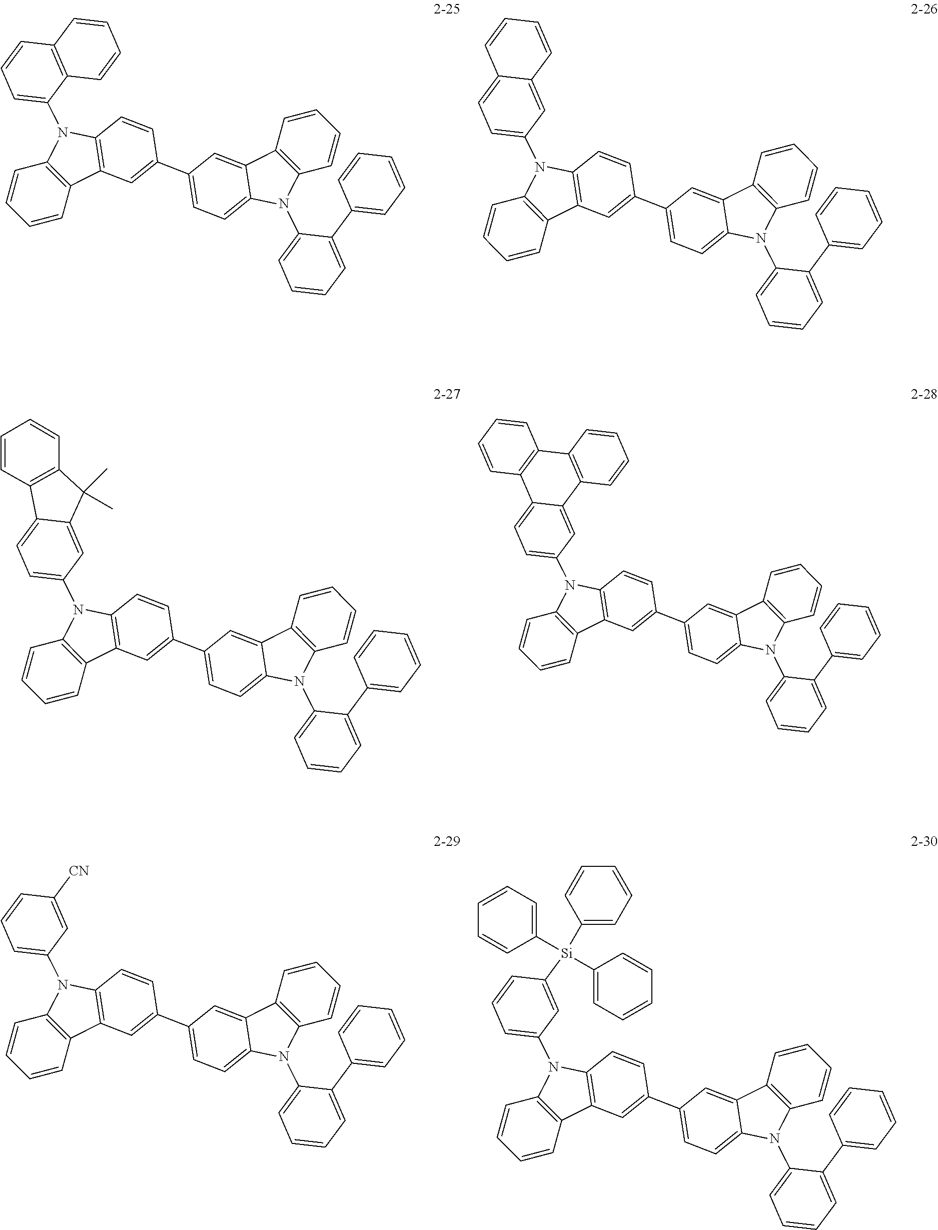

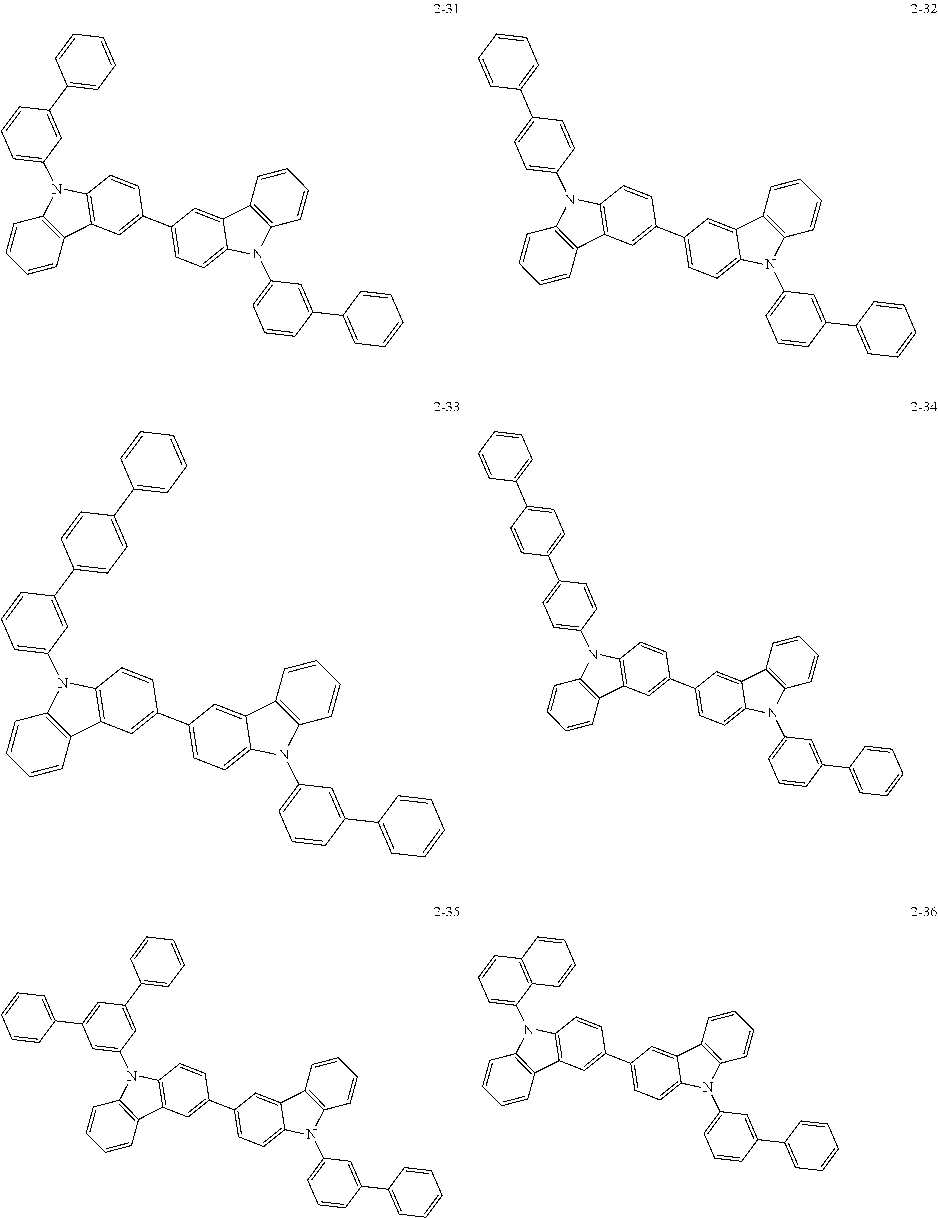

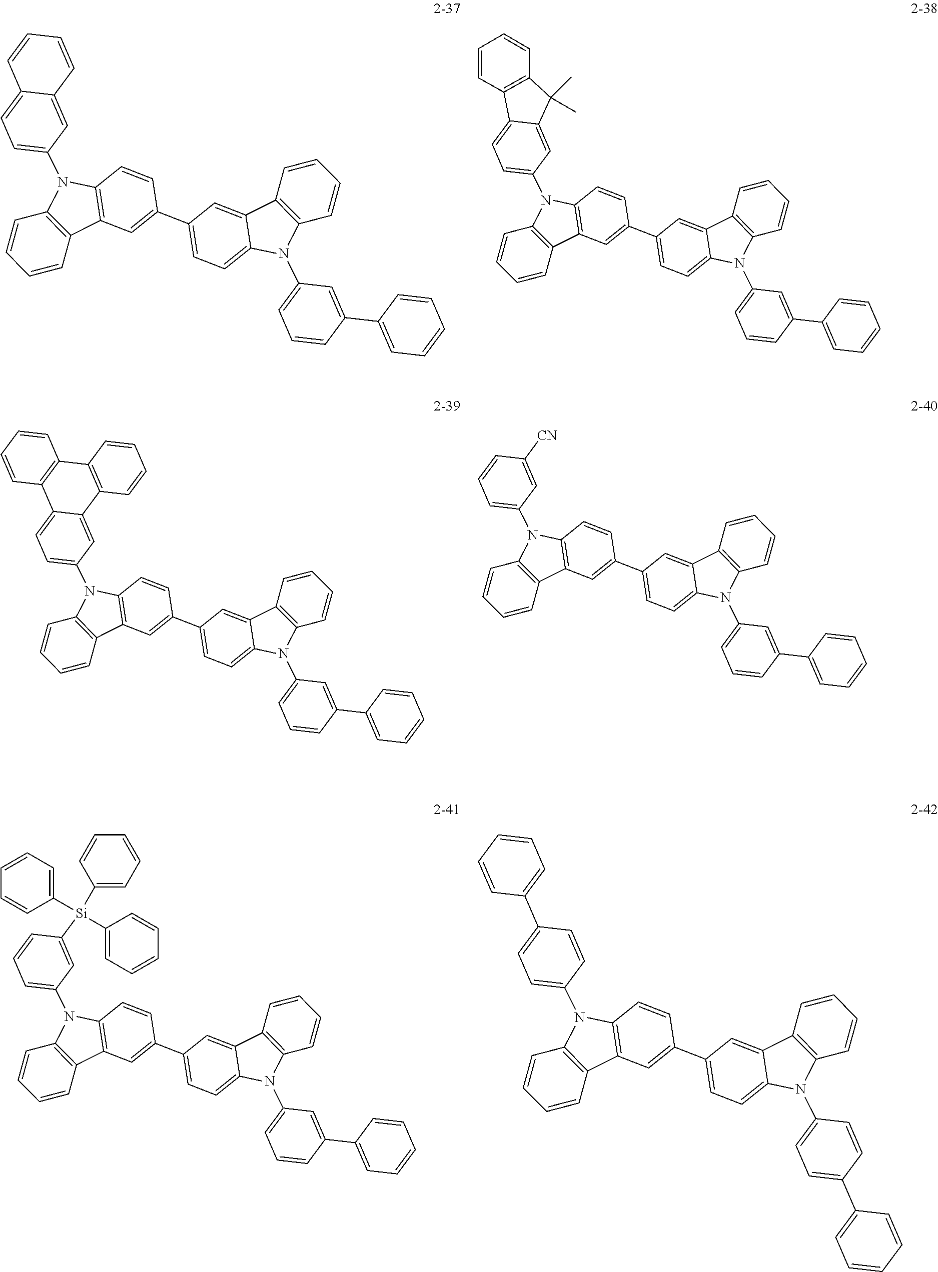

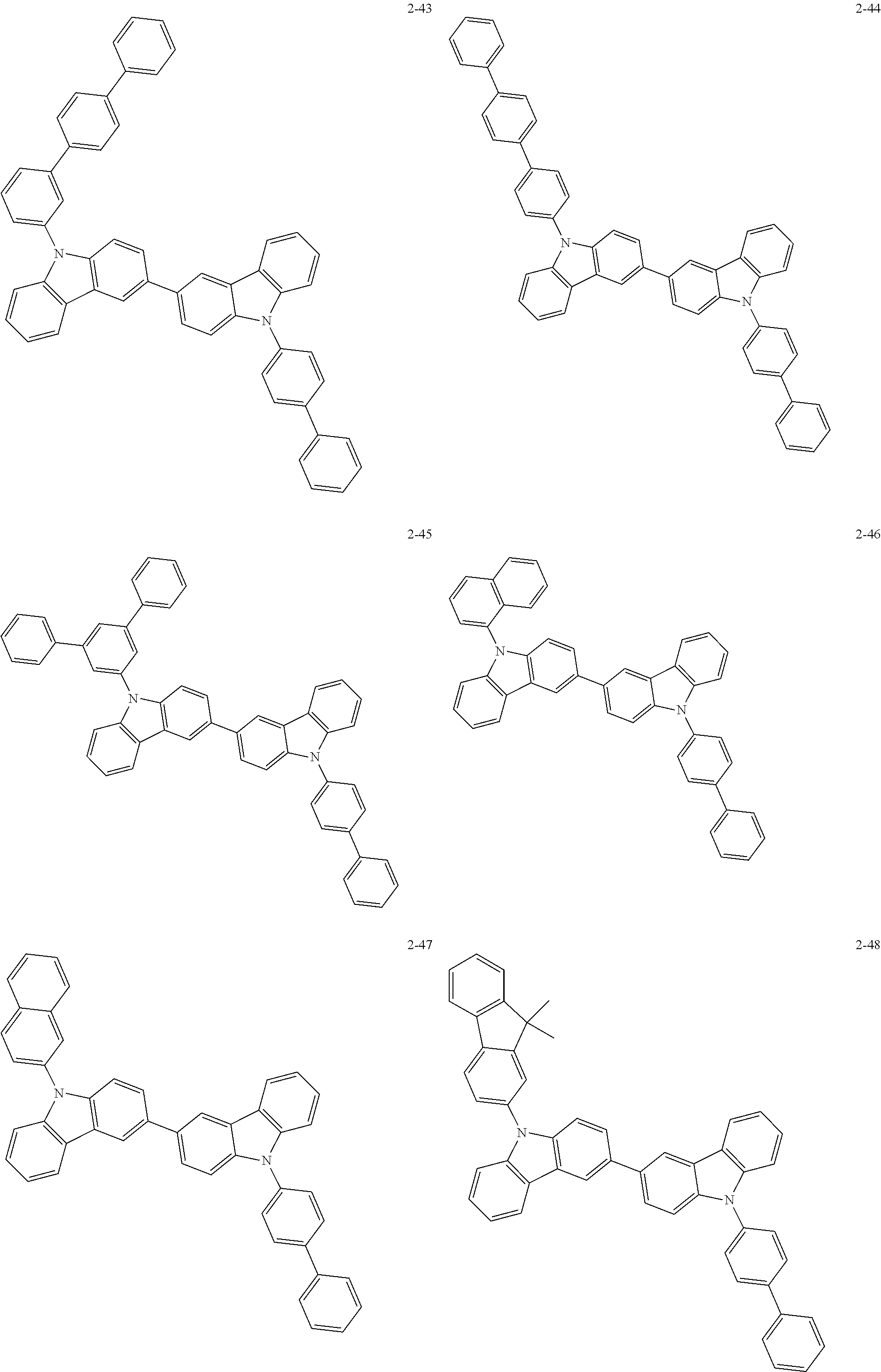

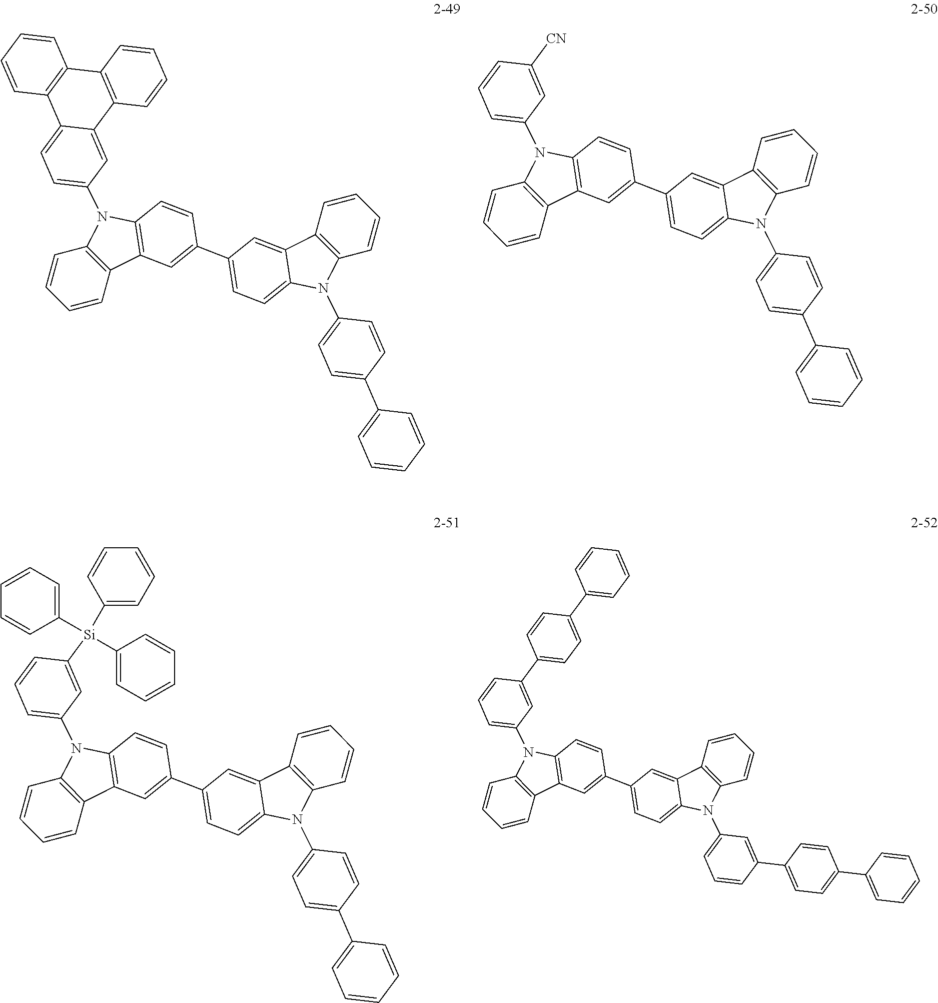 [SMILES: c1ccc(-c2ccc(-n3c4ccccc4c4cc(-c5ccc6c(c5)c5ccccc5n6-c5ccc6c7ccccc7c7ccccc7c6c5)ccc43)cc2)cc1 ; N#Cc1cccc(-n2c3ccccc3c3cc(-c4ccc5c(c4)c4ccccc4n5-c4ccc(-c5ccccc5)cc4)ccc32)c1 ; c1ccc(-c2ccc(-n3c4ccccc4c4cc(-c5ccc6c(c5)c5ccccc5n6-c5cccc([Si](c6ccccc6)(c6ccccc6)c6ccccc6)c5)ccc43)cc2)cc1 ; c1ccc(-c2ccc(-c3cccc(-n4c5ccccc5c5cc(-c6ccc7c(c6)c6ccccc6n7-c6cccc(-c7ccc(-c8ccccc8)cc7)c6)ccc54)c3)cc2)cc1]

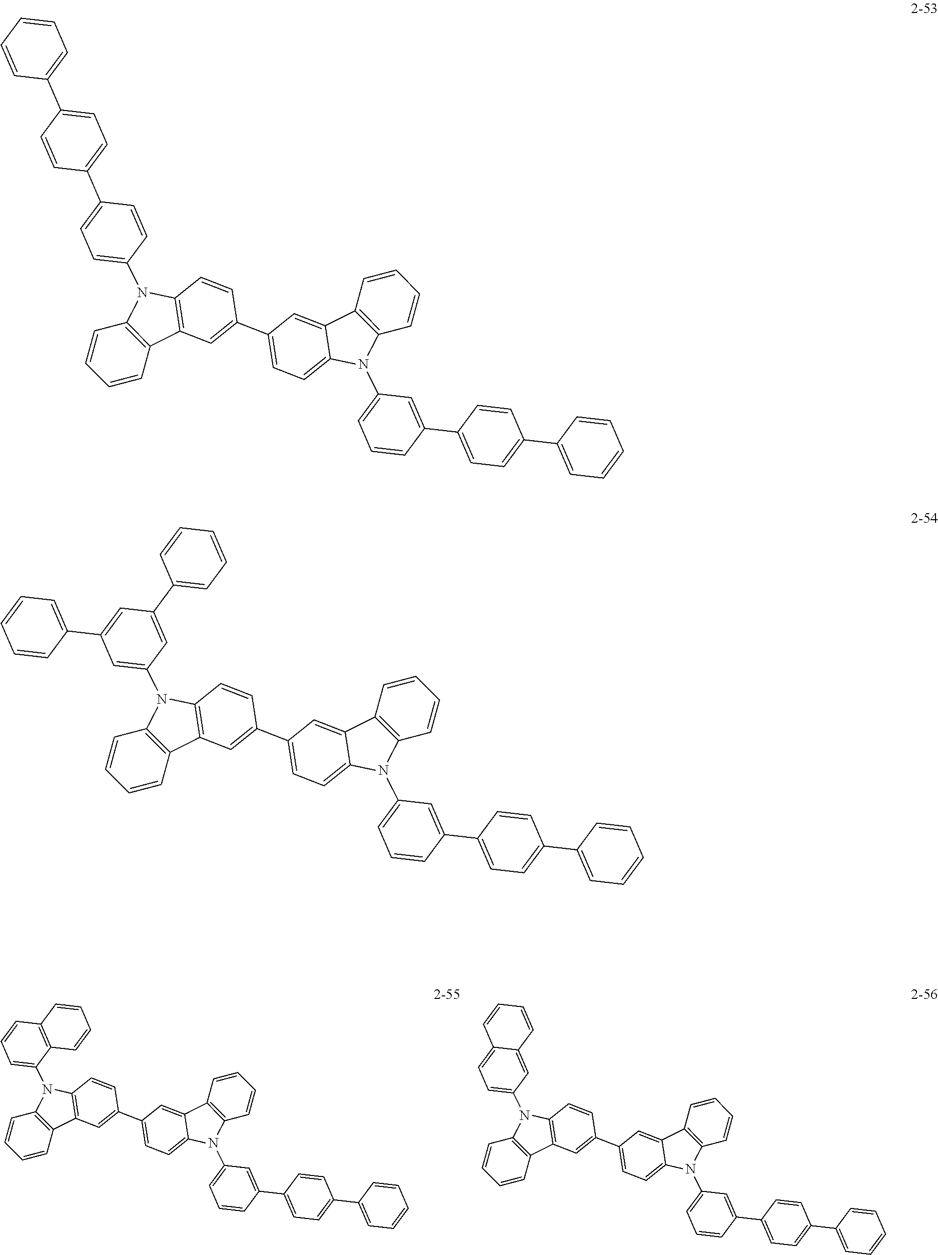 [SMILES: c1ccc(-c2ccc(-c3ccc(-n4c5ccccc5c5cc(-c6ccc7c(c6)c6ccccc6n7-c6cccc(-c7ccc(-c8ccccc8)cc7)c6)ccc54)cc3)cc2)cc1 ; c1ccc(-c2ccc(-c3cccc(-n4c5ccccc5c5cc(-c6ccc7c(c6)c6ccccc6n7-c6cc(-c7ccccc7)cc(-c7ccccc7)c6)ccc54)c3)cc2)cc1 ; c1ccc(-c2ccc(-c3cccc(-n4c5ccccc5c5cc(-c6ccc7c(c6)c6ccccc6n7-c6cccc7ccccc67)ccc54)c3)cc2)cc1 ; c1ccc(-c2ccc(-c3cccc(-n4c5ccccc5c5cc(-c6ccc7c(c6)c6ccccc6n7-c6ccc7ccccc7c6)ccc54)c3)cc2)cc1]

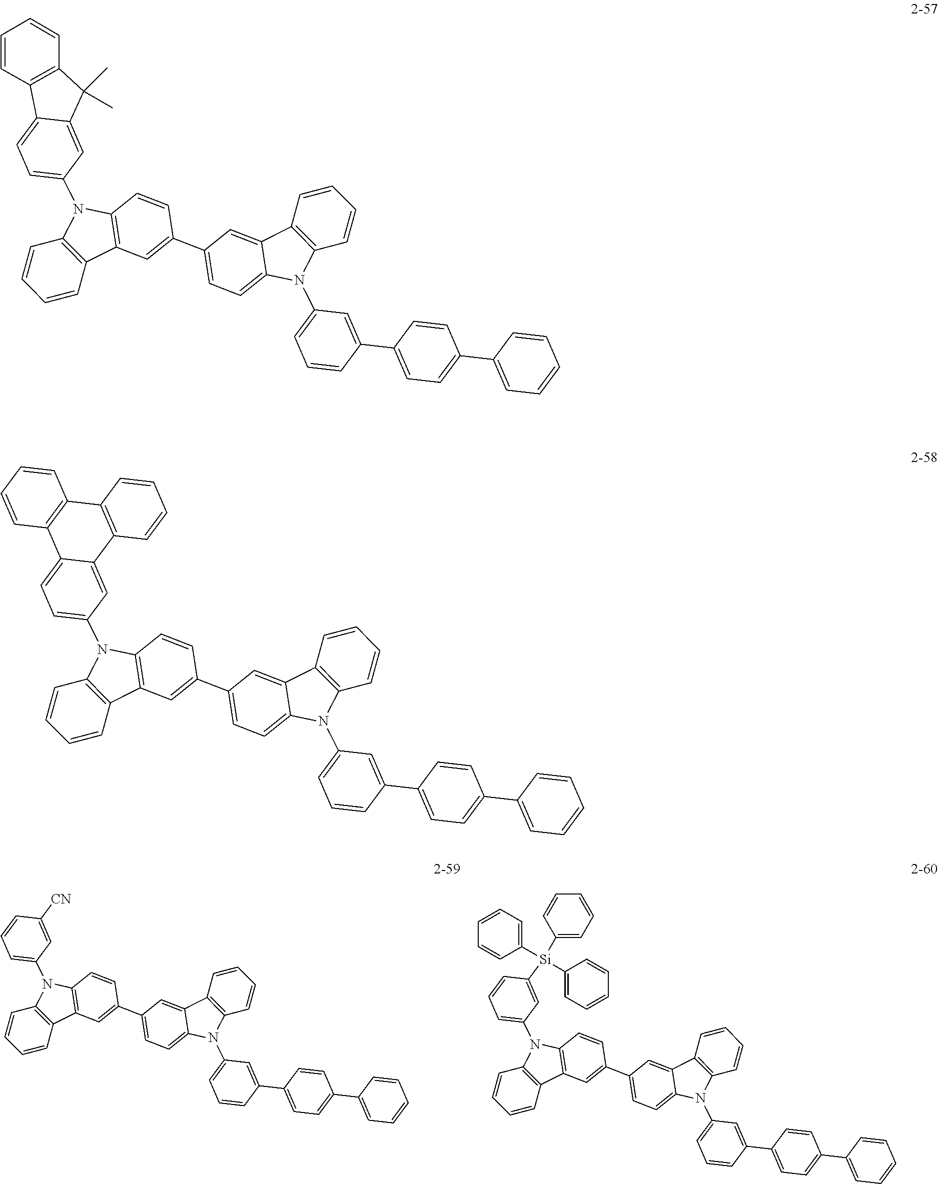 [SMILES: CC1(C)c2ccccc2-c2ccc(-n3c4ccccc4c4cc(-c5ccc6c(c5)c5ccccc5n6-c5cccc(-c6ccc(-c7ccccc7)cc6)c5)ccc43)cc21 ; c1ccc(-c2ccc(-c3cccc(-n4c5ccccc5c5cc(-c6ccc7c(c6)c6ccccc6n7-c6ccc7c8ccccc8c8ccccc8c7c6)ccc54)c3)cc2)cc1 ; N#Cc1cccc(-n2c3ccccc3c3cc(-c4ccc5c(c4)c4ccccc4n5-c4cccc(-c5ccc(-c6ccccc6)cc5)c4)ccc32)c1 ; c1ccc(-c2ccc(-c3cccc(-n4c5ccccc5c5cc(-c6ccc7c(c6)c6ccccc6n7-c6cccc([Si](c7ccccc7)(c7ccccc7)c7ccccc7)c6)ccc54)c3)cc2)cc1]

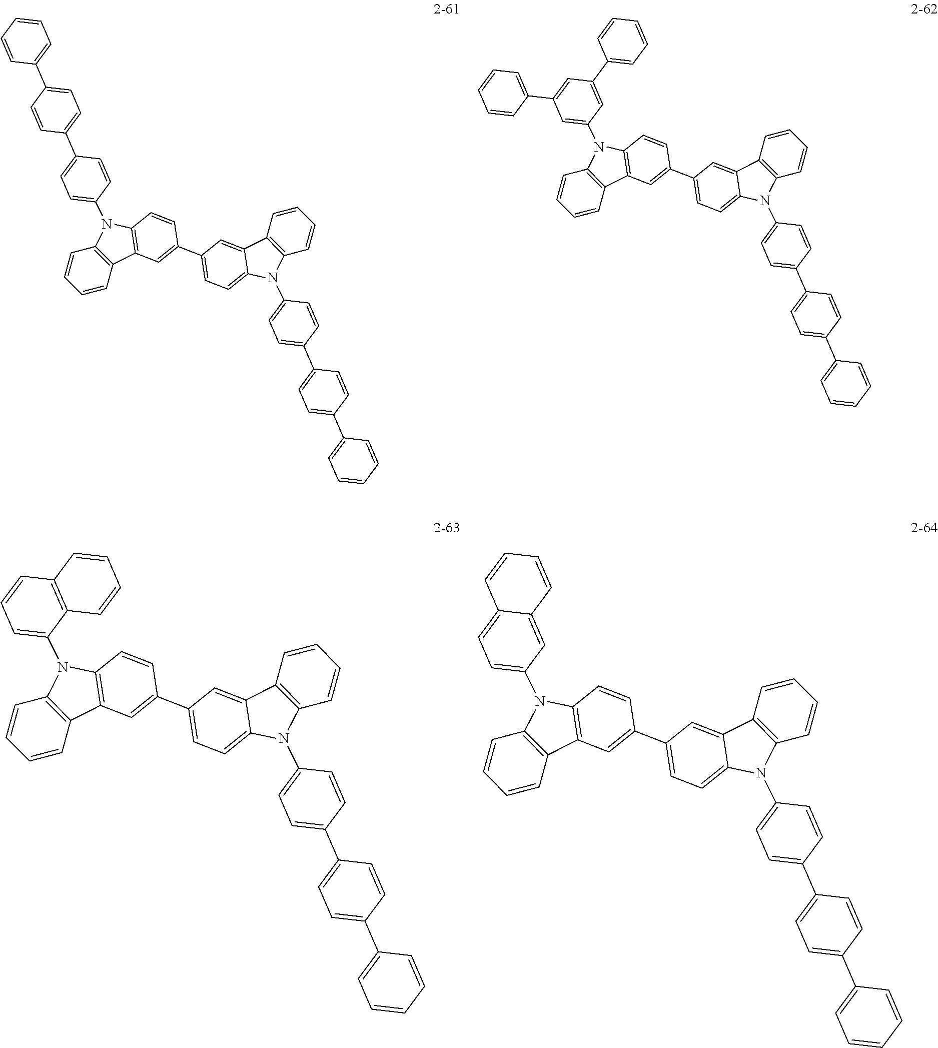 [SMILES: c1ccc(-c2ccc(-c3ccc(-n4c5ccccc5c5cc(-c6ccc7c(c6)c6ccccc6n7-c6ccc(-c7ccc(-c8ccccc8)cc7)cc6)ccc54)cc3)cc2)cc1 ; c1ccc(-c2ccc(-c3ccc(-n4c5ccccc5c5cc(-c6ccc7c(c6)c6ccccc6n7-c6cc(-c7ccccc7)cc(-c7ccccc7)c6)ccc54)cc3)cc2)cc1 ; c1ccc(-c2ccc(-c3ccc(-n4c5ccccc5c5cc(-c6ccc7c(c6)c6ccccc6n7-c6cccc7ccccc67)ccc54)cc3)cc2)cc1 ; c1ccc(-c2ccc(-c3ccc(-n4c5ccccc5c5cc(-c6ccc7c(c6)c6ccccc6n7-c6ccc7ccccc7c6)ccc54)cc3)cc2)cc1]

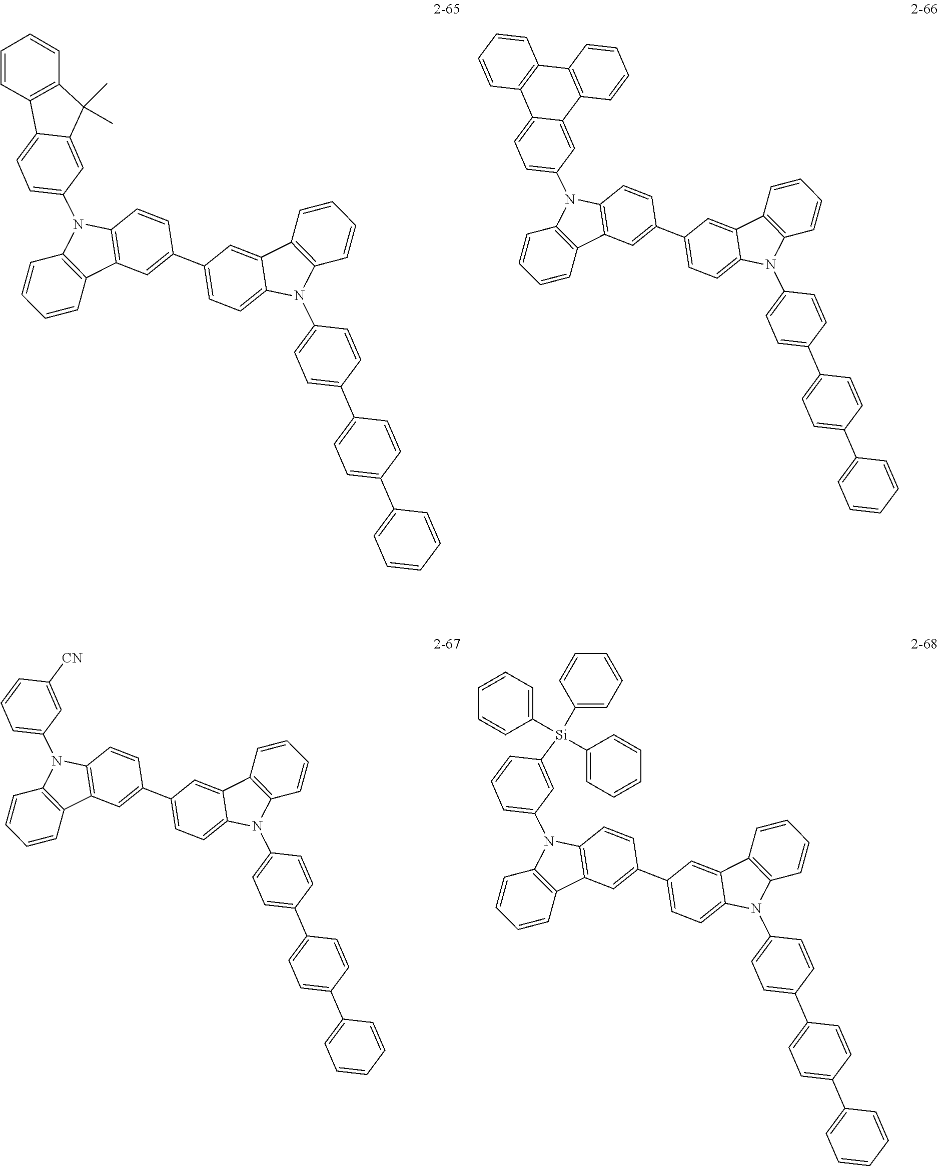 [SMILES: CC1(C)c2ccccc2-c2ccc(-n3c4ccccc4c4cc(-c5ccc6c(c5)c5ccccc5n6-c5ccc(-c6ccc(-c7ccccc7)cc6)cc5)ccc43)cc21 ; c1ccc(-c2ccc(-c3ccc(-n4c5ccccc5c5cc(-c6ccc7c(c6)c6ccccc6n7-c6ccc7c8ccccc8c8ccccc8c7c6)ccc54)cc3)cc2)cc1 ; N#Cc1cccc(-n2c3ccccc3c3cc(-c4ccc5c(c4)c4ccccc4n5-c4ccc(-c5ccc(-c6ccccc6)cc5)cc4)ccc32)c1 ; c1ccc(-c2ccc(-c3ccc(-n4c5ccccc5c5cc(-c6ccc7c(c6)c6ccccc6n7-c6cccc([Si](c7ccccc7)(c7ccccc7)c7ccccc7)c6)ccc54)cc3)cc2)cc1]

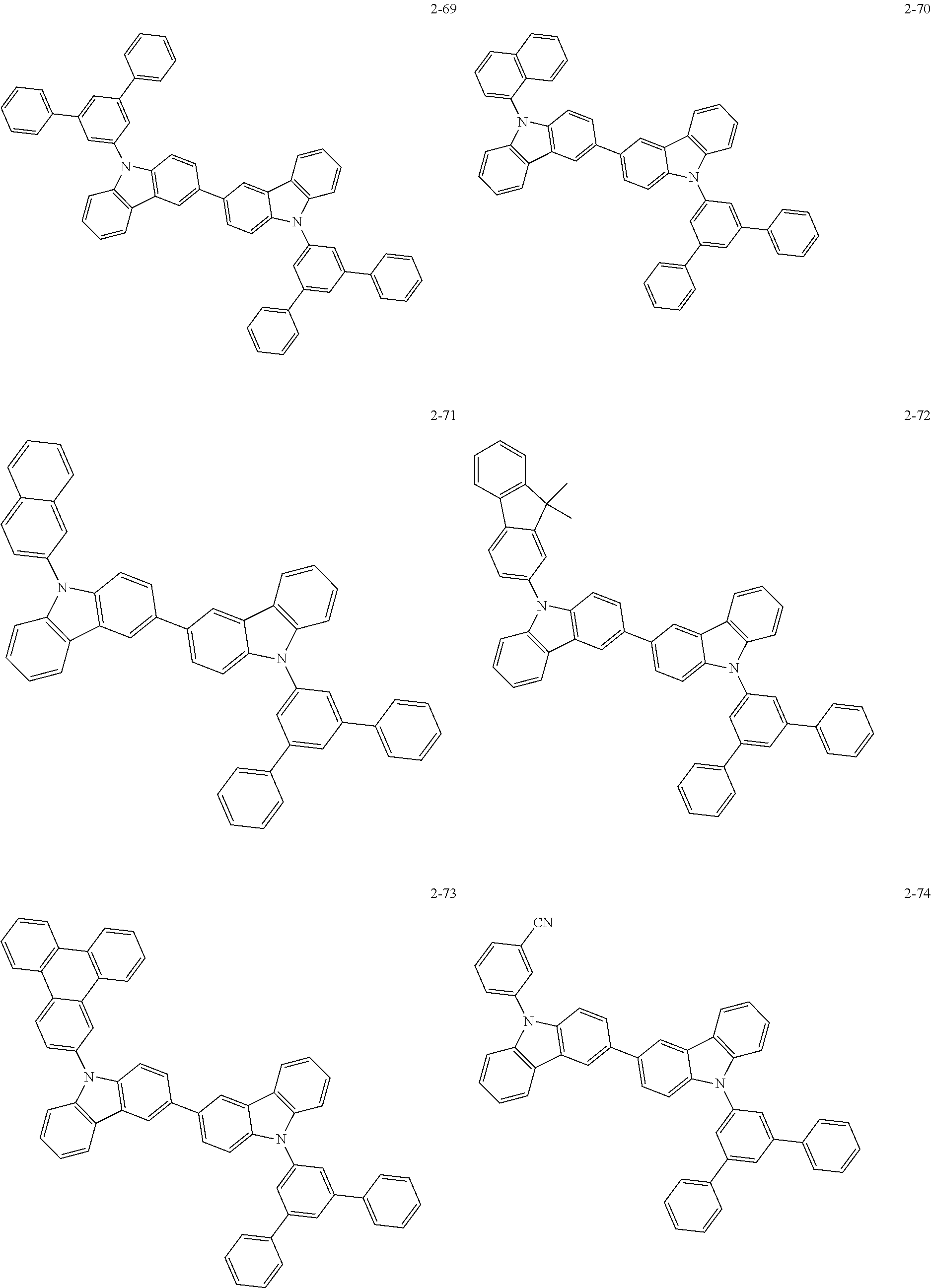

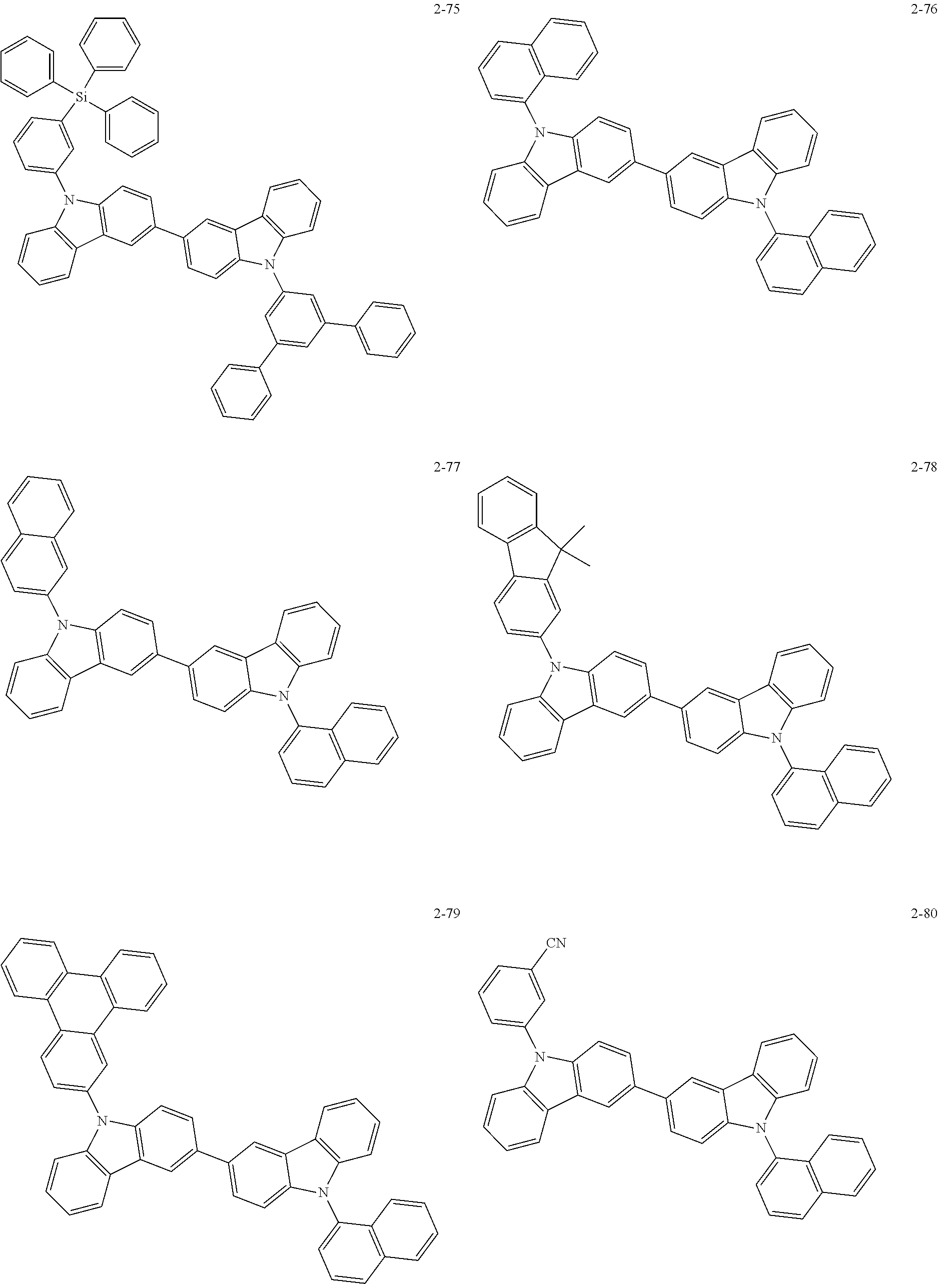

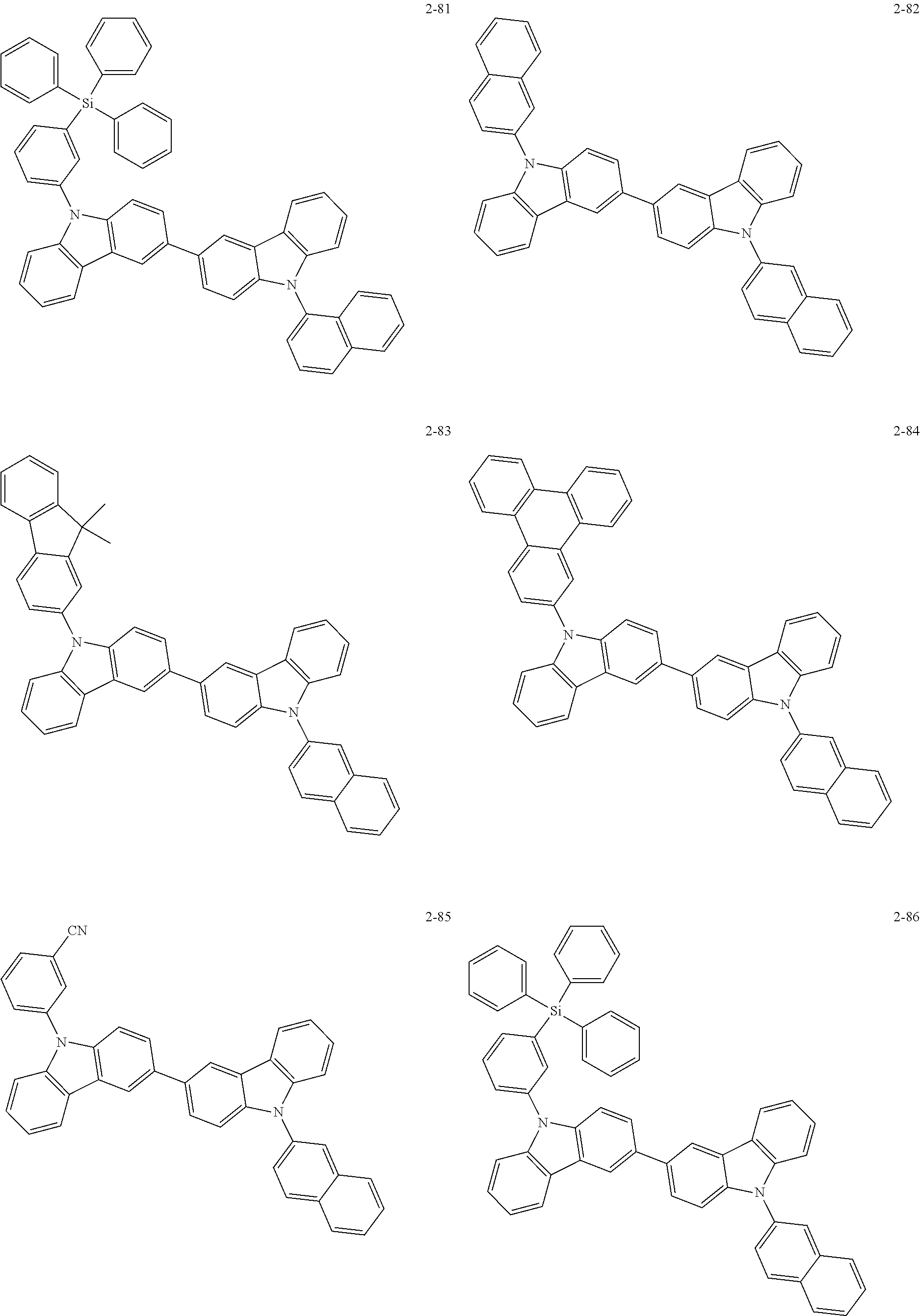

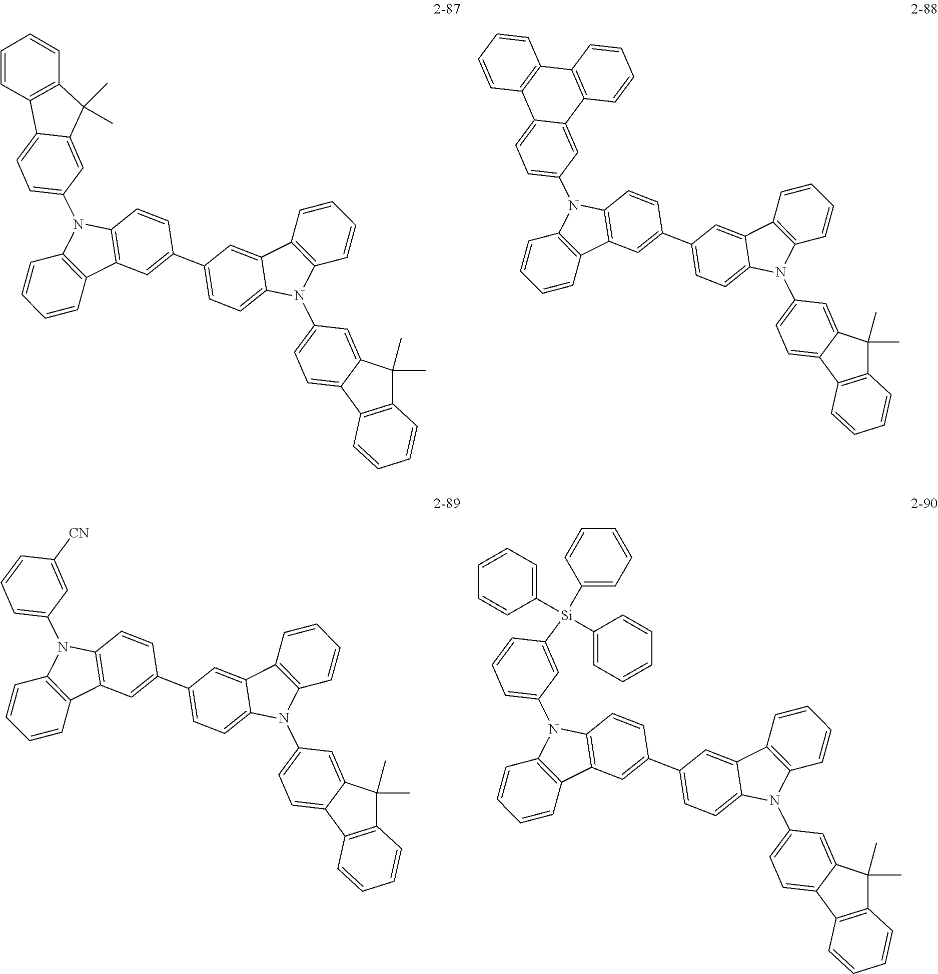 [SMILES: CC1(C)c2ccccc2-c2ccc(-n3c4ccccc4c4cc(-c5ccc6c(c5)c5ccccc5n6-c5ccc6c(c5)C(C)(C)c5ccccc5-6)ccc43)cc21 ; CC1(C)c2ccccc2-c2ccc(-n3c4ccccc4c4cc(-c5ccc6c(c5)c5ccccc5n6-c5ccc6c7ccccc7c7ccccc7c6c5)ccc43)cc21 ; CC1(C)c2ccccc2-c2ccc(-n3c4ccccc4c4cc(-c5ccc6c(c5)c5ccccc5n6-c5cccc(C#N)c5)ccc43)cc21 ; CC1(C)c2ccccc2-c2ccc(-n3c4ccccc4c4cc(-c5ccc6c(c5)c5ccccc5n6-c5cccc([Si](c6ccccc6)(c6ccccc6)c6ccccc6)c5)ccc43)cc21]

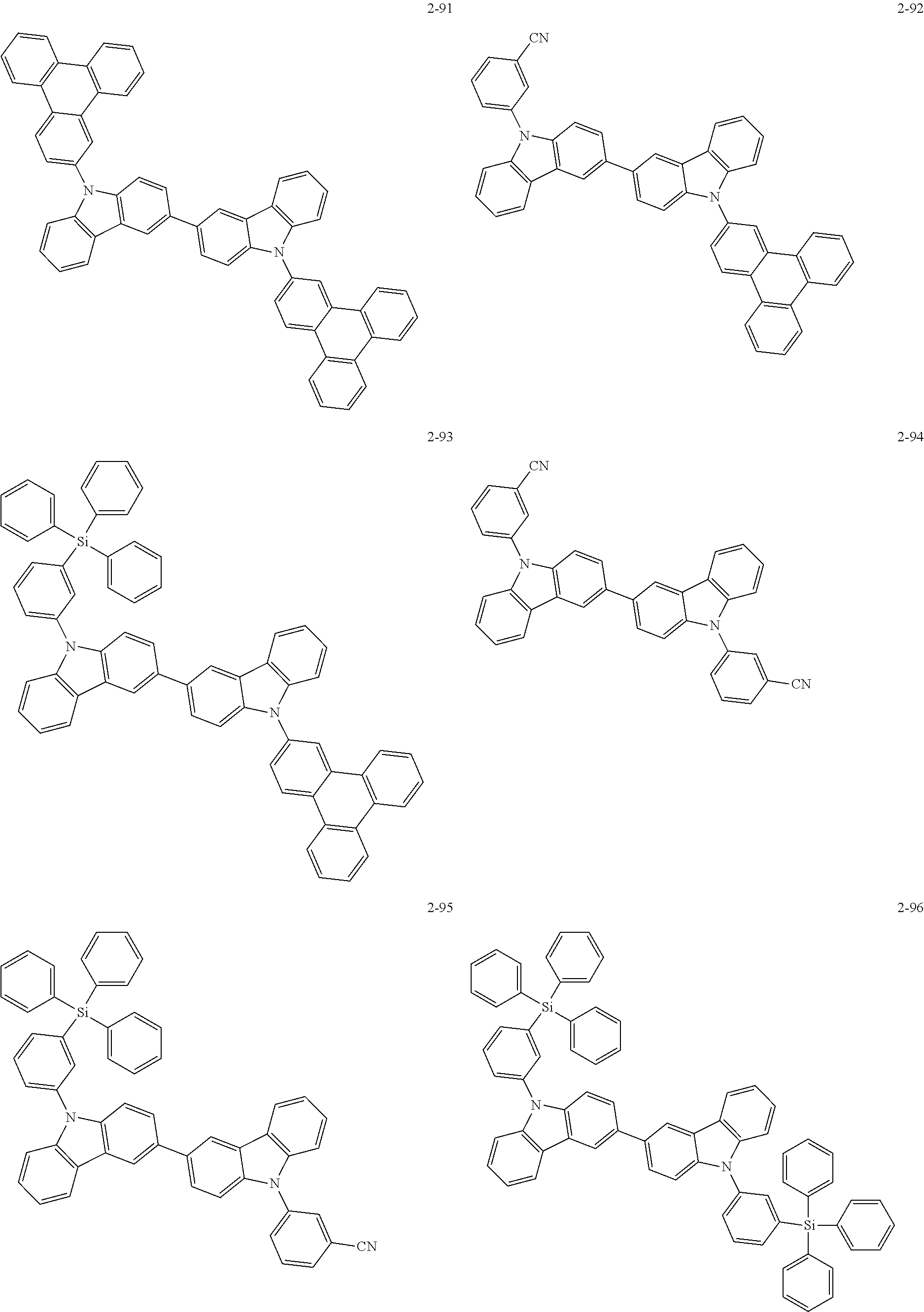

In one embodiment of the present specification, the organic material layer may include the heterocyclic compound and the compound of Chemical Formula 2 in a weight ratio of 1:10 to 10:1.

In one embodiment of the present specification, the organic material layer may include the heterocyclic compound and the compound of Chemical Formula 2 in a weight ratio of 1:5 to 5:1.

In one embodiment of the present specification, the organic material layer may include the heterocyclic compound and the compound of Chemical Formula 2 in a weight ratio of 1:2 to 2:1.

The organic light emitting device of the present disclosure may further include one, two or more layers selected from the group consisting of a light emitting layer, a hole injection layer, a hole transfer layer, an electron injection layer, an electron transfer layer, an electron blocking layer and a hole blocking layer.

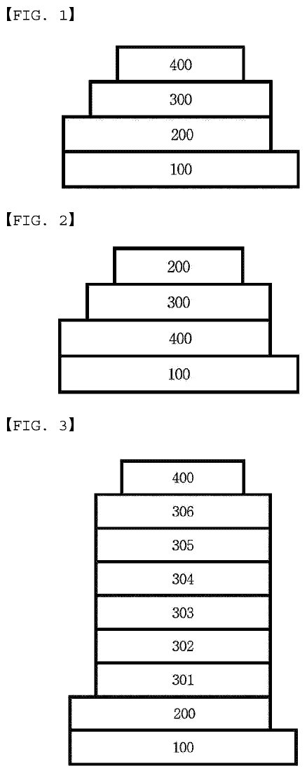

FIG. 1 to FIG. 3 illustrate a lamination order of electrodes and organic material layers of the organic light emitting device according to one embodiment of the present specification. However, the scope of the present application is not limited to these diagrams, and structures of organic light emitting devices known in the art may also be used in the present application.

FIG. 1 illustrates an organic light emitting device in which an anode (200), an organic material layer (300) and a cathode (400) are consecutively laminated on a substrate (100). However, the structure is not limited to such a structure, and as illustrated in FIG. 2, an organic light emitting device in which a cathode, an organic material layer and an anode are consecutively laminated on a substrate may also be obtained.

FIG. 3 illustrates a case of the organic material layer being a multilayer. The organic light emitting device according to FIG. 3 includes a hole injection layer (301), a hole transfer layer (302), a light emitting layer (303), a hole blocking layer (304), an electron transfer layer (305) and an electron injection layer (306). However, the scope of the present application is not limited to such a lamination structure, and as necessary, the layers other than the light emitting layer may not be included, and other necessary functional layers may be further added.

The organic material layer including the heterocyclic compound of Chemical Formula 1 may further include other materials as necessary.

One embodiment of the present specification provides a composition for an organic material layer of an organic light emitting device, the composition including the heterocyclic compound of Chemical Formula 1.

The composition for an organic material layer according to one embodiment of the present specification may further include the compound of Chemical Formula 2.

In one embodiment of the present specification, the composition for an organic material layer may include the heterocyclic compound of Chemical Formula 1 and the compound of Chemical Formula 2 in a weight ratio of 1:10 to 10:1.

In one embodiment of the present specification, the composition for an organic material layer may include the heterocyclic compound of Chemical Formula 1 and the compound of Chemical Formula 2 in a weight ratio of 1:5 to 5:1.

In one embodiment of the present specification, the composition for an organic material layer may include the heterocyclic compound of Chemical Formula 1 and the compound of Chemical Formula 2 in a weight ratio of 1:2 to 2:1.

One embodiment of the present specification provides a method for manufacturing an organic light emitting device, the method including preparing a substrate; forming a first electrode on the substrate; forming one or more organic material layers on the first electrode; and forming a second electrode on the organic material layers, wherein the forming of organic material layers includes forming one or more organic material layers using the composition for an organic material layer including the heterocyclic compound of Chemical Formula 1.

A method for manufacturing an organic light emitting device according to another embodiment includes preparing a substrate; forming a first electrode on the substrate; forming one or more organic material layers on the first electrode; and forming a second electrode on the organic material layers, wherein the forming of organic material layers includes forming one or more organic material layers using the composition for an organic material layer including the heterocyclic compound of Chemical Formula 1 and the compound of Chemical Formula 2, and the forming of organic material layers may be forming using a thermal vacuum deposition method after pre-mixing the heterocyclic compound of Chemical Formula 1; and the compound of Chemical Formula 2.

The pre-mixing means first mixing the heterocyclic compound of Chemical Formula 1 and the compound of Chemical Formula 2 in one source of supply before depositing on the organic material layer.

The pre-mixed material may be referred to as the composition for an organic material layer according to one embodiment of the present specification.

In the organic light emitting device according to one embodiment of the present specification, materials other than the compounds of Chemical Formulae 1 and 2 are illustrated below, however, these are for illustrative purposes only and not for limiting the scope of the present application, and these materials may be replaced by materials known in the art.

As the anode material, materials having relatively large work function may be used, and transparent conductive oxides, metals, conductive polymers or the like may be used. Specific examples of the anode material include metals such as vanadium, chromium, copper, zinc and gold, or alloys thereof; metal oxides such as zinc oxide, indium oxide, indium tin oxide (ITO) and indium zinc oxide (IZO); combinations of metals and oxides such as ZnO:Al or SnO₂:Sb; conductive polymers such as poly(3-methylthiophene), poly[3,4-(ethylene-1,2-dioxy)thiophene](PEDOT), polypyrrole and polyaniline, and the like, but are not limited thereto.

As the cathode material, materials having relatively small work function may be used, and metals, metal oxides, conductive polymers or the like may be used. Specific examples of the cathode material include metals such as magnesium, calcium, sodium, potassium, titanium, indium, yttrium, lithium, gadolinium, aluminum, silver, tin and lead, or alloys thereof; multilayer structure materials such as LiF/Al or LiO₂/Al, and the like, but are not limited thereto.

As the hole injection material, known hole injection materials may be used, and for example, phthalocyanine compounds such as copper phthalocyanine disclosed in U.S. Pat. No. 4,356,429, or starburst-type amine derivatives such as tris(4-carbazoyl-9-ylphenyl)amine (TCTA), 4,4′,4″-tri[phenyl(m-tolyl) amino]triphenylamine (m-MTDATA), 1,3,5-tris[4-(3-methylphenylphenylamino)phenyl]benzene (m-MTDAPB) or 4,4′,4″-tris[2-naphthyl(phenyl)amino]triphenylamine (2-TNATA) described in the literature [Advanced Material, 6, p. 677 (1994)], polyaniline/dodecylbenzene sulfonic acid, poly(3,4-ethylenedioxythiophene)/poly(4-styrenesulfonate), polyaniline/camphor sulfonic acid or polyaniline/poly(4-styrenesulfonate) that are conductive polymers having solubility, and the like, may be used.

As the hole transfer material, pyrazoline derivatives, arylamine-based derivatives, stilbene derivatives, triphenyldiamine derivatives and the like may be used, and low molecular or high molecular materials may also be used. For example, N,N′-di(1-naphthyl)-N,N′-diphenyl-(1,1′-biphenyl)-4,4′-diamine (NPB) may be used.

As the hole blocking material, BCP (bathocuproine) may be used, however, the hole blocking material is not limited thereto.

As the electron transfer material, metal complexes of oxadiazole derivatives, anthraquinodimethane and derivatives thereof, benzoquinone and derivatives thereof, naphthoquinone and derivatives thereof, anthraquinone and derivatives thereof, tetracyanoanthraquinodimethane and derivatives thereof, fluorenone derivatives, diphenyldicyanoethylene and derivatives thereof, diphenoquinone derivatives, 8-hydroxyquinoline and derivatives thereof, and the like, may be used, and high molecular materials may also be used as well as low molecular materials. For example, tris(8-hydroxyquinolinato)aluminum (Alq₃) may be used.

As examples of the electron injection material, LiF is typically used in the art, however, the present application is not limited thereto.

As the light emitting material, red, green or blue light emitting materials may be used, and as necessary, two or more light emitting materials may be mixed and used. Herein, the two or more light emitting materials may be deposited with individual sources of supply or pre-mixed and deposited with one source of supply when used. In addition, fluorescent materials may also be used as the light emitting material, however, phosphorescent materials may also be used. As the light emitting material, materials emitting light by bonding holes and electrons injected from an anode and a cathode, respectively, may be used alone, however, materials having a host material and a dopant material involving together in light emission may also be used.

In one embodiment of the present specification, the heterocyclic compound of Chemical Formula 1 may be used alone as the host material.

In one embodiment of the present specification, the heterocyclic compound of Chemical Formula 1 and the compound of Chemical Formula 2 may be mixed and used as the host material, and additional materials may be further included.

In one embodiment of the present specification, a green phosphorescent dopant may be used as the dopant material.

In one embodiment of the present specification, Ir(ppy)₃ may be used as the dopant material, however, the dopant material is not limited thereto.

When mixing light emitting material hosts, same series hosts may be mixed, or different series hosts may be mixed. For example, any two or more types of materials among N-type host materials or P-type host materials may be selected and used as a host material of a light emitting layer.

The organic light emitting device according to one embodiment of the present specification may be a top-emission type, a bottom-emission type or a dual-emission type depending on the materials used.

The compound according to one embodiment of the present specification may also be used in an organic electronic device including an organic solar cell, an organic photo conductor, an organic transistor and the like under a similar principle used in the organic light emitting device.

Hereinafter, the present specification will be described in more detail with reference to examples, however, these are for illustrative purposes only, and the scope of the present application is not limited thereto.

### <Preparation Example 1> Preparation of Compound 1-1

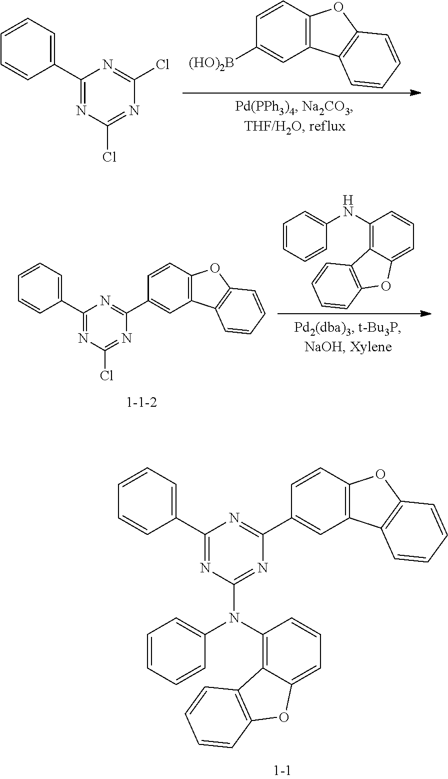 [SMILES: Clc1nc(Cl)nc(-c2ccccc2)n1 ; OB(O)c1ccc2oc3ccccc3c2c1 ; Clc1nc(-c2ccccc2)nc(-c2ccc3oc4ccccc4c3c2)n1 ; c1ccc(Nc2cccc3oc4ccccc4c23)cc1 ; c1ccc(-c2nc(-c3ccc4oc5ccccc5c4c3)nc(N(c3ccccc3)c3cccc4oc5ccccc5c34)n2)cc1]

### 1) Preparation of Compound 1-1-2

2,4-Dichloro-6-phenyl-1,3,5-triazine [A] (27.6 g, 122.2 mM), dibenzo[b,d]furan-2-ylboronic acid [B] (20.0 g, 94.0 mM), Pd(PPh₃)₄(tetrakis(triphenylphosphine)palladium(0)) (5.4 g, 4.7 mM) and Na₂CO₃ (19.9 g, 188.0 mM) were dissolved in tetrahydrofuran (THF)/H₂O (200 mL/40 mL), and refluxed for 24 hours. After the reaction was completed, the result was extracted by introducing distilled water and dichloromethane (DCM) thereto at room temperature, and after drying the organic layer with MgSO₄, the solvent was removed using a rotary evaporator. The reaction material was purified by column chromatography (DCM:hexane=1:2) to obtain Compound 1-1-2 (20.2 g, 60%).

### 2) Preparation of Compound 1-1

Compound 1-1-2 (20 g, 55.0 mM), N-phenyldibenzo[b,d]furan-1-amine [C] (14.3 g, 55.0 mM), Pd₂(dba)₃ (tris(dibenzylideneacetone)dipalladium(0)) (2.5 g, 2.8 mM), t-Bu₃P (tri-tert-butylphosphine) (2.5 mL, 5.5 mM) and NaOH (4.4 g, 110.0 mM) were dissolved in xylene (200 mL), and refluxed for 1 hour. After the reaction was completed, the result was extracted by introducing distilled water and DCM thereto at room temperature, and after drying the organic layer with MgSO₄, the solvent was removed using a rotary evaporator. The reaction material was purified by column chromatography (DCM:Hex=1:3) to obtain target Compound 1-1 (26.2 g, 82%).

Target Compound D1 was synthesized in the same manner as in Preparation Example 1 except that Compound A1 of the following Table 1 was used instead of 2,4-dichloro-6-phenyl-1,3,5-triazine, Compound B1 of the following Table 1 was used instead of dibenzo[b,d]furan-2-ylboronic acid, and Compound C1 of the following Table 1 was used instead of N-phenyldibenzo[b,d]furan-1-amine.

TABLE 1

| Com- | | | | | |
|---|---|---|---|---|---|
| pound | Compound | | | Target Compound | |
| No. | A1 | Compound B1 | Compound C1 | D1 | Yield |
| 1-17 | 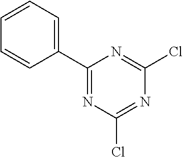 [SMILES: Clc1nc(Cl)nc(-c2ccccc2)n1] | 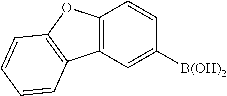 [SMILES: OB(O)c1ccc2oc3ccccc3c2c1] | 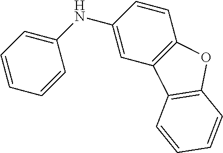 [SMILES: c1ccc(Nc2ccc3oc4ccccc4c3c2)cc1] | 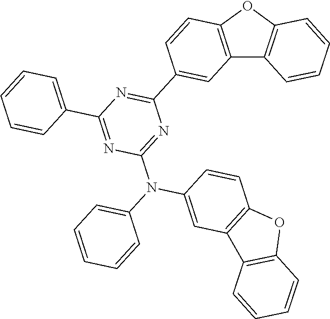 [SMILES: c1ccc(-c2nc(-c3ccc4oc5ccccc5c4c3)nc(N(c3ccccc3)c3ccc4oc5ccccc5c4c3)n2)cc1] | 51% |
| 1-40 | 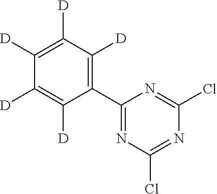 [SMILES: [2H]c1c([2H])c([2H])c(-c2nc(Cl)nc(Cl)n2)c([2H])c1[2H]] | 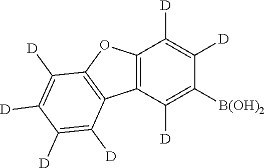 [SMILES: [2H]c1c([2H])c([2H])c2c(oc3c([2H])c([2H])c(B(O)O)c([2H])c32)c1[2H]] | 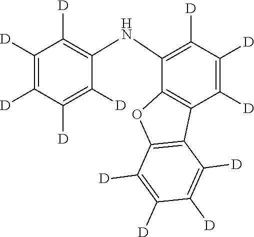 [SMILES: [2H]c1c([2H])c([2H])c(Nc2c([2H])c([2H])c([2H])c3c2oc2c([2H])c([2H])c([2H])c([2H])c23)c([2H])c1[2H]] | 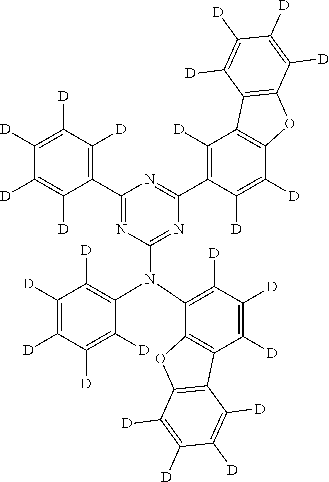 [SMILES: [2H]c1c([2H])c([2H])c(-c2nc(-c3c([2H])c([2H])c4oc5c([2H])c([2H])c([2H])c([2H])c5c4c3[2H])nc(N(c3c([2H])c([2H])c([2H])c([2H])c3[2H])c3c([2H])c([2H])c([2H])c4c3oc3c([2H])c([2H])c([2H])c([2H])c34)n2)c([2H])c1[2H]] | 48% |
| 1-46 | 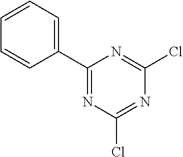 [SMILES: Clc1nc(Cl)nc(-c2ccccc2)n1] | 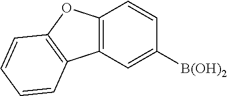 [SMILES: OB(O)c1ccc2oc3ccccc3c2c1] | 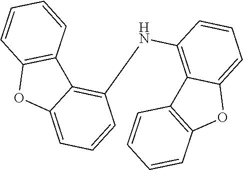 [SMILES: c1ccc2c(c1)oc1cccc(Nc3cccc4oc5ccccc5c34)c12] | 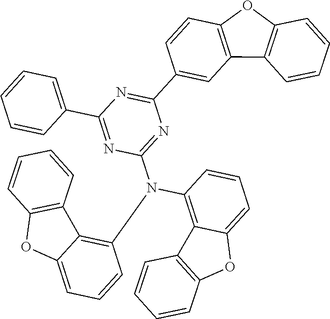 [SMILES: c1ccc(-c2nc(-c3ccc4oc5ccccc5c4c3)nc(N(c3cccc4oc5ccccc5c34)c3cccc4oc5ccccc5c34)n2)cc1] | 49% |
| 1-58 | 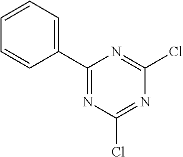 [SMILES: Clc1nc(Cl)nc(-c2ccccc2)n1] | 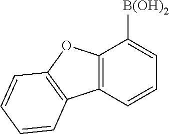 [SMILES: OB(O)c1cccc2c1oc1ccccc12] | 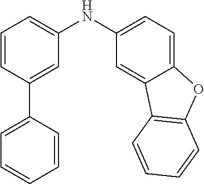 [SMILES: c1ccc(-c2cccc(Nc3ccc4oc5ccccc5c4c3)c2)cc1] | 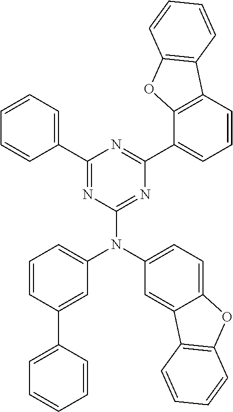 [SMILES: c1ccc(-c2cccc(N(c3ccc4oc5ccccc5c4c3)c3nc(-c4ccccc4)nc(-c4cccc5c4oc4ccccc45)n3)c2)cc1] | 50% |
| 1-77 | 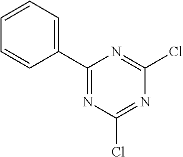 [SMILES: Clc1nc(Cl)nc(-c2ccccc2)n1] | 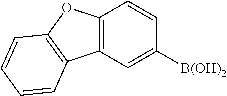 [SMILES: OB(O)c1ccc2oc3ccccc3c2c1] | 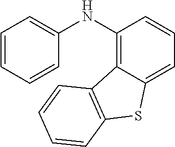 [SMILES: c1ccc(Nc2cccc3sc4ccccc4c23)cc1] | 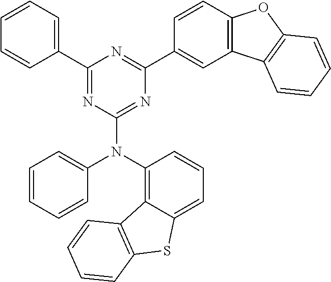 [SMILES: c1ccc(-c2nc(-c3ccc4oc5ccccc5c4c3)nc(N(c3ccccc3)c3cccc4sc5ccccc5c34)n2)cc1] | 50% |
| 1-121 | 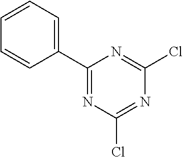 [SMILES: Clc1nc(Cl)nc(-c2ccccc2)n1] | 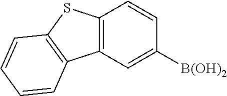 [SMILES: OB(O)c1ccc2sc3ccccc3c2c1] | 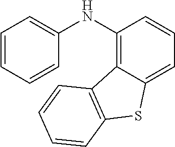 [SMILES: c1ccc(Nc2cccc3sc4ccccc4c23)cc1] | 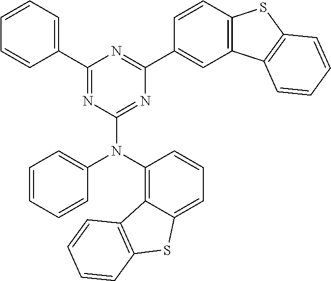 [SMILES: c1ccc(-c2nc(-c3ccc4sc5ccccc5c4c3)nc(N(c3ccccc3)c3cccc4sc5ccccc5c34)n2)cc1] | 52% |
| 1-189 | 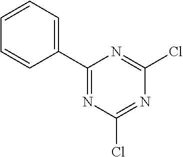 [SMILES: Clc1nc(Cl)nc(-c2ccccc2)n1] | 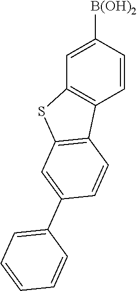 [SMILES: OB(O)c1ccc2c(c1)sc1cc(-c3ccccc3)ccc12] | 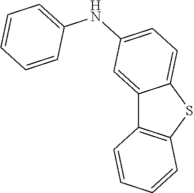 [SMILES: c1ccc(Nc2ccc3sc4ccccc4c3c2)cc1] | 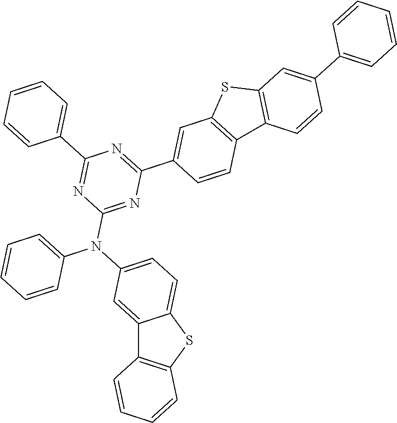 [SMILES: c1ccc(-c2ccc3c(c2)sc2cc(-c4nc(-c5ccccc5)nc(N(c5ccccc5)c5ccc6sc7ccccc7c6c5)n4)ccc23)cc1] | 50% |
| 1-199 | 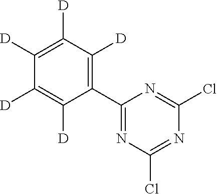 [SMILES: [2H]c1c([2H])c([2H])c(-c2nc(Cl)nc(Cl)n2)c([2H])c1[2H]] | 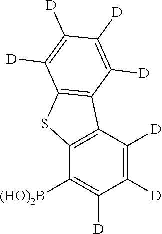 [SMILES: [2H]c1c([2H])c([2H])c2c(sc3c(B(O)O)c([2H])c([2H])c([2H])c32)c1[2H]] | 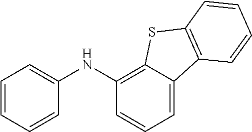 [SMILES: c1ccc(Nc2cccc3c2sc2ccccc23)cc1] | 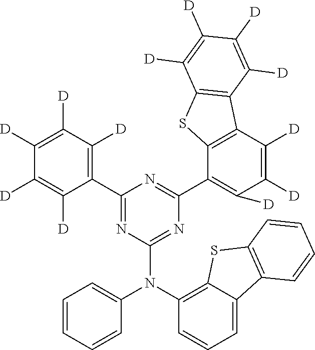 [SMILES: [2H]c1c([2H])c([2H])c(-c2nc(-c3c([2H])c([2H])c([2H])c4c3sc3c([2H])c([2H])c([2H])c([2H])c34)nc(N(c3ccccc3)c3cccc4c3sc3ccccc34)n2)c([2H])c1[2H]] | 50% |
| 1-201 | 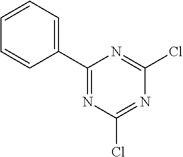 [SMILES: Clc1nc(Cl)nc(-c2ccccc2)n1] | 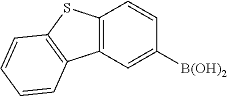 [SMILES: OB(O)c1ccc2sc3ccccc3c2c1] | 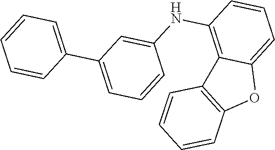 [SMILES: c1ccc(-c2cccc(Nc3cccc4oc5ccccc5c34)c2)cc1] | 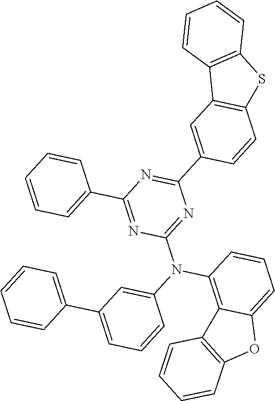 [SMILES: c1ccc(-c2cccc(N(c3nc(-c4ccccc4)nc(-c4ccc5sc6ccccc6c5c4)n3)c3cccc4oc5ccccc5c34)c2)cc1] | 51% |
| 1-224 | 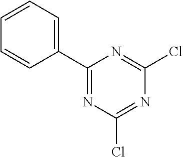 [SMILES: Clc1nc(Cl)nc(-c2ccccc2)n1] | 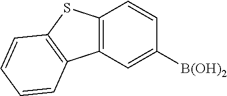 [SMILES: OB(O)c1ccc2sc3ccccc3c2c1] | 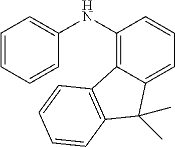 [SMILES: CC1(C)c2ccccc2-c2c(Nc3ccccc3)cccc21] | 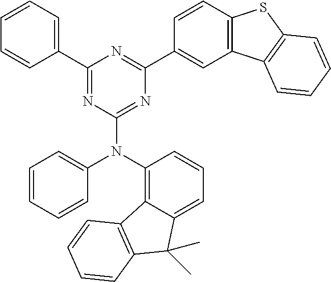 [SMILES: CC1(C)c2ccccc2-c2c(N(c3ccccc3)c3nc(-c4ccccc4)nc(-c4ccc5sc6ccccc6c5c4)n3)cccc21] | 54% |
| 1-280 | 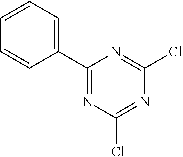 [SMILES: Clc1nc(Cl)nc(-c2ccccc2)n1] | 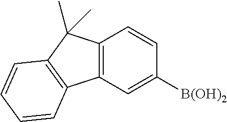 [SMILES: CC1(C)c2ccccc2-c2cc(B(O)O)ccc21] | 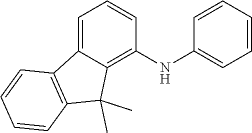 [SMILES: CC1(C)c2ccccc2-c2cccc(Nc3ccccc3)c21] | 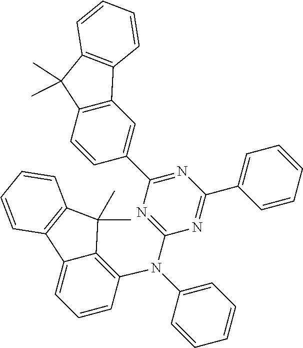 [SMILES: CC1(C)c2ccccc2-c2cc(-c3nc(-c4ccccc4)nc(N(c4ccccc4)c4cccc5c4C(C)(C)c4ccccc4-5)n3)ccc21] | 55% |
| 1-286 | 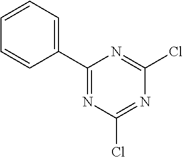 [SMILES: Clc1nc(Cl)nc(-c2ccccc2)n1] | 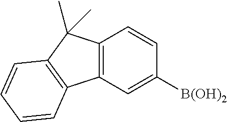 [SMILES: CC1(C)c2ccccc2-c2cc(B(O)O)ccc21] | 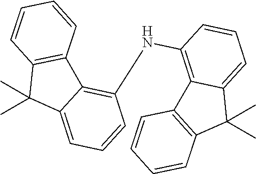 | 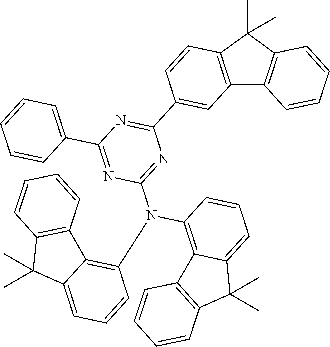 [SMILES: CC1(C)c2ccccc2-c2cc(-c3nc(-c4ccccc4)nc(N(c4cccc5c4-c4ccccc4C5(C)C)c4cccc5c4-c4ccccc4C5(C)C)n3)ccc21] | 53% |
| 1-295 | 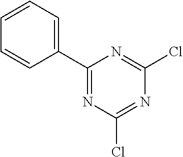 [SMILES: Clc1nc(Cl)nc(-c2ccccc2)n1] | 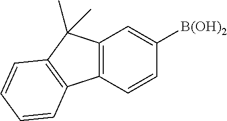 [SMILES: CC1(C)c2ccccc2-c2ccc(B(O)O)cc21] | 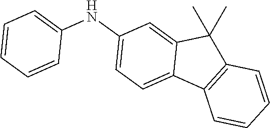 [SMILES: CC1(C)c2ccccc2-c2ccc(Nc3ccccc3)cc21] | 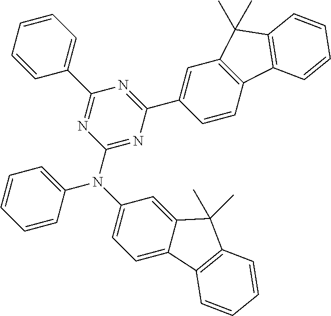 [SMILES: CC1(C)c2ccccc2-c2ccc(-c3nc(-c4ccccc4)nc(N(c4ccccc4)c4ccc5c(c4)C(C)(C)c4ccccc4-5)n3)cc21] | 54% |
| 1-301 | 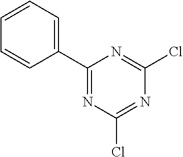 [SMILES: Clc1nc(Cl)nc(-c2ccccc2)n1] | 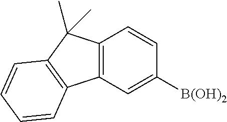 [SMILES: CC1(C)c2ccccc2-c2cc(B(O)O)ccc21] | 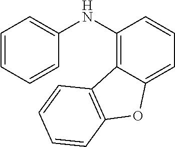 [SMILES: c1ccc(Nc2cccc3oc4ccccc4c23)cc1] | 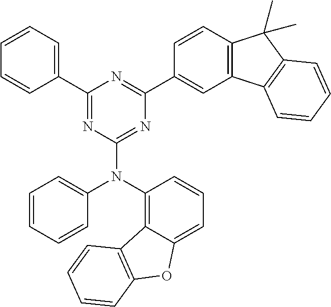 [SMILES: CC1(C)c2ccccc2-c2cc(-c3nc(-c4ccccc4)nc(N(c4ccccc4)c4cccc5oc6ccccc6c45)n3)ccc21] | 53% |
| 1-325 | 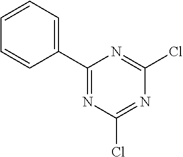 [SMILES: Clc1nc(Cl)nc(-c2ccccc2)n1] | 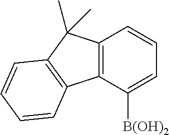 [SMILES: CC1(C)c2ccccc2-c2c(B(O)O)cccc21] | 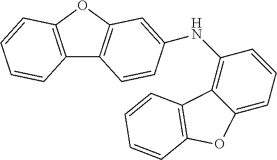 [SMILES: c1ccc2c(c1)oc1cc(Nc3cccc4oc5ccccc5c34)ccc12] | 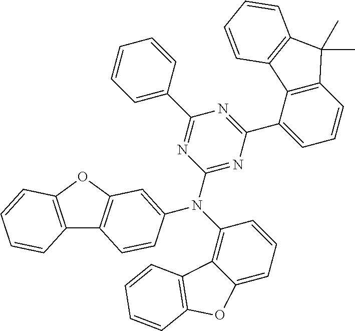 [SMILES: CC1(C)c2ccccc2-c2c(-c3nc(-c4ccccc4)nc(N(c4ccc5c(c4)oc4ccccc45)c4cccc5oc6ccccc6c45)n3)cccc21] | 55% |
| 1-326 | 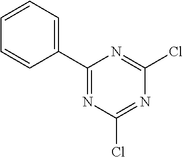 [SMILES: Clc1nc(Cl)nc(-c2ccccc2)n1] | 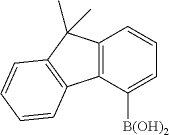 [SMILES: CC1(C)c2ccccc2-c2c(B(O)O)cccc21] | 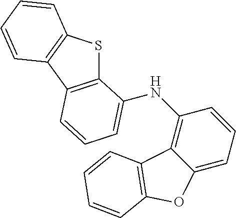 [SMILES: c1ccc2c(c1)oc1cccc(Nc3cccc4c3sc3ccccc34)c12] | 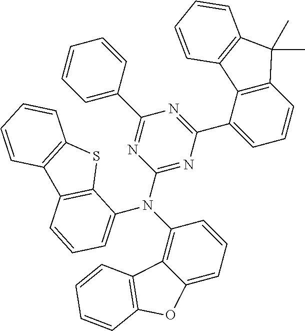 [SMILES: CC1(C)c2ccccc2-c2c(-c3nc(-c4ccccc4)nc(N(c4cccc5c4sc4ccccc45)c4cccc5oc6ccccc6c45)n3)cccc21] | 54% |
| 1-338 | 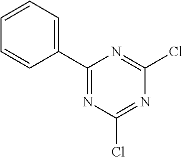 [SMILES: Clc1nc(Cl)nc(-c2ccccc2)n1] | 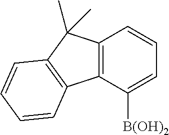 [SMILES: CC1(C)c2ccccc2-c2c(B(O)O)cccc21] | 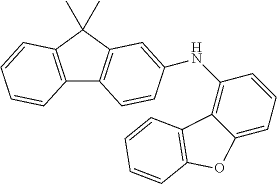 [SMILES: CC1(C)c2ccccc2-c2ccc(Nc3cccc4oc5ccccc5c34)cc21] | 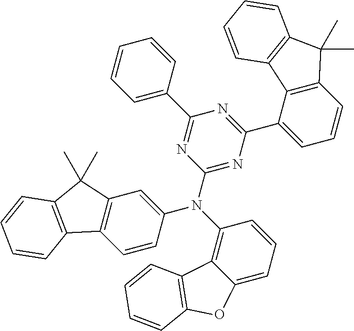 [SMILES: CC1(C)c2ccccc2-c2ccc(N(c3nc(-c4ccccc4)nc(-c4cccc5c4-c4ccccc4C5(C)C)n3)c3cccc4oc5ccccc5c34)cc21] | 56% |
| 1-341 | 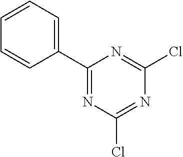 [SMILES: Clc1nc(Cl)nc(-c2ccccc2)n1] | 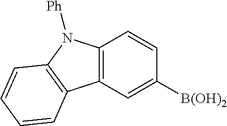 [SMILES: OB(O)c1ccc2c(c1)c1ccccc1n2-c1ccccc1] | 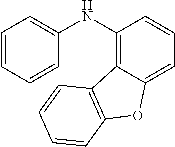 [SMILES: c1ccc(Nc2cccc3oc4ccccc4c23)cc1] | 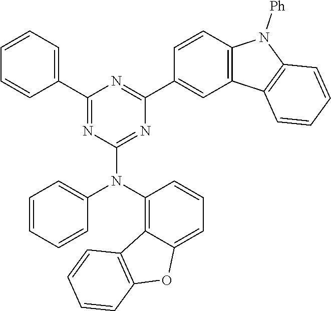 [SMILES: c1ccc(-c2nc(-c3ccc4c(c3)c3ccccc3n4-c3ccccc3)nc(N(c3ccccc3)c3cccc4oc5ccccc5c34)n2)cc1] | 52% |
| 1-362 | 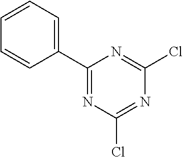 [SMILES: Clc1nc(Cl)nc(-c2ccccc2)n1] | 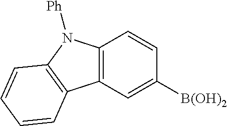 [SMILES: OB(O)c1ccc2c(c1)c1ccccc1n2-c1ccccc1] | 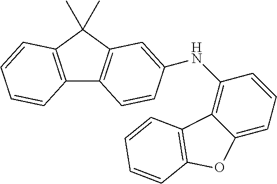 [SMILES: CC1(C)c2ccccc2-c2ccc(Nc3cccc4oc5ccccc5c34)cc21] | 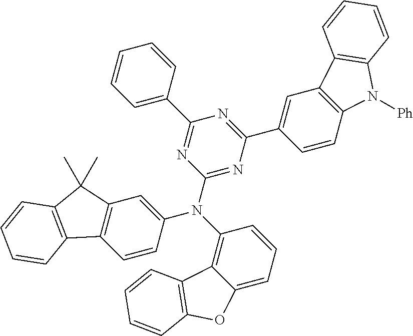 [SMILES: CC1(C)c2ccccc2-c2ccc(N(c3nc(-c4ccccc4)nc(-c4ccc5c(c4)c4ccccc4n5-c4ccccc4)n3)c3cccc4oc5ccccc5c34)cc21] | 51% |
| 1-366 | 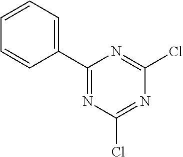 [SMILES: Clc1nc(Cl)nc(-c2ccccc2)n1] | 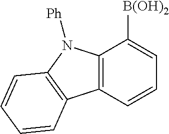 [SMILES: OB(O)c1cccc2c3ccccc3n(-c3ccccc3)c12] | 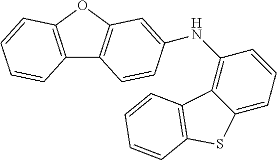 [SMILES: c1ccc2c(c1)oc1cc(Nc3cccc4sc5ccccc5c34)ccc12] | 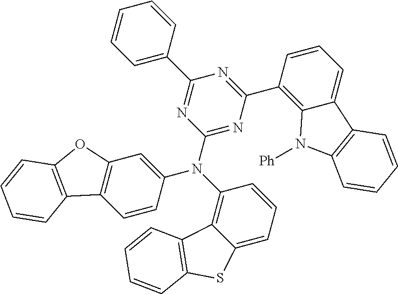 [SMILES: c1ccc(-c2nc(-c3cccc4c5ccccc5n(-c5ccccc5)c34)nc(N(c3ccc4c(c3)oc3ccccc34)c3cccc4sc5ccccc5c34)n2)cc1] | 53% |
| 1-371 | 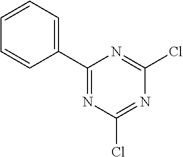 [SMILES: Clc1nc(Cl)nc(-c2ccccc2)n1] | 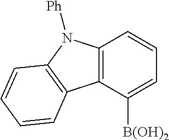 [SMILES: OB(O)c1cccc2c1c1ccccc1n2-c1ccccc1] | 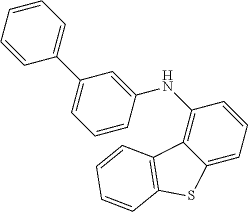 [SMILES: c1ccc(-c2cccc(Nc3cccc4sc5ccccc5c34)c2)cc1] | 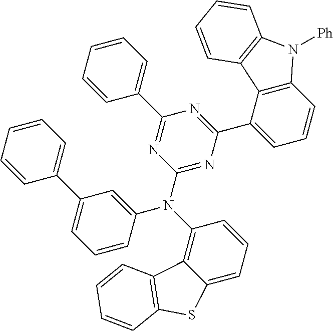 [SMILES: c1ccc(-c2cccc(N(c3nc(-c4ccccc4)nc(-c4cccc5c4c4ccccc4n5-c4ccccc4)n3)c3cccc4sc5ccccc5c34)c2)cc1] | 55% |

### <Preparation Example 2> Preparation of Compound 1-3

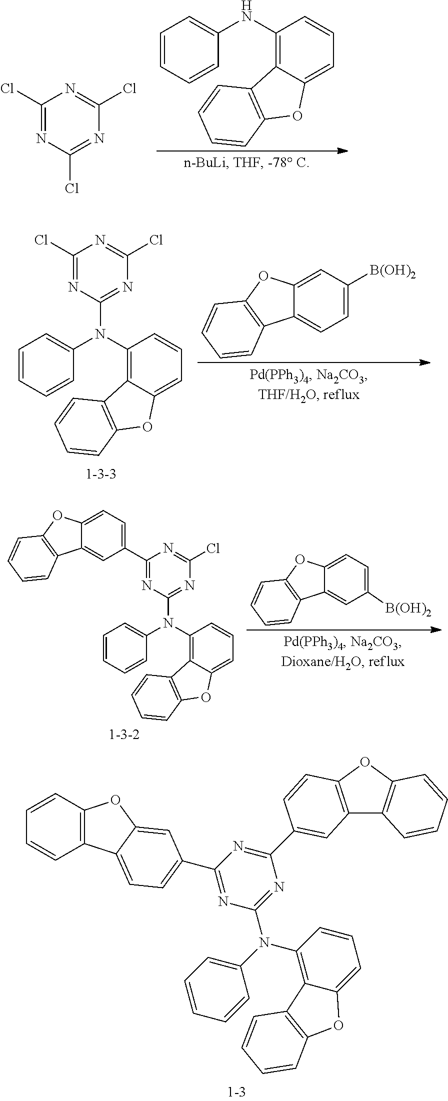

### 1) Preparation of Compound 1-3-3

After dissolving N-phenyldibenzo[b,d]furan-1-amine [A](20.0 g, 77.0 mM) in THE (200 mL), n-BuLi (n-butyllithium) (37.0 mL, 92.4 mM) were introduced thereto at −78° C., and then 2,4,6-trichloro-1,3,5-triazine (14.2 g, 77.0 mM) were introduced thereto, and the mixture was refluxed for 2 hours at room temperature. After the reaction was completed, the result was extracted by introducing distilled water and DCM thereto at room temperature, and after drying the organic layer with MgSO₄, the solvent was removed using a rotary evaporator. The reaction material was purified by column chromatography (DCM:Hex=1:4) to obtain Compound 1-3-3 (21.9 g, 70%).

### 2) Preparation of Compound 1-3-2

Compound 1-3-3 (21.0 g, 51.0 mM), dibenzo[b,d]furan-3-ylboronic acid [B] (8.3 g, 39.2 mM), Pd(PPh₃)₄ (2.3 g, 2.0 mM) and Na₂CO₃ (8.3 g, 78.4 mM) were dissolved in THF/H₂O (80 mL/15 mL), and refluxed for 24 hours. After the reaction was completed, the result was extracted by introducing distilled water and DCM thereto at room temperature, and after drying the organic layer with MgSO₄, the solvent was removed using a rotary evaporator. The reaction material was purified by column chromatography (DCM:Hex=1:4), and recrystallized with methanol to obtain Compound 1-3-2 (15.4 g, 73%).

### 3) Preparation of Compound 1-3

Compound 1-3-2 (15.0 g, 27.0 mM), dibenzo[b,d]furan-2-ylboronic acid [C] (5.7 g, 27.0 mM), Pd(PPh₃)₄ (1.6 g, 1.4 mM) and K₂CO₃ (7.5 g, 54.0 mM) were dissolved in 1,4-dioxane/H₂O (150 mL/30 mL), and refluxed for 24 hours. After the reaction was completed, the result was extracted by introducing distilled water and DCM thereto at room temperature, and after drying the organic layer with MgSO₄, the solvent was removed using a rotary evaporator. The reaction material was purified by column chromatography (DCM:Hex=1:3), and recrystallized with methanol to obtain target Compound 1-3 (13.9 g, 77%).

Target Compound D2 was synthesized in the same manner as in Preparation Example 2 except that Compound A2 of the following Table 2 was used instead of N-phenyldibenzo[b,d]furan-1-amine, Compound B2 of the following Table 2 was used instead of dibenzo[b,d]furan-3-ylboronic acid, and Compound C2 of the following Table 2 was used instead of dibenzo[b,d]furan-2-ylboronic acid.

TABLE 2

| Com- | | | | | |
|---|---|---|---|---|---|
| pound | | | | Target Compound | |
| No. | Compound A2 | Compound B2 | Compound C2 | D2 | Yield |
| 1-12 | 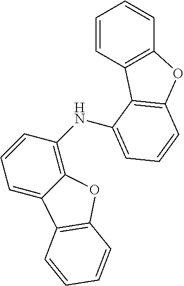 [SMILES: c1ccc2c(c1)oc1c(Nc3cccc4oc5ccccc5c34)cccc12] | 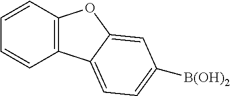 [SMILES: OB(O)c1ccc2c(c1)oc1ccccc12] | 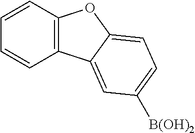 [SMILES: OB(O)c1ccc2oc3ccccc3c2c1] | 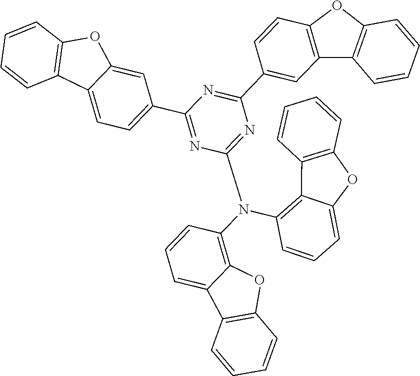 [SMILES: c1ccc2c(c1)oc1cc(-c3nc(-c4ccc5oc6ccccc6c5c4)nc(N(c4cccc5c4oc4ccccc45)c4cccc5oc6ccccc6c45)n3)ccc12] | 41% |
| 1-20 | 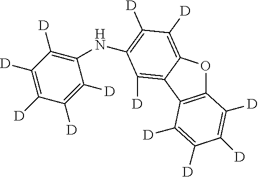 [SMILES: [2H]c1c([2H])c([2H])c(Nc2c([2H])c([2H])c3oc4c([2H])c([2H])c([2H])c([2H])c4c3c2[2H])c([2H])c1[2H]] | 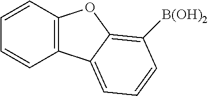 [SMILES: OB(O)c1cccc2c1oc1ccccc12] | 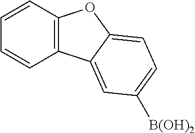 [SMILES: OB(O)c1ccc2oc3ccccc3c2c1] | 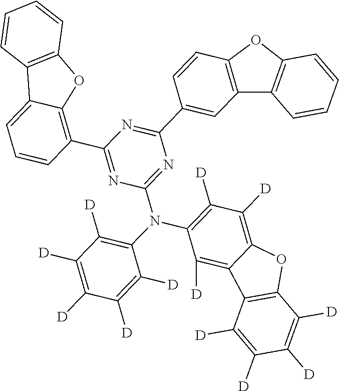 [SMILES: [2H]c1c([2H])c([2H])c(N(c2nc(-c3ccc4oc5ccccc5c4c3)nc(-c3cccc4c3oc3ccccc34)n2)c2c([2H])c([2H])c3oc4c([2H])c([2H])c([2H])c([2H])c4c3c2[2H])c([2H])c1[2H]] | 39% |
| 1-42 | 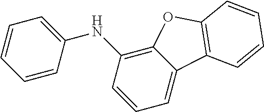 [SMILES: c1ccc(Nc2cccc3c2oc2ccccc23)cc1] | 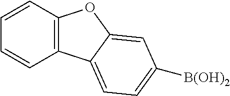 [SMILES: OB(O)c1ccc2c(c1)oc1ccccc12] | 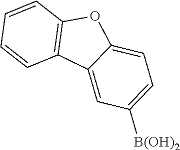 [SMILES: OB(O)c1ccc2oc3ccccc3c2c1] | 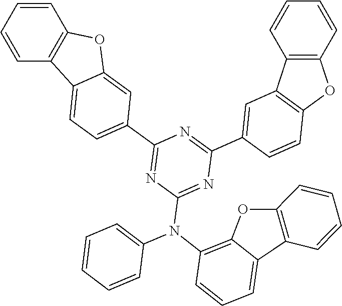 [SMILES: c1ccc(N(c2nc(-c3ccc4c(c3)oc3ccccc34)nc(-c3ccc4oc5ccccc5c4c3)n2)c2cccc3c2oc2ccccc23)cc1] | 40% |
| 1-61 | 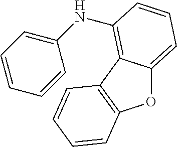 [SMILES: c1ccc(Nc2cccc3oc4ccccc4c23)cc1] | 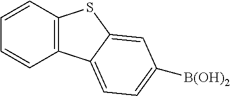 [SMILES: OB(O)c1ccc2c(c1)sc1ccccc12] | 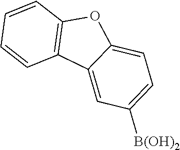 [SMILES: OB(O)c1ccc2oc3ccccc3c2c1] | 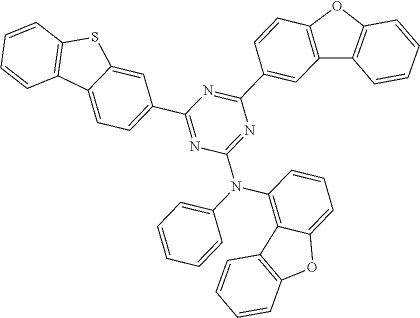 [SMILES: c1ccc(N(c2nc(-c3ccc4c(c3)sc3ccccc34)nc(-c3ccc4oc5ccccc5c4c3)n2)c2cccc3oc4ccccc4c23)cc1] | 39% |
| 1-67 | 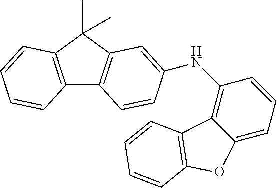 [SMILES: CC1(C)c2ccccc2-c2ccc(Nc3cccc4oc5ccccc5c34)cc21] | 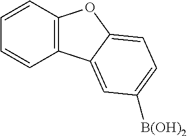 [SMILES: OB(O)c1ccc2oc3ccccc3c2c1] | 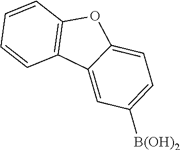 [SMILES: OB(O)c1ccc2oc3ccccc3c2c1] | 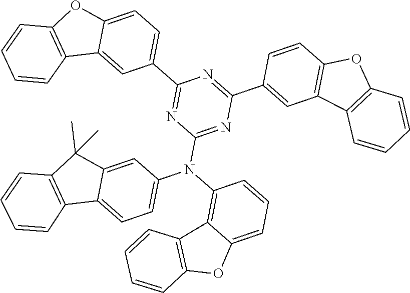 [SMILES: CC1(C)c2ccccc2-c2ccc(N(c3nc(-c4ccc5oc6ccccc6c5c4)nc(-c4ccc5oc6ccccc6c5c4)n3)c3cccc4oc5ccccc5c34)cc21] | 40% |
| 1-69 | 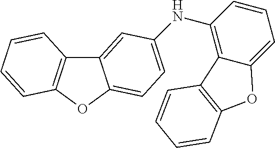 [SMILES: c1ccc2c(c1)oc1ccc(Nc3cccc4oc5ccccc5c34)cc12] | 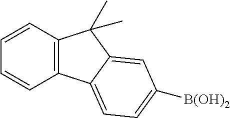 [SMILES: CC1(C)c2ccccc2-c2ccc(B(O)O)cc21] | 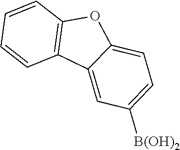 [SMILES: OB(O)c1ccc2oc3ccccc3c2c1] | 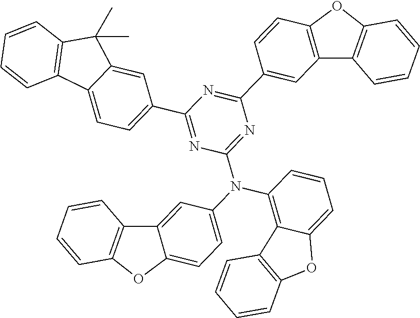 [SMILES: CC1(C)c2ccccc2-c2ccc(-c3nc(-c4ccc5oc6ccccc6c5c4)nc(N(c4ccc5oc6ccccc6c5c4)c4cccc5oc6ccccc6c45)n3)cc21] | 41% |
| 1-81 | 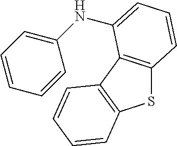 [SMILES: c1ccc(Nc2cccc3sc4ccccc4c23)cc1] | 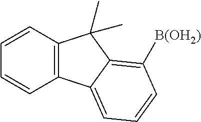 | 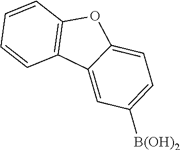 [SMILES: OB(O)c1ccc2oc3ccccc3c2c1] | 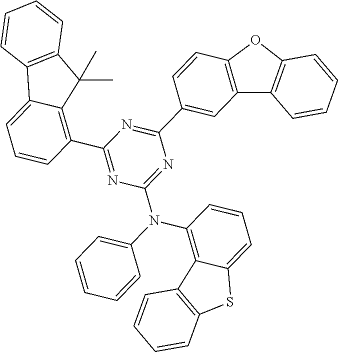 [SMILES: CC1(C)c2ccccc2-c2cccc(-c3nc(-c4ccc5oc6ccccc6c5c4)nc(N(c4ccccc4)c4cccc5sc6ccccc6c45)n3)c21] | 42% |
| 1-89 | 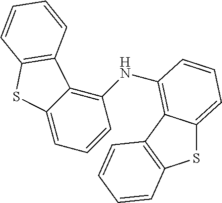 [SMILES: c1ccc2c(c1)sc1cccc(Nc3cccc4sc5ccccc5c34)c12] | 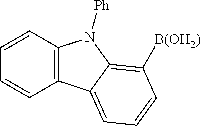 | 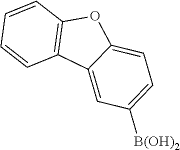 [SMILES: OB(O)c1ccc2oc3ccccc3c2c1] | 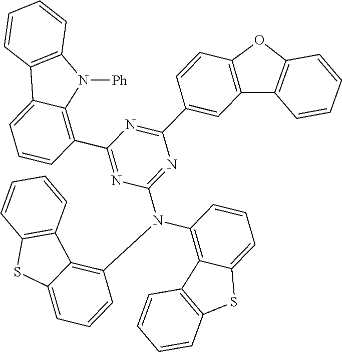 [SMILES: c1ccc(-n2c3ccccc3c3cccc(-c4nc(-c5ccc6oc7ccccc7c6c5)nc(N(c5cccc6sc7ccccc7c56)c5cccc6sc7ccccc7c56)n4)c32)cc1] | 42% |
| 1-102 | 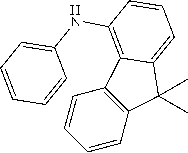 [SMILES: CC1(C)c2ccccc2-c2c(Nc3ccccc3)cccc21] | 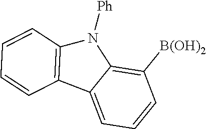 [SMILES: OB(O)c1cccc2c3ccccc3n(-c3ccccc3)c12] | 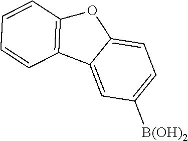 [SMILES: OB(O)c1ccc2oc3ccccc3c2c1] | 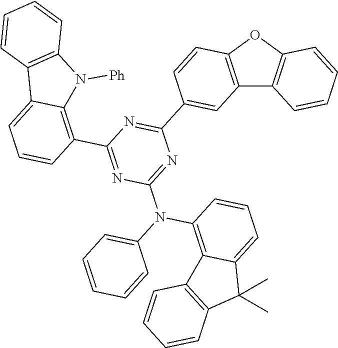 [SMILES: CC1(C)c2ccccc2-c2c(N(c3ccccc3)c3nc(-c4ccc5oc6ccccc6c5c4)nc(-c4cccc5c6ccccc6n(-c6ccccc6)c45)n3)cccc21] | 45% |
| 1-106 | 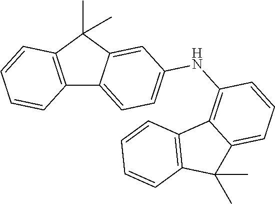 [SMILES: CC1(C)c2ccccc2-c2ccc(Nc3cccc4c3-c3ccccc3C4(C)C)cc21] | 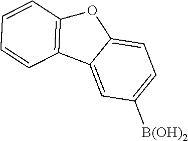 [SMILES: OB(O)c1ccc2oc3ccccc3c2c1] | 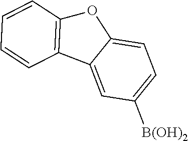 [SMILES: OB(O)c1ccc2oc3ccccc3c2c1] | 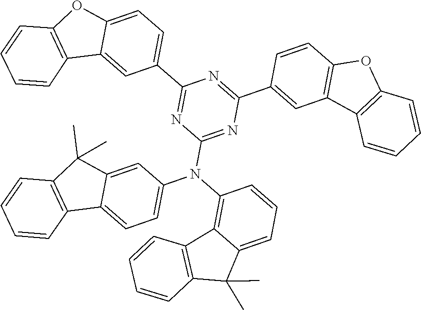 [SMILES: CC1(C)c2ccccc2-c2ccc(N(c3nc(-c4ccc5oc6ccccc6c5c4)nc(-c4ccc5oc6ccccc6c5c4)n3)c3cccc4c3-c3ccccc3C4(C)C)cc21] | 42% |
| 1-109 | 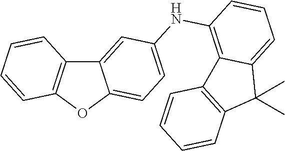 [SMILES: CC1(C)c2ccccc2-c2c(Nc3ccc4oc5ccccc5c4c3)cccc21] | 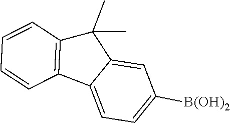 [SMILES: CC1(C)c2ccccc2-c2ccc(B(O)O)cc21] | 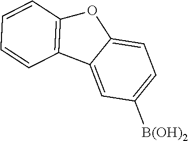 [SMILES: OB(O)c1ccc2oc3ccccc3c2c1] | 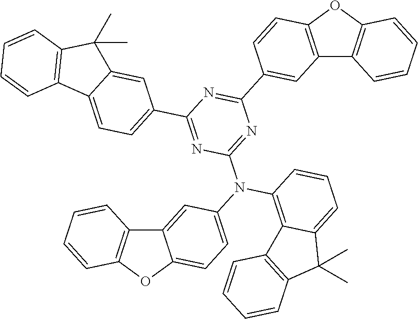 [SMILES: CC1(C)c2ccccc2-c2ccc(-c3nc(-c4ccc5oc6ccccc6c5c4)nc(N(c4ccc5oc6ccccc6c5c4)c4cccc5c4-c4ccccc4C5(C)C)n3)cc21] | 41% |
| 1-116 | 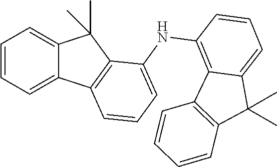 [SMILES: CC1(C)c2ccccc2-c2c(Nc3cccc4c3C(C)(C)c3ccccc3-4)cccc21] | 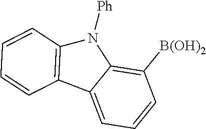 [SMILES: OB(O)c1cccc2c3ccccc3n(-c3ccccc3)c12] | 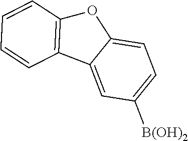 [SMILES: OB(O)c1ccc2oc3ccccc3c2c1] | 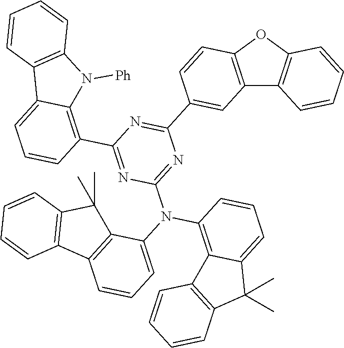 [SMILES: CC1(C)c2ccccc2-c2c(N(c3nc(-c4ccc5oc6ccccc6c5c4)nc(-c4cccc5c6ccccc6n(-c6ccccc6)c45)n3)c3cccc4c3C(C)(C)c3ccccc3-4)cccc21] | 39% |
| 1-118 | 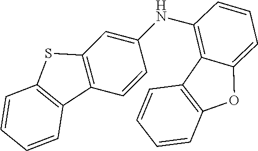 [SMILES: c1ccc2c(c1)oc1cccc(Nc3ccc4c(c3)sc3ccccc34)c12] | 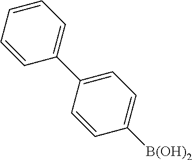 [SMILES: OB(O)c1ccc(-c2ccccc2)cc1] | 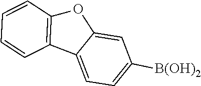 [SMILES: OB(O)c1ccc2c(c1)oc1ccccc12] | 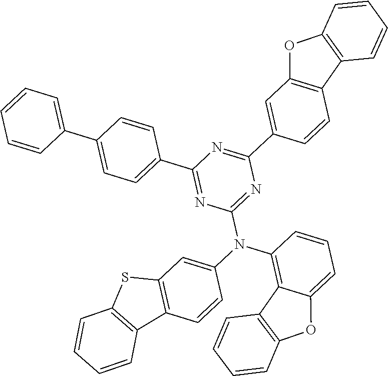 [SMILES: c1ccc(-c2ccc(-c3nc(-c4ccc5c(c4)oc4ccccc45)nc(N(c4ccc5c(c4)sc4ccccc45)c4cccc5oc6ccccc6c45)n3)cc2)cc1] | 42% |
| 1-125 | 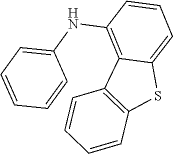 [SMILES: c1ccc(Nc2cccc3sc4ccccc4c23)cc1] | 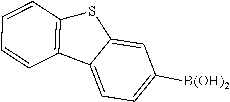 [SMILES: OB(O)c1ccc2c(c1)sc1ccccc12] | 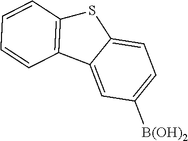 [SMILES: OB(O)c1ccc2sc3ccccc3c2c1] | 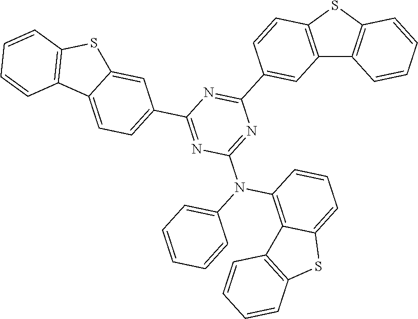 [SMILES: c1ccc(N(c2nc(-c3ccc4c(c3)sc3ccccc34)nc(-c3ccc4sc5ccccc5c4c3)n2)c2cccc3sc4ccccc4c23)cc1] | 41% |
| 1-136 | 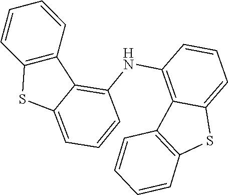 [SMILES: c1ccc2c(c1)sc1cccc(Nc3cccc4sc5ccccc5c34)c12] | 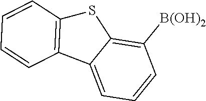 [SMILES: OB(O)c1cccc2c1sc1ccccc12] | 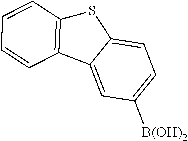 [SMILES: OB(O)c1ccc2sc3ccccc3c2c1] | 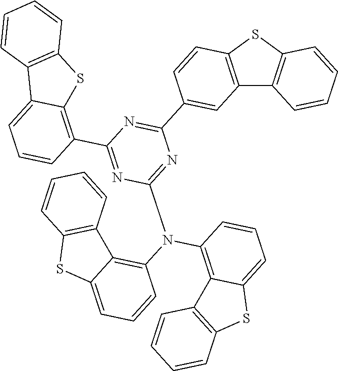 [SMILES: c1ccc2c(c1)sc1ccc(-c3nc(-c4cccc5c4sc4ccccc45)nc(N(c4cccc5sc6ccccc6c45)c4cccc5sc6ccccc6c45)n3)cc12] | 42% |
| 1-142 | 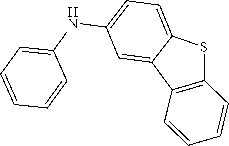 [SMILES: c1ccc(Nc2ccc3sc4ccccc4c3c2)cc1] | 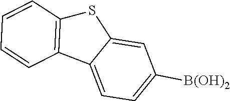 [SMILES: OB(O)c1ccc2c(c1)sc1ccccc12] | 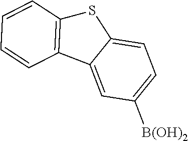 [SMILES: OB(O)c1ccc2sc3ccccc3c2c1] | 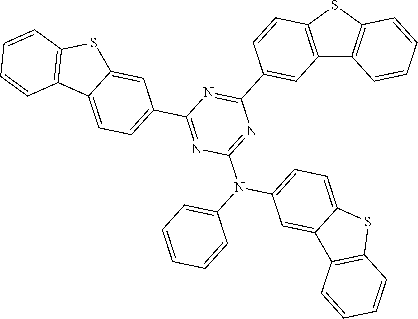 [SMILES: c1ccc(N(c2ccc3sc4ccccc4c3c2)c2nc(-c3ccc4c(c3)sc3ccccc34)nc(-c3ccc4sc5ccccc5c4c3)n2)cc1] | 43% |
| 1-163 | 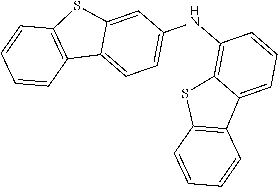 [SMILES: c1ccc2c(c1)sc1cc(Nc3cccc4c3sc3ccccc34)ccc12] | 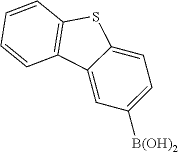 [SMILES: OB(O)c1ccc2sc3ccccc3c2c1] | 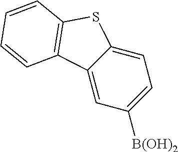 [SMILES: OB(O)c1ccc2sc3ccccc3c2c1] | 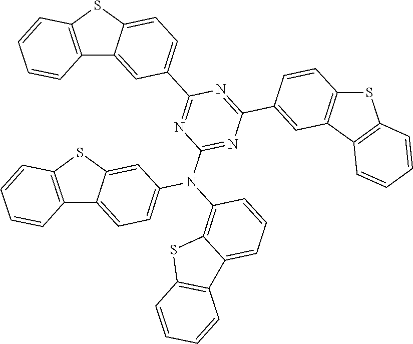 [SMILES: c1ccc2c(c1)sc1cc(N(c3nc(-c4ccc5sc6ccccc6c5c4)nc(-c4ccc5sc6ccccc6c5c4)n3)c3cccc4c3sc3ccccc34)ccc12] | 44% |
| 1-178 | 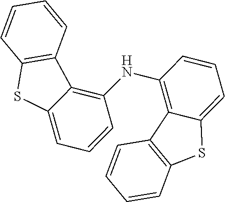 [SMILES: c1ccc2c(c1)sc1cccc(Nc3cccc4sc5ccccc5c34)c12] | 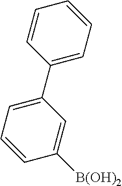 [SMILES: OB(O)c1cccc(-c2ccccc2)c1] | 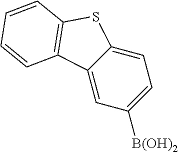 [SMILES: OB(O)c1ccc2sc3ccccc3c2c1] | 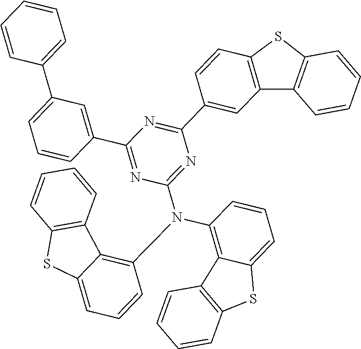 [SMILES: c1ccc(-c2cccc(-c3nc(-c4ccc5sc6ccccc6c5c4)nc(N(c4cccc5sc6ccccc6c45)c4cccc5sc6ccccc6c45)n3)c2)cc1] | 46% |
| 1-202 | 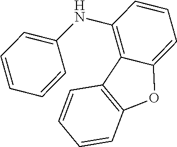 [SMILES: c1ccc(Nc2cccc3oc4ccccc4c23)cc1] | 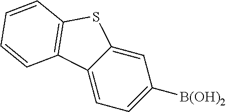 [SMILES: OB(O)c1ccc2c(c1)sc1ccccc12] | 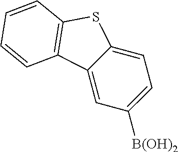 [SMILES: OB(O)c1ccc2sc3ccccc3c2c1] | 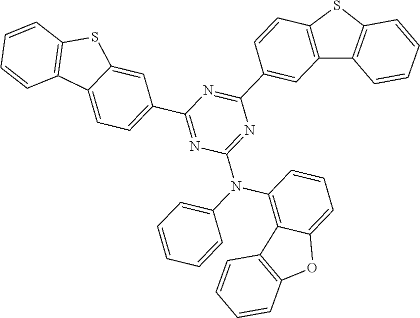 [SMILES: c1ccc(N(c2nc(-c3ccc4c(c3)sc3ccccc34)nc(-c3ccc4sc5ccccc5c4c3)n2)c2cccc3oc4ccccc4c23)cc1] | 42% |
| 1-207 | 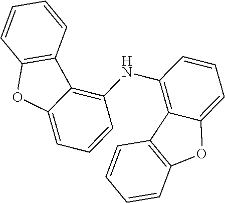 [SMILES: c1ccc2c(c1)oc1cccc(Nc3cccc4oc5ccccc5c34)c12] | 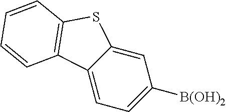 [SMILES: OB(O)c1ccc2c(c1)sc1ccccc12] | 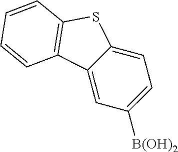 [SMILES: OB(O)c1ccc2sc3ccccc3c2c1] | 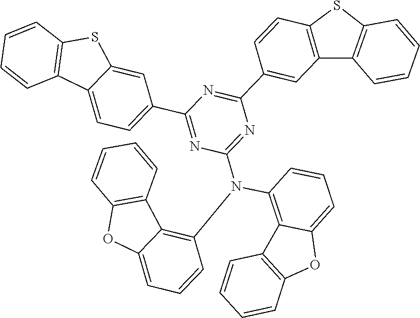 [SMILES: c1ccc2c(c1)oc1cccc(N(c3nc(-c4ccc5c(c4)sc4ccccc45)nc(-c4ccc5sc6ccccc6c5c4)n3)c3cccc4oc5ccccc5c34)c12] | 45% |
| 1-211 | 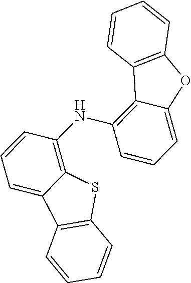 [SMILES: c1ccc2c(c1)oc1cccc(Nc3cccc4c3sc3ccccc34)c12] | 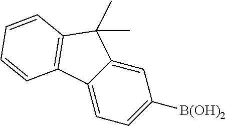 [SMILES: CC1(C)c2ccccc2-c2ccc(B(O)O)cc21] | 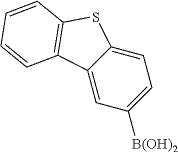 [SMILES: OB(O)c1ccc2sc3ccccc3c2c1] | 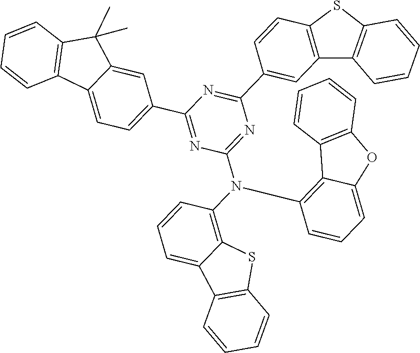 [SMILES: CC1(C)c2ccccc2-c2ccc(-c3nc(-c4ccc5sc6ccccc6c5c4)nc(N(c4cccc5c4sc4ccccc45)c4cccc5oc6ccccc6c45)n3)cc21] | 40% |
| 1-230 | 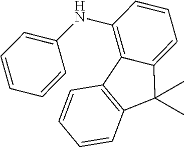 [SMILES: CC1(C)c2ccccc2-c2c(Nc3ccccc3)cccc21] | 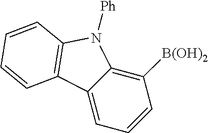 [SMILES: OB(O)c1cccc2c3ccccc3n(-c3ccccc3)c12] | 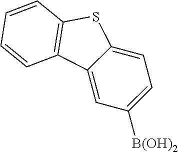 [SMILES: OB(O)c1ccc2sc3ccccc3c2c1] | 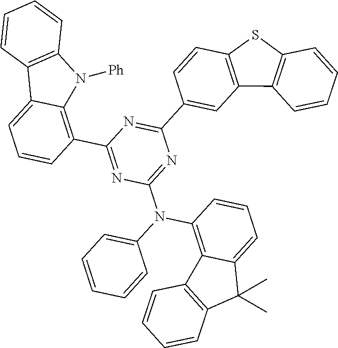 [SMILES: CC1(C)c2ccccc2-c2c(N(c3ccccc3)c3nc(-c4ccc5sc6ccccc6c5c4)nc(-c4cccc5c6ccccc6n(-c6ccccc6)c45)n3)cccc21] | 39% |
| 1-233 | 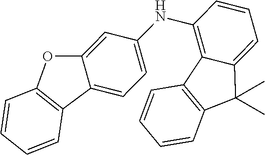 [SMILES: CC1(C)c2ccccc2-c2c(Nc3ccc4c(c3)oc3ccccc34)cccc21] | 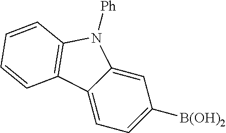 [SMILES: OB(O)c1ccc2c3ccccc3n(-c3ccccc3)c2c1] | 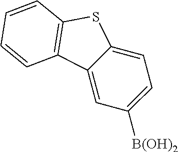 [SMILES: OB(O)c1ccc2sc3ccccc3c2c1] | 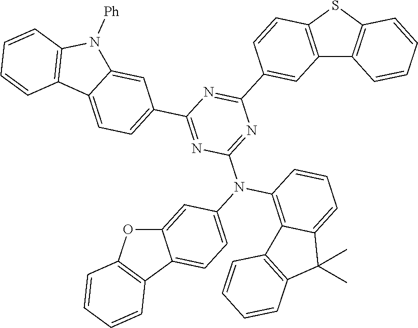 [SMILES: CC1(C)c2ccccc2-c2c(N(c3ccc4c(c3)oc3ccccc34)c3nc(-c4ccc5sc6ccccc6c5c4)nc(-c4ccc5c6ccccc6n(-c6ccccc6)c5c4)n3)cccc21] | 38% |
| 1-237 | 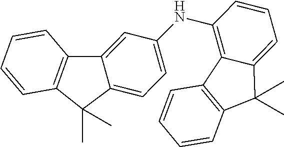 [SMILES: CC1(C)c2ccccc2-c2cc(Nc3cccc4c3-c3ccccc3C4(C)C)ccc21] | 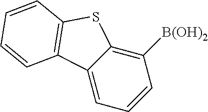 [SMILES: OB(O)c1cccc2c1sc1ccccc12] | 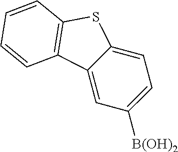 [SMILES: OB(O)c1ccc2sc3ccccc3c2c1] | 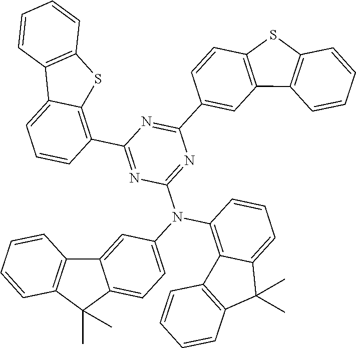 [SMILES: CC1(C)c2ccccc2-c2cc(N(c3nc(-c4ccc5sc6ccccc6c5c4)nc(-c4cccc5c4sc4ccccc45)n3)c3cccc4c3-c3ccccc3C4(C)C)ccc21] | 45% |
| 1-239 | 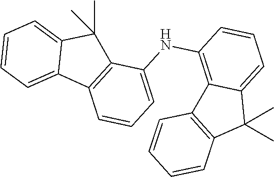 [SMILES: CC1(C)c2ccccc2-c2c(Nc3cccc4c3C(C)(C)c3ccccc3-4)cccc21] | 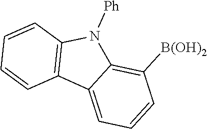 [SMILES: OB(O)c1cccc2c3ccccc3n(-c3ccccc3)c12] | 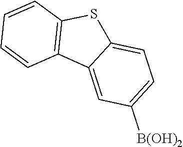 [SMILES: OB(O)c1ccc2sc3ccccc3c2c1] | 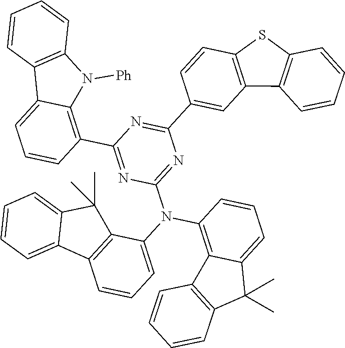 [SMILES: CC1(C)c2ccccc2-c2c(N(c3nc(-c4ccc5sc6ccccc6c5c4)nc(-c4cccc5c6ccccc6n(-c6ccccc6)c45)n3)c3cccc4c3C(C)(C)c3ccccc3-4)cccc21] | 40% |
| 1-244 | 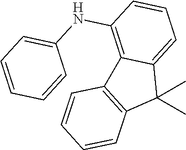 [SMILES: CC1(C)c2ccccc2-c2c(Nc3ccccc3)cccc21] | 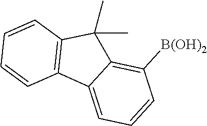 [SMILES: CC1(C)c2ccccc2-c2cccc(B(O)O)c21] | 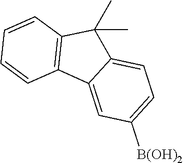 [SMILES: CC1(C)c2ccccc2-c2cc(B(O)O)ccc21] | 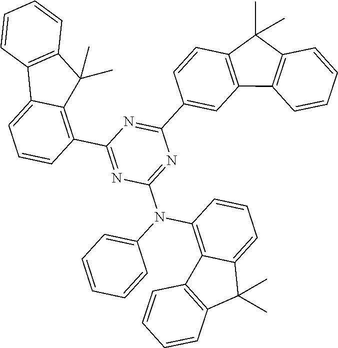 [SMILES: CC1(C)c2ccccc2-c2cc(-c3nc(-c4cccc5c4C(C)(C)c4ccccc4-5)nc(N(c4ccccc4)c4cccc5c4-c4ccccc4C5(C)C)n3)ccc21] | 45% |
| 1-249 | 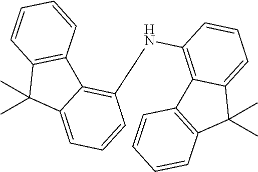 | 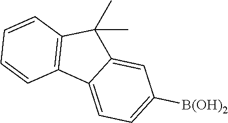 [SMILES: CC1(C)c2ccccc2-c2ccc(B(O)O)cc21] | 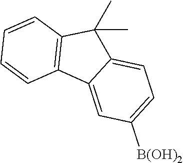 [SMILES: CC1(C)c2ccccc2-c2cc(B(O)O)ccc21] | 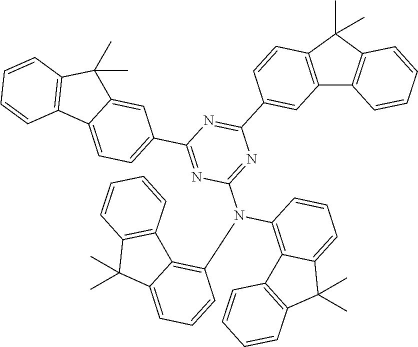 [SMILES: CC1(C)c2ccccc2-c2cc(-c3nc(-c4ccc5c(c4)C(C)(C)c4ccccc4-5)nc(N(c4cccc5c4-c4ccccc4C5(C)C)c4cccc5c4-c4ccccc4C5(C)C)n3)ccc21] | 44% |
| 1-259 | 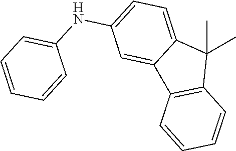 [SMILES: CC1(C)c2ccccc2-c2cc(Nc3ccccc3)ccc21] | 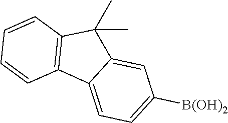 [SMILES: CC1(C)c2ccccc2-c2ccc(B(O)O)cc21] | 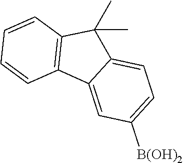 [SMILES: CC1(C)c2ccccc2-c2cc(B(O)O)ccc21] | 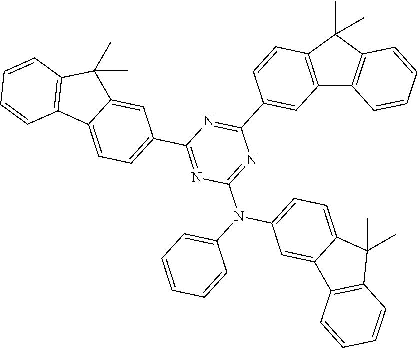 [SMILES: CC1(C)c2ccccc2-c2cc(-c3nc(-c4ccc5c(c4)C(C)(C)c4ccccc4-5)nc(N(c4ccccc4)c4ccc5c(c4)-c4ccccc4C5(C)C)n3)ccc21] | 43% |
| 1-293 | 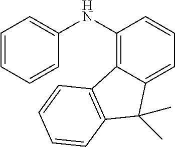 [SMILES: CC1(C)c2ccccc2-c2c(Nc3ccccc3)cccc21] | 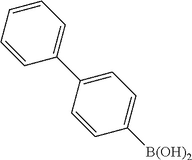 [SMILES: OB(O)c1ccc(-c2ccccc2)cc1] | 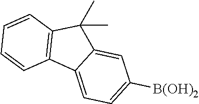 [SMILES: CC1(C)c2ccccc2-c2ccc(B(O)O)cc21] | 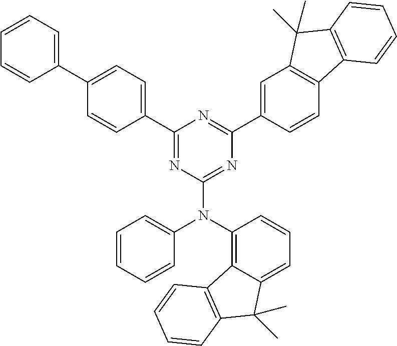 [SMILES: CC1(C)c2ccccc2-c2ccc(-c3nc(-c4ccc(-c5ccccc5)cc4)nc(N(c4ccccc4)c4cccc5c4-c4ccccc4C5(C)C)n3)cc21] | 42% |
| 1-306 | 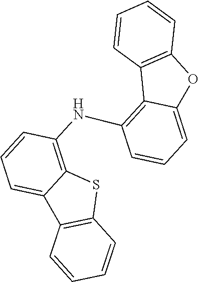 [SMILES: c1ccc2c(c1)oc1cccc(Nc3cccc4c3sc3ccccc34)c12] | 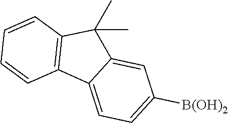 [SMILES: CC1(C)c2ccccc2-c2ccc(B(O)O)cc21] | 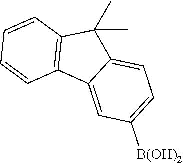 [SMILES: CC1(C)c2ccccc2-c2cc(B(O)O)ccc21] | 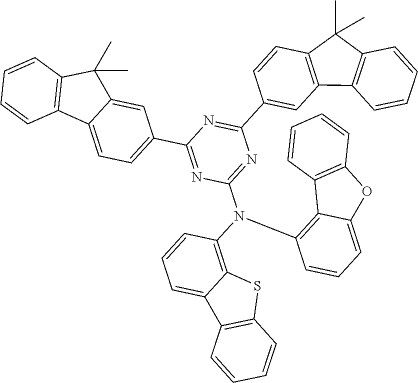 [SMILES: CC1(C)c2ccccc2-c2cc(-c3nc(-c4ccc5c(c4)C(C)(C)c4ccccc4-5)nc(N(c4cccc5c4sc4ccccc45)c4cccc5oc6ccccc6c45)n3)ccc21] | 41% |
| 1-309 | 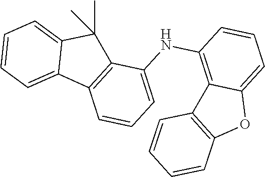 [SMILES: CC1(C)c2ccccc2-c2cccc(Nc3cccc4oc5ccccc5c34)c21] | 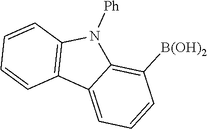 [SMILES: OB(O)c1cccc2c3ccccc3n(-c3ccccc3)c12] | 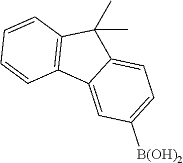 [SMILES: CC1(C)c2ccccc2-c2cc(B(O)O)ccc21] | 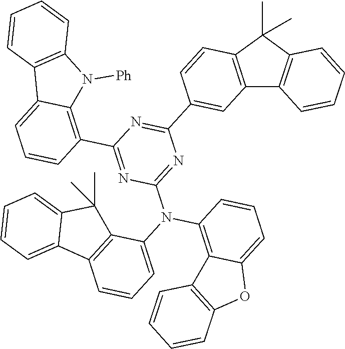 [SMILES: CC1(C)c2ccccc2-c2cc(-c3nc(-c4cccc5c6ccccc6n(-c6ccccc6)c45)nc(N(c4cccc5c4C(C)(C)c4ccccc4-5)c4cccc5oc6ccccc6c45)n3)ccc21] | 39% |
| 1-312 | 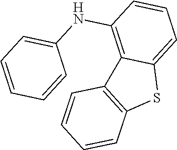 [SMILES: c1ccc(Nc2cccc3sc4ccccc4c23)cc1] | 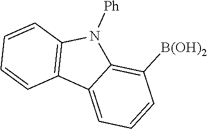 [SMILES: OB(O)c1cccc2c3ccccc3n(-c3ccccc3)c12] | 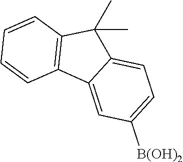 [SMILES: CC1(C)c2ccccc2-c2cc(B(O)O)ccc21] | 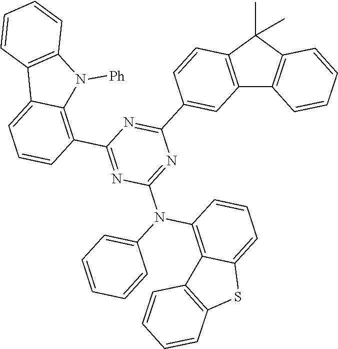 [SMILES: CC1(C)c2ccccc2-c2cc(-c3nc(-c4cccc5c6ccccc6n(-c6ccccc6)c45)nc(N(c4ccccc4)c4cccc5sc6ccccc6c45)n3)ccc21] | 46% |
| 1-320 | 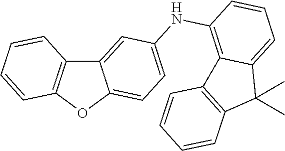 [SMILES: CC1(C)c2ccccc2-c2c(Nc3ccc4oc5ccccc5c4c3)cccc21] | 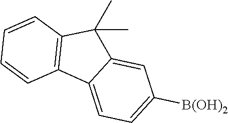 [SMILES: CC1(C)c2ccccc2-c2ccc(B(O)O)cc21] | 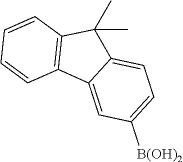 [SMILES: CC1(C)c2ccccc2-c2cc(B(O)O)ccc21] | 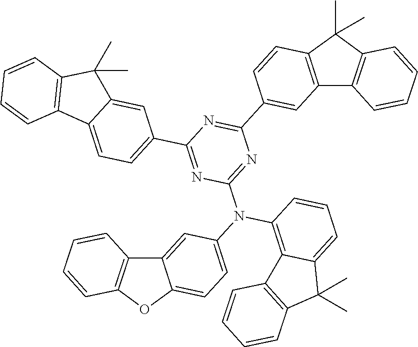 [SMILES: CC1(C)c2ccccc2-c2cc(-c3nc(-c4ccc5c(c4)C(C)(C)c4ccccc4-5)nc(N(c4ccc5oc6ccccc6c5c4)c4cccc5c4-c4ccccc4C5(C)C)n3)ccc21] | 44% |
| 1-323 | 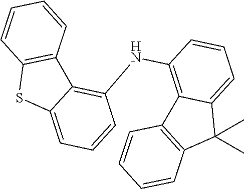 [SMILES: CC1(C)c2ccccc2-c2c(Nc3cccc4sc5ccccc5c34)cccc21] | 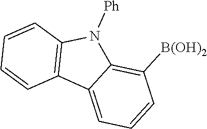 [SMILES: OB(O)c1cccc2c3ccccc3n(-c3ccccc3)c12] | 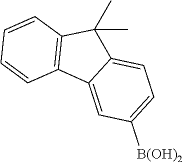 [SMILES: CC1(C)c2ccccc2-c2cc(B(O)O)ccc21] | 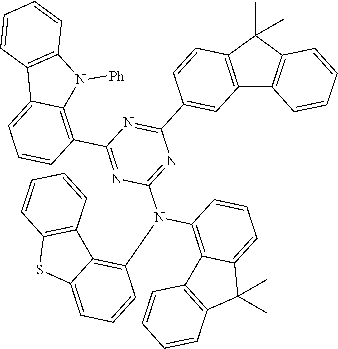 [SMILES: CC1(C)c2ccccc2-c2cc(-c3nc(-c4cccc5c6ccccc6n(-c6ccccc6)c45)nc(N(c4cccc5c4-c4ccccc4C5(C)C)c4cccc5sc6ccccc6c45)n3)ccc21] | 41% |
| 1-333 | 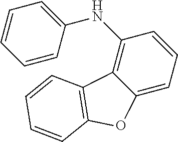 [SMILES: c1ccc(Nc2cccc3oc4ccccc4c23)cc1] | 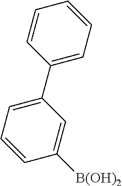 [SMILES: OB(O)c1cccc(-c2ccccc2)c1] | 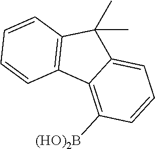 [SMILES: CC1(C)c2ccccc2-c2c(B(O)O)cccc21] | 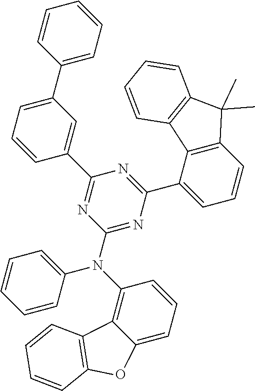 [SMILES: CC1(C)c2ccccc2-c2c(-c3nc(-c4cccc(-c5ccccc5)c4)nc(N(c4ccccc4)c4cccc5oc6ccccc6c45)n3)cccc21] | 43% |
| 1-336 | 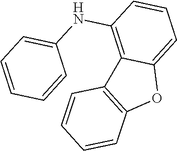 [SMILES: c1ccc(Nc2cccc3oc4ccccc4c23)cc1] | 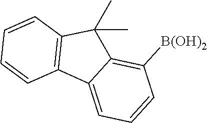 [SMILES: CC1(C)c2ccccc2-c2cccc(B(O)O)c21] | 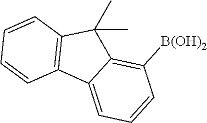 [SMILES: CC1(C)c2ccccc2-c2cccc(B(O)O)c21] | 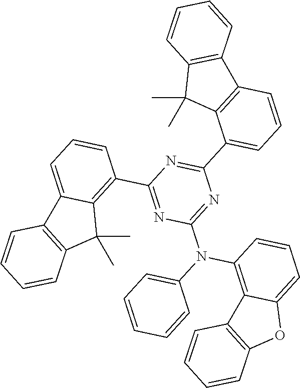 [SMILES: CC1(C)c2ccccc2-c2cccc(-c3nc(-c4cccc5c4C(C)(C)c4ccccc4-5)nc(N(c4ccccc4)c4cccc5oc6ccccc6c45)n3)c21] | 40% |
| 1-342 | 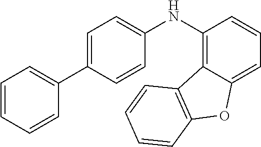 [SMILES: c1ccc(-c2ccc(Nc3cccc4oc5ccccc5c34)cc2)cc1] | 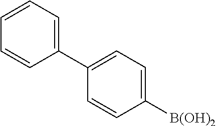 [SMILES: OB(O)c1ccc(-c2ccccc2)cc1] | 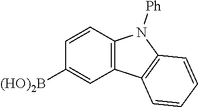 [SMILES: OB(O)c1ccc2c(c1)c1ccccc1n2-c1ccccc1] | 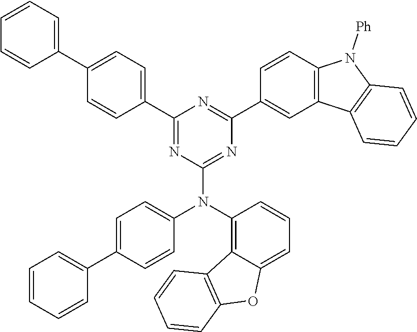 [SMILES: c1ccc(-c2ccc(-c3nc(-c4ccc5c(c4)c4ccccc4n5-c4ccccc4)nc(N(c4ccc(-c5ccccc5)cc4)c4cccc5oc6ccccc6c45)n3)cc2)cc1] | 40% |
| 1-349 | 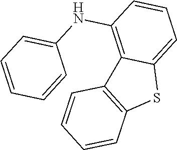 [SMILES: c1ccc(Nc2cccc3sc4ccccc4c23)cc1] | 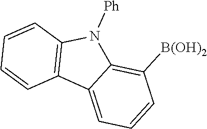 [SMILES: OB(O)c1cccc2c3ccccc3n(-c3ccccc3)c12] | 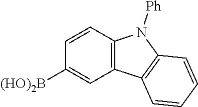 [SMILES: OB(O)c1ccc2c(c1)c1ccccc1n2-c1ccccc1] | 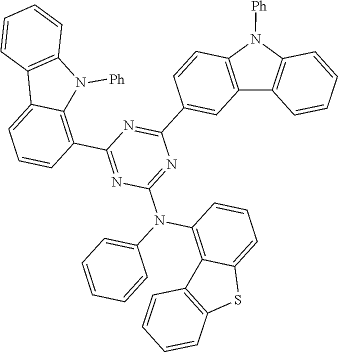 [SMILES: c1ccc(N(c2nc(-c3ccc4c(c3)c3ccccc3n4-c3ccccc3)nc(-c3cccc4c5ccccc5n(-c5ccccc5)c34)n2)c2cccc3sc4ccccc4c23)cc1] | 42% |
| 1-350 | 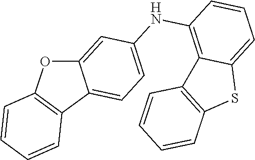 [SMILES: c1ccc2c(c1)oc1cc(Nc3cccc4sc5ccccc5c34)ccc12] | 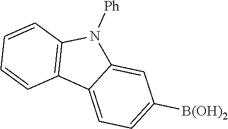 [SMILES: OB(O)c1ccc2c3ccccc3n(-c3ccccc3)c2c1] | 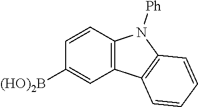 [SMILES: OB(O)c1ccc2c(c1)c1ccccc1n2-c1ccccc1] | 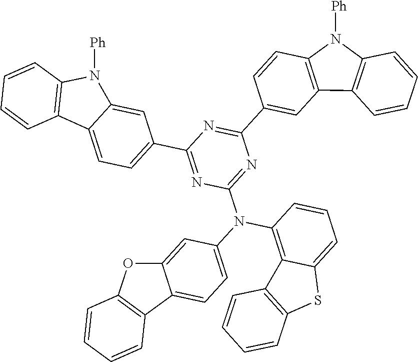 [SMILES: c1ccc(-n2c3ccccc3c3cc(-c4nc(-c5ccc6c7ccccc7n(-c7ccccc7)c6c5)nc(N(c5ccc6c(c5)oc5ccccc56)c5cccc6sc7ccccc7c56)n4)ccc32)cc1] | 44% |
| 1-360 | 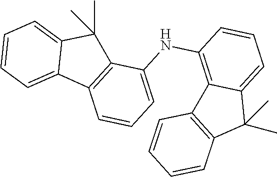 [SMILES: CC1(C)c2ccccc2-c2c(Nc3cccc4c3C(C)(C)c3ccccc3-4)cccc21] | 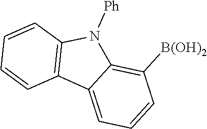 [SMILES: OB(O)c1cccc2c3ccccc3n(-c3ccccc3)c12] | 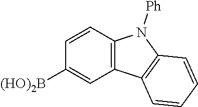 [SMILES: OB(O)c1ccc2c(c1)c1ccccc1n2-c1ccccc1] | 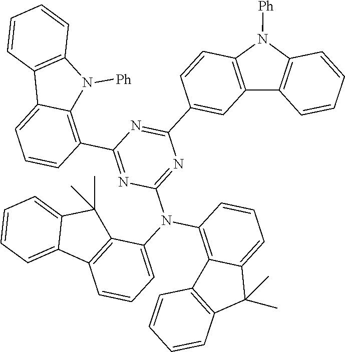 [SMILES: CC1(C)c2ccccc2-c2c(N(c3nc(-c4ccc5c(c4)c4ccccc4n5-c4ccccc4)nc(-c4cccc5c6ccccc6n(-c6ccccc6)c45)n3)c3cccc4c3C(C)(C)c3ccccc3-4)cccc21] | 41% |
| 1-368 | 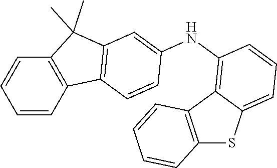 [SMILES: CC1(C)c2ccccc2-c2ccc(Nc3cccc4sc5ccccc5c34)cc21] | 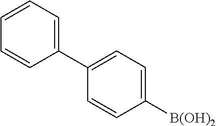 [SMILES: OB(O)c1ccc(-c2ccccc2)cc1] | 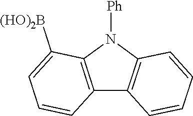 [SMILES: OB(O)c1cccc2c3ccccc3n(-c3ccccc3)c12] | 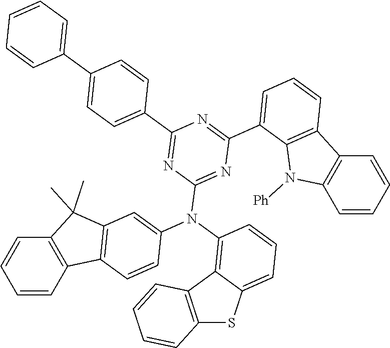 [SMILES: CC1(C)c2ccccc2-c2ccc(N(c3nc(-c4ccc(-c5ccccc5)cc4)nc(-c4cccc5c6ccccc6n(-c6ccccc6)c45)n3)c3cccc4sc5ccccc5c34)cc21] | 43% |
| 1-370 | 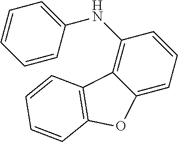 [SMILES: c1ccc(Nc2cccc3oc4ccccc4c23)cc1] | 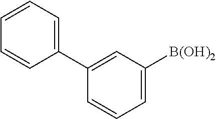 [SMILES: OB(O)c1cccc(-c2ccccc2)c1] | 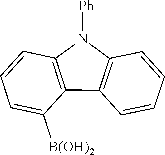 [SMILES: OB(O)c1cccc2c1c1ccccc1n2-c1ccccc1] | 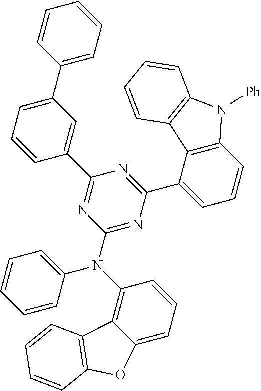 [SMILES: c1ccc(-c2cccc(-c3nc(-c4cccc5c4c4ccccc4n5-c4ccccc4)nc(N(c4ccccc4)c4cccc5oc6ccccc6c45)n3)c2)cc1] | 45% |

### <Preparation Example 2> Preparation of Compound 1-60

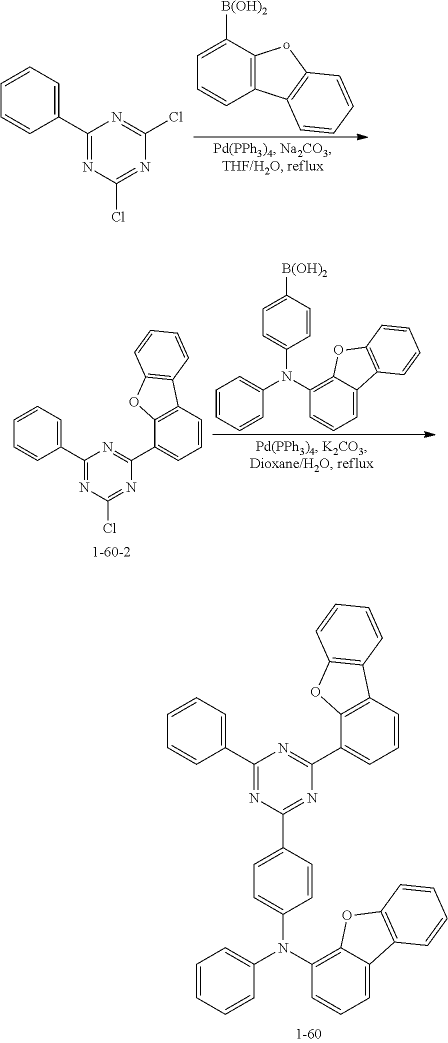

### 1) Preparation of Compound 1-60-2

2,3-Dichloro-6-phenyl-1,3,5-triazine (27.6 g, 122.2 mM), dibenzo[b,d]furan-4-ylboronic acid [A] (20.0 g, 94.0 mM), Pd(PPh₃)₄ (5.4 g, 4.7 mM) and Na₂CO₃ (19.9 g, 188.0 mM) were dissolved in THF/H₂O (200 mL/40 mL), and refluxed for 24 hours. After the reaction was completed, the result was extracted by introducing distilled water and DCM thereto at room temperature, and after drying the organic layer with MgSO₄, the solvent was removed using a rotary evaporator. The reaction material was purified by column chromatography (DCM:hexane=1:2) to obtain Compound 1-60-2 (20.5 g, 61%).

### 2) Preparation of Compound 1-60

Compound 1-60-2 (20.0 g, 55.0 mM), (4-(dibenzo[b,d]furan-4-yl(phenyl)amino)phenyl) boronic acid [B] (20.1 g, 55.0 mM), Pd(PPh₃)₄ (3.2 g, 2.8 mM) and K₂CO₃ (15.2 g, 110.0 mM) were dissolved in 1,4-dioxane/H₂O (200 mL/40 mL), and refluxed for 24 hours. After the reaction was completed, the result was extracted by introducing distilled water and DCM thereto at room temperature, and after drying the organic layer with MgSO₄, the solvent was removed using a rotary evaporator. The reaction material was purified by column chromatography (DCM:Hex=1:4), and recrystallized with methanol to obtain target Compound 1-60 (28.5 g, 79%).

Target Compound C3 was synthesized in the same manner as in Preparation Example 3 except that Compound A3 of the following Table 3 was used instead of dibenzo[b,d]furan-4-ylboronic acid, and Compound B3 of the following Table 3 was used instead of (4-(dibenzo[b,d]furan-4-yl(phenyl)amino)phenyl)boronic acid.

TABLE 3

| Compound | | | | |
|---|---|---|---|---|
| No. | Compound A3 | Compound B3 | Target Compound C3 | Yield |
| 1-192 | 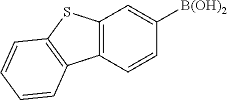 [SMILES: OB(O)c1ccc2c(c1)sc1ccccc12] | 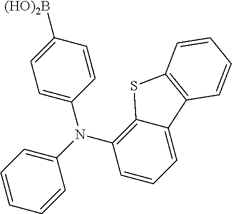 [SMILES: OB(O)c1ccc(N(c2ccccc2)c2cccc3c2sc2ccccc23)cc1] | 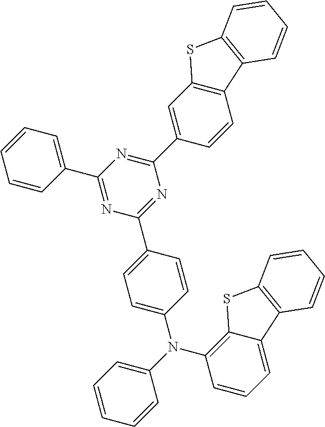 [SMILES: c1ccc(-c2nc(-c3ccc(N(c4ccccc4)c4cccc5c4sc4ccccc45)cc3)nc(-c3ccc4c(c3)sc3ccccc34)n2)cc1] | 52% |
| 1-240 | 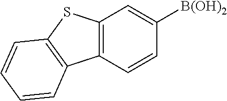 [SMILES: OB(O)c1ccc2c(c1)sc1ccccc12] | 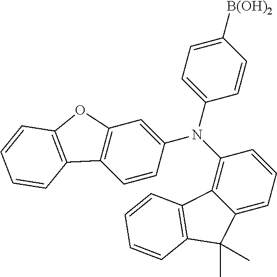 [SMILES: CC1(C)c2ccccc2-c2c(N(c3ccc(B(O)O)cc3)c3ccc4c(c3)oc3ccccc34)cccc21] | 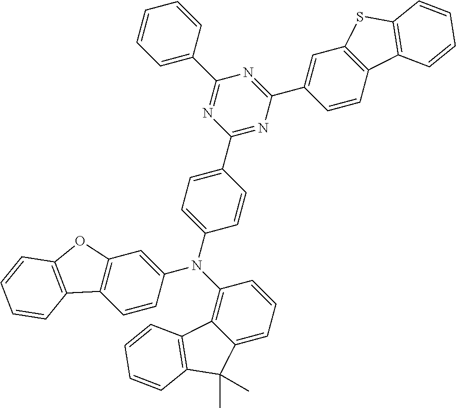 [SMILES: CC1(C)c2ccccc2-c2c(N(c3ccc(-c4nc(-c5ccccc5)nc(-c5ccc6c(c5)sc5ccccc56)n4)cc3)c3ccc4c(c3)oc3ccccc34)cccc21] | 50% |
| 1-299 | 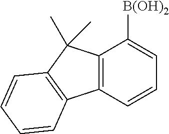 [SMILES: CC1(C)c2ccccc2-c2cccc(B(O)O)c21] | 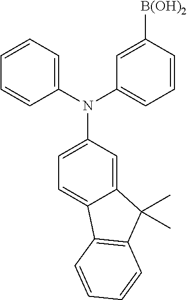 [SMILES: CC1(C)c2ccccc2-c2ccc(N(c3ccccc3)c3cccc(B(O)O)c3)cc21] | 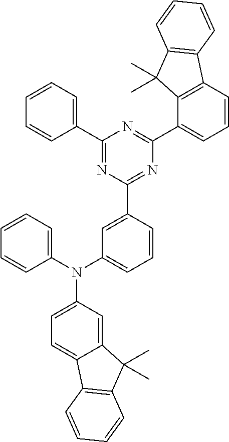 [SMILES: CC1(C)c2ccccc2-c2ccc(N(c3ccccc3)c3cccc(-c4nc(-c5ccccc5)nc(-c5cccc6c5C(C)(C)c5ccccc5-6)n4)c3)cc21] | 54% |
| 1-340 | 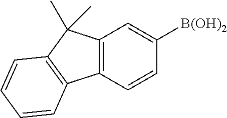 [SMILES: CC1(C)c2ccccc2-c2ccc(B(O)O)cc21] | 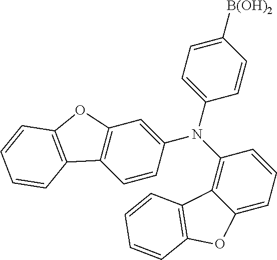 [SMILES: OB(O)c1ccc(N(c2ccc3c(c2)oc2ccccc23)c2cccc3oc4ccccc4c23)cc1] | 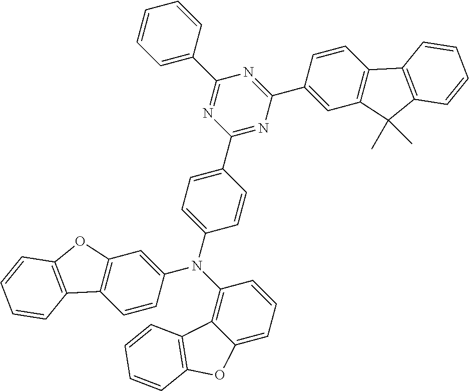 [SMILES: CC1(C)c2ccccc2-c2ccc(-c3nc(-c4ccccc4)nc(-c4ccc(N(c5ccc6c(c5)oc5ccccc56)c5cccc6oc7ccccc7c56)cc4)n3)cc21] | 53% |
| 1-355 | 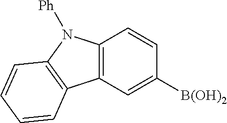 [SMILES: OB(O)c1ccc2c(c1)c1ccccc1n2-c1ccccc1] | 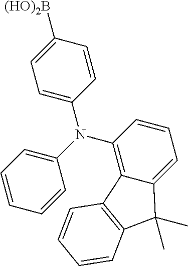 [SMILES: CC1(C)c2ccccc2-c2c(N(c3ccccc3)c3ccc(B(O)O)cc3)cccc21] | 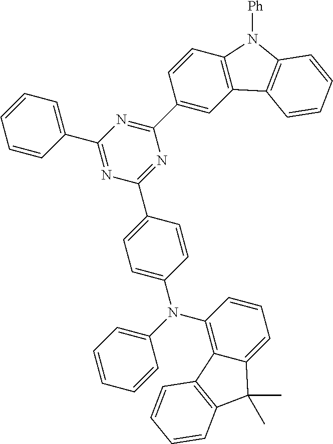 [SMILES: CC1(C)c2ccccc2-c2c(N(c3ccccc3)c3ccc(-c4nc(-c5ccccc5)nc(-c5ccc6c(c5)c5ccccc5n6-c5ccccc5)n4)cc3)cccc21] | 50% |
| 1-376 | 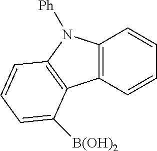 [SMILES: OB(O)c1cccc2c1c1ccccc1n2-c1ccccc1] | 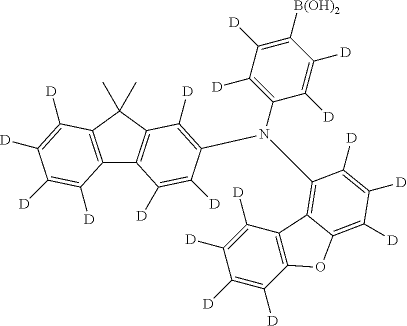 [SMILES: [2H]c1c([2H])c([2H])c2c(c1[2H])-c1c([2H])c([2H])c(N(c3c([2H])c([2H])c(B(O)O)c([2H])c3[2H])c3c([2H])c([2H])c([2H])c4oc5c([2H])c([2H])c([2H])c([2H])c5c34)c([2H])c1C2(C)C] | 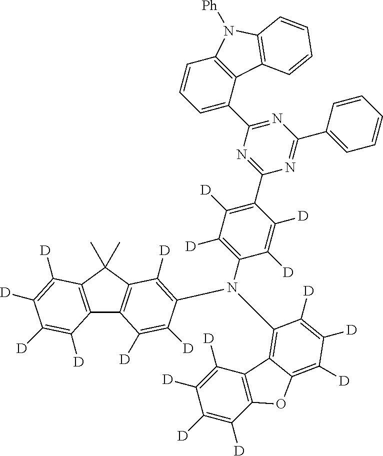 [SMILES: [2H]c1c([2H])c([2H])c2c(c1[2H])-c1c([2H])c([2H])c(N(c3c([2H])c([2H])c(-c4nc(-c5ccccc5)nc(-c5cccc6c5c5ccccc5n6-c5ccccc5)n4)c([2H])c3[2H])c3c([2H])c([2H])c([2H])c4oc5c([2H])c([2H])c([2H])c([2H])c5c34)c([2H])c1C2(C)C] | 49% |
| 1-379 | 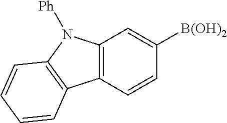 [SMILES: OB(O)c1ccc2c3ccccc3n(-c3ccccc3)c2c1] | 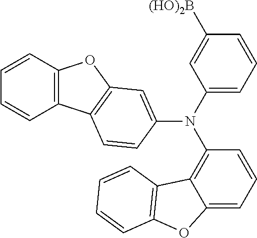 [SMILES: OB(O)c1cccc(N(c2ccc3c(c2)oc2ccccc23)c2cccc3oc4ccccc4c23)c1] | 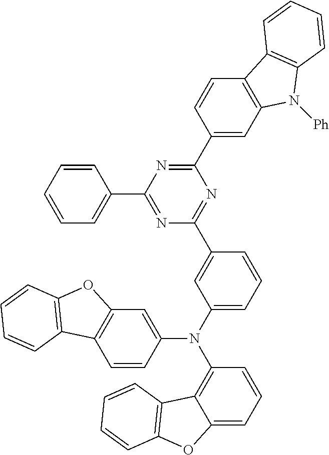 [SMILES: c1ccc(-c2nc(-c3cccc(N(c4ccc5c(c4)oc4ccccc45)c4cccc5oc6ccccc6c45)c3)nc(-c3ccc4c5ccccc5n(-c5ccccc5)c4c3)n2)cc1] | 52% |

### <Preparation Example 4> Preparation of Compound 2-3

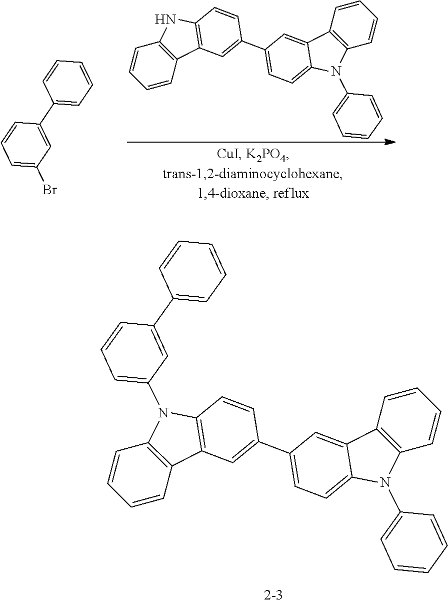

3-Bromo-1,1′-biphenyl(3.7 g, 15.8 mM), 9-phenyl-9H,9′H-3,3′-bicarbazole (6.5 g, 15.8 mM), CuI (3.0 g, 15.8 mM), trans-1,2-diaminocyclohexane (1.9 mL, 15.8 mM) and K₃PO₄ (3.3 g, 31.6 mM) were dissolved in 1,4-dioxane (100 mL), and refluxed for 24 hours. After the reaction was completed, the result was extracted by introducing distilled water and DCM thereto at room temperature, and after drying the organic layer with MgSO₄, the solvent was removed using a rotary evaporator. The reaction material was purified by column chromatography (DCM:Hex=1:3), and recrystallized with methanol to obtain target Compound 2-3 (7.5 g, 85%).

Target Compound C4 was synthesized in the same manner as in Preparation Example 4 except that Compound A4 of the following Table 4 was used instead of 3-bromo-1,1′-biphenyl, and Compound B4 of the following Table 4 was used instead of 9-phenyl-9H, 9′H-3,3′-bicarbazole.

TABLE 4

| Compound | | | | |
|---|---|---|---|---|
| No. | Compound A4 | Compound B4 | Target Compound C4 | Yield |
| 2-4 | 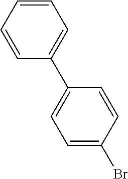 [SMILES: Brc1ccc(-c2ccccc2)cc1] | 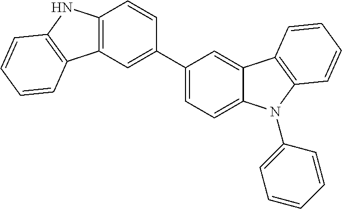 [SMILES: c1ccc(-n2c3ccccc3c3cc(-c4ccc5[nH]c6ccccc6c5c4)ccc32)cc1] | 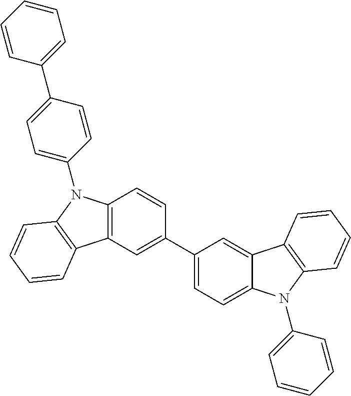 [SMILES: c1ccc(-c2ccc(-n3c4ccccc4c4cc(-c5ccc6c(c5)c5ccccc5n6-c5ccccc5)ccc43)cc2)cc1] | 83% |
| 2-7 | 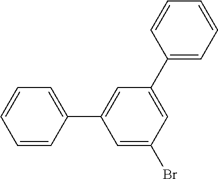 [SMILES: Brc1cc(-c2ccccc2)cc(-c2ccccc2)c1] | 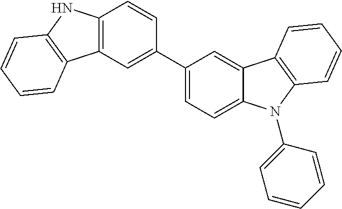 [SMILES: c1ccc(-n2c3ccccc3c3cc(-c4ccc5[nH]c6ccccc6c5c4)ccc32)cc1] | 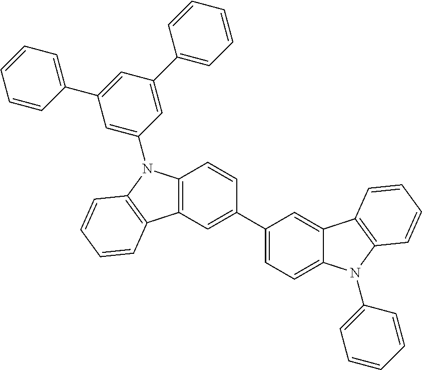 [SMILES: c1ccc(-c2cc(-c3ccccc3)cc(-n3c4ccccc4c4cc(-c5ccc6c(c5)c5ccccc5n6-c5ccccc5)ccc43)c2)cc1] | 84% |
| 2-31 | 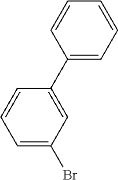 [SMILES: Brc1cccc(-c2ccccc2)c1] | 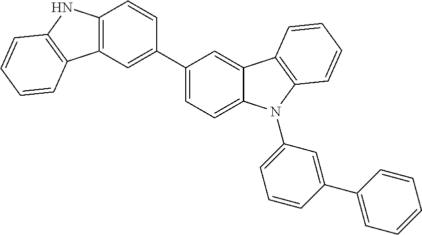 [SMILES: c1ccc(-c2cccc(-n3c4ccccc4c4cc(-c5ccc6[nH]c7ccccc7c6c5)ccc43)c2)cc1] | 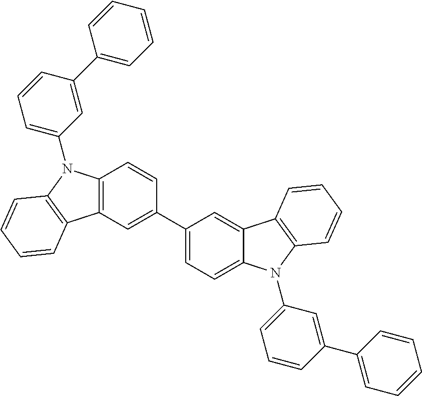 [SMILES: c1ccc(-c2cccc(-n3c4ccccc4c4cc(-c5ccc6c(c5)c5ccccc5n6-c5cccc(-c6ccccc6)c5)ccc43)c2)cc1] | 81% |
| 2-32 | 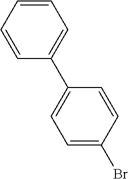 [SMILES: Brc1ccc(-c2ccccc2)cc1] | 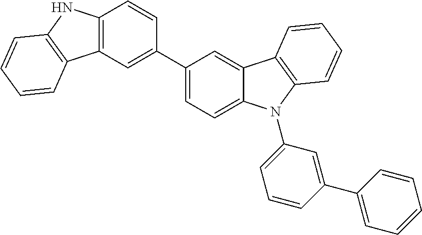 [SMILES: c1ccc(-c2cccc(-n3c4ccccc4c4cc(-c5ccc6[nH]c7ccccc7c6c5)ccc43)c2)cc1] | 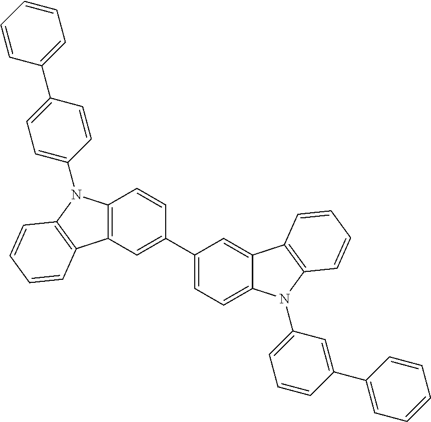 [SMILES: c1ccc(-c2ccc(-n3c4ccccc4c4cc(-c5ccc6c(c5)c5ccccc5n6-c5cccc(-c6ccccc6)c5)ccc43)cc2)cc1] | 80% |
| 2-42 | 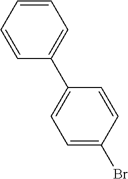 [SMILES: Brc1ccc(-c2ccccc2)cc1] | 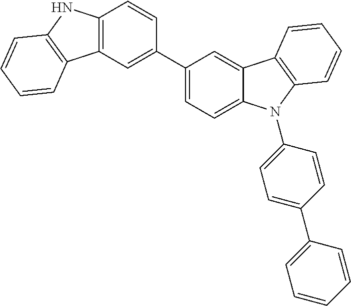 [SMILES: c1ccc(-c2ccc(-n3c4ccccc4c4cc(-c5ccc6[nH]c7ccccc7c6c5)ccc43)cc2)cc1] | 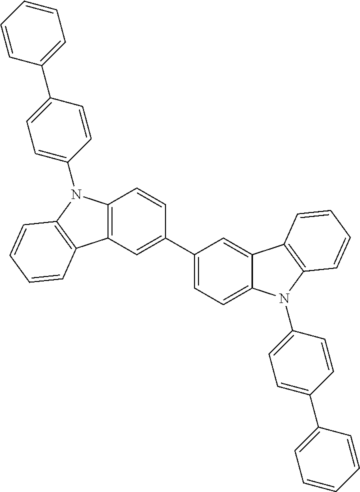 [SMILES: c1ccc(-c2ccc(-n3c4ccccc4c4cc(-c5ccc6c(c5)c5ccccc5n6-c5ccc(-c6ccccc6)cc5)ccc43)cc2)cc1] | 82% |

The rest of the compounds other than the compounds described in Table 1 to Table 4 were also prepared in the same manner as in the methods described in the preparation examples described above.

Synthesis identification results for the compounds prepared above are shown in the following Table 5 and Table 6. Table 5 shows measurement values of ¹H NMR (CDCl₃, 200 Mz), and Table 6 shows measurement values of ED-mass spectrometry (FD-MS: field desorption mass spectrometry).

TABLE 5

| Compound | |
|---|---|
| No. | ¹H NMR (CDCl₃, 200 Mz) |
| 1-1 | δ = 8.28(2H, d), 7.89-7.66(7H, m), 7.32-7.51(7H, m), |
| | 7.20(2H, t), 7.13(1H, t), 7.02(1H, d), 6.81(1H, d), |
| | 6.63(2H, d), 6.33(1H, d) |
| 1-3 | δ = 7.64-7.95(12H, m), 7.38(3H, d), 7.32(3H, d), |
| | 7.20(2H, t), 7.13(1H, t), 7.02(1H, d), 6.81(1H, t), |
| | 6.63(2H, d), 6.33(1H, d) |
| 1-12 | δ = 7.66-7.95(14H, m), 7.25-7.38(9H, m), 7.02-7.13(3H, |
| | m), 6.39(1H, d), 6.33(1H, d) |
| 1-17 | δ = 8.28(2H, d), 7.89(2H, d), 7.81(1H, d), 7.72-7.65(5H, |
| | m), 7.32-7.51(8H, m), 7.20(2H, t), 6.80(1H, t), |
| | 6.63(2H, d), 6.39(1H, d) |
| 1-20 | δ = 7.66-7.89(9H, m), 7.38(3H, d), 7.32(2H, d) |
| 1-42 | δ = 7.64-7.95(12H, m), 7.25-7.38(9H, m), 7.07(1H, t), |
| | 6.81(1H, t), 6.63(2H, d), 6.39(1H, d) |
| 1-46 | δ = 8.28(2H, d), 7.89(3H, d), 8.81(1H, d), 7.72(1H, d), |
| | 7.71(1H, d), 7.66(3H, d), 7.32-7.51(9H, m), 7.32(1H, |
| | t), 7.13(1H, t), 7.02(2H, d), 6.33(2H, d) |
| 1-58 | δ = 8.28(2H, d), 7.89(2H, d), 7.85(1H, d), 7.81(1H, d), |
| | 7.66(2H, d), 7.65(1H, s), 7.32-7.52(15H, m), 6.89(1H, |
| | s), 6.88(1H, d), 6.59(1H, d), 6.39(1H, d) |
| 1-60 | δ = 8.28(2H, d), 7.90(2H, d), 7.89(2H, d), 7.85(1H, d), |
| | 7.81(1H, d), 7.66(2H, d), 7.20-7.51(11H, m), 7.07(1H, |
| | t), 6.81(1H, t), 6.69(2H, d), 6.63(2H, d), 6.39(1H, d) |
| 1-61 | δ = 8.45(1H, d), 8.11(1H, d), 8.08(1H, s), 7.66-7.98(9H, |
| | m), 7.52(1H, t), 7.50(1H, t), 7.38(2H, t), 7.32(2H, |
| | t), 7.20(2H, t), 7.13(1H, t), 7.02(1H, d), 6.81(1H, |
| | t), 6.63(2H, d), 6.33(1H, d) |
| 1-67 | δ = 7.89(3H, d), 7.87(1H, d), 7.81(2H, d), 7.62-7.72(8H, |
| | m), 7.55(1H, d), 7.28-7.38(8H, m), 7.13(1H, t), |
| | 7.02(1H, d), 6.75(1H, s), 6.58(1H, d), 6.33(1H, d), |
| | 1.72(6H, s), |
| 1-69 | δ = 7.63-7.93(14H, m), 7.55(1H, d), 7.28-7.41(9H, m), |
| | 7.13(1H, t), 7.02(1H, d), 6.39(1H, d), 6.33(1H, d), |
| | 1.72(6H, s) |
| 1-77 | δ = 8.45(1H, d), 8.28(2H, d), 7.98(1H, d), 7.89(1H, d), |
| | 7.81(1H, d), 7.72(1H, d), 7.71(1H, s), 7.66(1H, d), |
| | 7.20-7.51(11H, m), 6.88(1H, d), 6.81(1H, t), 6.63(2H, |
| | d) |
| 1-81 | δ = 8.45(1H, d), 7.98(1H, d), 7.81-7.89(4H, m), |
| | 7.72(1H, d), 7.71(1H, s), 7.66(1H, d), 7.20-7.55 |
| | (13H, m), 6.88(1H, d), 6.81(1H, t), |
| | 6.63(2H, d), 1.72(6H, s) |
| 1-89 | δ = 8.51(1H, d), 8.45(2H, d), 8.18(1H, d), 8.12(1H, d), |
| | 7.98(2H, d), 7.89(1H, d), 7.81(1H, d), 7.25-7.71(22H, |
| | m), 6.88(2H, d) |
| 1-102 | δ = 8.51(1H, d), 8.18(1H, d), 8.12(1H, d), 7.89(1H, d), |
| | 7.87(1H, d), 7.81(1H, d), 7.20-7.71(19H, m), 7.03(1H, |
| | t), 6.91(1H, d), 6.81(1H, t), 6.63(2H, d), 6.58(1H, |
| | d), 1.72(6H, s) |
| 1-106 | δ = 7.89(2H, d), 7.87(2H, d), 7.81(2H, d), 7.62-7.72(7H, |
| | m), 7.55(2H, d), 7.28-7.38(8H, m), 7.03(1H, t), 6.91 |
| | (1H, d), 6.75(1H, s), 6.58(2H, d), 1.72(12H, s) |
| 1-109 | δ = 7.66-7.93(13H, m), 7.55(2H, d), 7.28-7.41(9H, m), |
| | 7.03(1H, t), 6.91(1H, d), 6.58(1H, d), 6.39(1H, d), |
| | 1.72(12H, s) |
| 1-116 | δ = 8.51(1H, d), 8.18(1H, d), 8.12(1H, d), 7.89(1H, d), |
| | 7.87(2H, d), 7.81(1H, d), 7.23-7.72(21H, m), 7.13(1H, |
| | t), 7.03(1H, t), 6.91(1H, d), 6.58(1H, d), 6.48(1H, |
| | d), 1.72(12H, s) |
| 1-118 | δ = 8.45(1H, d), 7.75-7.98(8H, m), 7.66 |
| | (2H, d), 7.64(1H, s), 7.25-7.52(13H, m), 7.02-7.13 |
| | (3H, m), 6.88(1H, d), 6.33(1H, d) |
| 1-121 | δ = 8.45(2H, d), 8.28(2H, d), 8.00(1H, s), 7.98(2H, d), |
| | 7.86(1H, d), 7.80(1H, d), 7.34-7.52(8H, m), 7.25(1H, |
| | t), 7.20(2H, t), 6.88(1H, d), 6.81(1H, t), 6.63(2H, d) |
| 1-125 | δ = 8.45(3H, d), 8.11(1H, d), 8.08(1H, s), 8.00(1H, s), |
| | 7.98(3H, d), 7.80-7.86(3H, m), 7.52(3H, d), 7.50(3H, |
| | d), 7.34(1H, d), 7.25(1H, t), 7.20(2H, t), 6.88(1H, |
| | d), 6.81(1H, t), 6.63(2H, d) |
| 1-136 | δ = 8.45(4H, d), 8.41(1H, d), 8.00(1H, s), 7.98(4H, d), |
| | 7.86(1H, d), 7. 80(2H, d), 7.58(1H, t), 7.52(4H, d), |
| | 7.50(4H, d), 7.34(2H, d), 7.52(1H, t), 7.25(1H, t), |
| | 6.88(2H, d) |
| 1-142 | δ = 8.45(3H, d), 8.11(1H, d), 8.08(1H, s), 8.00(1H, s), |
| | 7.98(3H, d), 7.73-7.86(4H, m), 7.52(3H, d), 7.50(3H, |
| | d), 7.40(1H, s), 7.20(2H, t), 6.86(1H, d), 6.81(1H, |
| | t), 6.63(2H, d) |
| 1-163 | δ = 8.45(4H, d), 8.00(2H, s), 7.98(4H, d), 7.86(2H, d), |
| | 7.81(1H, d), 7.80(3H, d), 7.52(4H, d), 7.50(4H, d), |
| | 7.27(1H, t), 7.06(1H, s), 6.88(1H, d), 6.86(1H, d) |
| 1-178 | δ = 8.45(3H, d), 8.24(1H, d), 8.00(1H, s), 7.98(3H, d), |
| | 7.86(1H, d), 7. 80(1H, d), 7.70(1H, s), 7.34-7.52(15H, |
| | m), 7.25(2H, t), 6.68(2H, d) |
| 1-189 | δ = 8.45(1H, d), 8.28(2H, d), 7.98-8.11(6H, m), |
| | 7.82(1H, d), 7.73(1H, d), 7.40-7.52(11H, m), |
| | 7.20(2H, t), 6.86(1H, d), 6.81(1H, t), 6.63(2H, d) |
| 1-192 | δ = 8.45(2H, d), 8.28(2H, d), 8.11(1H, d), 8.08(1H, s), |
| | 7.81-7.98(6H, m), 7.41-7.52(7H, m), 7.27(2H, t), |
| | 7.20(1H, t), 6.86(1H, d), 6.81(1H, t), 6.69(2H, d), |
| | 6.63(2H, d) |
| 1-199 | δ = 8.45(1H, d), 7.98(1H, d), 7.81(1H, d), 7.52(1H, d), |
| | 7.50(1H, d), 7.27(1H, t), 7.20(2H, t), 6.86(1H, d), |
| | 6.81(1H, t), 6.33(2H, d) |
| 1-201 | δ = 8.45(1H, d), 8.28(2H, d), 8.00(1H, s), 7.98(1H, d), |
| | 7.80-7.89(3H, m), 7.66(1H, d), 7.38-7.52(13H, m), |
| | 7.13(1H, t), 7.02(1H, d), 6.88(1H, s), 6.88(1H, d), |
| | 6.59(1H, d), 6.33(1H, d) |
| 1-202 | δ = 8.45(2H, d), 8.11(1H, d), 8.08(1H, s), 7.80-8.00(7H, |
| | m), 7.66(1H, d), 7.52(2H, d), 7.50(2H, d), 7.38(1H, |
| | d), 7.32(1H, d), 7.20(2H, t), 7.13(1H, t), 7.02(1H, |
| | d), 6.81(1H, t), 6.63(2H, d), 6.33(1H, d) |
| 1-207 | δ = 8.45(2H, d), 8.11(1H, d), 8.08(1H, s), 7.80-8.00(8H, |
| | m), 7.66(2H, d), 7.52(2H, d), 7.50(2H, d), 7.38(2H, |
| | d), 7.32(2H, d), 7.13(2H, t), 7.02(2H, d), 6.33(2H, d) |
| 1-211 | δ = 8.45(2H, d), 7.77-8.00(10H, m), 7.66(1H, d), |
| | 7.50-7.55(5H, m), 7.27-7.38(5H, m), |
| | 7.63(1H, d), 7.50-7.55(5H, m), 7.27-7.38(5H, m), |
| | 7.13(1H, t), 7.02(1H, d), 6.86(1H, d), 6.33(1H, d), |
| | 1.72(6H, s) |
| 1-224 | δ = 8.45(1H, d), 8.28(2H, d), 8.00(1H, s), 7.98(1H, d), |
| | 7.80-7.87(3H, m), 7.38-7.55(7H, m), 7.28(1H, t), |
| | 7.20(2H, t), 7.03(1H, t), 6.91(1H, d), 6.81(1H, t), |
| | 6.63(2H, d), 6.58(1H, d), 1.72(6H, s) |
| 1-230 | δ = 8.51(1H, d), 8.45(1H, d), 8.18(1H, d), 8.12(1H, d), |
| | 8.00(1H, s), 7.98(1H, d), 7.80-7.87(3H, m), 7.20-7.63 |
| | (16H, m), 7.03(1H, t), 6.91(1H, d), 6.81(1H, t), |
| | 6.63(2H, d), 6.58(1H, d), 1.72(6H, s) |
| 1-233 | δ = 8.49(1H, d), 8.45(1H, d), 8.12(1H, d), 8.10(1H, d), |
| | 8.00(1H, s), 7.98(1H, d), 7.80-7.89(4H, m), 7.28-7.66 |
| | (19H, m), 7.03(1H, t), 6.91(1H, d), 6.58(1H, d), |
| | 6.33(1H, d), 1.72(6H, s) |
| 1-237 | δ = 8.45(2H, d), 8.41(1H, d), 8.00(1H, s), 7.98(2H, d), |
| | 7.87-7.80(5H, m), 7.50-7.58(7H, m), 7.38(2H, t), |
| | 7.30(1H, d), 7.28(2H, t), 7.04(1H, s), 7.03(1H, t), |
| | 6.91(1H, d), 6.58(1H, d), 6.48(1H, d), 1.72(12H, s) |
| 1-239 | δ = 8.51(1H, d), 8.45(1H, d), 8.18(1H, d), 8.12(1H, d), |
| | 8.00(1H, s), 7.98(1H, d), 7.87-7.80(4H, m), |
| | 7.23-7.86(18H, m), 7.13(1H, t), 7.03(1H, t), 6.91(1H, d), |
| | 6.58(1H, d), 6.48(1H, d), 1.72(12H, s) |
| 1-240 | δ = 8.45(1H, d), 8.28(2H, d), 8.11(1H, d), 8.08(1H, s), |
| | 7.98(1H, d), 7.87-7.90(5H, m), 6.66(1H, d), 6.64(1H, |
| | d), 7.28-7.55(11H, m), 7.03(1H, t), 6.91(1H, d), |
| | 6.69(2H, d), 6.58(1H, d), 6.33(1H, d), 1.72(6H, s) |
| 1-244 | δ = 8.06(1H, s), 7.87(3H, d), 7.83(1H, d), 7.53-7.61(6H, |
| | m), 7.44(1H, t), 7.38(3H, t), 7.28(3H, t), 7.20(2H, |
| | t), 7.03(1H, t), 6.91(1H, d), 6.81(1H, t), 6.63(2H, |
| | d), 6.58(1H, d), 1.72(18H, s) |
| 1-249 | δ = 8.06(1H, s), 7.93(1H, d), 7.87(4H, d), 7.77(1H, d), |
| | 7.53-7.61(7H, m), 7.38(4H, t), 7.28(4H, t), 7.03 |
| | (2H, t), 6.91(2H, d), 6.58(2H, d), 1.72(24H, s) |
| 1-259 | δ = 8.06(1H, s), 7.93(1H, d), 7.87(3H, d), 7.77(1H, s), |
| | 7.53-7.63(6H, m), 7.38(3H, t), 7.20-7.30(6H, m), |
| | 7.04(1H, s), 6.81(1H, t), 6.63(2H, d), 6.48(1H, d), |
| | 1.72(18H, s) |
| 1-280 | δ = 8.28(2H, d), 8.06(1H, s), 7.87(2H, d), 7.51-7.55(6H, |
| | 7.41(1H, t), 7.38(2H, t), 7.13-7.28(6H, m), |
| | m), 7.41(1H, t), 7.38(2H, t), 7.13-7.28(6H, m), |
| | 6.81(1H, t), 6.63(2H, d), 6.48(1H, d), 1.72(12H, s) |
| 1-286 | δ = 8.28(2H, d), 8.06(1H, s), 7.87(3H, d), 7.51-7.61(7H, |
| | m), 7.41(1H, t), 7.38(3H, t), 7.28(3H, t), 7.03(2H, |
| | t), 6.91(2H, d), 6.58(2H, d), 1.72(18H, s) |
| 1-293 | δ = 7.93(1H, d), 7.87(2H, d), 7.85(2H, d), 7.77(1H, s), |
| | 7.63(1H, d), 7.51-7.55(6H, m), 7.41(1H, t), 7.38(2H, |
| | t), 7.20-7.28(6H, m), 7.03(1H, t), 6.91(1H, d), |
| | 6.81(1H, t), 6.63(2H, d), 6.58(1H, d), 1.72(12H, s) |
| 1-295 | δ = 8.28(2H, d), 7.93(1H, d), 7.87(2H, d), 7.77(1H, s), |
| | 7.63(1H, d), 7.62(1H, d), 7.55(2H, d), 7.51(2H, t), |
| | 7.41(1H, t), 7.38(2H, t), 7.28(2H, t), 7.20(2H, t), |
| | 6.81(1H, t), 6.75(1H, s), 6.63(2H, d), 6.58(1H, d), |
| | 1.72(12H, s) |
| 1-299 | δ = 8.28(2H, d), 7.87(2H, d), 7.83(1H, d), 7.64(1H, d), |
| | 7.62(1H, d), 7.38-7.55(10H, m), 7.28(2H, t), 7.20(2H, |
| | t), 6.89(1H, s), 6.81(1H, t), 6.75(1H, s), 6.58- |
| | 6.63(4H, m), 1.72(12H, s) |
| 1-301 | δ = 8.28(2H, d), 8.06(1H, s), 7.89(1H, d), 7.87(1H, d), |
| | 7.51-7.66(6H, m), 7.20-7.41(8H, m), 7.02(1H, d), |
| | 6.81(1H, t), 6.63(2H, d), 6.33(1H, d), 1.72(6H, s) |
| 1-306 | δ = 8.45(1H, d), 8.08(1H, s), 7.77-7.98(7H, m), |
| | 7.50-7.66(8H, m), 7.27-7.38(7H, m), 7.13(1H, t), |
| | 7.02(1H, d), 6.86(1H, d), 6.33(1H, d), 1.72(12H, s) |
| 1-309 | δ = 8.51(1H, d), 8.18(1H, d), 8.12(1H, d), 8.06(1H, s), |
| | 7.89(1H, d), 7.87(2H, d), 7.13-7.63(21H, m), 7.13(2H, |
| | t), 7.02(1H, d), 6.48(1H, d), 6.33(1H, d), 1.72(12H, |
| | s) |
| 1-312 | δ = 8.51(1H, d), 8.45(1H, d), 8.18(1H, d), 8.12(1H, d), |
| | 8.06(1H, s), 7.98(1H, d), 7.87(1H, d), 7.20-7.63(20H, |
| | m), 6.88(1H, d), 6.81(1H, t), 6.63(2H, d), 1.72(6H, s) |
| 1-320 | δ = 8.06(1H, s), 7.87-7.93(5H, m), 7.77(1H, s), |
| | 7.53-7.66(8H, m), 7.28-7.41(9H, m), 7.03(1H, t), |
| | 6.91(1H, d), 6.58(1H, d), 6.39(1H, d), 1.72(18H, s) |
| 1-323 | δ = 8.51(1H, d), 8.45(1H, d), 8.18(1H, d), 8.12(1H, d), |
| | 8.06(1H, s), 7.98(1H, d), 7.87(2H, d), 7.25-7.63(21H, |
| | m), 7.03(1H, t), 6.88(1H, d), 6.91(1H, d), 6.58(1H, |
| | d), 1.72(12H, s) |
| 1-325 | δ = 8.28(2H, d), 7.98(2H, d), 7.87(1H, d), 7.63-7.66(4H, |
| | 7.28-7.55(13H, m), |
| | 7.13(1H, t), 7.02(1H, d), |
| | m), 7.28-7.55(13H, m), 7.13(1H, t), 7.02(1H, d), |
| | 6.33(2H, d), 1.72(6H, s) |
| 1-326 | δ = 8.45(1H, d), 8.28(2H, d), 7.98(1H, d), 7.81-7.89(3H, |
| | m), 7.66(1H, d), 7.63(1H, d), 7.28-7.55(13H, m), |
| | 7.13(1H, t), 7.02(1H, d), 6.86(1H, d), 6.33(1H, d), |
| | 1.72(6H, s) |
| 1-333 | δ = 8.24(1H, d), 7.89(1H, d), 7.87(1H, d), |
| | 7.13-7.70(20H, m), 7.02(1H, d), 6.81(1H, t), 6.63(2H, d), |
| | 6.33(1H, d), 1.72(6H, s) |
| 1-336 | δ = 7.83-7.89(5H, m), 7.66(1H, d), 7.55(2H, |
| | d), 7.53(2H, d), 7.13-7.38(11H, m), 7.02(1H, d), |
| | 6.81(1H, t), 6.63(2H, d), 6.33(1H, d), 1.72(12H, s) |
| 1-338 | δ = 8.28(2H, d), 7.89(1H, d), 7.87(2H, d), 7.62-7.66(3H, |
| | m), 7.55(2H, d), 7.51(3H, d), 7.28-7.41(8H, m), |
| | 7.13(1H, t), 7.02(1H, d), 6.75(1H, s), 6.58(1H, d), |
| | 6.33(1H, d), 1.72(12H, s) |
| 1-340 | δ = 8.28(2H, d), 7.87-7.93(6H, m), 7.77(1H, s), |
| | 7.63-7.66(4H, m), 7.55(1H, d), 7.51(2H, d), |
| | 7.28-7.41(8H, m), 7.13(1H, t), 7.02(1H, d), 6.69 |
| | (2H, d), 6.33(2H, d), 1.72(6H, s) |
| 1-341 | δ = 8.55(1H, d), 8.28(2H, d), 7.87-7.94 |
| | (3H, m), 7.77(1H, s), 7.13-7.69(17H, m), 7.02 |
| | (1H, d), 6.81(1H, t), 6.63(2H, d), 6.33(1H, d) |
| 1-342 | δ = 8.55(1H, d), 7.85-7.94(5H, m), 7.77(1H, s), |
| | 7.25-7.69(25H, m), 7.13(1H, t), 7.02(1H, d), |
| | 6.69(2H, d), 6.33(1H, d) |
| 1-349 | δ = 8.45-8.55(3H, m), 8.18(1H, d), 8.12(1H, d), |
| | 7.98(1H, d), 7.94(1H, d), 7.87(1H, d), 7.77(1H, s), |
| | 7.45-7.69(15H, m), 7.20-7.34(8H, m), 8.88 |
| | (1H, d), 6.81(1H, t), 6.63(2H, d) |
| 1-350 | δ = 8.45-8.55(3H, m), 8.12(1H, d), 8.10(1H, d), |
| | 7.87-7.98(4H, m), 7.77(1H, s), 7.25-7.69 |
| | (26H, m), 6.88(1H, d), 6.33(1H, d) |
| 1-355 | δ = 8.55(1H, d), 8.28(2H, d), 7.87-7.94 |
| | (5H, m), 7.77(1H, s), 7.69(1H, d), 7.20-7.58(15H, m), |
| | 7.03(1H, t), 6.91(1H, d), 6.81(1H, t), |
| | 6.58-6.69(5H, m), 1.72(6H, s) |
| 1-360 | δ = 8.55(1H, d), 8.51(1H, d), 8.18(1H, d), 8.12(1H, d), |
| | 7.94(1H, d), 7.87(3H, d), 7.77(1H, s), 7.23-7.69(24H, |
| | m), 7.13(1H, t), 7.03(1H, t), 6.91(1H, d), 6.58(1H, |
| | d), 6.48(1H, d), 1.72(12H, s) |
| 1-362 | δ = 8.55(1H, d), 8.28(2H, d), |
| | 7.87-7.94(4H, m), 7.77(1H, |
| | s), 7.25-7.69(18H, m), 7.13(1H, t), 7.02(1H, d), |
| | 6.75(1H, s), 6.58(1H, d), 6.33(1H, d), 1.72(6H, s) |
| 1-366 | δ = 8.55(1H, d), 8.45(1H, d), 8.28(2H, d), 8.08(1H, d), |
| | 7.87-7.98(4H, m), 7.25-7.66(20H, m), 6.88(1H, d), |
| | 6.33(1H, d) |
| 1-368 | δ = 8.55(1H, d), 8.45(1H, d), 8.08(1H, d), 7.85-7.98(6H, |
| | m), 7.25-7.62(23H, m), 6.88(1H, d), 6.75(1H, s), |
| | 6.58(1H, d), 1.72(6H, s) |
| 1-370 | δ = 8.55(1H, d), 8.24(1H, d), 7.94(1H, d), 7.89(1H, d), |
| | 7.79(1H, d), 7.20-7.70(23H, m), 7.02(1H, d), 6.81(1H, |
| | t), 6.63(2H, d), 6.33(1H, d) |
| 1-371 | δ = 8.55(1H, d), 8.45(1H, d), 8.28(2H, d), 7.98(1H, d), |
| | 7.94(1H, d), 7.79(1H, d), 7.25-7.59(22H, m), 6.89(1H, |
| | s), 6.88(2H, d), 6.59(1H, d) |
| 1-376 | δ = 8.55(1H, d), 8.28(2H, d), 7.94(1H, d), 7.79(1H, d), |
| | 7.25-7.59(12H, m), 1.72(6H, s) |
| 1-379 | δ = 8.55(1H, d), 8.28(2H, d), 8.18(1H, d), 7.94(1H, d), |
| | 7.89(2H, d), 7.79(1H, d), 7.25-7.66(21H, m), 7.13(1H, |
| | t), 7.02(1H, d), 6.89(1H, s), 6.59(1H, d), 6.33(2H, d) |
| 2-3 | δ = 8.55(1H, d), 8.30(1H, d), 8.21-8. 13(3H, m), |
| | 7.99-7.89(3H, m), 7.77-7.35(17H, m), 7.20-7.16(2H, m) |
| 2-4 | δ = 8.55(1H, d), 8.30(1H, d), 8.19-8.13(2H, m), |
| | 7.99-7.89(8H, m), 7.77-7.75(3H, m), 7.62-7.35(11H, m), |
| | 7.20-7.16(2H, m) |
| 2-7 | δ = 8.55(1H, d), 8.31-8.30(3H, d), 8.19-8.13(2H, m), |
| | 7.99-7.89(5H, m), 7.77-7.75(5H, m), 7.62-7.35(14H, m), |
| | 7.20-7.16(2H, m) |
| 2-31 | δ = 8.55(1H, d), 8.30(1H, d), 8.21-8.13(4H, m), |
| | 7.99-7.89(4H, m), 7.77-7.35(20H, m), 7.20-7.16(2H, m) |
| 2-32 | δ = 8.55(1H, d), 8.30(1H, d), 8.21-8.13(3H, m), |
| | 7.99-7.89(8H, m), 7.77-7.35(17H, m), 7.20-7.16(2H, m) |
| 2-42 | δ = 8.55(1H, d), 8.30(1H, d), 8.19(1H, d), 8.13(1H, d), |
| | 7.99-7.89(12H, m), 7.77-7.75(5H, m), 7.58(1H, d), |
| | 7.49-7.35(8H, m), 7.20-7.16(2H, m) |

TABLE 6

| Compound | FD-MS | Compound | FD-MS |
|---|---|---|---|
| 1-1 | m/z = 580.19 | 1-3 | m/z = 670.20 |
| | (C₃₉H₂₄N₄O₂ = 580.63) | | (C₄₅H₂₆N₄O₃ = 670.71) |
| 1-12 | m/z = 760.21 | 1-17 | m/z = 580.19 |
| | (C₅₁H₂₈N₄O₄ = 760.79) | | (C₃₉H₂₄N₄O₂ = 580.63) |
| 1-20 | m/z = 682.28 | 1-40 | m/z = 604.34 |
| | (C₄₅H₁₄D₁₂N₄O₃ = 682.79) | | (C₃₉D₂₄N₄O₂ = 604.78) |
| 1-42 | m/z = 670.20 | 1-46 | m/z = 670.20 |
| | (C₄₅H₂₆N₄O₃ = 670.71) | | (C₄₅H₂₆N₄O₃ = 670.71) |
| 1-58 | m/z = 656.22 | 1-60 | m/z = 656.22 |
| | (C₄₅H₂₈N₄O₂ = 656.73) | | (C₄₅H₂₈N₄O₂ = 656.73) |
| 1-61 | m/z = 686.18 | 1-67 | m/z = 786.26 |
| | (C₄₅H₂₆N₄O₂S = 686.78) | | (C₅₄H₃₄N₄O₃ = 786.87) |
| 1-69 | m/z = 786.26 | 1-77 | m/z = 596.17 |
| | (C₅₄H₃₄N₄O₃ = 786.87) | | (C₃₉H₂₄N₄OS = 596.70) |
| 1-81 | m/z = 712.23 | 1-89 | m/z = 867.21 |
| | (C₄₈H₃₂N₄OS = 712.86) | | (C₅₇H₃₃N₅OS₂ = 868.03) |
| 1-102 | m/z = 771.30 | 1-106 | m/z = 812.32 |
| | (C₅₄H₃₇N₅O = 771.90) | | (C₅₇H₄₀N₄O₂ = 812.95) |
| 1-109 | m/z = 812.32 | 1-116 | m/z = 887.36 |
| | (C₅₇H₄₀N₄O₂ = 812.95) | | (C₆₃H₄₅N₅O = 888.06) |
| 1-118 | m/z = 762.21 | 1-121 | m/z = 612.14 |
| | (C₅₁H₃₀N₄O₂S = 762.87) | | (C₃₉H₂₄N₄O₂ = 612.76) |
| 1-125 | m/z = 718.13 | 1-136 | m/z = 824.12 |
| | (C₄₅H₂₆N₄O₃ = 718.91) | | (C₅₁H₂₈N₄S₄ = 825.05) |
| 1-142 | m/z = 718.13 | 1-163 | m/z = 824.12 |
| | (C₄₅H₂₆N₄S₃ = 718.91) | | (C₅₁H₂₈N₄S₄ = 825.05) |
| 1-178 | m/z = 794.16 | 1-189 | m/z = 688.18 |
| | (C₅₁H₃₀N₄O₃ = 795.01) | | (C₄₅H₂₈N₄O₂ = 688.86) |
| 1-192 | m/z = 688.18 | 1-199 | m/z = 624.22 |
| | (C₄₅H₂₈N₄O₂ = 688.86) | | (C₃₉H₁₂D₁₂N₄S₂ = 624.84) |
| 1-201 | m/z = 672.20 | 1-202 | m/z = 702.15 |
| | (C₄₅H₂₈N₄OS = 672.80) | | (C₄₅H₂₆N₄OS₂ = 702.84) |
| 1-207 | m/z = 792.17 | 1-211 | m/z = 818.22 |
| | (C₅₁H₂₈N₄O₂S₂ = 792.92) | | (C₅₄H₃₄N₄OS₂ = 819.00) |
| 1-224 | m/z = 622.20 | 1-230 | m/z = 787.28 |
| | (C₄₂H₃₀N₄S = 622.78) | | (C₅₄H₃₇N₅S = 787.97) |
| 1-233 | m/z = 877.29 | 1-237 | m/z = 844.27 |
| | (C₆₀H₃₉N₅OS = 878.05) | | (C₅₇H₄₀N₄S = 845.08) |
| 1-239 | m/z = 903.34 | 1-240 | m/z = 788.26 |
| | (C₆₃H₄₅N₅S = 904.13) | | (C₅₄H₃₆N₄OS = 788.95) |
| 1-244 | m/z = 748.35 | 1-249 | m/z = 864.42 |
| | (C₅₄H₄₄N₄ = 748.95) | | (C₆₃H₅₂N₄ = 865.11) |
| 1-259 | m/z = 748.35 | 1-280 | m/z = 632.29 |
| | (C₅₄H₄₄N₄ = 748.95) | | (C₄₅H₃₆N₄ = 632.79) |
| 1-286 | m/z = 748.36 | 1-293 | m/z = 708.33 |
| | (C₅₄H₄₄N₄ = 748.95) | | (C₅₁H₄₀N₄ = 708.89) |
| 1-295 | m/z = 632.29 | 1-299 | m/z = 708.33 |
| | (C₄₅H₃₆N₄ = 632.79) | | (C₅₁H₄₀N₄ = 708.89) |
| 1-301 | m/z = 606.24 | 1-306 | m/z = 828.29 |
| | (C₄₂H₃₀N₄O = 606.71) | | (C₅₇H₄₀N₄OS = 829.02) |
| 1-309 | m/z = 887.36 | 1-312 | m/z = 787.28 |
| | (C₆₃H₄₅N₅O = 888.06) | | (C₅₄H₃₇N₅S = 787.97) |
| 1-320 | m/z = 838.37 | 1-323 | m/z = 903.34 |
| | (C₆₀H₄₆N₄O = 839.03) | | (C₆₃H₄₅N₅S = 904.13) |
| 1-325 | m/z = 696.25 | 1-326 | m/z = 712.23 |
| | (C₄₈H₃₂N₄O₂ = 696.79) | | (C₄₈H₃₂N₄OS = 712.86) |
| 1-333 | m/z = 682.27 | 1-336 | m/z = 722.30 |
| | (C₄₈H₃₄N₄O = 682.81) | | (C₅₁H₃₈N₄O = 722.87) |
| 1-338 | m/z = 722.30 | 1-340 | m/z = 777.28 |
| | (C₅₁H₃₈N₄O = 722.87) | | (C₅₄H₃₆N₄O₂ = 777.89) |
| 1-341 | m/z = 655.24 | 1-342 | m/z = 807.30 |
| | (C₄₅H₂₉N₅O = 655.74) | | (C₅₇H₃₇N₅O = 807.94) |
| 1-349 | m/z = 836.27 | 1-350 | m/z = 926.28 |
| | (C₅₇H₃₆N₆S = 837.00) | | (C₆₃H₃₈N₆OS = 927.08) |
| 1-355 | m/z = 757.32 | 1-360 | m/z = 962.41 |
| | (C₅₄H₃₉N₅ = 757.92) | | (C₆₉H₅₀N₆ = 963.18) |
| 1-362 | m/z = 771.30 | 1-366 | m/z = 761.22 |
| | (C₅₄H₃₇N₅O = 771.90) | | (C₅₁H₃₁N₅OS = 761.89) |
| 1-368 | m/z = 864.31 | 1-370 | m/z = 731.27 |
| | (C₆₀H₄₁N₅S = 864.07) | | (C₅₁H₃₃N₅O = 731.84) |
| 1-371 | m/z = 747.25 | 1-376 | m/z = 865.44 |
| | (C₅₁H₃₃N₅S = 747.91) | | (C₆₀H₂₃D₁₈N₅O = 866.11) |
| 1-379 | m/z = 821.28 | 2-3 | m/z = 560.23 |
| | (C₅₇H₃₅N₅O₂ = 821.92) | | (C₄₂H₂₈N₂ = 560.70) |
| 2-4 | m/z = 560.23 | 2-7 | m/z = 636.26 |
| | (C₄₂H₂₈N₂ = 560.70) | | (C₄₈H₃₂N₂ = 636.80) |
| 2-31 | m/z = 636.26 | 2-32 | m/z = 636.26 |
| | (C₄₈H₃₂N₂ = 636.80) | | (C₄₈H₃₂N₂ = 636.80) |
| 2-42 | m/z = 636.26 | | |
| | (C₄₈H₃₂N₂ = 636.80) | | |

### Experimental Example 1

### 1) Manufacture of Organic Light Emitting Device

### Comparative Example 1

A glass substrate on which ITO was coated as a thin film to a thickness of 1,500 Å was cleaned with distilled water ultrasonic waves. After the cleaning with distilled water was finished, the substrate was ultrasonic cleaned with solvents such as acetone, methanol and isopropyl alcohol, then dried, and UVO treatment was conducted for 5 minutes using UV in a UV cleaner. After that, the substrate was transferred to a plasma cleaner (PT), and after conducting plasma treatment under vacuum for ITO work function and residual film removal, the substrate was transferred to a thermal deposition apparatus for organic deposition.

On the transparent ITO electrode (anode), a hole injection layer 2-TNATA (4,4′,4″-tris[2-naphthyl(phenyl)amino]triphenylamine) and a hole transfer layer NPB (N,N′-di(1-naphthyl)-N,N′-diphenyl-(1,1′-biphenyl)-4,4′-diamine), which are common layers, were formed.

A light emitting layer was thermal vacuum deposited thereon as follows. As the light emitting layer, Compound 2-3 was deposited to 400 Å as a host, and as a green phosphorescent dopant, Ir(ppy)₃ was doped and deposited by 7% of the deposited thickness of the light emitting layer. After that, BCP was deposited to 60 Å as a hole blocking layer, and Alq₃ was deposited to 200 Å thereon as an electron transfer layer. Lastly, an electron injection layer was formed on the electron transfer layer by depositing lithium fluoride (LiF) to a thickness of 10 Å, and then a cathode was formed on the electron injection layer by depositing an aluminum (Al) cathode to a thickness of 1,200 Å, and as a result, an organic light emitting device of Comparative Example 1 was manufactured.

### Comparative Examples 2 to 14 and Examples 1 to 73

Organic light emitting devices of Comparative Examples 2 to 14 and Examples 1 to 73 were additionally manufactured in the same manner as in the method for manufacturing an organic light emitting device of Comparative Example 1 except that, in the process for manufacturing an organic light emitting device of Comparative Example 1, compounds described in the following Tables 7 and 8 were used as the host of the light emitting layer instead of using Compound 2-3.

Meanwhile, all the organic compounds required to manufacture the OLED were vacuum sublimation purified under 10⁻⁸ torr to 10⁻⁶ torr for each material to be used in the OLED manufacture.

### 2) Evaluation on Organic Light Emitting Device

For each of the organic light emitting devices of Comparative Examples 1 to 14 and Examples 1 to 73 manufactured as above, electroluminescent (EL) properties were measured using M7000 manufactured by McScience Inc., and with the measurement results, T₉₀ was measured when standard luminance was 6,000 cd/m² through a lifetime measurement system (M6000) manufactured by McScience Inc. The results are described in the following Tables 7 and 8. Herein, T₉₀ means a lifetime (unit: h, hour), time taken to become 90% with respect to initial luminance.

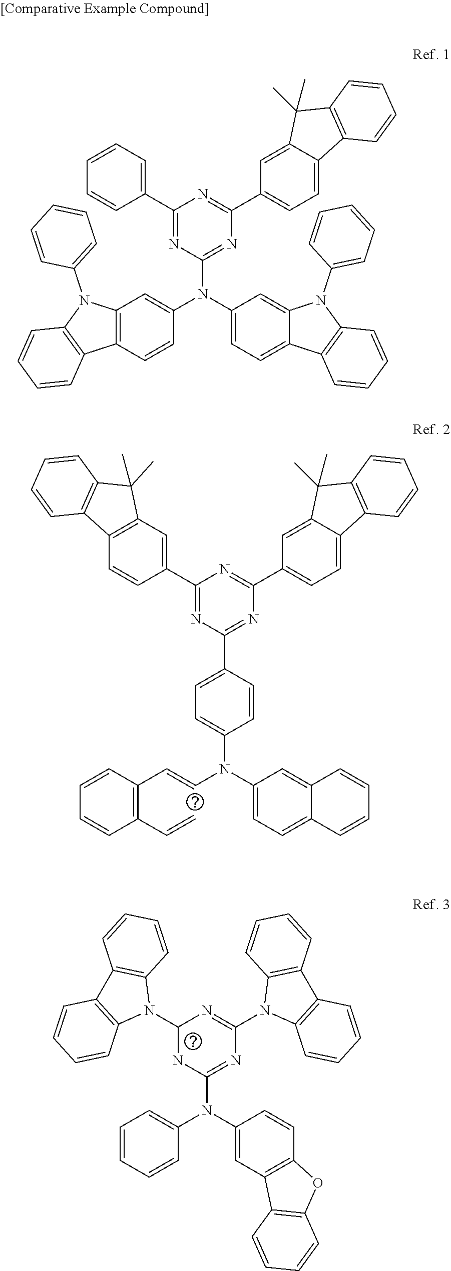 [SMILES: CC1(C)c2ccccc2-c2ccc(-c3nc(-c4ccccc4)nc(N(c4ccc5c6ccccc6n(-c6ccccc6)c5c4)c4ccc5c6ccccc6n(-c6ccccc6)c5c4)n3)cc21 ; C=Cc1ccccc1/C=C/N(c1ccc(-c2nc(-c3ccc4c(c3)C(C)(C)c3ccccc3-4)nc(-c3ccc4c(c3)C(C)(C)c3ccccc3-4)n2)cc1)c1ccc2ccccc2c1 ; c1ccc(N(C2=NC(n3c4ccccc4c4ccccc43)=NC(n3c4ccccc4c4ccccc43)N2)c2ccc3oc4ccccc4c3c2)cc1]

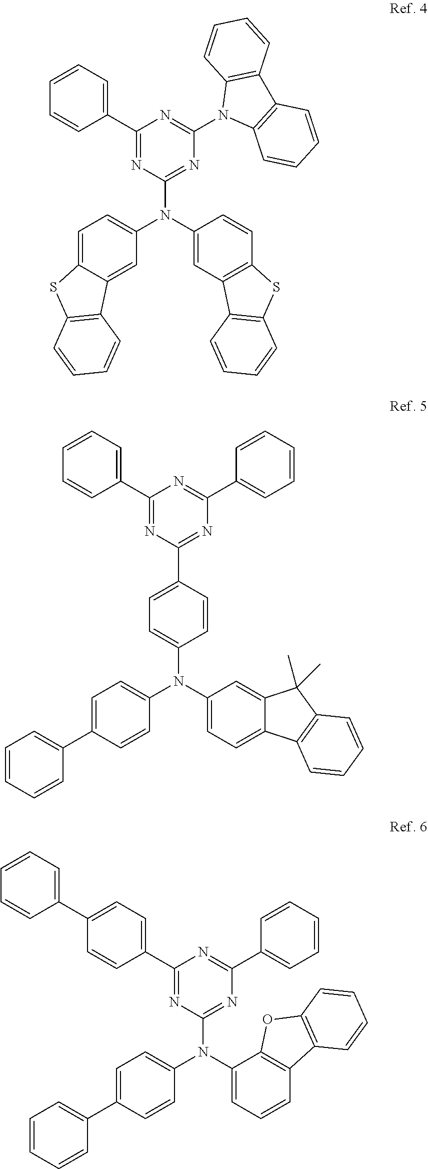 [SMILES: c1ccc(-c2nc(N(c3ccc4sc5ccccc5c4c3)c3ccc4sc5ccccc5c4c3)nc(-n3c4ccccc4c4ccccc43)n2)cc1 ; CC1(C)c2ccccc2-c2ccc(N(c3ccc(-c4ccccc4)cc3)c3ccc(-c4nc(-c5ccccc5)nc(-c5ccccc5)n4)cc3)cc21 ; c1ccc(-c2ccc(-c3nc(-c4ccccc4)nc(N(c4ccc(-c5ccccc5)cc4)c4cccc5c4oc4ccccc45)n3)cc2)cc1]

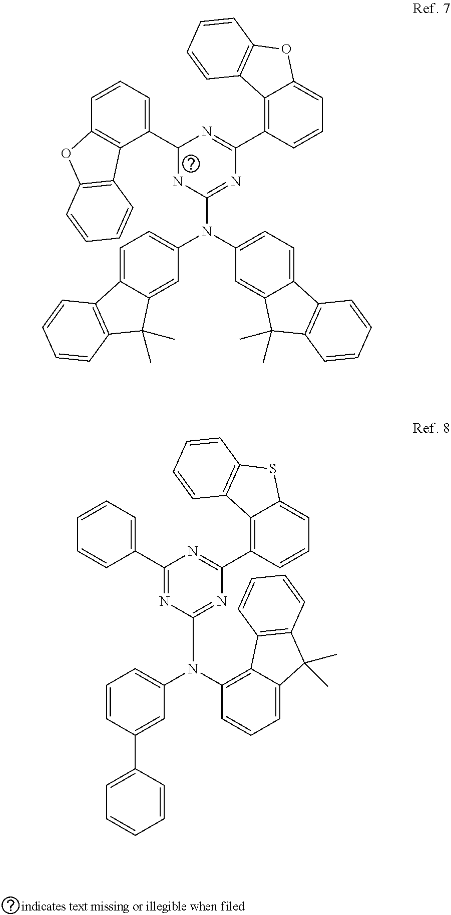 [SMILES: CC1(C)c2ccccc2-c2ccc(N(C3=NC(c4cccc5oc6ccccc6c45)=NC(c4cccc5oc6ccccc6c45)N3)c3ccc4c(c3)C(C)(C)c3ccccc3-4)cc21 ; CC1(C)c2ccccc2-c2c(N(c3cccc(-c4ccccc4)c3)c3nc(-c4ccccc4)nc(-c4cccc5sc6ccccc6c45)n3)cccc21]

TABLE 7

| | Light | | Light | | |
|---|---|---|---|---|---|
| | Emitting | Driving | Emission | Color | Life- |
| | Layer | Voltage | Efficiency | Coordinate | time |
| | Compound | (V) | (cd/A) | (x, y) | (T₉₀) |
| Comparative | 2-3 | 2.63 | 29.2 | (0.233, 0.671) | 45 |
| Example 1 | | | | | |
| Comparative | 2-4 | 2.59 | 31.3 | (0.231, 0.673) | 48 |
| Example 2 | | | | | |
| Comparative | 2-7 | 2.61 | 30.4 | (0.237, 0.677) | 44 |
| Example 3 | | | | | |
| Comparative | 2-31 | 2.65 | 30.8 | (0.234, 0.674) | 43 |
| Example 4 | | | | | |
| Comparative | 2-32 | 2.58 | 29.8 | (0.231, 0.681) | 44 |
| Example 5 | | | | | |
| Comparative | 2-42 | 2.59 | 30.1 | (0.233, 0.682) | 43 |
| Example 6 | | | | | |
| Comparative | Ref. 1 | 6.08 | 42.3 | (0.231, 0.671) | 141 |
| Example 7 | | | | | |
| Comparative | Ref. 2 | 5.81 | 43.7 | (0.230, 0.673) | 145 |
| Example 8 | | | | | |
| Comparative | Ref. 3 | 6.26 | 47.7 | (0.232, 0.664) | 157 |
| Example 9 | | | | | |
| Comparative | Ref. 4 | 6.30 | 47.2 | (0.235, 0.681) | 161 |
| Example 10 | | | | | |
| Comparative | Ref. 5 | 6.11 | 43.1 | (0.233, 0.663) | 151 |
| Example 11 | | | | | |
| Comparative | Ref. 6 | 6.19 | 43.7 | (0.230, 0.671) | 152 |
| Example 12 | | | | | |
| Comparative | Ref. 7 | 6.14 | 45.4 | (0.234, 0.667) | 147 |
| Example 13 | | | | | |
| Comparative | Ref. 8 | 6.15 | 44.3 | (0.237, 0.680) | 150 |
| Example 14 | | | | | |

TABLE 8

| | Light | | Light | | |
|---|---|---|---|---|---|
| | Emitting | Driving | Emission | | |
| | Layer | Voltage | Efficiency | Color Coordinate | Lifetime |
| | Compound | (V) | (cd/A) | (x, y) | (T₉₀) |
| Example 1 | 1-1 | 4.29 | 57.8 | (0.242, 0.672) | 294 |
| Example 2 | 1-3 | 4.47 | 56.4 | (0.245, 0.675) | 278 |
| Example 3 | 1-12 | 4.30 | 57.3 | (0.229, 0.682) | 280 |
| Example 4 | 1-17 | 4.30 | 57.5 | (0.242, 0.672) | 293 |
| Example 5 | 1-20 | 4.22 | 64.3 | (0.243, 0.675) | 300 |
| Example 6 | 1-40 | 4.17 | 65.7 | (0.241, 0.672) | 311 |
| Example 7 | 1-42 | 4.45 | 56.7 | (0.243, 0.671) | 277 |
| Example 8 | 1-46 | 4.31 | 57.9 | (0.233, 0.671) | 299 |
| Example 9 | 1-58 | 4.37 | 57.4 | (0.243, 0.676) | 294 |
| Example 10 | 1-60 | 4.45 | 51.9 | (0.233, 0.669) | 235 |
| Example 11 | 1-61 | 4.37 | 56.4 | (0.240, 0.671) | 275 |
| Example 12 | 1-67 | 4.50 | 55.0 | (0.243, 0.670) | 271 |
| Example 13 | 1-69 | 4.50 | 56.2 | (0.238, 0.670) | 271 |
| Example 14 | 1-77 | 4.43 | 57.9 | (0.239, 0.678) | 289 |
| Example 15 | 1-81 | 4.47 | 54.7 | (0.238, 0.670) | 260 |
| Example 16 | 1-89 | 4.50 | 54.1 | (0.235, 0.671) | 262 |
| Example 17 | 1-102 | 4.49 | 54.2 | (0.242, 0.665) | 255 |
| Example 18 | 1-106 | 4.59 | 53.9 | (0.240, 0.672) | 249 |
| Example 19 | 1-109 | 4.38 | 55.8 | (0.242, 0.662) | 272 |
| Example 20 | 1-116 | 4.58 | 53.5 | (0.243, 0.661) | 248 |
| Example 21 | 1-118 | 4.43 | 55.1 | (0.240, 0.661) | 270 |
| Example 22 | 1-121 | 4.37 | 57.0 | (0.243, 0.672) | 289 |
| Example 23 | 1-125 | 4.58 | 54.6 | (0.245, 0.662) | 259 |
| Example 24 | 1-136 | 4.49 | 53.8 | (0.234, 0.669) | 260 |
| Example 25 | 1-142 | 4.48 | 54.5 | (0.245, 0.662) | 259 |
| Example 26 | 1-163 | 4.47 | 53.4 | (0.244, 0.661) | 259 |
| Example 27 | 1-178 | 4.49 | 54.8 | (0.239, 0.679) | 256 |
| Example 28 | 1-189 | 4.20 | 57.8 | (0.247, 0.685) | 289 |
| Example 29 | 1-192 | 4.43 | 52.5 | (0.240, 0.660) | 235 |
| Example 30 | 1-199 | 4.20 | 65.0 | (0.240, 0.671) | 305 |
| Example 31 | 1-201 | 4.28 | 57.1 | (0.239, 0.673) | 291 |
| Example 32 | 1-202 | 4.46 | 55.7 | (0.244, 0.673) | 270 |
| Example 33 | 1-207 | 4.38 | 56.6 | (0.245, 0.662) | 279 |
| Example 34 | 1-211 | 4.41 | 55.9 | (0.242, 0.666) | 269 |
| Example 35 | 1-224 | 4.39 | 57.5 | (0.241, 0.672) | 286 |
| Example 36 | 1-230 | 4.52 | 52.0 | (0.235, 0.675) | 245 |
| Example 37 | 1-233 | 4.49 | 56.2 | (0.244, 0.671) | 274 |
| Example 38 | 1-237 | 4.45 | 52.9 | (0.247, 0.666) | 240 |
| Example 39 | 1-239 | 4.37 | 53.6 | (0.244, 0.670) | 238 |
| Example 40 | 1-240 | 4.53 | 52.5 | (0.240, 0.670) | 233 |
| Example 41 | 1-244 | 4.41 | 52.9 | (0.242, 0.666) | 239 |
| Example 42 | 1-249 | 4.52 | 53.8 | (0.245, 0.665) | 250 |
| Example 43 | 1-259 | 4.46 | 51.5 | (0.240, 0.666) | 235 |
| Example 44 | 1-280 | 4.31 | 57.0 | (0.231, 0.674) | 285 |
| Example 45 | 1-286 | 4.35 | 57.1 | (0.232, 0.685) | 287 |
| Example 46 | 1-293 | 4.40 | 52.7 | (0.242, 0.665) | 239 |
| Example 47 | 1-295 | 4.32 | 58.3 | (0.238, 0.675) | 287 |
| Example 48 | 1-299 | 4.53 | 52.5 | (0.240, 0.670) | 233 |
| Example 49 | 1-301 | 4.16 | 58.5 | (0.231, 0.675) | 293 |
| Example 50 | 1-306 | 4.44 | 56.7 | (0.238, 0.670) | 270 |
| Example 51 | 1-309 | 4.49 | 56.0 | (0.237, 0.671) | 269 |
| Example 52 | 1-312 | 4.45 | 53.9 | (0.233, 0.669) | 260 |
| Example 53 | 1-320 | 4.45 | 55.5 | (0.244, 0.661) | 267 |
| Example 54 | 1-323 | 4.51 | 54.0 | (0.242, 0.671) | 256 |
| Example 55 | 1-325 | 4.35 | 61.0 | (0.233, 0.678) | 299 |
| Example 56 | 1-326 | 4.26 | 59.1 | (0.233, 0.678) | 298 |
| Example 57 | 1-333 | 4.43 | 55.1 | (0.243, 0.666) | 265 |
| Example 58 | 1-336 | 4.46 | 54.5 | (0.240, 0.666) | 265 |
| Example 59 | 1-338 | 4.25 | 59.6 | (0.232, 0.678) | 295 |
| Example 60 | 1-340 | 4.49 | 54.0 | (0.233, 0.669) | 238 |
| Example 61 | 1-341 | 4.27 | 56.3 | (0.231, 0.684) | 281 |
| Example 62 | 1-342 | 4.53 | 55.0 | (0.242, 0.670) | 262 |
| Example 63 | 1-349 | 4.47 | 52.7 | (0.238, 0.670) | 255 |
| Example 64 | 1-350 | 4.58 | 55.3 | (0.245, 0.670) | 261 |
| Example 65 | 1-355 | 4.36 | 51.2 | (0.243, 0.669) | 231 |
| Example 66 | 1-360 | 4.39 | 53.8 | (0.244, 0.666) | 239 |
| Example 67 | 1-362 | 4.47 | 55.5 | (0.243, 0.666) | 280 |
| Example 68 | 1-366 | 4.37 | 57.7 | (0.244, 0.673) | 283 |
| Example 69 | 1-368 | 4.50 | 53.7 | (0.242, 0.664) | 254 |
| Example 70 | 1-370 | 4.49 | 53.5 | (0.241, 0.664) | 261 |
| Example 71 | 1-371 | 4.28 | 58.7 | (0.242, 0.672) | 288 |
| Example 72 | 1-376 | 4.25 | 63.8 | (0.243, 0.675) | 299 |
| Example 73 | 1-379 | 4.43 | 52.5 | (0.231, 0.672) | 237 |

As seen from the results of Table 7 and Table 8, the organic electroluminescent device using the organic electroluminescent device light emitting layer material of the present disclosure had lower driving voltage, enhanced light emission efficiency, and significantly improved lifetime compared to Comparative Examples 1 to 14.

Particularly, it was seen that Examples 5, 6 and 72 using the compound substituted with deuterium generally had lower driving voltage, higher light emission efficiency and longer lifetime compared to other examples.

It was identified that, when an amine group in the core structure of the heterocyclic compound of the present disclosure has a carbazole substituent or an aryl substituent as in the heterocyclic compounds of Comparative Examples 7 and 8, the HOMO was localized breaking a balance between holes and electrons, which reduced lifetime and efficiency.

It was identified that, when triazine in the core structure of the heterocyclic compound of the present disclosure bonds to nitrogen of carbazole as in the heterocyclic compounds of Comparative Examples 9 and 10, T1 (energy level value in triplet state) was low of 2.48, which increased a driving voltage.

It was identified that, when triazine in the core structure of the heterocyclic compound of the present disclosure has an aryl substituent as in the heterocyclic compounds of Comparative Examples 11 and 12, the LUMO was less spread reducing electron mobility and thereby breaking a balance between holes and electrons in the light emitting layer, and lifetime and efficiency were reduced.

It was identified that, when triazine in the core structure of the heterocyclic compound of the present disclosure bonds to a No. 1 position of dibenzofuran or dibenzothiophene as in the heterocyclic compounds of Comparative Examples 13 and 14, Td (decomposition temperature) was low and structural destabilization occurred, which reduced a lifetime.

### Experimental Example 2

### 1) Manufacture of Organic Light Emitting Device

### Comparative Example 15

A glass substrate on which ITO was coated as a thin film to a thickness of 1,500 Å was cleaned with distilled water ultrasonic waves. After the cleaning with distilled water was finished, the substrate was ultrasonic cleaned with solvents such as acetone, methanol and isopropyl alcohol, then dried, and UVO treatment was conducted for 5 minutes using UV in a UV cleaner. After that, the substrate was transferred to a plasma cleaner (PT), and after conducting plasma treatment under vacuum for ITO work function and residual film removal, the substrate was transferred to a thermal deposition apparatus for organic deposition.

On the transparent ITO electrode (anode), a hole injection layer 2-TNATA (4,4′,4″-tris[2-naphthyl(phenyl)amino]triphenylamine) and a hole transfer layer NPB (N,N′-di(1-naphthyl)-N,N′-diphenyl-(1,1′-biphenyl)-4,4′-diamine), which are common layers, were formed.

A light emitting layer was thermal vacuum deposited thereon as follows. As the light emitting layer, Compound Ref. 1 and Compound 2-32 were pre-mixed in a weight ratio of 1:2 and then deposited to 400 Å in one source of supply as a host, and as a green phosphorescent dopant, Ir(ppy)₃ was doped and deposited by 7% of the deposited thickness of the light emitting layer. After that, BCP was deposited to 60 Å as a hole blocking layer, and Alq₃ was deposited to 200 Å thereon as an electron transfer layer. Lastly, an electron injection layer was formed on the electron transfer layer by depositing lithium fluoride (LiF) to a thickness of 10 Å, and then a cathode was formed on the electron injection layer by depositing an aluminum (Al) cathode to a thickness of 1,200 Å, and as a result, an organic light emitting device of Comparative Example 15 was manufactured.

### Comparative Examples 16 to 20 and Examples 74 to 112

Organic light emitting devices of Comparative Examples 16 to 20 and Examples 74 to 112 were additionally manufactured in the same manner as in the method for manufacturing an organic light emitting device of Comparative Example 15 except that, in the process for manufacturing an organic light emitting device of Comparative Example 15, compounds described in the following Table 9 were used in weight ratios described in Table 9 as the host of the light emitting layer instead of using Compound Ref. 1 and Compound 2-32.

Meanwhile, all the organic compounds required to manufacture the OLED were vacuum sublimation purified under 10⁻⁸ torr to 10⁻⁶ torr for each material to be used in the OLED manufacture.

### 2) Evaluation on Organic Light Emitting Device

For each of the organic light emitting devices of Comparative Examples 15 to 20 and Examples 74 to 112 manufactured as above, electroluminescent (EL) properties were measured using M7000 manufactured by McScience Inc., and with the measurement results, T₉₀ was measured when standard luminance was 6,000 cd/m² through a lifetime measurement system (M6000) manufactured by McScience Inc. The results are described in the following Table 9. Herein, T₉₀ means a lifetime (unit: h, hour), time taken to become 90% with respect to initial luminance.

TABLE 9

| | Light | | | Light | | |
|---|---|---|---|---|---|---|
| | Emitting | Ratio | Driving | Emission | Color | |
| | Layer | (Weight | Voltage | Efficiency | Coordinate | Lifetime |
| | Compound | Ratio) | (V) | (cd/A) | (x, y) | (T₉₀) |
| Comparative | Ref. 1:2-32 | 1:2 | 4.69 | 67.7 | (0.241, 0.711) | 441 |
| Example 15 | | | | | | |
| Comparative | Ref. 1:2-32 | 1:1 | 4.61 | 65.4 | (0.242, 0.724) | 430 |
| Example 16 | | | | | | |
| Comparative | Ref. 1:2-32 | 2:1 | 4.58 | 64.8 | (0.243, 0.710) | 422 |
| Example 17 | | | | | | |
| Comparative | Ref. 5:2-32 | 1:2 | 4.65 | 68.4 | (0.241, 0.723) | 449 |
| Example 18 | | | | | | |
| Comparative | Ref. 5:2-32 | 1:1 | 4.63 | 66.8 | (0.252, 0.714) | 437 |
| Example 19 | | | | | | |
| Comparative | Ref. 5:2-32 | 2:1 | 4.48 | 65.2 | (0.231, 0.712) | 433 |
| Example 20 | | | | | | |
| Example 74 | 1-1:2-31 | 1:2 | 4.10 | 82.7 | (0.251, 0.725) | 583 |
| Example 75 | 1-1:2-31 | 1:1 | 3.93 | 79.5 | (0.240, 0.710) | 522 |
| Example 76 | 1-1:2-31 | 2:1 | 3.80 | 75.5 | (0.242, 0.717) | 497 |
| Example 77 | 1-3:2-42 | 1:2 | 4.22 | 79.0 | (0.252, 0.715) | 557 |
| Example 78 | 1-3:2-42 | 1:1 | 4.20 | 77.5 | (0.232, 0.711) | 511 |
| Example 79 | 1-3:2-42 | 2:1 | 4.19 | 71.8 | (0.234, 0.712) | 499 |
| Example 80 | 1-12:2-31 | 1:2 | 4.27 | 79.6 | (0.241, 0.714) | 569 |
| Example 81 | 1-12:2-31 | 1:1 | 4.21 | 77.8 | (0.243, 0.715) | 507 |
| Example 82 | 1-12:2-31 | 2:1 | 4.10 | 71.6 | (0.243, 0.712) | 487 |
| Example 83 | 1-46:2-32 | 1:2 | 4.22 | 85.3 | (0.231, 0.710) | 593 |
| Example 84 | 1-46:2-32 | 1:1 | 3.97 | 80.3 | (0.233, 0.712) | 537 |
| Example 85 | 1-46:2-32 | 2:1 | 3.96 | 78.2 | (0.253, 0.711) | 531 |
| Example 86 | 1-60:2-42 | 1:2 | 4.23 | 76.7 | (0.231, 0.710) | 528 |
| Example 87 | 1-60:2-42 | 1:1 | 4.18 | 75.2 | (0.233, 0.712) | 493 |
| Example 88 | 1-60:2-42 | 2:1 | 4.109 | 70.1 | (0.253, 0.711) | 471 |
| Example 89 | 1-136:2-3 | 1:8 | 4.35 | 67.6 | (0.243, 0.713) | 412 |
| Example 90 | 1-136:2-3 | 1:5 | 4.31 | 70.4 | (0.233, 0.714) | 449 |
| Example 91 | 1-136:2-3 | 1:2 | 4.25 | 78.2 | (0.212, 0.714) | 554 |
| Example 92 | 1-136:2-3 | 1:1 | 4.19 | 76.3 | (0.221, 0.721) | 507 |
| Example 93 | 1-136:2-3 | 2:1 | 3.98 | 70.7 | (0.231, 0.711) | 492 |
| Example 94 | 1-136:2-3 | 5:1 | 4.42 | 70.1 | (0.231, 0.715) | 457 |
| Example 95 | 1-136:2-3 | 8:1 | 4.50 | 67.0 | (0.252, 0.711) | 404 |
| Example 96 | 1-189:2-31 | 1:2 | 4.21 | 80.2 | (0.244, 0.710) | 576 |
| Example 97 | 1-189:2-31 | 1:1 | 4.17 | 79.1 | (0.231, 0.711) | 521 |
| Example 98 | 1-189:2-31 | 2:1 | 4.08 | 74.7 | (0.233, 0.714) | 493 |
| Example 99 | 1-192:2-42 | 1:2 | 4.21 | 75.6 | (0.252, 0.716) | 506 |
| Example 100 | 1-192:2-42 | 1:1 | 4.01 | 74.8 | (0.234, 0.713) | 481 |
| Example 101 | 1-192:2-42 | 2:1 | 3.93 | 70.9 | (0.232, 0.711) | 463 |
| Example 102 | 1-306:2-7 | 1:2 | 4.20 | 77.4 | (0.242, 0.724) | 549 |
| Example 103 | 1-306:2-7 | 1:1 | 4.12 | 75.3 | (0.232, 0.711) | 503 |
| Example 104 | 1-306:2-7 | 2:1 | 3.86 | 70.3 | (0.233, 0.720) | 484 |
| Example 105 | 1-341:2-4 | 1:3 | 4.31 | 70.6 | (0.244, 0.710) | 462 |
| Example 106 | 1-341:2-4 | 1:2 | 4.18 | 79.8 | (0.232, 0.710) | 572 |
| Example 107 | 1-341:2-4 | 1:1 | 4.20 | 78.2 | (0.230, 0.711) | 512 |
| Example 108 | 1-341:2-4 | 2:1 | 3.99 | 72.3 | (0.233, 0.713) | 500 |
| Example 109 | 1-341:2-4 | 3:1 | 4.31 | 70.1 | (0.244, 0.710) | 467 |
| Example 110 | 1-379:2-7 | 1:2 | 4.18 | 77.2 | (0.242, 0.714) | 543 |
| Example 111 | 1-379:2-7 | 1:1 | 4.03 | 76.1 | (0.231, 0.713) | 499 |
| Example 112 | 1-379:2-7 | 2:1 | 3.92 | 69.7 | (0.251, 0.713) | 473 |

As seen from the results of Table 8 and Table 9, effects of more superior efficiency and lifetime are obtained when including the heterocyclic compound and the compound of Chemical Formula 2 of the present disclosure at the same time. Such results may lead to a forecast that an exciplex phenomenon occurs when including the two compounds at the same time.

The exciplex phenomenon is a phenomenon of releasing energy having sizes of a donor (p-host) HOMO level and an acceptor (n-host) LUMO level due to electron exchanges between two molecules. When the exciplex phenomenon occurs between two molecules, reverse intersystem crossing (RISC) occurs, and as a result, internal quantum efficiency of fluorescence may increase up to 100%. When a donor (p-host) having a favorable hole transfer ability and an acceptor (n-host) having a favorable electron transfer ability are used as a host of a light emitting layer, holes are injected to the p-host and electrons are injected to the n-host, and therefore, a driving voltage may be lowered, which resultantly helps with enhancement in the lifetime.

From the results, it was identified that superior device properties were obtained when using the compound of Chemical Formula 2 of the present application performing a donor role and the heterocyclic compound of Chemical Formula 1 of the present application performing an acceptor role as a host of the light emitting layer.

1. A heterocyclic compound of the following Chemical Formula 1: 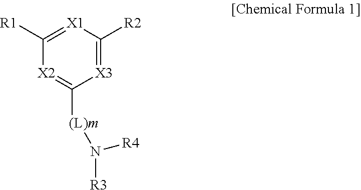
in Chemical Formula 1,
X1 to X3 are each independently N; or CH, and at least two thereof are N,
L is a direct bond; or a substituted or unsubstituted C6 to C60 arylene group,
m is an integer of 1 to 5, and when 2 or greater, Ls are the same as or different from each other,
R1 and R2 are the same as or different from each other and each independently a substituted or unsubstituted C6 to C12 aryl group; a substituted or unsubstituted fluorenyl group; or a substituted or unsubstituted C2 to C60 heteroaryl group, and at least one of R1 and R2 is a substituted or unsubstituted fluorenyl group; represented by the following Chemical Formula A; or represented by the following Chemical Formula B, and
R3 and R4 are the same as or different from each other and each independently a substituted or unsubstituted C6 to C12 aryl group; a substituted or unsubstituted fluorenyl group; or a heteroaryl group substituted or unsubstituted and including O or S, and at least one of R3 and R4 is a substituted or unsubstituted fluorenyl group; or represented by the following Chemical Formula C, 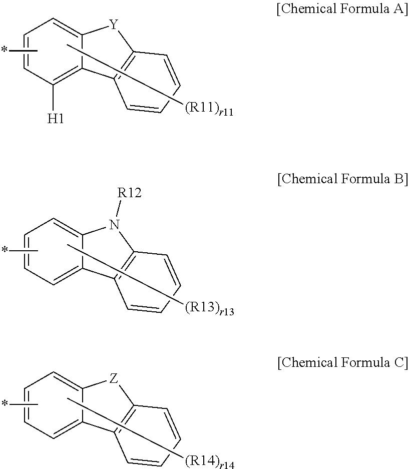
in Chemical Formulae A to C,
Y and Z are each independently O; or S,
H1 is hydrogen; or deuterium,
R11 to R14 are each independently hydrogen; deuterium; a substituted or unsubstituted C1 to C60 alkyl group; a substituted or unsubstituted C6 to C60 aryl group; or a substituted or unsubstituted C2 to C60 heteroaryl group,
r11 is an integer of 0 to 6, and when 2 or greater, R11s are the same as or different from each other,
r13 is an integer of 0 to 7, and when 2 or greater, R13s are the same as or different from each other, and
r14 is an integer of 0 to 7, and when 2 or greater, R14s are the same as or different from each other.

2. The heterocyclic compound of claim 1, wherein, as for R1 and R2, i) any one of R1 and R2 is a substituted or unsubstituted fluorenyl group; represented by Chemical Formula A; or represented by Chemical Formula B, and the other one is a substituted or unsubstituted C6 to C12 aryl group, or
ii) R1 and R2 are each independently a substituted or unsubstituted fluorenyl group; represented by Chemical Formula A; or represented by Chemical Formula B.

3. The heterocyclic compound of claim 1, wherein, as for R3 and R4, i) any one of R3 and R4 is a substituted or unsubstituted fluorenyl group; or represented by Chemical Formula C, and the other one is a substituted or unsubstituted C6 to C12 aryl group, or
ii) R3 and R4 are each independently a substituted or unsubstituted fluorenyl group; or represented by Chemical Formula C.

4. The heterocyclic compound of claim 1, wherein X1 to X3 are N.

5. The heterocyclic compound of claim 1, wherein L is a direct bond; or a substituted or unsubstituted phenylene group.

6. The heterocyclic compound of claim 1, which has a deuterium content of either 0%, or 30% to 100%.

7. The heterocyclic compound of claim 1, wherein Chemical Formula 1 is represented by any one of the following compounds: 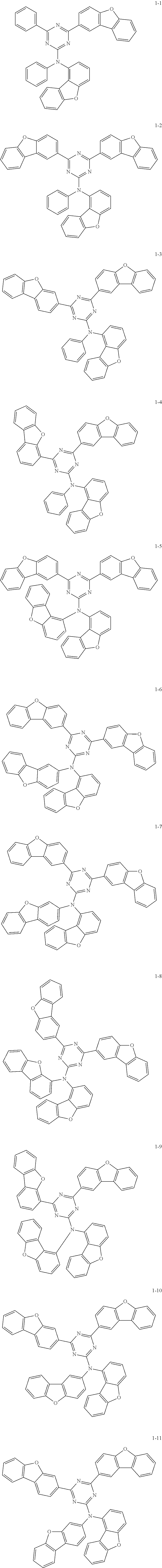 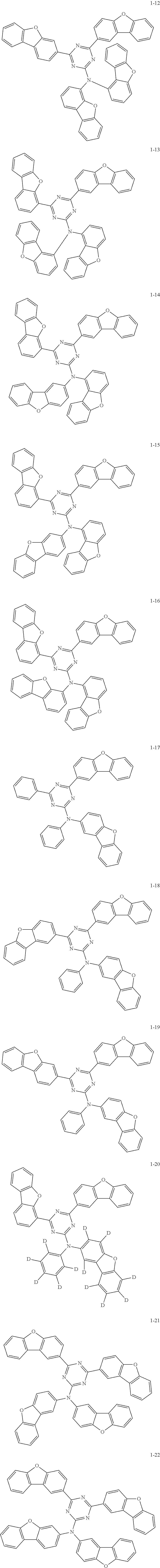 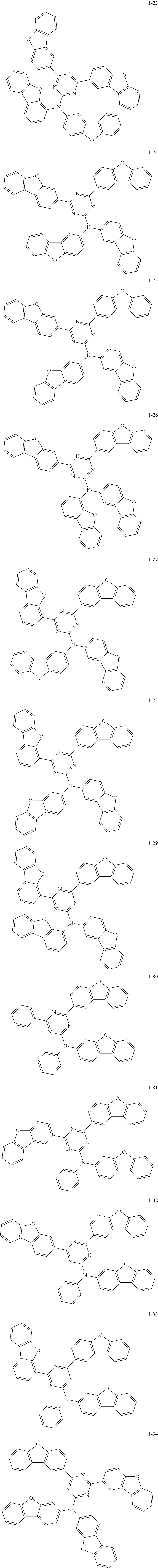 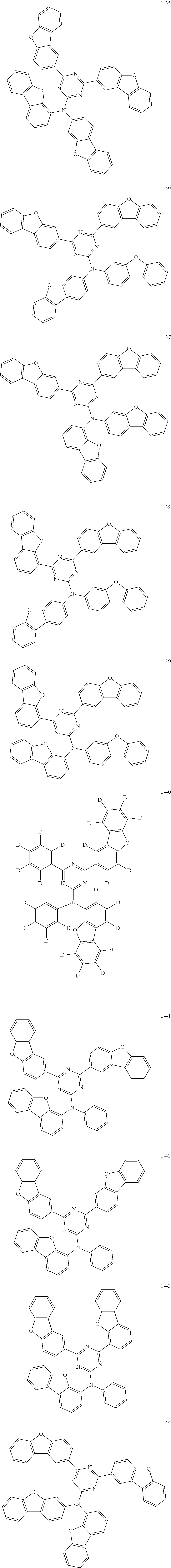 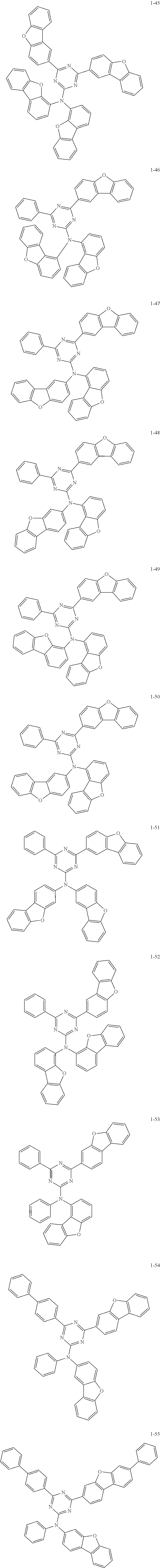 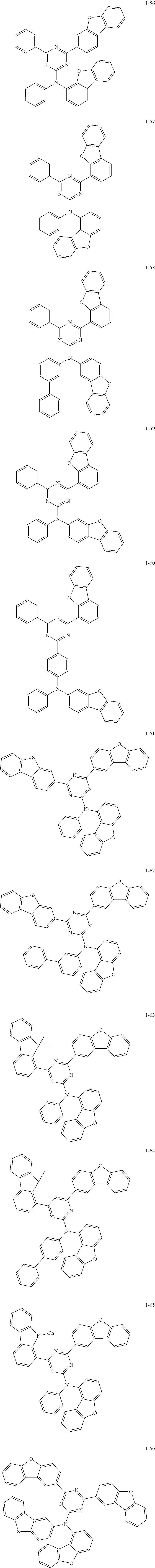 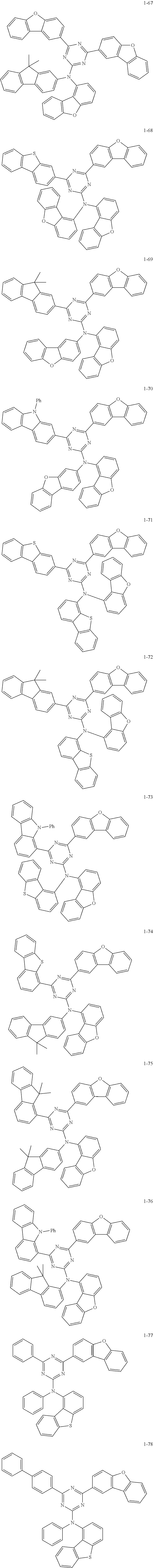 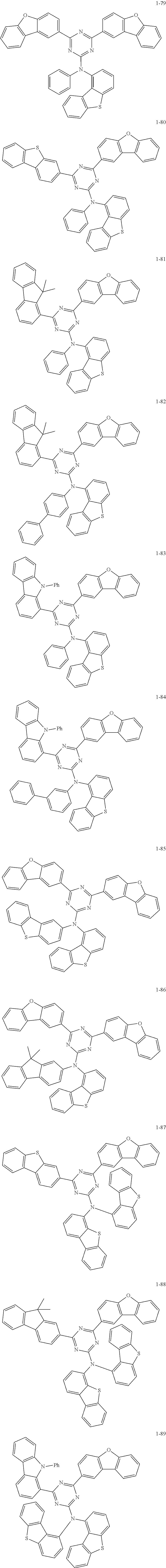 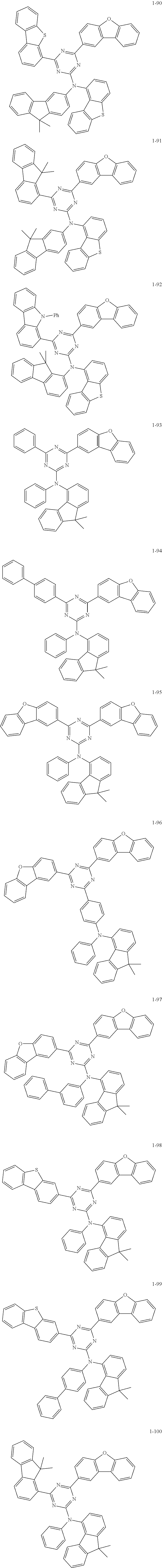 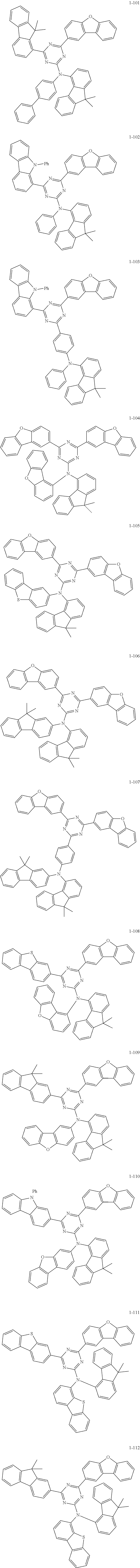 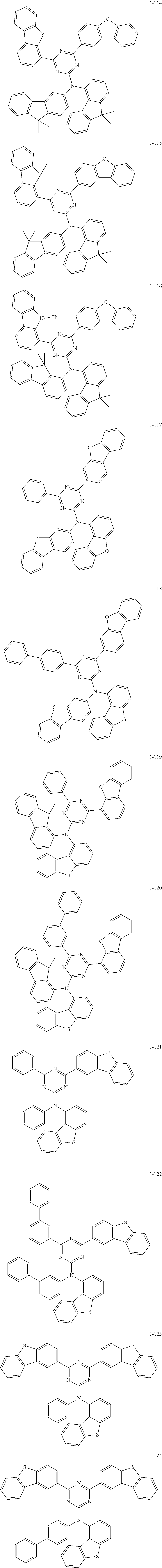 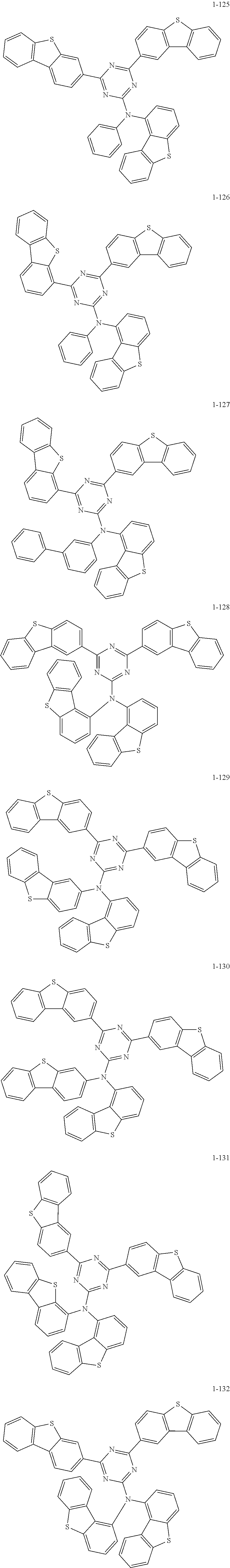 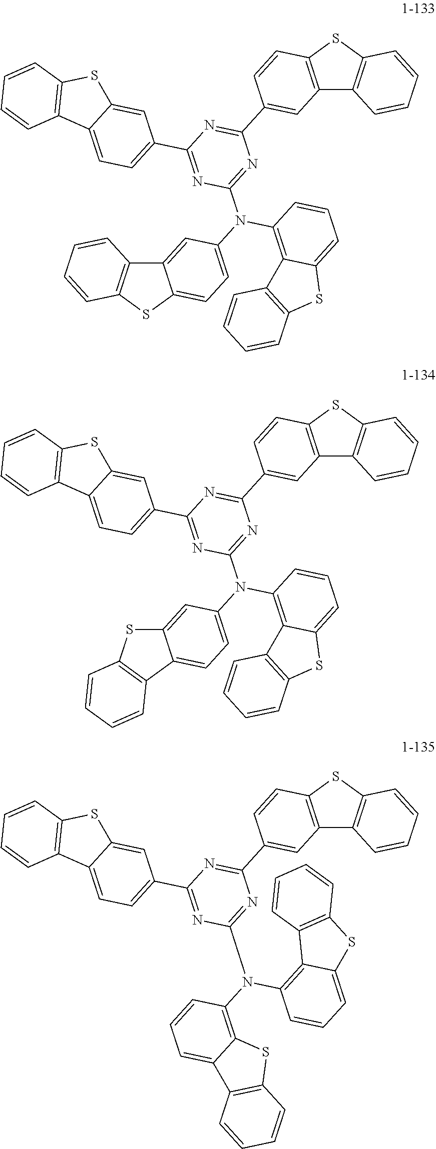 [SMILES: c1ccc2c(c1)sc1cc(-c3nc(-c4ccc5sc6ccccc6c5c4)nc(N(c4ccc5sc6ccccc6c5c4)c4cccc5sc6ccccc6c45)n3)ccc12 ; c1ccc2c(c1)sc1cc(-c3nc(-c4ccc5sc6ccccc6c5c4)nc(N(c4ccc5c(c4)sc4ccccc45)c4cccc5sc6ccccc6c45)n3)ccc12 ; c1ccc2c(c1)sc1cc(-c3nc(-c4ccc5sc6ccccc6c5c4)nc(N(c4cccc5c4sc4ccccc45)c4cccc5sc6ccccc6c45)n3)ccc12] 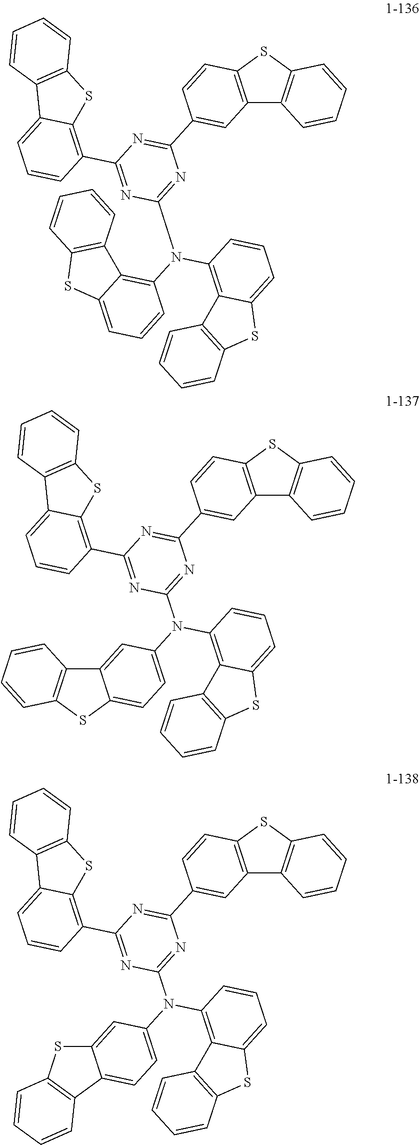 [SMILES: c1ccc2c(c1)sc1ccc(-c3nc(-c4cccc5c4sc4ccccc45)nc(N(c4cccc5sc6ccccc6c45)c4cccc5sc6ccccc6c45)n3)cc12 ; c1ccc2c(c1)sc1ccc(-c3nc(-c4cccc5c4sc4ccccc45)nc(N(c4ccc5sc6ccccc6c5c4)c4cccc5sc6ccccc6c45)n3)cc12 ; c1ccc2c(c1)sc1cc(N(c3nc(-c4ccc5sc6ccccc6c5c4)nc(-c4cccc5c4sc4ccccc45)n3)c3cccc4sc5ccccc5c34)ccc12] 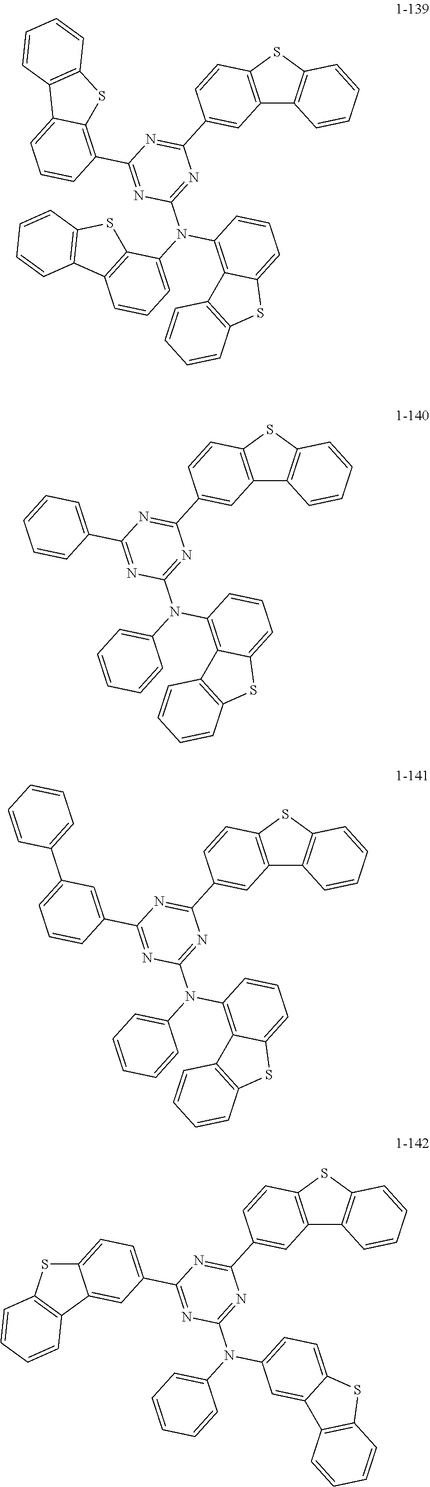 [SMILES: c1ccc2c(c1)sc1ccc(-c3nc(-c4cccc5c4sc4ccccc45)nc(N(c4cccc5c4sc4ccccc45)c4cccc5sc6ccccc6c45)n3)cc12 ; c1ccc(-c2nc(-c3ccc4sc5ccccc5c4c3)nc(N(c3ccccc3)c3cccc4sc5ccccc5c34)n2)cc1 ; c1ccc(-c2cccc(-c3nc(-c4ccc5sc6ccccc6c5c4)nc(N(c4ccccc4)c4cccc5sc6ccccc6c45)n3)c2)cc1 ; c1ccc(N(c2ccc3sc4ccccc4c3c2)c2nc(-c3ccc4sc5ccccc5c4c3)nc(-c3ccc4sc5ccccc5c4c3)n2)cc1] 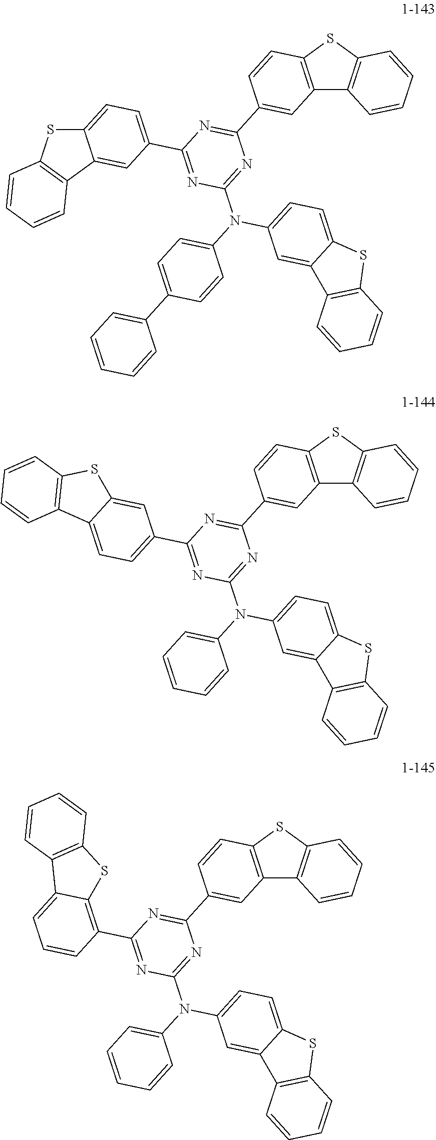 [SMILES: c1ccc(-c2ccc(N(c3ccc4sc5ccccc5c4c3)c3nc(-c4ccc5sc6ccccc6c5c4)nc(-c4ccc5sc6ccccc6c5c4)n3)cc2)cc1 ; c1ccc(N(c2ccc3sc4ccccc4c3c2)c2nc(-c3ccc4c(c3)sc3ccccc34)nc(-c3ccc4sc5ccccc5c4c3)n2)cc1 ; c1ccc(N(c2ccc3sc4ccccc4c3c2)c2nc(-c3ccc4sc5ccccc5c4c3)nc(-c3cccc4c3sc3ccccc34)n2)cc1] 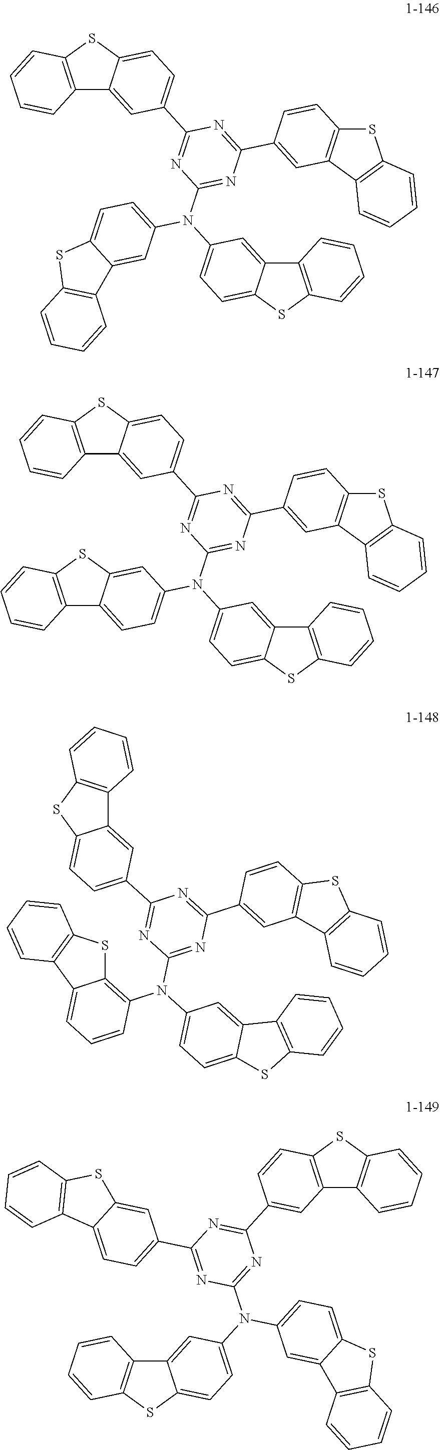 [SMILES: c1ccc2c(c1)sc1ccc(-c3nc(-c4ccc5sc6ccccc6c5c4)nc(N(c4ccc5sc6ccccc6c5c4)c4ccc5sc6ccccc6c5c4)n3)cc12 ; c1ccc2c(c1)sc1cc(N(c3ccc4sc5ccccc5c4c3)c3nc(-c4ccc5sc6ccccc6c5c4)nc(-c4ccc5sc6ccccc6c5c4)n3)ccc12 ; c1ccc2c(c1)sc1ccc(-c3nc(-c4ccc5sc6ccccc6c5c4)nc(N(c4ccc5sc6ccccc6c5c4)c4cccc5c4sc4ccccc45)n3)cc12 ; c1ccc2c(c1)sc1cc(-c3nc(-c4ccc5sc6ccccc6c5c4)nc(N(c4ccc5sc6ccccc6c5c4)c4ccc5sc6ccccc6c5c4)n3)ccc12] 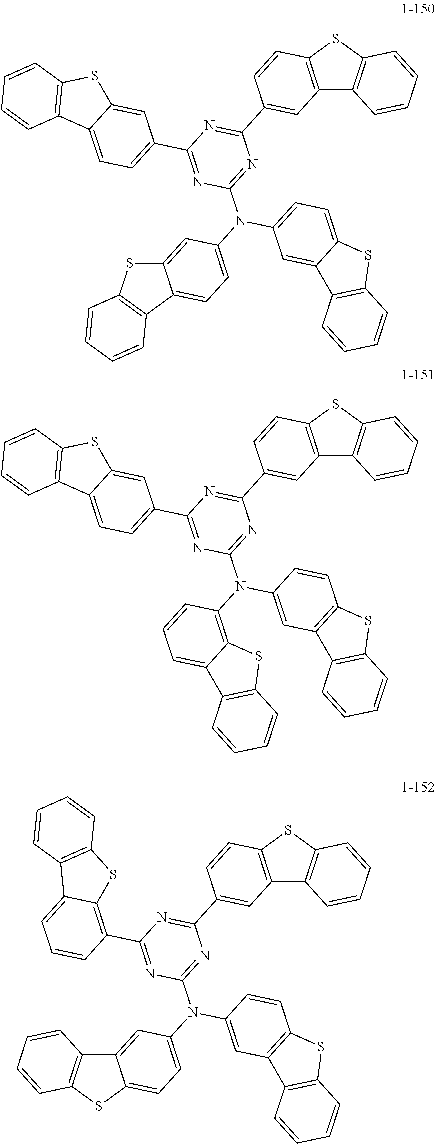 [SMILES: c1ccc2c(c1)sc1cc(-c3nc(-c4ccc5sc6ccccc6c5c4)nc(N(c4ccc5c(c4)sc4ccccc45)c4ccc5sc6ccccc6c5c4)n3)ccc12 ; c1ccc2c(c1)sc1cc(-c3nc(-c4ccc5sc6ccccc6c5c4)nc(N(c4ccc5sc6ccccc6c5c4)c4cccc5c4sc4ccccc45)n3)ccc12 ; c1ccc2c(c1)sc1ccc(-c3nc(-c4cccc5c4sc4ccccc45)nc(N(c4ccc5sc6ccccc6c5c4)c4ccc5sc6ccccc6c5c4)n3)cc12] 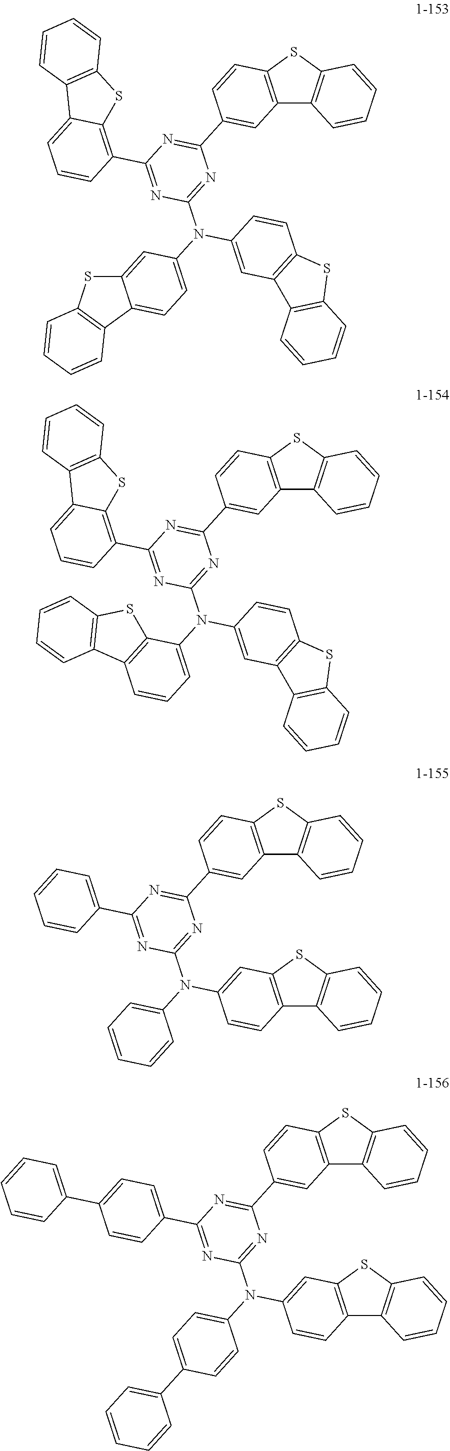 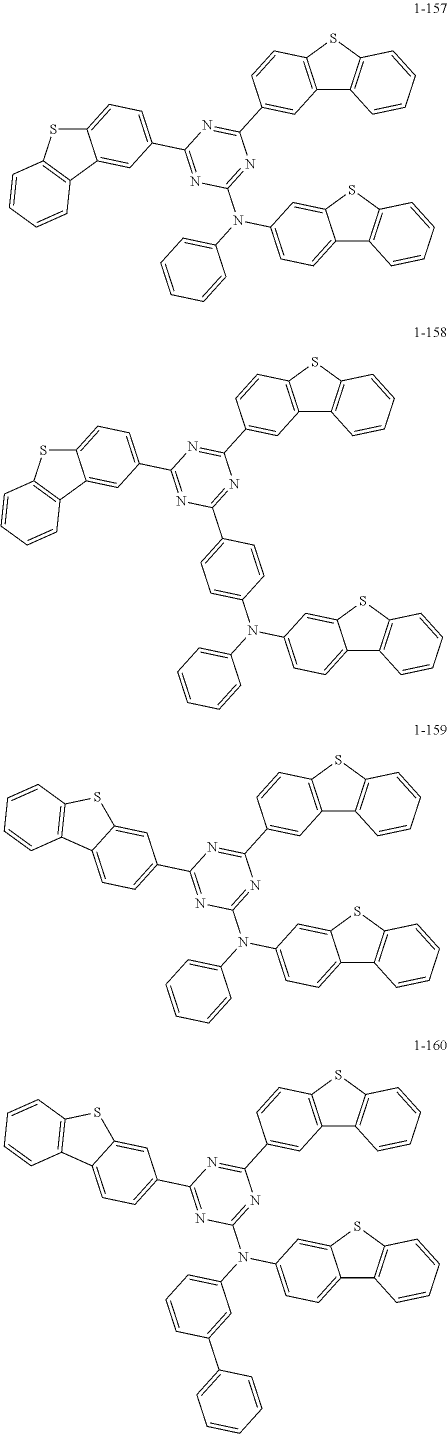 [SMILES: c1ccc(N(c2ccc3c(c2)sc2ccccc23)c2nc(-c3ccc4sc5ccccc5c4c3)nc(-c3ccc4sc5ccccc5c4c3)n2)cc1 ; c1ccc(N(c2ccc(-c3nc(-c4ccc5sc6ccccc6c5c4)nc(-c4ccc5sc6ccccc6c5c4)n3)cc2)c2ccc3c(c2)sc2ccccc23)cc1 ; c1ccc(N(c2ccc3c(c2)sc2ccccc23)c2nc(-c3ccc4c(c3)sc3ccccc34)nc(-c3ccc4sc5ccccc5c4c3)n2)cc1 ; c1ccc(-c2cccc(N(c3ccc4c(c3)sc3ccccc34)c3nc(-c4ccc5c(c4)sc4ccccc45)nc(-c4ccc5sc6ccccc6c5c4)n3)c2)cc1] 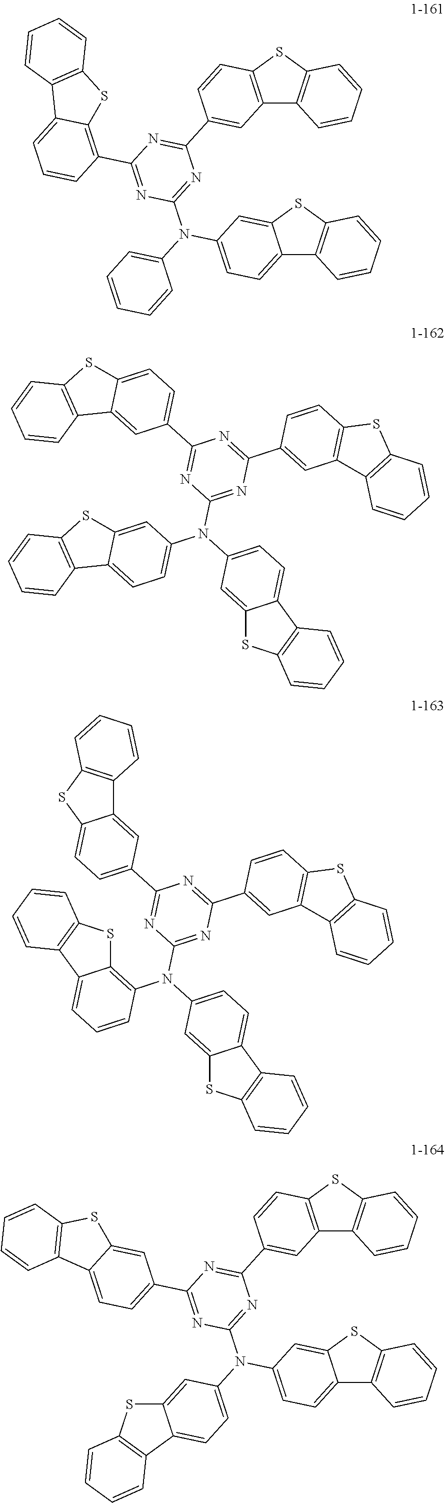 [SMILES: c1ccc(N(c2ccc3c(c2)sc2ccccc23)c2nc(-c3ccc4sc5ccccc5c4c3)nc(-c3cccc4c3sc3ccccc34)n2)cc1 ; c1ccc2c(c1)sc1cc(N(c3ccc4c(c3)sc3ccccc34)c3nc(-c4ccc5sc6ccccc6c5c4)nc(-c4ccc5sc6ccccc6c5c4)n3)ccc12 ; c1ccc2c(c1)sc1cc(N(c3nc(-c4ccc5sc6ccccc6c5c4)nc(-c4ccc5sc6ccccc6c5c4)n3)c3cccc4c3sc3ccccc34)ccc12 ; c1ccc2c(c1)sc1cc(-c3nc(-c4ccc5sc6ccccc6c5c4)nc(N(c4ccc5c(c4)sc4ccccc45)c4ccc5c(c4)sc4ccccc45)n3)ccc12] 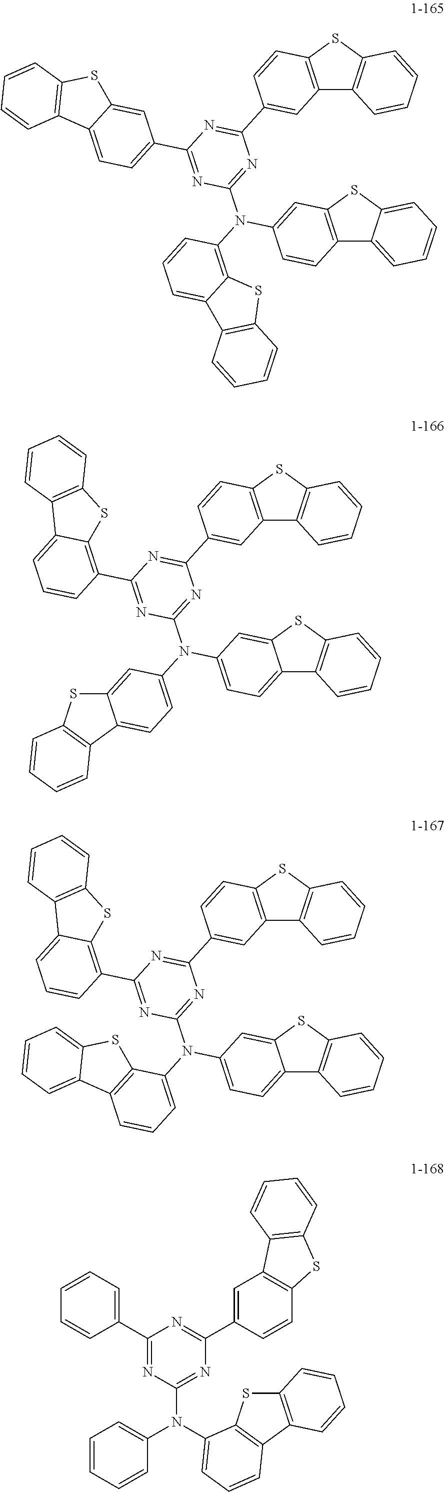 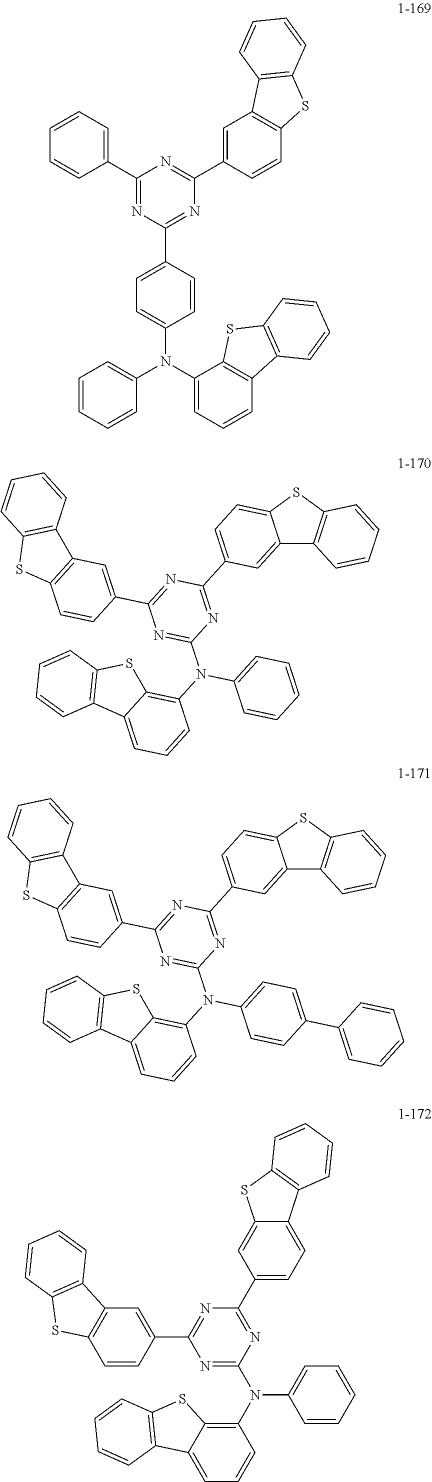 [SMILES: c1ccc(-c2nc(-c3ccc(N(c4ccccc4)c4cccc5c4sc4ccccc45)cc3)nc(-c3ccc4sc5ccccc5c4c3)n2)cc1 ; c1ccc(N(c2nc(-c3ccc4sc5ccccc5c4c3)nc(-c3ccc4sc5ccccc5c4c3)n2)c2cccc3c2sc2ccccc23)cc1 ; c1ccc(-c2ccc(N(c3nc(-c4ccc5sc6ccccc6c5c4)nc(-c4ccc5sc6ccccc6c5c4)n3)c3cccc4c3sc3ccccc34)cc2)cc1 ; c1ccc(N(c2nc(-c3ccc4c(c3)sc3ccccc34)nc(-c3ccc4sc5ccccc5c4c3)n2)c2cccc3c2sc2ccccc23)cc1] 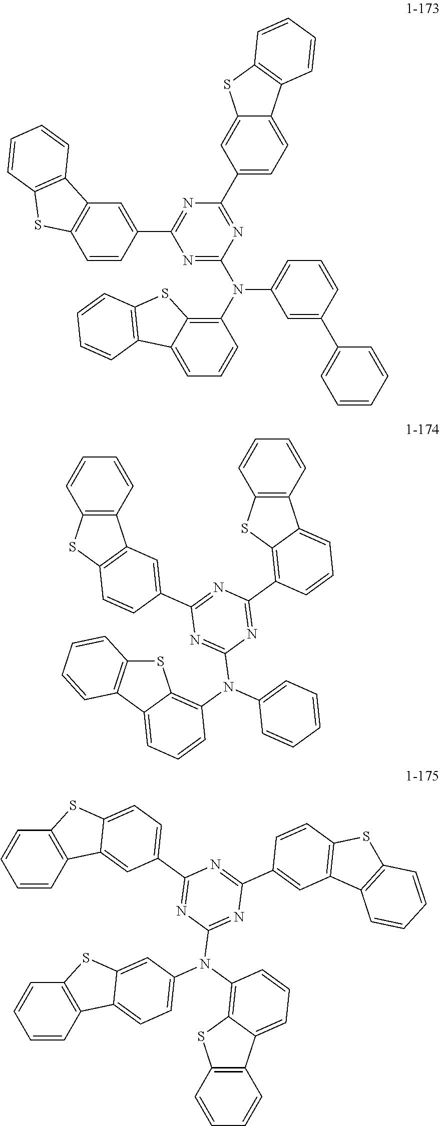 [SMILES: c1ccc(-c2cccc(N(c3nc(-c4ccc5c(c4)sc4ccccc45)nc(-c4ccc5sc6ccccc6c5c4)n3)c3cccc4c3sc3ccccc34)c2)cc1 ; c1ccc(N(c2nc(-c3ccc4sc5ccccc5c4c3)nc(-c3cccc4c3sc3ccccc34)n2)c2cccc3c2sc2ccccc23)cc1 ; c1ccc2c(c1)sc1cc(N(c3nc(-c4ccc5sc6ccccc6c5c4)nc(-c4ccc5sc6ccccc6c5c4)n3)c3cccc4c3sc3ccccc34)ccc12] 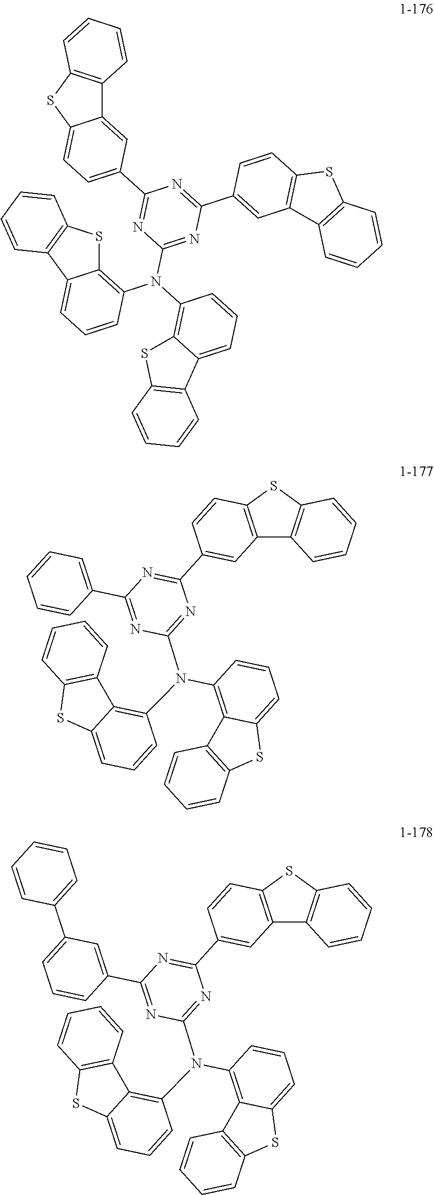 [SMILES: c1ccc2c(c1)sc1ccc(-c3nc(-c4ccc5sc6ccccc6c5c4)nc(N(c4cccc5c4sc4ccccc45)c4cccc5c4sc4ccccc45)n3)cc12 ; c1ccc(-c2nc(-c3ccc4sc5ccccc5c4c3)nc(N(c3cccc4sc5ccccc5c34)c3cccc4sc5ccccc5c34)n2)cc1 ; c1ccc(-c2cccc(-c3nc(-c4ccc5sc6ccccc6c5c4)nc(N(c4cccc5sc6ccccc6c45)c4cccc5sc6ccccc6c45)n3)c2)cc1] 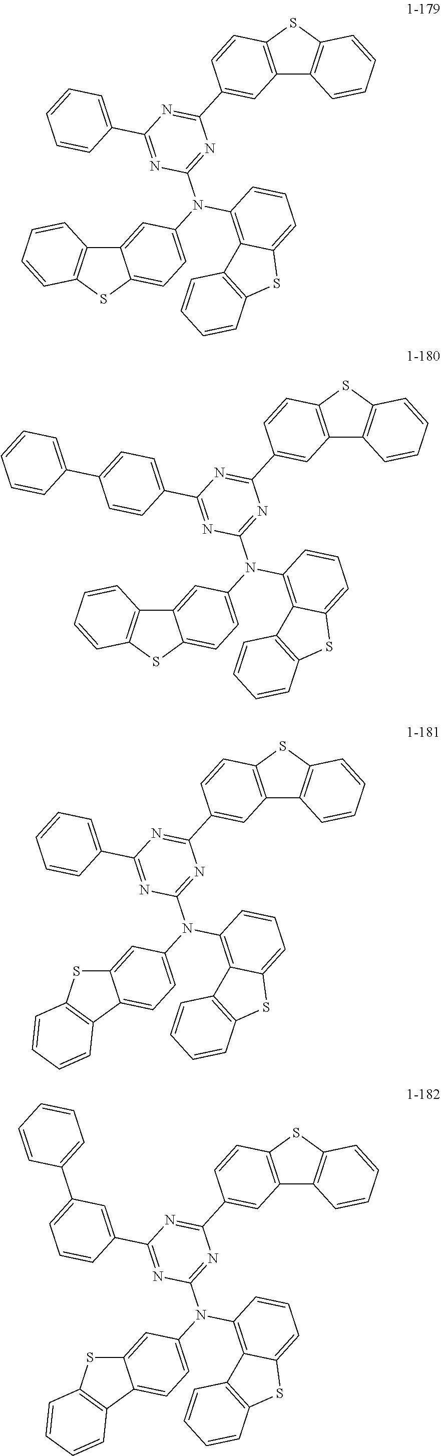 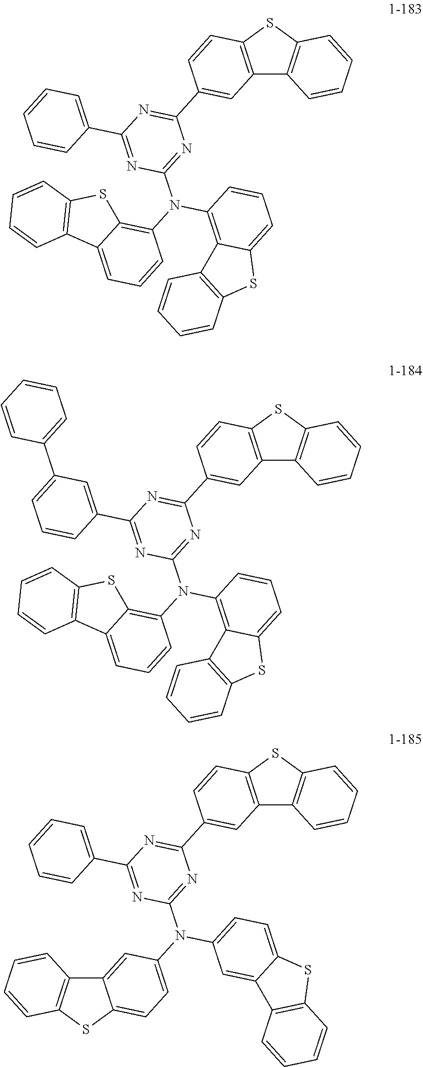 [SMILES: c1ccc(-c2nc(-c3ccc4sc5ccccc5c4c3)nc(N(c3cccc4c3sc3ccccc34)c3cccc4sc5ccccc5c34)n2)cc1 ; c1ccc(-c2cccc(-c3nc(-c4ccc5sc6ccccc6c5c4)nc(N(c4cccc5c4sc4ccccc45)c4cccc5sc6ccccc6c45)n3)c2)cc1 ; c1ccc(-c2nc(-c3ccc4sc5ccccc5c4c3)nc(N(c3ccc4sc5ccccc5c4c3)c3ccc4sc5ccccc5c4c3)n2)cc1] 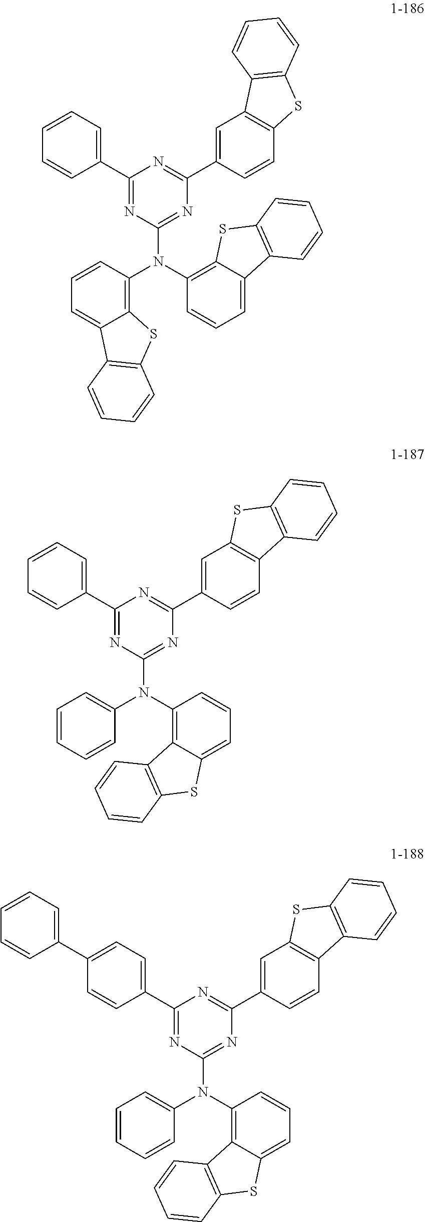 [SMILES: c1ccc(-c2nc(-c3ccc4sc5ccccc5c4c3)nc(N(c3cccc4c3sc3ccccc34)c3cccc4c3sc3ccccc34)n2)cc1 ; c1ccc(-c2nc(-c3ccc4c(c3)sc3ccccc34)nc(N(c3ccccc3)c3cccc4sc5ccccc5c34)n2)cc1 ; c1ccc(-c2ccc(-c3nc(-c4ccc5c(c4)sc4ccccc45)nc(N(c4ccccc4)c4cccc5sc6ccccc6c45)n3)cc2)cc1] 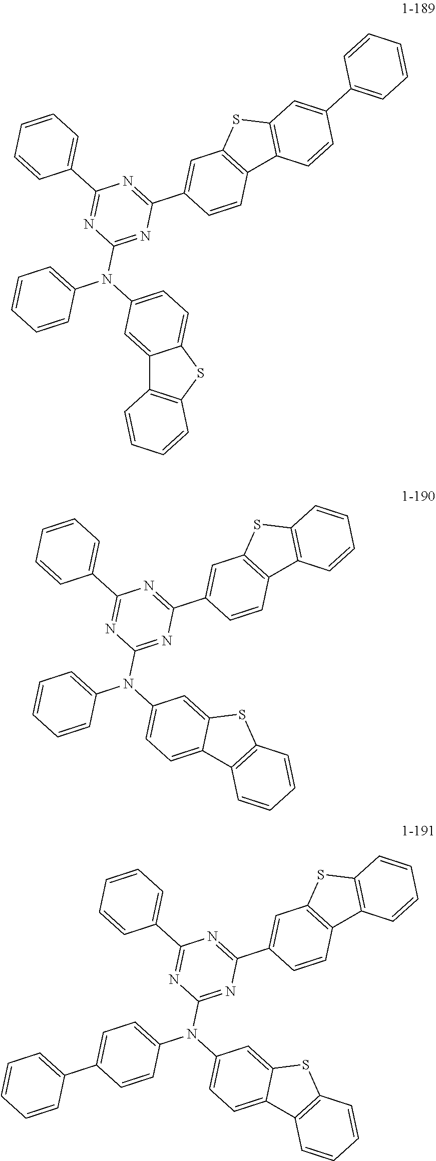 [SMILES: c1ccc(-c2ccc3c(c2)sc2cc(-c4nc(-c5ccccc5)nc(N(c5ccccc5)c5ccc6sc7ccccc7c6c5)n4)ccc23)cc1 ; c1ccc(-c2nc(-c3ccc4c(c3)sc3ccccc34)nc(N(c3ccccc3)c3ccc4c(c3)sc3ccccc34)n2)cc1 ; c1ccc(-c2ccc(N(c3ccc4c(c3)sc3ccccc34)c3nc(-c4ccccc4)nc(-c4ccc5c(c4)sc4ccccc45)n3)cc2)cc1] 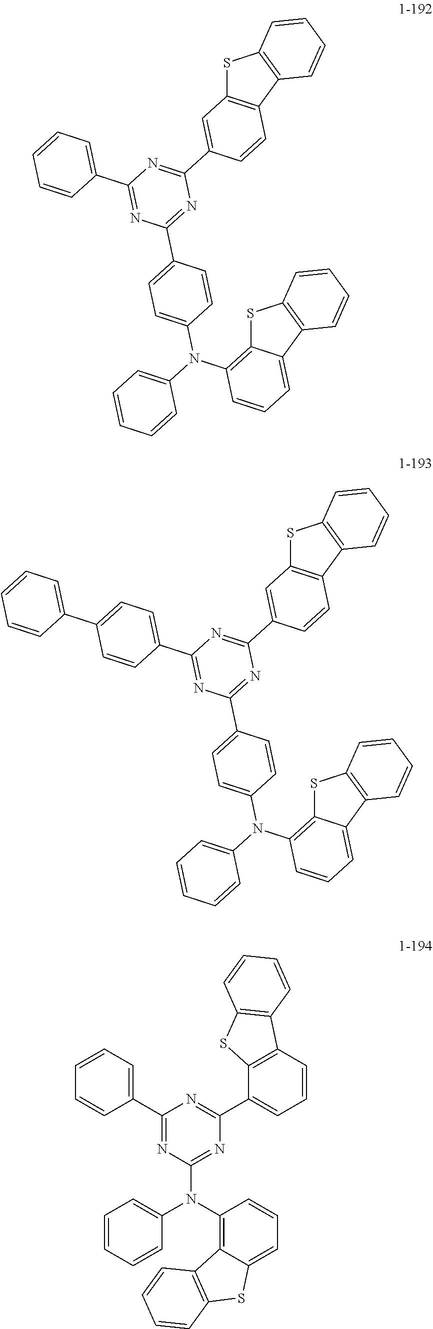 [SMILES: c1ccc(-c2nc(-c3ccc(N(c4ccccc4)c4cccc5c4sc4ccccc45)cc3)nc(-c3ccc4c(c3)sc3ccccc34)n2)cc1 ; c1ccc(-c2ccc(-c3nc(-c4ccc(N(c5ccccc5)c5cccc6c5sc5ccccc56)cc4)nc(-c4ccc5c(c4)sc4ccccc45)n3)cc2)cc1 ; c1ccc(-c2nc(-c3cccc4c3sc3ccccc34)nc(N(c3ccccc3)c3cccc4sc5ccccc5c34)n2)cc1] 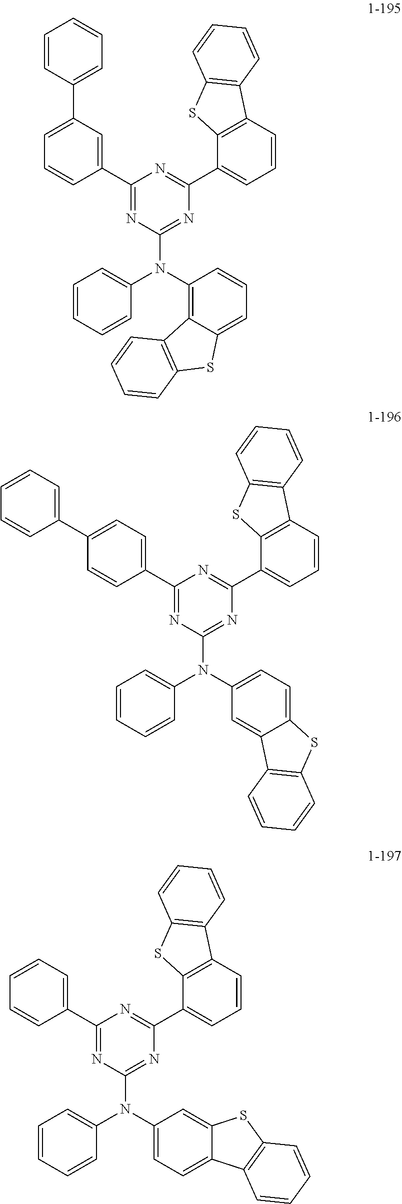 [SMILES: c1ccc(-c2cccc(-c3nc(-c4cccc5c4sc4ccccc45)nc(N(c4ccccc4)c4cccc5sc6ccccc6c45)n3)c2)cc1 ; c1ccc(-c2ccc(-c3nc(-c4cccc5c4sc4ccccc45)nc(N(c4ccccc4)c4ccc5sc6ccccc6c5c4)n3)cc2)cc1 ; c1ccc(-c2nc(-c3cccc4c3sc3ccccc34)nc(N(c3ccccc3)c3ccc4c(c3)sc3ccccc34)n2)cc1] 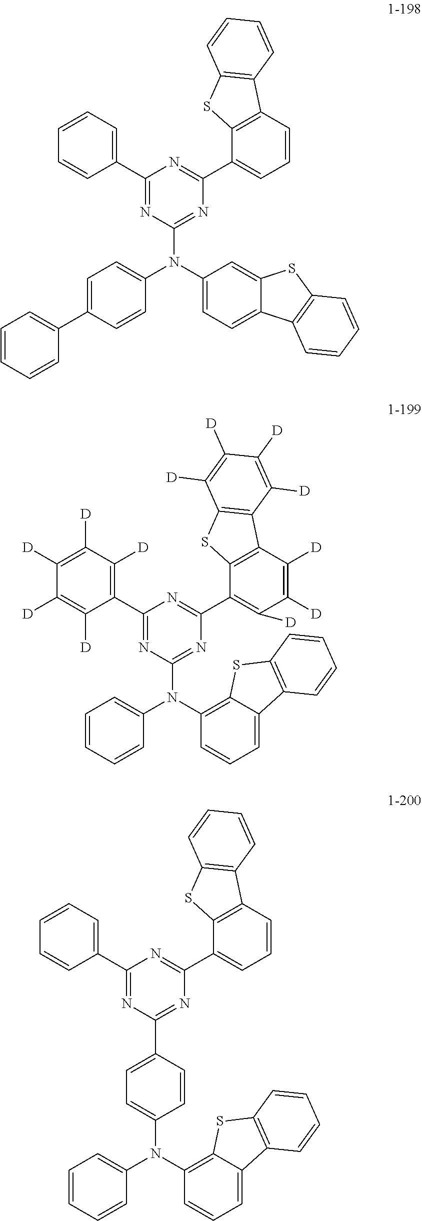 [SMILES: c1ccc(-c2ccc(N(c3ccc4c(c3)sc3ccccc34)c3nc(-c4ccccc4)nc(-c4cccc5c4sc4ccccc45)n3)cc2)cc1 ; [2H]c1c([2H])c([2H])c(-c2nc(-c3c([2H])c([2H])c([2H])c4c3sc3c([2H])c([2H])c([2H])c([2H])c34)nc(N(c3ccccc3)c3cccc4c3sc3ccccc34)n2)c([2H])c1[2H] ; c1ccc(-c2nc(-c3ccc(N(c4ccccc4)c4cccc5c4sc4ccccc45)cc3)nc(-c3cccc4c3sc3ccccc34)n2)cc1] 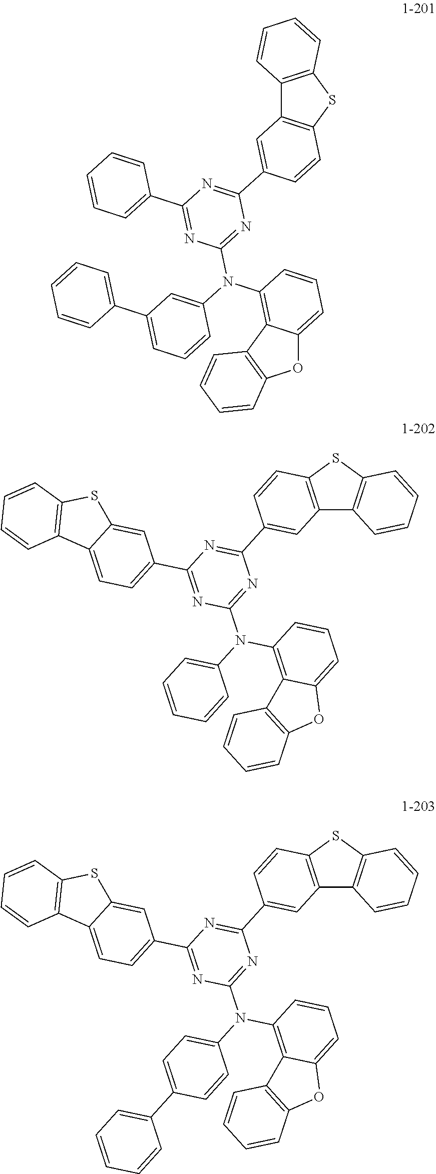 [SMILES: c1ccc(-c2cccc(N(c3nc(-c4ccccc4)nc(-c4ccc5sc6ccccc6c5c4)n3)c3cccc4oc5ccccc5c34)c2)cc1 ; c1ccc(N(c2nc(-c3ccc4c(c3)sc3ccccc34)nc(-c3ccc4sc5ccccc5c4c3)n2)c2cccc3oc4ccccc4c23)cc1 ; c1ccc(-c2ccc(N(c3nc(-c4ccc5c(c4)sc4ccccc45)nc(-c4ccc5sc6ccccc6c5c4)n3)c3cccc4oc5ccccc5c34)cc2)cc1] 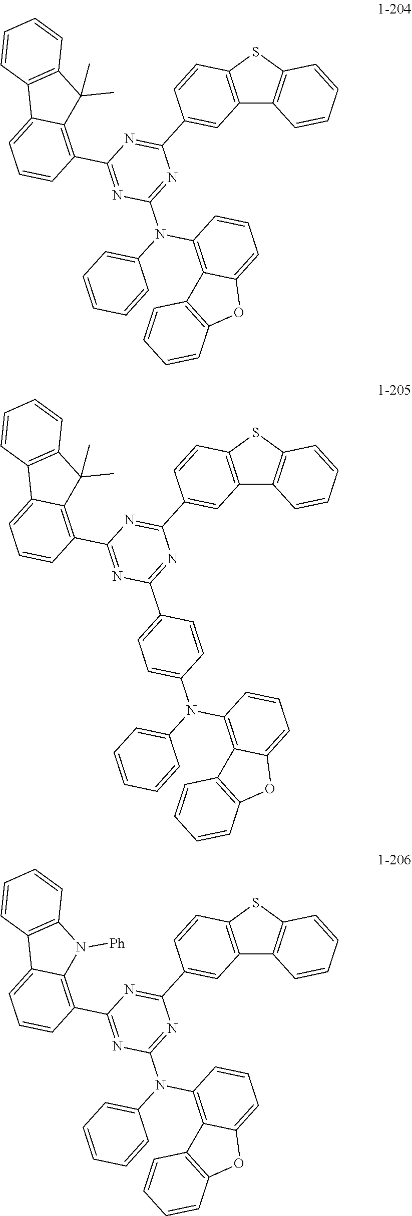 [SMILES: CC1(C)c2ccccc2-c2cccc(-c3nc(-c4ccc5sc6ccccc6c5c4)nc(N(c4ccccc4)c4cccc5oc6ccccc6c45)n3)c21 ; CC1(C)c2ccccc2-c2cccc(-c3nc(-c4ccc(N(c5ccccc5)c5cccc6oc7ccccc7c56)cc4)nc(-c4ccc5sc6ccccc6c5c4)n3)c21 ; c1ccc(N(c2nc(-c3ccc4sc5ccccc5c4c3)nc(-c3cccc4c5ccccc5n(-c5ccccc5)c34)n2)c2cccc3oc4ccccc4c23)cc1] 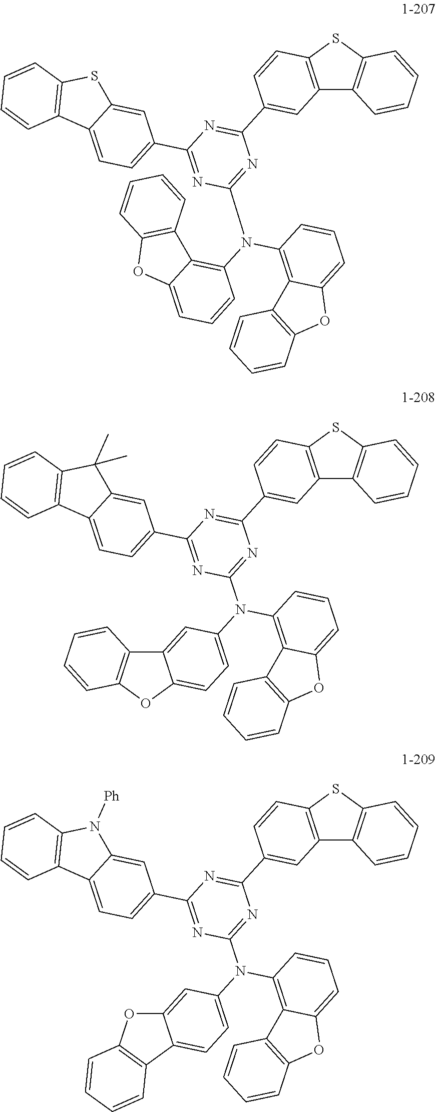 [SMILES: c1ccc2c(c1)oc1cccc(N(c3nc(-c4ccc5c(c4)sc4ccccc45)nc(-c4ccc5sc6ccccc6c5c4)n3)c3cccc4oc5ccccc5c34)c12 ; CC1(C)c2ccccc2-c2ccc(-c3nc(-c4ccc5sc6ccccc6c5c4)nc(N(c4ccc5oc6ccccc6c5c4)c4cccc5oc6ccccc6c45)n3)cc21 ; c1ccc(-n2c3ccccc3c3ccc(-c4nc(-c5ccc6sc7ccccc7c6c5)nc(N(c5ccc6c(c5)oc5ccccc56)c5cccc6oc7ccccc7c56)n4)cc32)cc1] 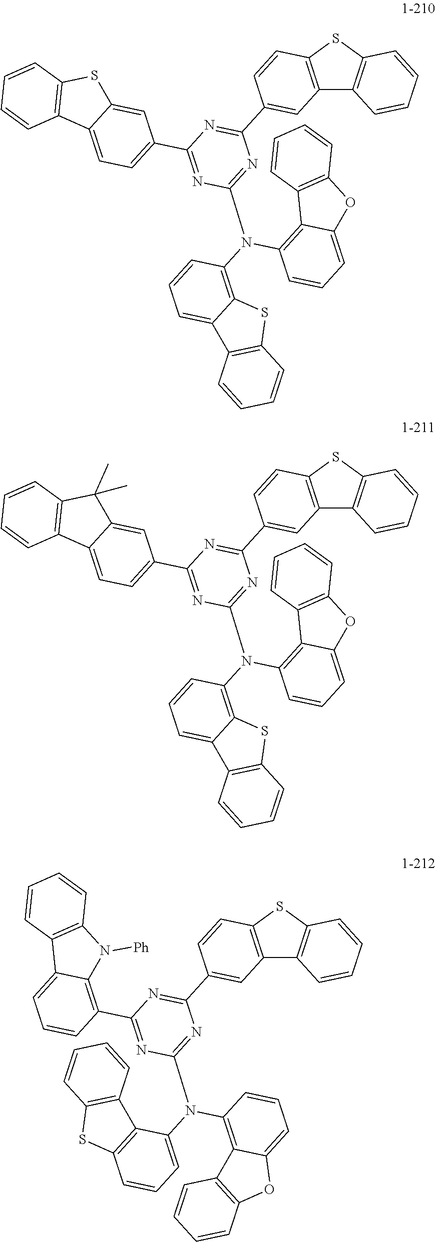 [SMILES: c1ccc2c(c1)oc1cccc(N(c3nc(-c4ccc5c(c4)sc4ccccc45)nc(-c4ccc5sc6ccccc6c5c4)n3)c3cccc4c3sc3ccccc34)c12 ; CC1(C)c2ccccc2-c2ccc(-c3nc(-c4ccc5sc6ccccc6c5c4)nc(N(c4cccc5c4sc4ccccc45)c4cccc5oc6ccccc6c45)n3)cc21 ; c1ccc(-n2c3ccccc3c3cccc(-c4nc(-c5ccc6sc7ccccc7c6c5)nc(N(c5cccc6oc7ccccc7c56)c5cccc6sc7ccccc7c56)n4)c32)cc1] 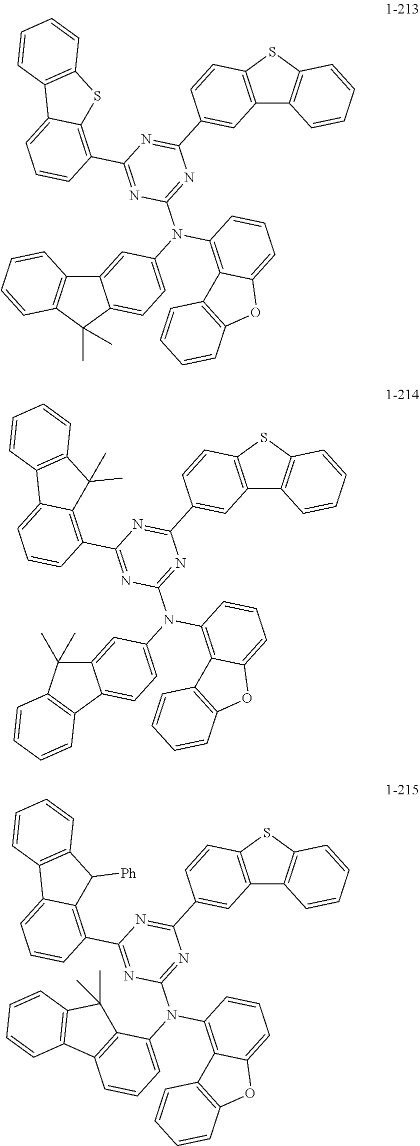 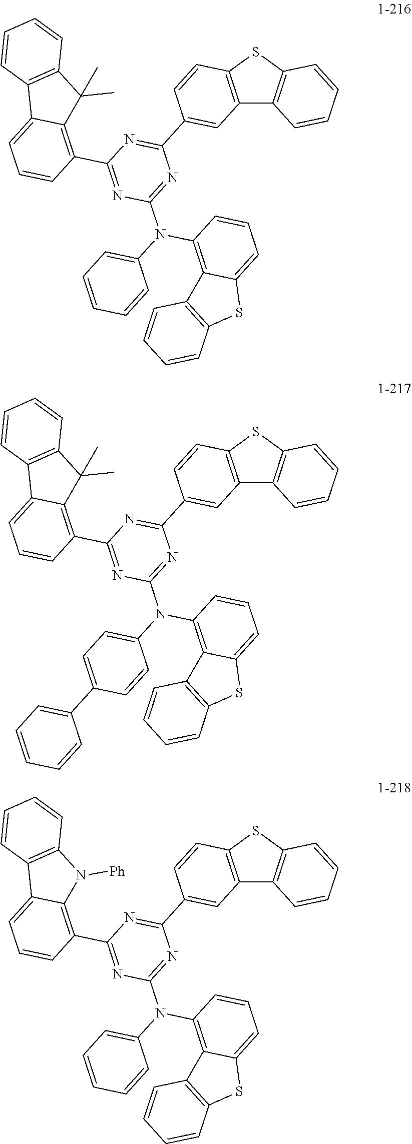 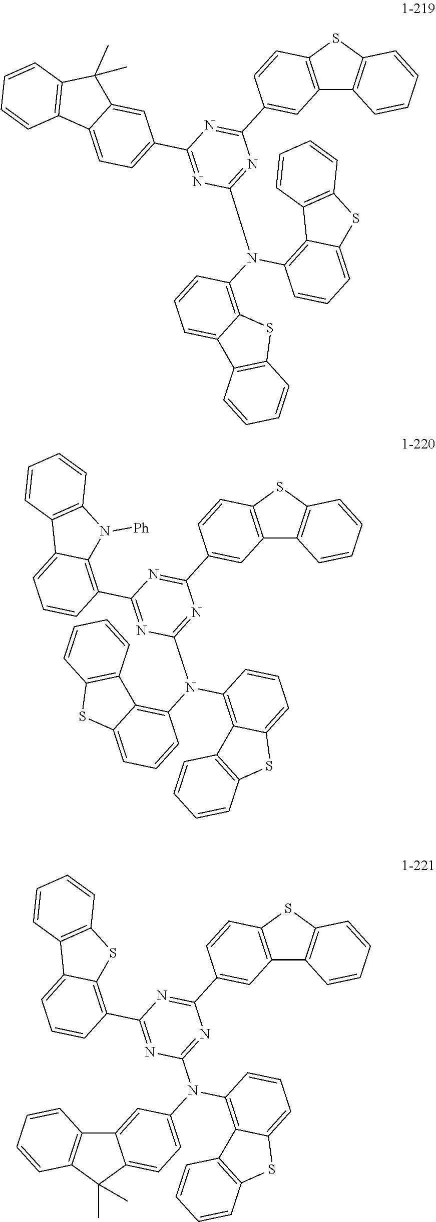 [SMILES: CC1(C)c2ccccc2-c2ccc(-c3nc(-c4ccc5sc6ccccc6c5c4)nc(N(c4cccc5c4sc4ccccc45)c4cccc5sc6ccccc6c45)n3)cc21 ; c1ccc(-n2c3ccccc3c3cccc(-c4nc(-c5ccc6sc7ccccc7c6c5)nc(N(c5cccc6sc7ccccc7c56)c5cccc6sc7ccccc7c56)n4)c32)cc1 ; CC1(C)c2ccccc2-c2cc(N(c3nc(-c4ccc5sc6ccccc6c5c4)nc(-c4cccc5c4sc4ccccc45)n3)c3cccc4sc5ccccc5c34)ccc21] 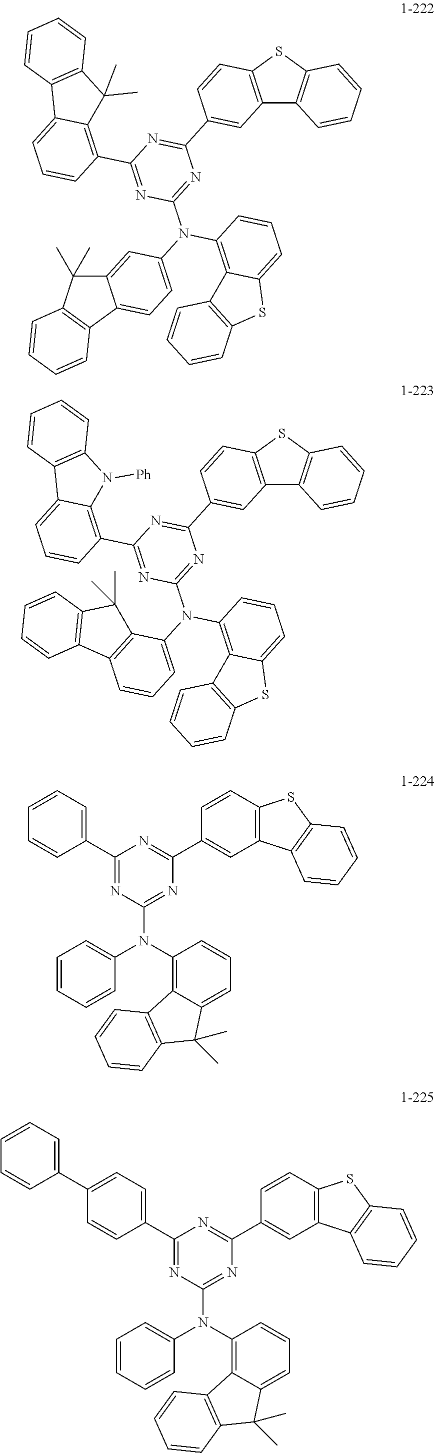 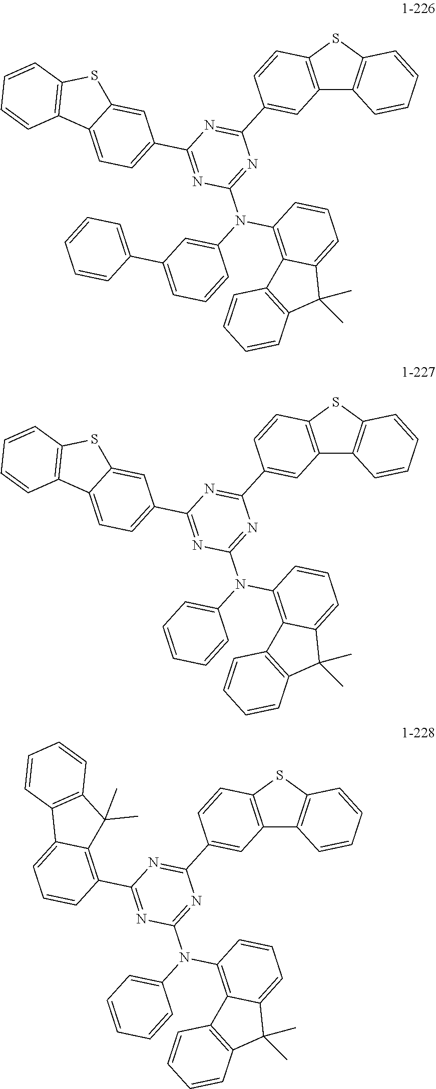 [SMILES: CC1(C)c2ccccc2-c2c(N(c3cccc(-c4ccccc4)c3)c3nc(-c4ccc5c(c4)sc4ccccc45)nc(-c4ccc5sc6ccccc6c5c4)n3)cccc21 ; CC1(C)c2ccccc2-c2c(N(c3ccccc3)c3nc(-c4ccc5c(c4)sc4ccccc45)nc(-c4ccc5sc6ccccc6c5c4)n3)cccc21 ; CC1(C)c2ccccc2-c2c(N(c3ccccc3)c3nc(-c4ccc5sc6ccccc6c5c4)nc(-c4cccc5c4C(C)(C)c4ccccc4-5)n3)cccc21] 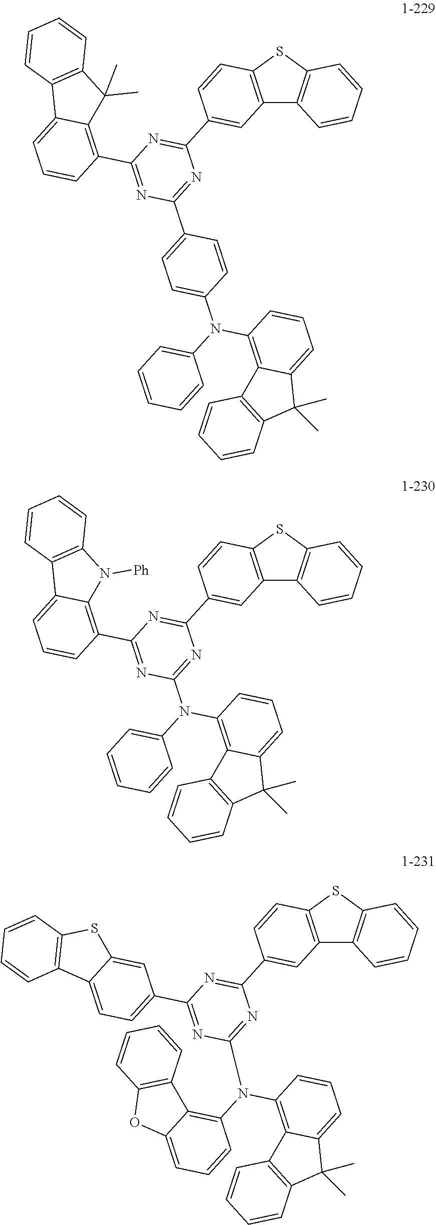 [SMILES: CC1(C)c2ccccc2-c2c(N(c3ccccc3)c3ccc(-c4nc(-c5ccc6sc7ccccc7c6c5)nc(-c5cccc6c5C(C)(C)c5ccccc5-6)n4)cc3)cccc21 ; CC1(C)c2ccccc2-c2c(N(c3ccccc3)c3nc(-c4ccc5sc6ccccc6c5c4)nc(-c4cccc5c6ccccc6n(-c6ccccc6)c45)n3)cccc21 ; CC1(C)c2ccccc2-c2c(N(c3nc(-c4ccc5c(c4)sc4ccccc45)nc(-c4ccc5sc6ccccc6c5c4)n3)c3cccc4oc5ccccc5c34)cccc21] 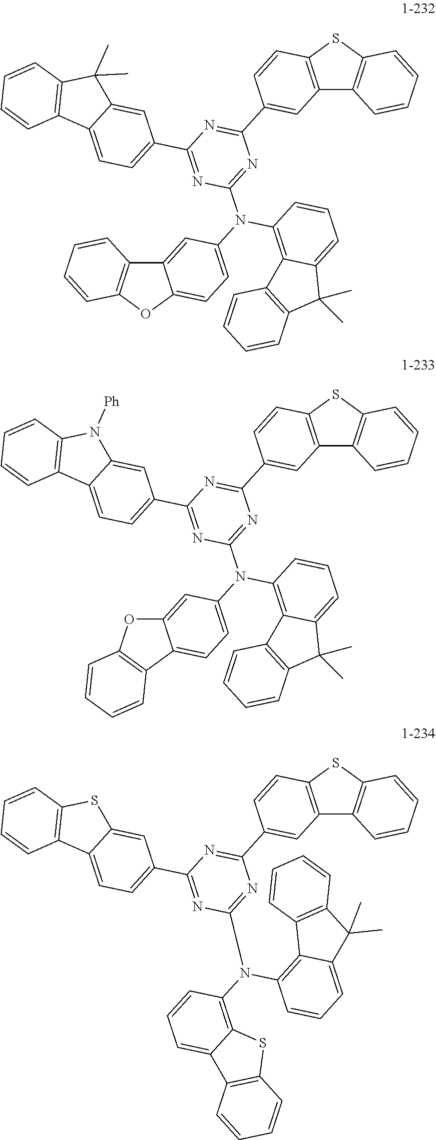 [SMILES: CC1(C)c2ccccc2-c2ccc(-c3nc(-c4ccc5sc6ccccc6c5c4)nc(N(c4ccc5oc6ccccc6c5c4)c4cccc5c4-c4ccccc4C5(C)C)n3)cc21 ; CC1(C)c2ccccc2-c2c(N(c3ccc4c(c3)oc3ccccc34)c3nc(-c4ccc5sc6ccccc6c5c4)nc(-c4ccc5c6ccccc6n(-c6ccccc6)c5c4)n3)cccc21 ; CC1(C)c2ccccc2-c2c(N(c3nc(-c4ccc5c(c4)sc4ccccc45)nc(-c4ccc5sc6ccccc6c5c4)n3)c3cccc4c3sc3ccccc34)cccc21] 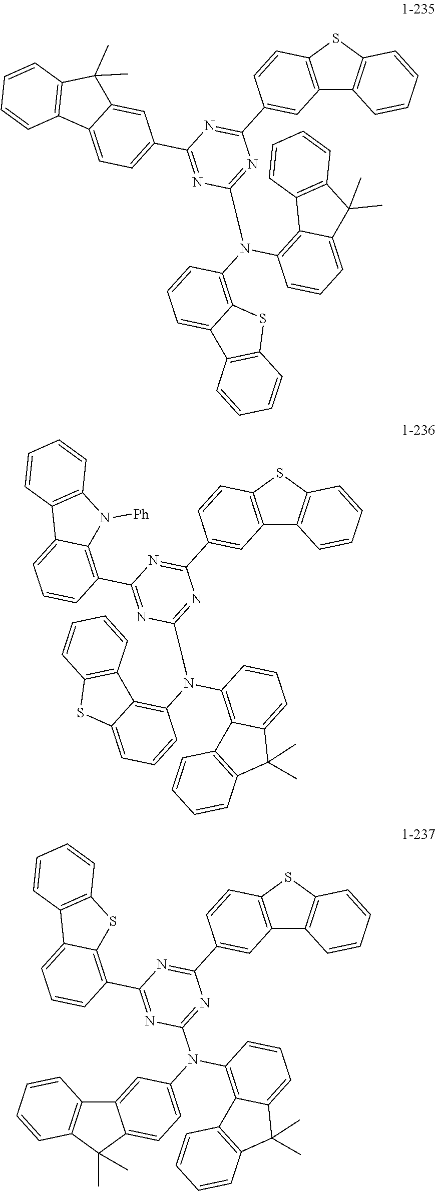 [SMILES: CC1(C)c2ccccc2-c2ccc(-c3nc(-c4ccc5sc6ccccc6c5c4)nc(N(c4cccc5c4-c4ccccc4C5(C)C)c4cccc5c4sc4ccccc45)n3)cc21 ; CC1(C)c2ccccc2-c2c(N(c3nc(-c4ccc5sc6ccccc6c5c4)nc(-c4cccc5c6ccccc6n(-c6ccccc6)c45)n3)c3cccc4sc5ccccc5c34)cccc21 ; CC1(C)c2ccccc2-c2cc(N(c3nc(-c4ccc5sc6ccccc6c5c4)nc(-c4cccc5c4sc4ccccc45)n3)c3cccc4c3-c3ccccc3C4(C)C)ccc21] 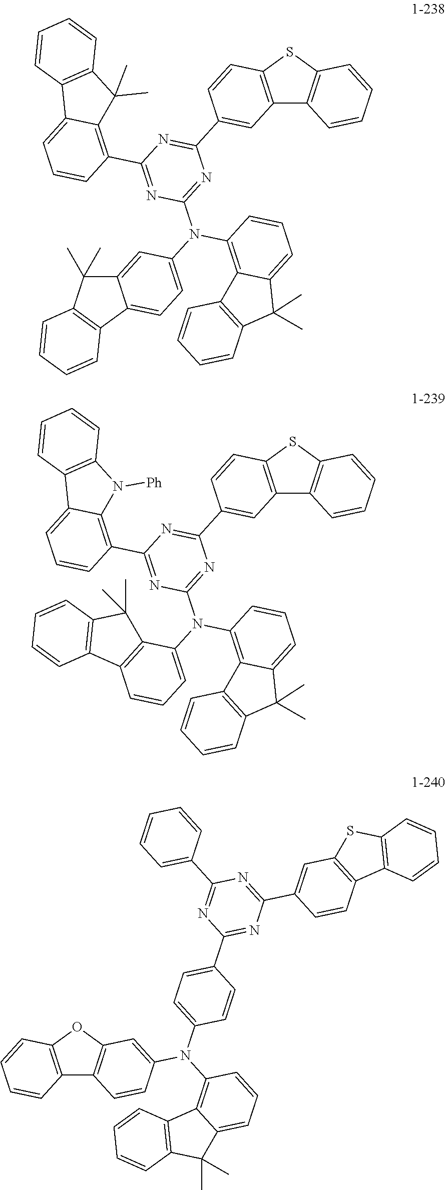 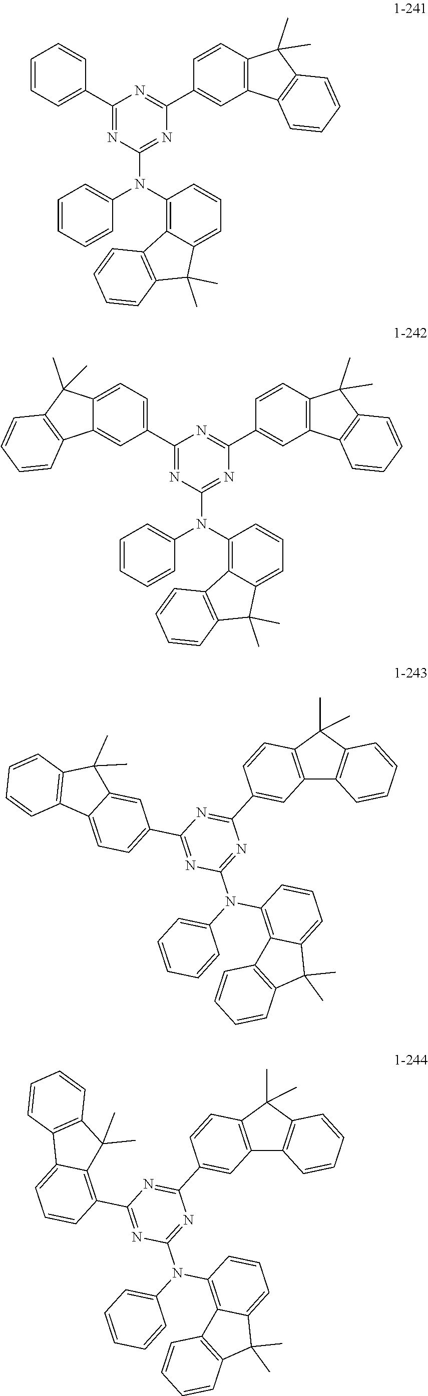 [SMILES: CC1(C)c2ccccc2-c2cc(-c3nc(-c4ccccc4)nc(N(c4ccccc4)c4cccc5c4-c4ccccc4C5(C)C)n3)ccc21 ; CC1(C)c2ccccc2-c2cc(-c3nc(-c4ccc5c(c4)-c4ccccc4C5(C)C)nc(N(c4ccccc4)c4cccc5c4-c4ccccc4C5(C)C)n3)ccc21 ; CC1(C)c2ccccc2-c2cc(-c3nc(-c4ccc5c(c4)C(C)(C)c4ccccc4-5)nc(N(c4ccccc4)c4cccc5c4-c4ccccc4C5(C)C)n3)ccc21 ; CC1(C)c2ccccc2-c2cc(-c3nc(-c4cccc5c4C(C)(C)c4ccccc4-5)nc(N(c4ccccc4)c4cccc5c4-c4ccccc4C5(C)C)n3)ccc21] 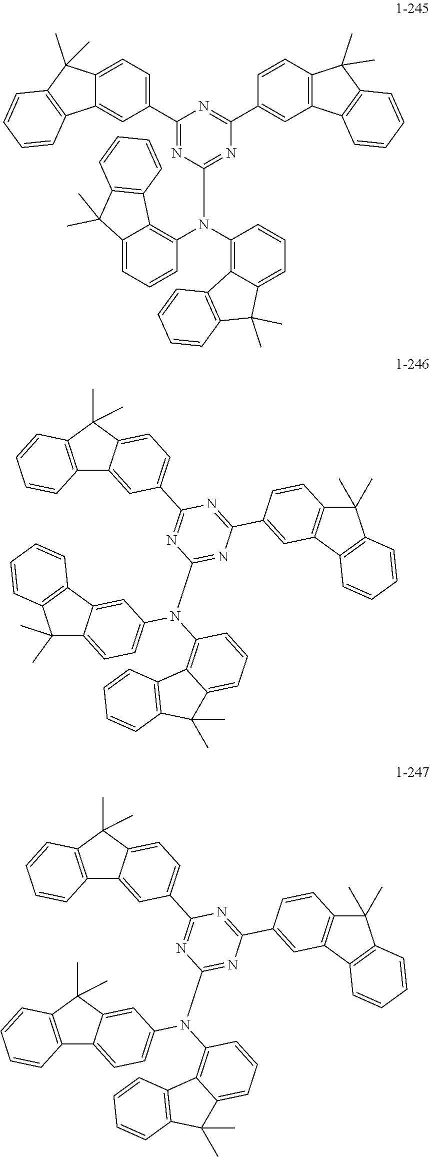 [SMILES: CC1(C)c2ccccc2-c2cc(-c3nc(-c4ccc5c(c4)-c4ccccc4C5(C)C)nc(N(c4cccc5c4-c4ccccc4C5(C)C)c4cccc5c4-c4ccccc4C5(C)C)n3)ccc21 ; CC1(C)c2ccccc2-c2cc(-c3nc(-c4ccc5c(c4)-c4ccccc4C5(C)C)nc(N(c4ccc5c(c4)-c4ccccc4C5(C)C)c4cccc5c4-c4ccccc4C5(C)C)n3)ccc21 ; CC1(C)c2ccccc2-c2cc(-c3nc(-c4ccc5c(c4)-c4ccccc4C5(C)C)nc(N(c4ccc5c(c4)C(C)(C)c4ccccc4-5)c4cccc5c4-c4ccccc4C5(C)C)n3)ccc21] 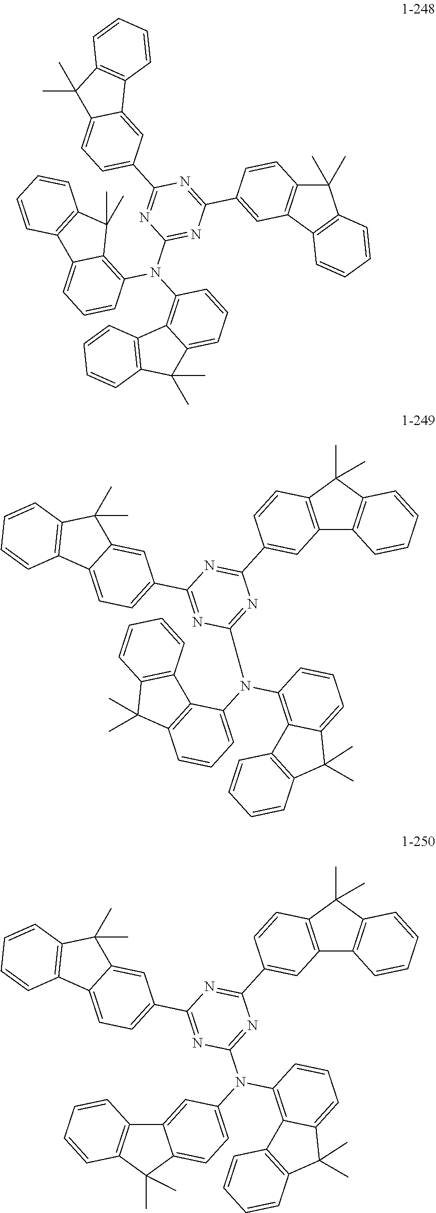 [SMILES: CC1(C)c2ccccc2-c2cc(-c3nc(-c4ccc5c(c4)-c4ccccc4C5(C)C)nc(N(c4cccc5c4-c4ccccc4C5(C)C)c4cccc5c4C(C)(C)c4ccccc4-5)n3)ccc21 ; CC1(C)c2ccccc2-c2cc(-c3nc(-c4ccc5c(c4)C(C)(C)c4ccccc4-5)nc(N(c4cccc5c4-c4ccccc4C5(C)C)c4cccc5c4-c4ccccc4C5(C)C)n3)ccc21 ; CC1(C)c2ccccc2-c2cc(-c3nc(-c4ccc5c(c4)C(C)(C)c4ccccc4-5)nc(N(c4ccc5c(c4)-c4ccccc4C5(C)C)c4cccc5c4-c4ccccc4C5(C)C)n3)ccc21] 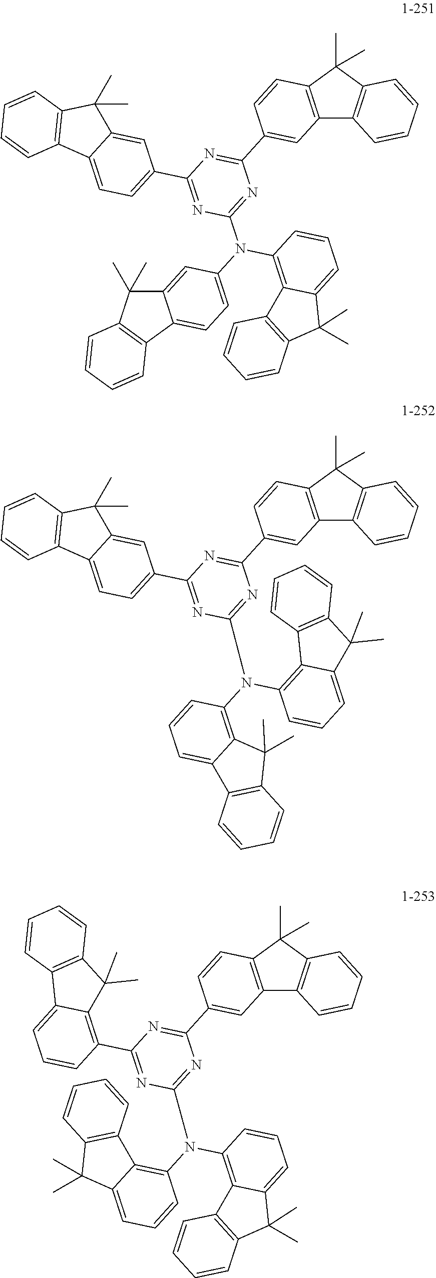 [SMILES: CC1(C)c2ccccc2-c2cc(-c3nc(-c4ccc5c(c4)C(C)(C)c4ccccc4-5)nc(N(c4ccc5c(c4)C(C)(C)c4ccccc4-5)c4cccc5c4-c4ccccc4C5(C)C)n3)ccc21 ; CC1(C)c2ccccc2-c2cc(-c3nc(-c4ccc5c(c4)C(C)(C)c4ccccc4-5)nc(N(c4cccc5c4-c4ccccc4C5(C)C)c4cccc5c4C(C)(C)c4ccccc4-5)n3)ccc21 ; CC1(C)c2ccccc2-c2cc(-c3nc(-c4cccc5c4C(C)(C)c4ccccc4-5)nc(N(c4cccc5c4-c4ccccc4C5(C)C)c4cccc5c4-c4ccccc4C5(C)C)n3)ccc21] 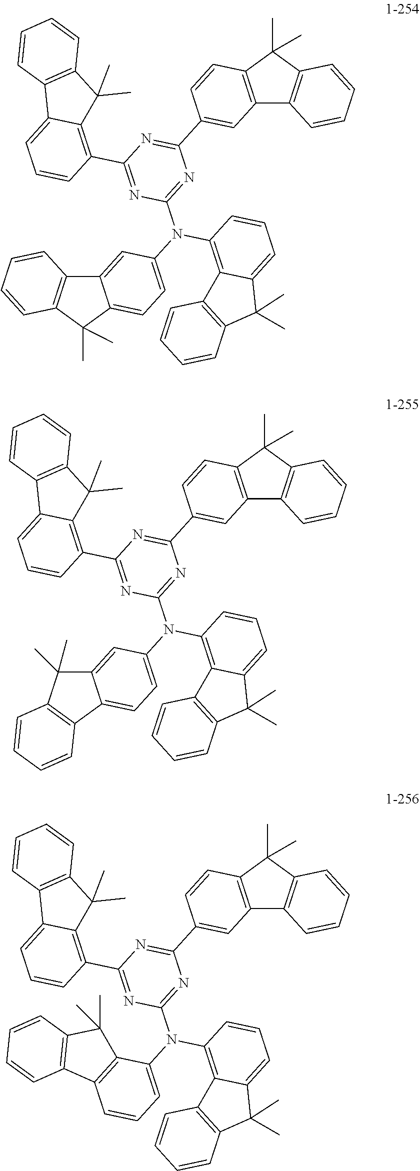 [SMILES: CC1(C)c2ccccc2-c2cc(-c3nc(-c4cccc5c4C(C)(C)c4ccccc4-5)nc(N(c4ccc5c(c4)-c4ccccc4C5(C)C)c4cccc5c4-c4ccccc4C5(C)C)n3)ccc21 ; CC1(C)c2ccccc2-c2cc(-c3nc(-c4cccc5c4C(C)(C)c4ccccc4-5)nc(N(c4ccc5c(c4)C(C)(C)c4ccccc4-5)c4cccc5c4-c4ccccc4C5(C)C)n3)ccc21 ; CC1(C)c2ccccc2-c2cc(-c3nc(-c4cccc5c4C(C)(C)c4ccccc4-5)nc(N(c4cccc5c4-c4ccccc4C5(C)C)c4cccc5c4C(C)(C)c4ccccc4-5)n3)ccc21] 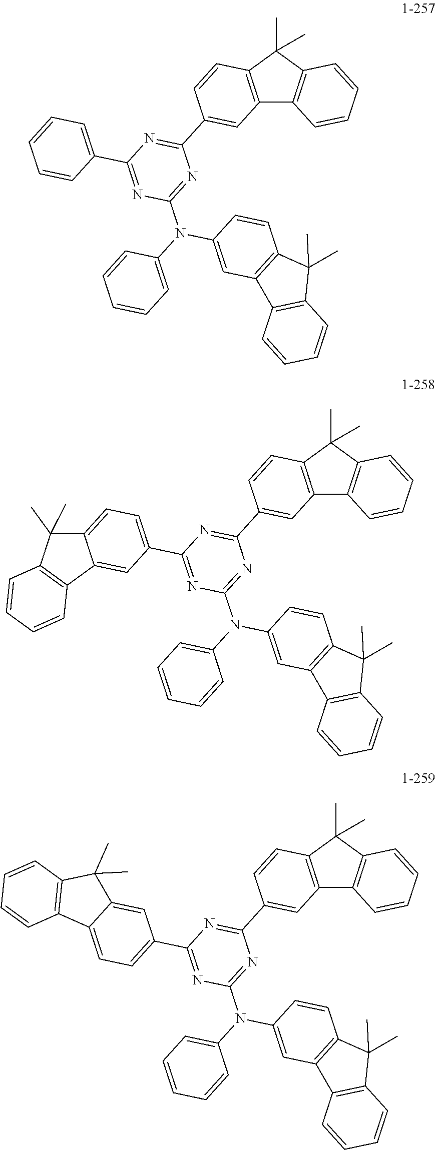 [SMILES: CC1(C)c2ccccc2-c2cc(-c3nc(-c4ccccc4)nc(N(c4ccccc4)c4ccc5c(c4)-c4ccccc4C5(C)C)n3)ccc21 ; CC1(C)c2ccccc2-c2cc(-c3nc(-c4ccc5c(c4)-c4ccccc4C5(C)C)nc(N(c4ccccc4)c4ccc5c(c4)-c4ccccc4C5(C)C)n3)ccc21 ; CC1(C)c2ccccc2-c2cc(-c3nc(-c4ccc5c(c4)C(C)(C)c4ccccc4-5)nc(N(c4ccccc4)c4ccc5c(c4)-c4ccccc4C5(C)C)n3)ccc21] 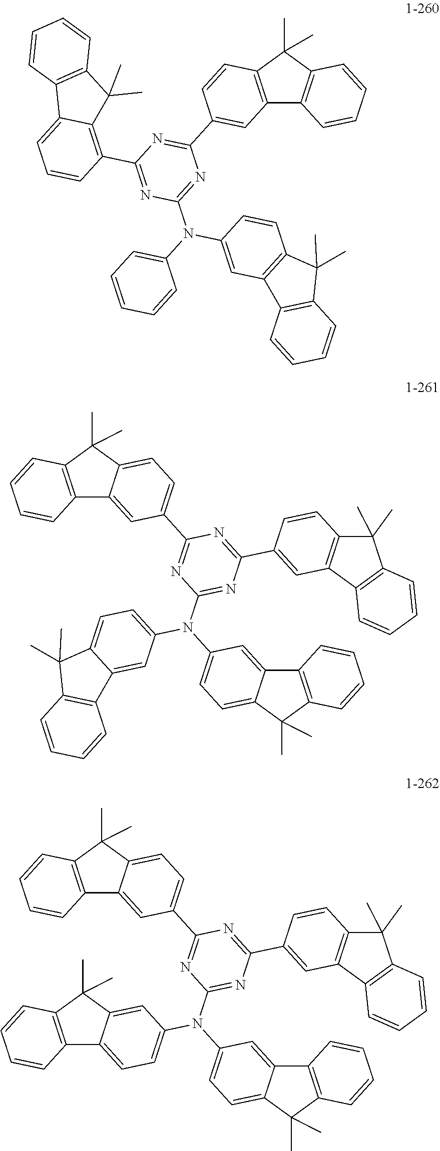 [SMILES: CC1(C)c2ccccc2-c2cc(-c3nc(-c4cccc5c4C(C)(C)c4ccccc4-5)nc(N(c4ccccc4)c4ccc5c(c4)-c4ccccc4C5(C)C)n3)ccc21 ; CC1(C)c2ccccc2-c2cc(-c3nc(-c4ccc5c(c4)-c4ccccc4C5(C)C)nc(N(c4ccc5c(c4)-c4ccccc4C5(C)C)c4ccc5c(c4)-c4ccccc4C5(C)C)n3)ccc21 ; CC1(C)c2ccccc2-c2cc(-c3nc(-c4ccc5c(c4)-c4ccccc4C5(C)C)nc(N(c4ccc5c(c4)-c4ccccc4C5(C)C)c4ccc5c(c4)C(C)(C)c4ccccc4-5)n3)ccc21] 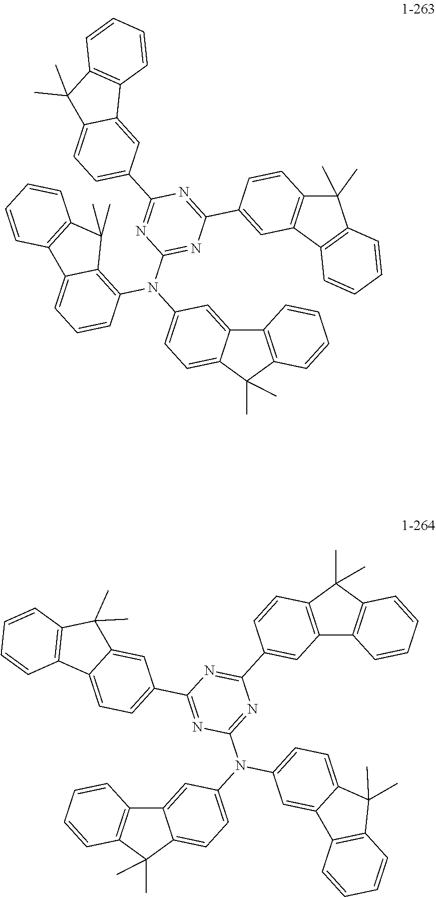 [SMILES: CC1(C)c2ccccc2-c2cc(-c3nc(-c4ccc5c(c4)-c4ccccc4C5(C)C)nc(N(c4ccc5c(c4)-c4ccccc4C5(C)C)c4cccc5c4C(C)(C)c4ccccc4-5)n3)ccc21 ; CC1(C)c2ccccc2-c2cc(-c3nc(-c4ccc5c(c4)C(C)(C)c4ccccc4-5)nc(N(c4ccc5c(c4)-c4ccccc4C5(C)C)c4ccc5c(c4)-c4ccccc4C5(C)C)n3)ccc21] 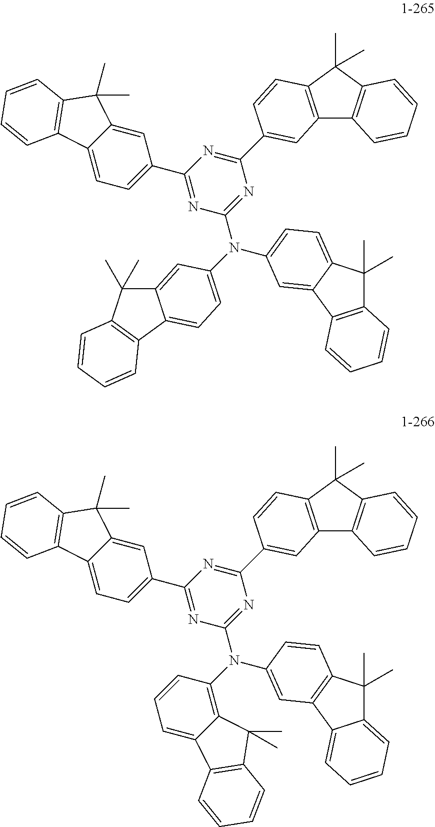 [SMILES: CC1(C)c2ccccc2-c2cc(-c3nc(-c4ccc5c(c4)C(C)(C)c4ccccc4-5)nc(N(c4ccc5c(c4)-c4ccccc4C5(C)C)c4ccc5c(c4)C(C)(C)c4ccccc4-5)n3)ccc21 ; CC1(C)c2ccccc2-c2cc(-c3nc(-c4ccc5c(c4)C(C)(C)c4ccccc4-5)nc(N(c4ccc5c(c4)-c4ccccc4C5(C)C)c4cccc5c4C(C)(C)c4ccccc4-5)n3)ccc21] 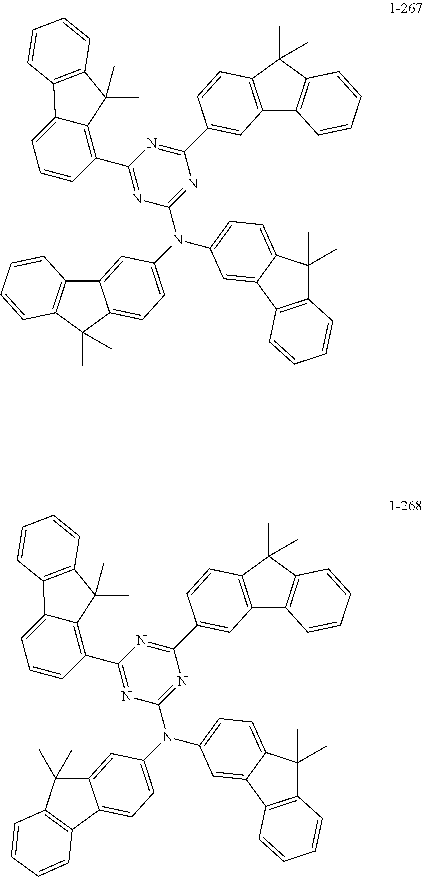 [SMILES: CC1(C)c2ccccc2-c2cc(-c3nc(-c4cccc5c4C(C)(C)c4ccccc4-5)nc(N(c4ccc5c(c4)-c4ccccc4C5(C)C)c4ccc5c(c4)-c4ccccc4C5(C)C)n3)ccc21 ; CC1(C)c2ccccc2-c2cc(-c3nc(-c4cccc5c4C(C)(C)c4ccccc4-5)nc(N(c4ccc5c(c4)-c4ccccc4C5(C)C)c4ccc5c(c4)C(C)(C)c4ccccc4-5)n3)ccc21] 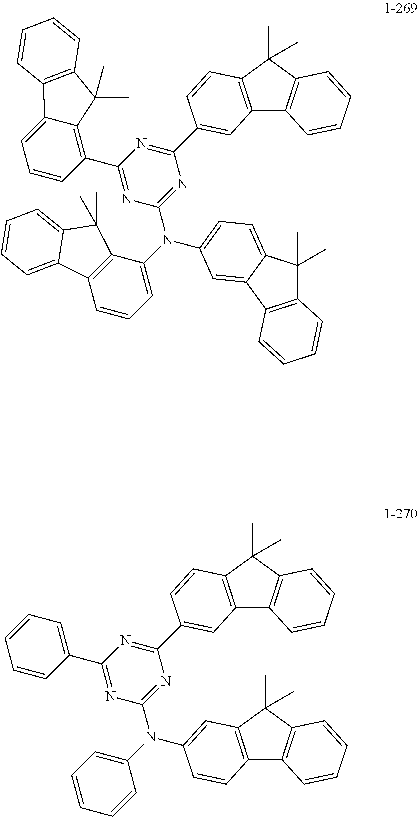 [SMILES: CC1(C)c2ccccc2-c2cc(-c3nc(-c4cccc5c4C(C)(C)c4ccccc4-5)nc(N(c4ccc5c(c4)-c4ccccc4C5(C)C)c4cccc5c4C(C)(C)c4ccccc4-5)n3)ccc21 ; CC1(C)c2ccccc2-c2cc(-c3nc(-c4ccccc4)nc(N(c4ccccc4)c4ccc5c(c4)C(C)(C)c4ccccc4-5)n3)ccc21] 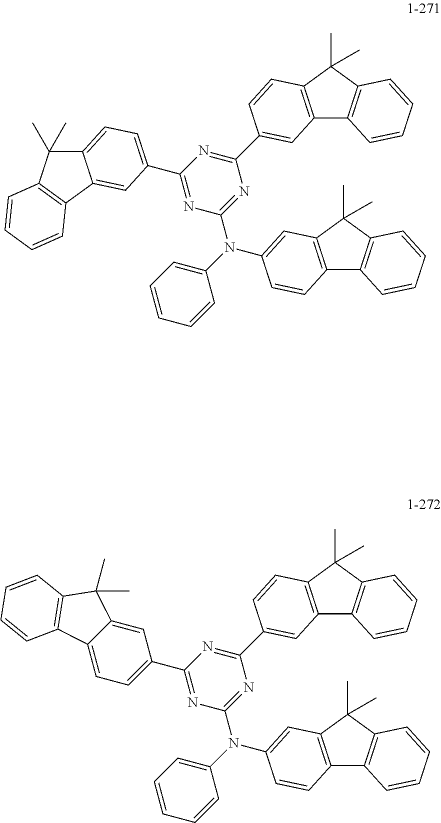 [SMILES: CC1(C)c2ccccc2-c2cc(-c3nc(-c4ccc5c(c4)-c4ccccc4C5(C)C)nc(N(c4ccccc4)c4ccc5c(c4)C(C)(C)c4ccccc4-5)n3)ccc21 ; CC1(C)c2ccccc2-c2cc(-c3nc(-c4ccc5c(c4)C(C)(C)c4ccccc4-5)nc(N(c4ccccc4)c4ccc5c(c4)C(C)(C)c4ccccc4-5)n3)ccc21] 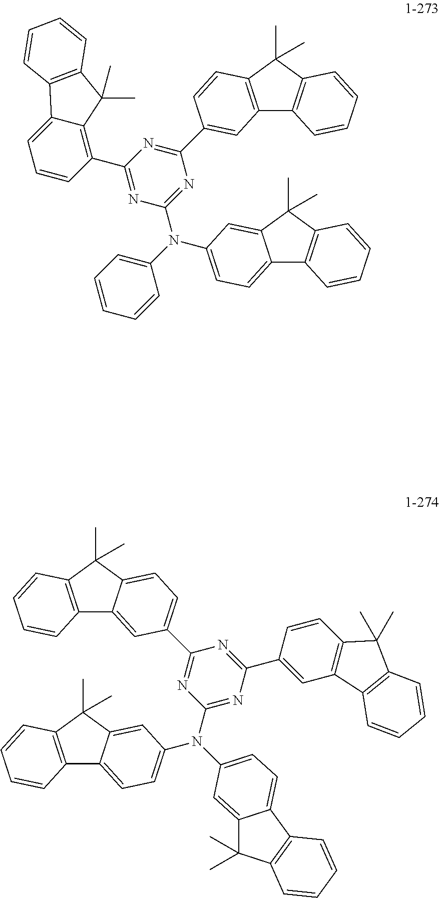 [SMILES: CC1(C)c2ccccc2-c2cc(-c3nc(-c4cccc5c4C(C)(C)c4ccccc4-5)nc(N(c4ccccc4)c4ccc5c(c4)C(C)(C)c4ccccc4-5)n3)ccc21 ; CC1(C)c2ccccc2-c2cc(-c3nc(-c4ccc5c(c4)-c4ccccc4C5(C)C)nc(N(c4ccc5c(c4)C(C)(C)c4ccccc4-5)c4ccc5c(c4)C(C)(C)c4ccccc4-5)n3)ccc21] 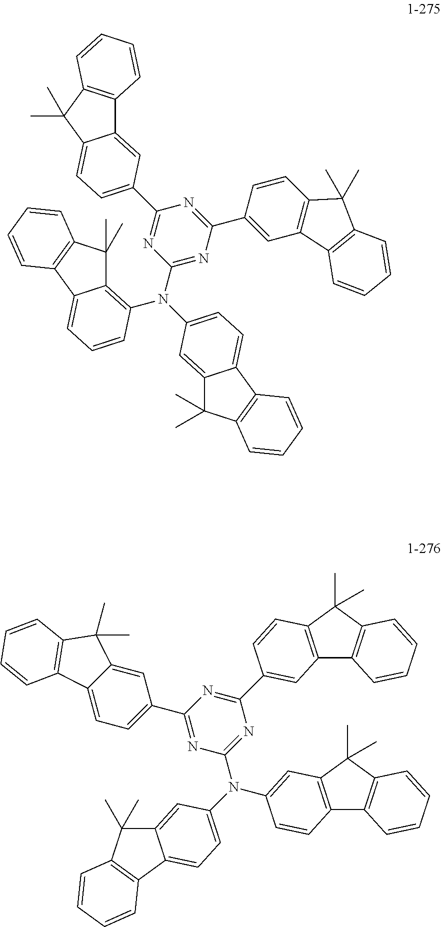 [SMILES: CC1(C)c2ccccc2-c2cc(-c3nc(-c4ccc5c(c4)-c4ccccc4C5(C)C)nc(N(c4ccc5c(c4)C(C)(C)c4ccccc4-5)c4cccc5c4C(C)(C)c4ccccc4-5)n3)ccc21 ; CC1(C)c2ccccc2-c2cc(-c3nc(-c4ccc5c(c4)C(C)(C)c4ccccc4-5)nc(N(c4ccc5c(c4)C(C)(C)c4ccccc4-5)c4ccc5c(c4)C(C)(C)c4ccccc4-5)n3)ccc21] 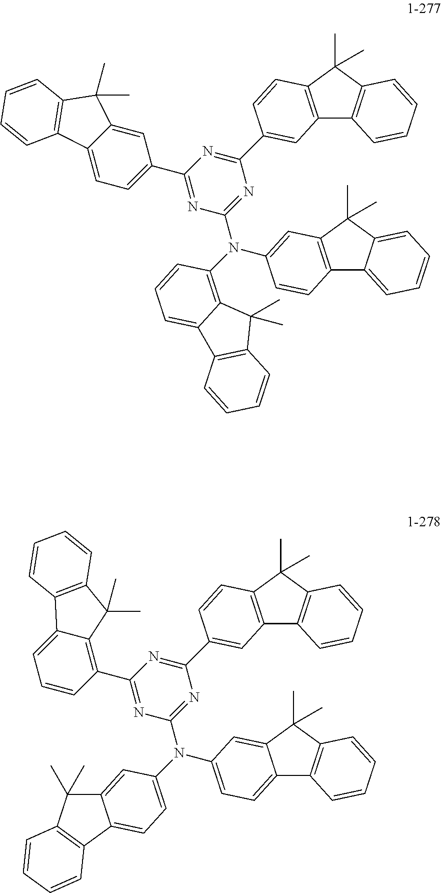 [SMILES: CC1(C)c2ccccc2-c2cc(-c3nc(-c4ccc5c(c4)C(C)(C)c4ccccc4-5)nc(N(c4ccc5c(c4)C(C)(C)c4ccccc4-5)c4cccc5c4C(C)(C)c4ccccc4-5)n3)ccc21 ; CC1(C)c2ccccc2-c2cc(-c3nc(-c4cccc5c4C(C)(C)c4ccccc4-5)nc(N(c4ccc5c(c4)C(C)(C)c4ccccc4-5)c4ccc5c(c4)C(C)(C)c4ccccc4-5)n3)ccc21] 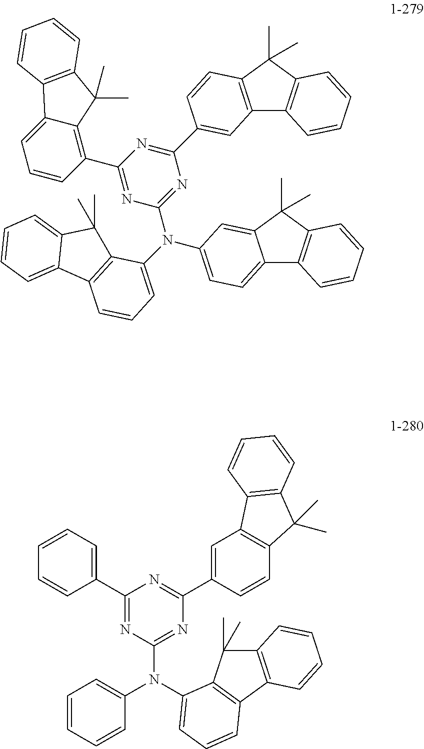 [SMILES: CC1(C)c2ccccc2-c2cc(-c3nc(-c4cccc5c4C(C)(C)c4ccccc4-5)nc(N(c4ccc5c(c4)C(C)(C)c4ccccc4-5)c4cccc5c4C(C)(C)c4ccccc4-5)n3)ccc21 ; CC1(C)c2ccccc2-c2cc(-c3nc(-c4ccccc4)nc(N(c4ccccc4)c4cccc5c4C(C)(C)c4ccccc4-5)n3)ccc21] 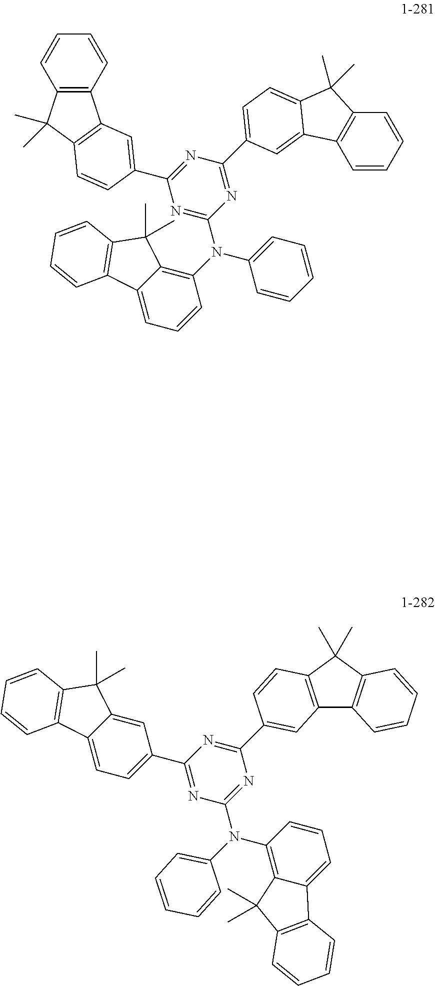 [SMILES: CC1(C)c2ccccc2-c2cc(-c3nc(-c4ccc5c(c4)-c4ccccc4C5(C)C)nc(N(c4ccccc4)c4cccc5c4C(C)(C)c4ccccc4-5)n3)ccc21 ; CC1(C)c2ccccc2-c2cc(-c3nc(-c4ccc5c(c4)C(C)(C)c4ccccc4-5)nc(N(c4ccccc4)c4cccc5c4C(C)(C)c4ccccc4-5)n3)ccc21] 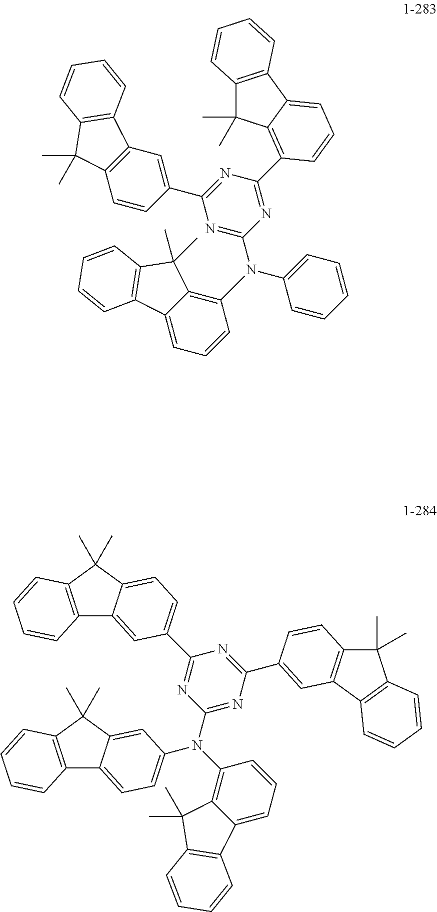 [SMILES: CC1(C)c2ccccc2-c2cc(-c3nc(-c4cccc5c4C(C)(C)c4ccccc4-5)nc(N(c4ccccc4)c4cccc5c4C(C)(C)c4ccccc4-5)n3)ccc21 ; CC1(C)c2ccccc2-c2cc(-c3nc(-c4ccc5c(c4)-c4ccccc4C5(C)C)nc(N(c4ccc5c(c4)C(C)(C)c4ccccc4-5)c4cccc5c4C(C)(C)c4ccccc4-5)n3)ccc21] 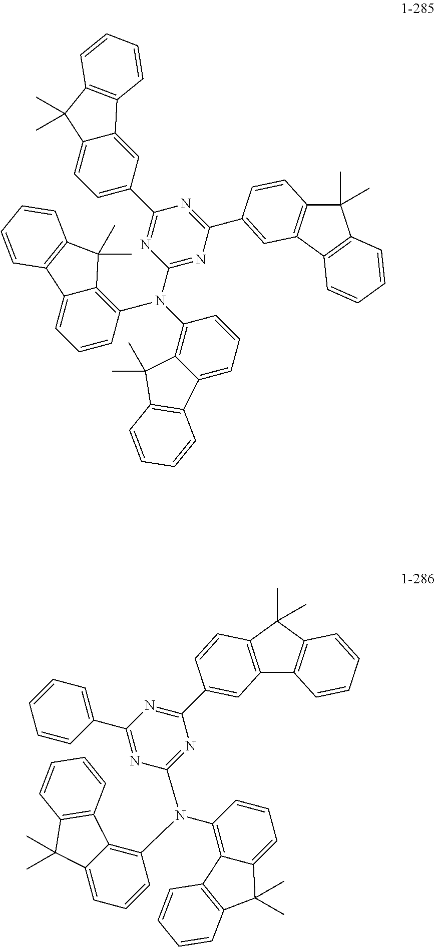 [SMILES: CC1(C)c2ccccc2-c2cc(-c3nc(-c4ccc5c(c4)-c4ccccc4C5(C)C)nc(N(c4cccc5c4C(C)(C)c4ccccc4-5)c4cccc5c4C(C)(C)c4ccccc4-5)n3)ccc21 ; CC1(C)c2ccccc2-c2cc(-c3nc(-c4ccccc4)nc(N(c4cccc5c4-c4ccccc4C5(C)C)c4cccc5c4-c4ccccc4C5(C)C)n3)ccc21] 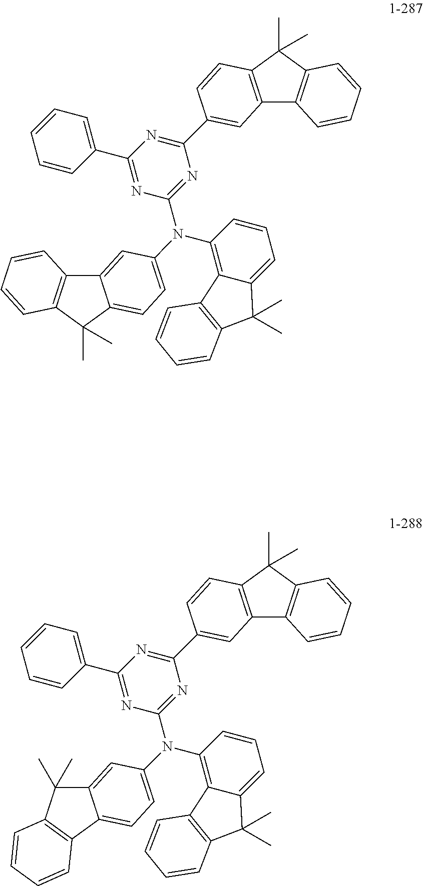 [SMILES: CC1(C)c2ccccc2-c2cc(-c3nc(-c4ccccc4)nc(N(c4ccc5c(c4)-c4ccccc4C5(C)C)c4cccc5c4-c4ccccc4C5(C)C)n3)ccc21 ; CC1(C)c2ccccc2-c2cc(-c3nc(-c4ccccc4)nc(N(c4ccc5c(c4)C(C)(C)c4ccccc4-5)c4cccc5c4-c4ccccc4C5(C)C)n3)ccc21] 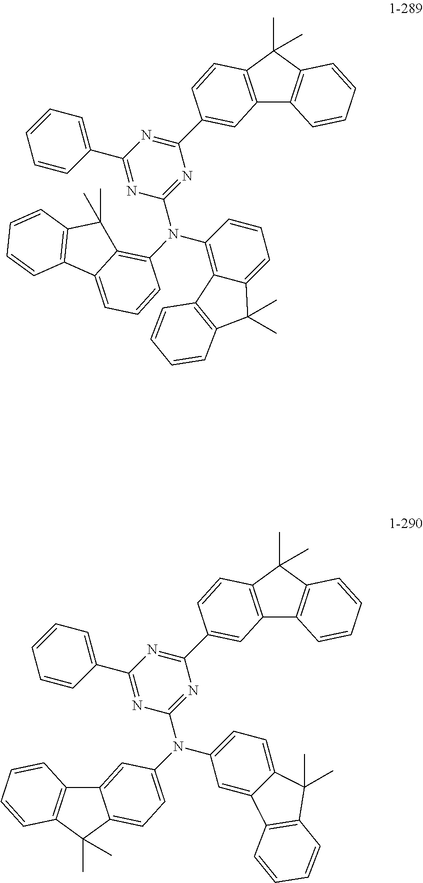 [SMILES: CC1(C)c2ccccc2-c2cc(-c3nc(-c4ccccc4)nc(N(c4cccc5c4-c4ccccc4C5(C)C)c4cccc5c4C(C)(C)c4ccccc4-5)n3)ccc21 ; CC1(C)c2ccccc2-c2cc(-c3nc(-c4ccccc4)nc(N(c4ccc5c(c4)-c4ccccc4C5(C)C)c4ccc5c(c4)-c4ccccc4C5(C)C)n3)ccc21] 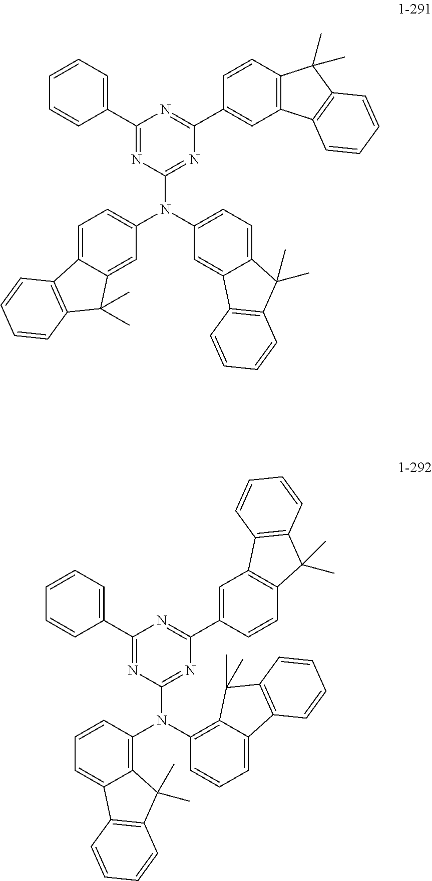 [SMILES: CC1(C)c2ccccc2-c2cc(-c3nc(-c4ccccc4)nc(N(c4ccc5c(c4)-c4ccccc4C5(C)C)c4ccc5c(c4)C(C)(C)c4ccccc4-5)n3)ccc21 ; CC1(C)c2ccccc2-c2cc(-c3nc(-c4ccccc4)nc(N(c4cccc5c4C(C)(C)c4ccccc4-5)c4cccc5c4C(C)(C)c4ccccc4-5)n3)ccc21] 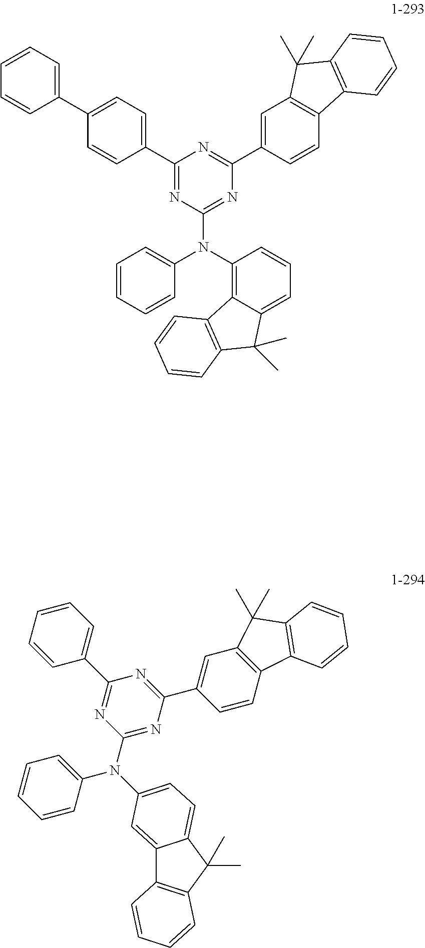 [SMILES: CC1(C)c2ccccc2-c2ccc(-c3nc(-c4ccc(-c5ccccc5)cc4)nc(N(c4ccccc4)c4cccc5c4-c4ccccc4C5(C)C)n3)cc21 ; CC1(C)c2ccccc2-c2cc(N(c3ccccc3)c3nc(-c4ccccc4)nc(-c4ccc5c(c4)C(C)(C)c4ccccc4-5)n3)ccc21] 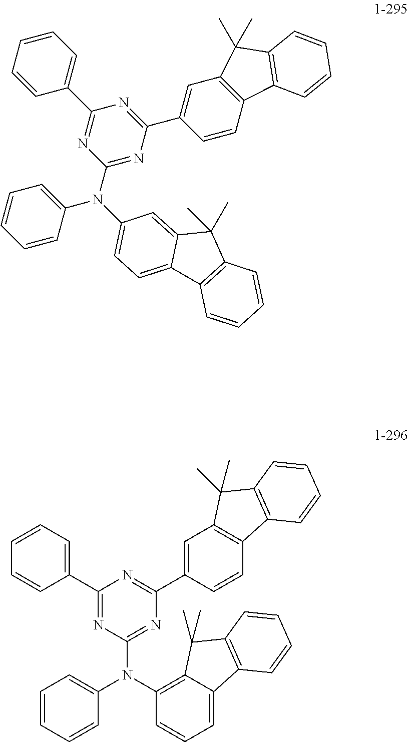 [SMILES: CC1(C)c2ccccc2-c2ccc(-c3nc(-c4ccccc4)nc(N(c4ccccc4)c4ccc5c(c4)C(C)(C)c4ccccc4-5)n3)cc21 ; CC1(C)c2ccccc2-c2ccc(-c3nc(-c4ccccc4)nc(N(c4ccccc4)c4cccc5c4C(C)(C)c4ccccc4-5)n3)cc21] 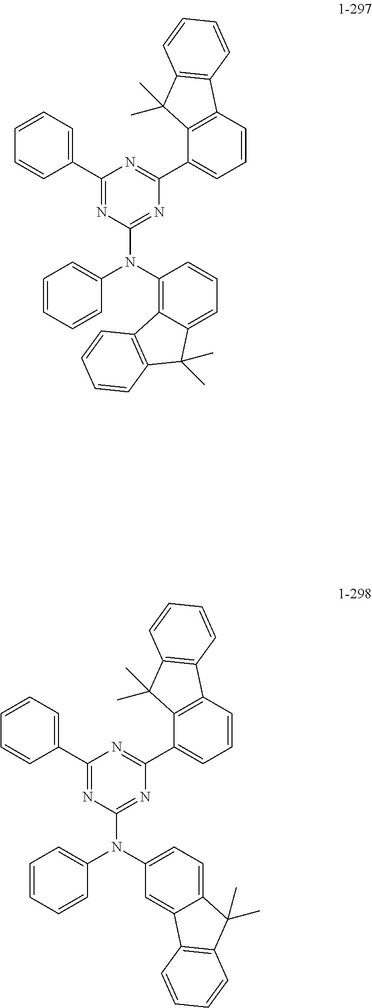 [SMILES: CC1(C)c2ccccc2-c2c(N(c3ccccc3)c3nc(-c4ccccc4)nc(-c4cccc5c4C(C)(C)c4ccccc4-5)n3)cccc21 ; CC1(C)c2ccccc2-c2cc(N(c3ccccc3)c3nc(-c4ccccc4)nc(-c4cccc5c4C(C)(C)c4ccccc4-5)n3)ccc21] 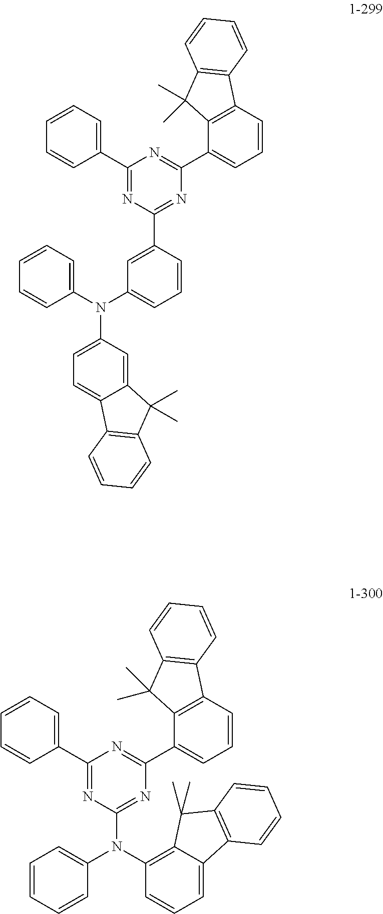 [SMILES: CC1(C)c2ccccc2-c2ccc(N(c3ccccc3)c3cccc(-c4nc(-c5ccccc5)nc(-c5cccc6c5C(C)(C)c5ccccc5-6)n4)c3)cc21 ; CC1(C)c2ccccc2-c2cccc(-c3nc(-c4ccccc4)nc(N(c4ccccc4)c4cccc5c4C(C)(C)c4ccccc4-5)n3)c21] 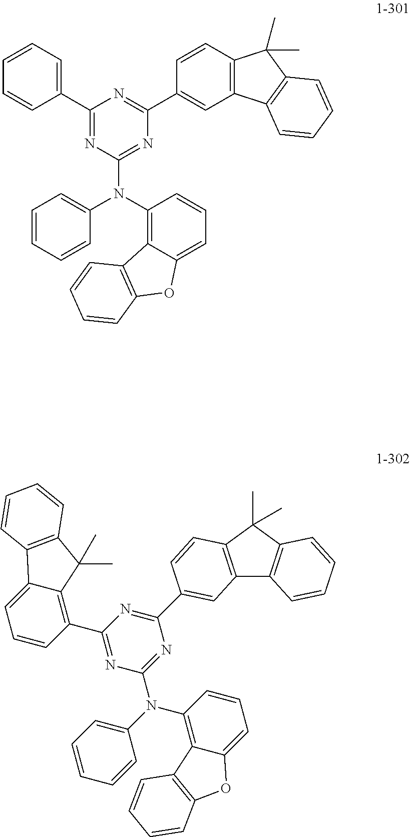 [SMILES: CC1(C)c2ccccc2-c2cc(-c3nc(-c4ccccc4)nc(N(c4ccccc4)c4cccc5oc6ccccc6c45)n3)ccc21 ; CC1(C)c2ccccc2-c2cc(-c3nc(-c4cccc5c4C(C)(C)c4ccccc4-5)nc(N(c4ccccc4)c4cccc5oc6ccccc6c45)n3)ccc21] 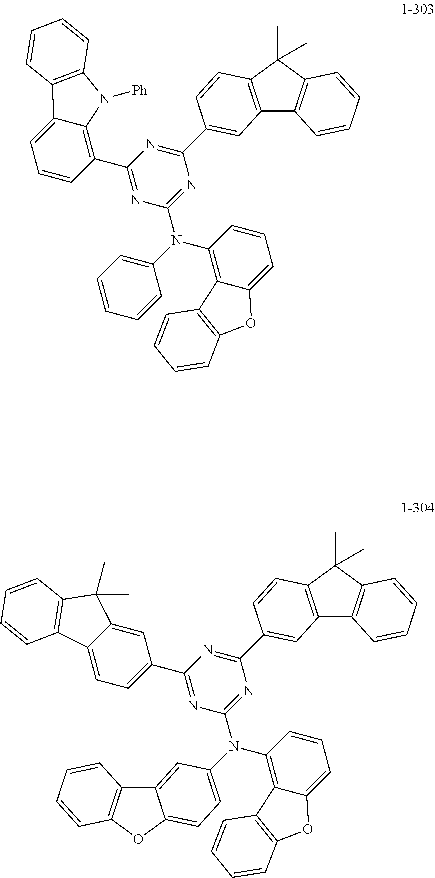 [SMILES: CC1(C)c2ccccc2-c2cc(-c3nc(-c4cccc5c6ccccc6n(-c6ccccc6)c45)nc(N(c4ccccc4)c4cccc5oc6ccccc6c45)n3)ccc21 ; CC1(C)c2ccccc2-c2cc(-c3nc(-c4ccc5c(c4)C(C)(C)c4ccccc4-5)nc(N(c4ccc5oc6ccccc6c5c4)c4cccc5oc6ccccc6c45)n3)ccc21] 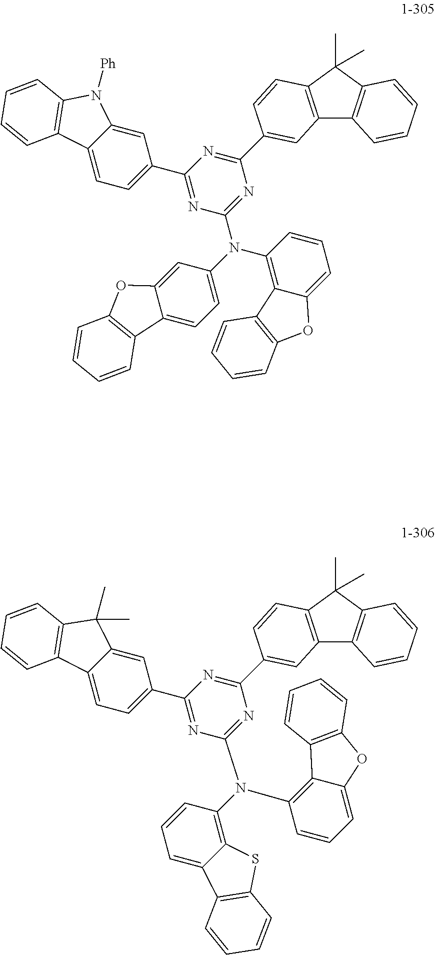 [SMILES: CC1(C)c2ccccc2-c2cc(-c3nc(-c4ccc5c6ccccc6n(-c6ccccc6)c5c4)nc(N(c4ccc5c(c4)oc4ccccc45)c4cccc5oc6ccccc6c45)n3)ccc21 ; CC1(C)c2ccccc2-c2cc(-c3nc(-c4ccc5c(c4)C(C)(C)c4ccccc4-5)nc(N(c4cccc5c4sc4ccccc45)c4cccc5oc6ccccc6c45)n3)ccc21] 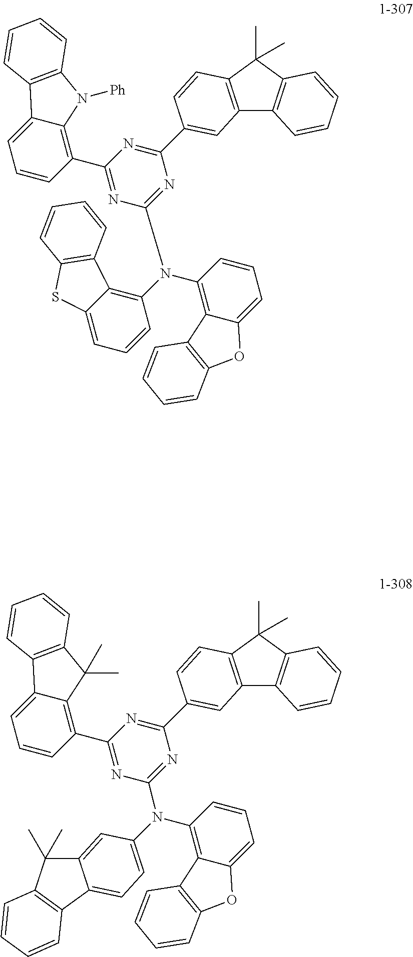 [SMILES: CC1(C)c2ccccc2-c2cc(-c3nc(-c4cccc5c6ccccc6n(-c6ccccc6)c45)nc(N(c4cccc5oc6ccccc6c45)c4cccc5sc6ccccc6c45)n3)ccc21 ; CC1(C)c2ccccc2-c2cc(-c3nc(-c4cccc5c4C(C)(C)c4ccccc4-5)nc(N(c4ccc5c(c4)C(C)(C)c4ccccc4-5)c4cccc5oc6ccccc6c45)n3)ccc21] 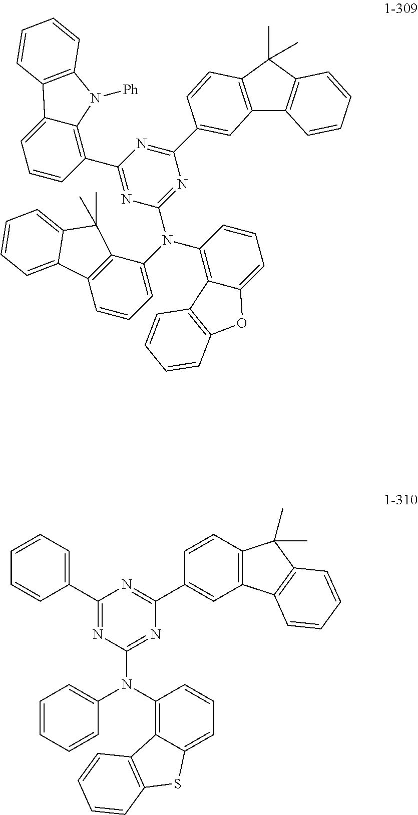 [SMILES: CC1(C)c2ccccc2-c2cc(-c3nc(-c4cccc5c6ccccc6n(-c6ccccc6)c45)nc(N(c4cccc5c4C(C)(C)c4ccccc4-5)c4cccc5oc6ccccc6c45)n3)ccc21 ; CC1(C)c2ccccc2-c2cc(-c3nc(-c4ccccc4)nc(N(c4ccccc4)c4cccc5sc6ccccc6c45)n3)ccc21] 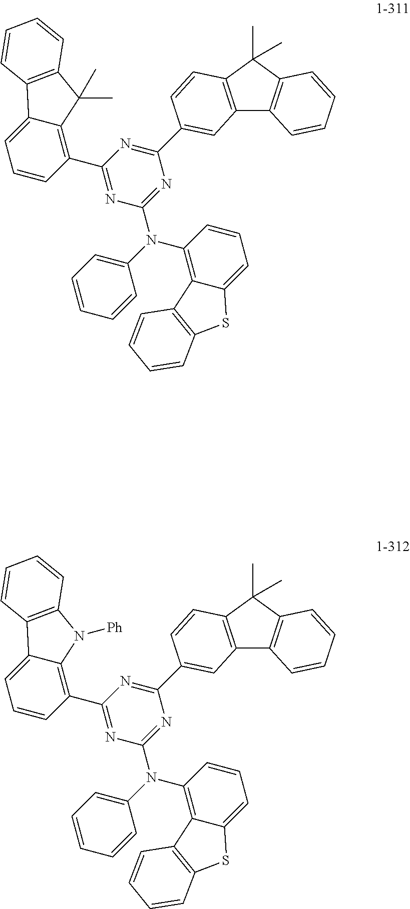 [SMILES: CC1(C)c2ccccc2-c2cc(-c3nc(-c4cccc5c4C(C)(C)c4ccccc4-5)nc(N(c4ccccc4)c4cccc5sc6ccccc6c45)n3)ccc21 ; CC1(C)c2ccccc2-c2cc(-c3nc(-c4cccc5c6ccccc6n(-c6ccccc6)c45)nc(N(c4ccccc4)c4cccc5sc6ccccc6c45)n3)ccc21] 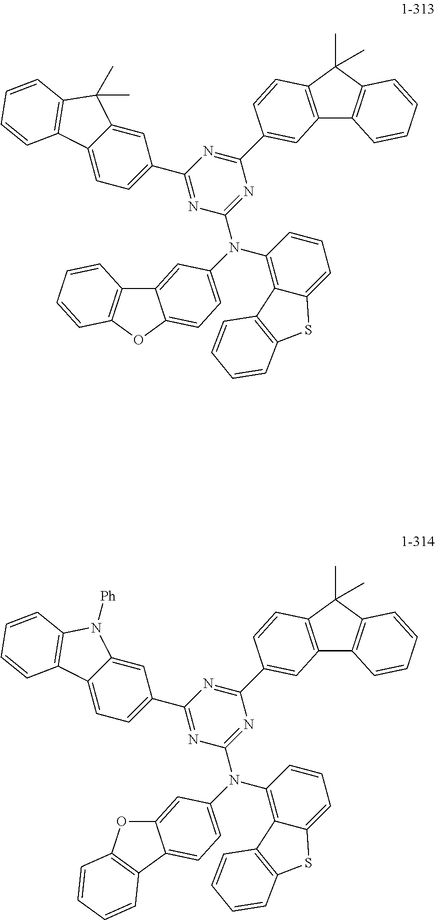 [SMILES: CC1(C)c2ccccc2-c2cc(-c3nc(-c4ccc5c(c4)C(C)(C)c4ccccc4-5)nc(N(c4ccc5oc6ccccc6c5c4)c4cccc5sc6ccccc6c45)n3)ccc21 ; CC1(C)c2ccccc2-c2cc(-c3nc(-c4ccc5c6ccccc6n(-c6ccccc6)c5c4)nc(N(c4ccc5c(c4)oc4ccccc45)c4cccc5sc6ccccc6c45)n3)ccc21] 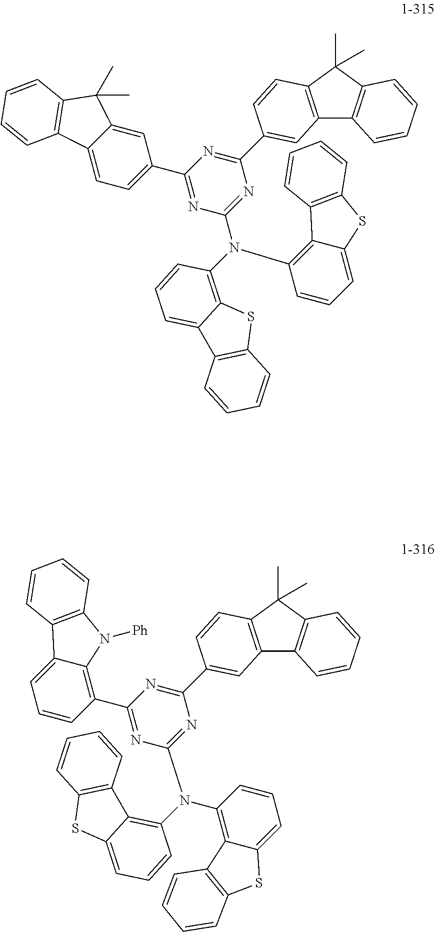 [SMILES: CC1(C)c2ccccc2-c2cc(-c3nc(-c4ccc5c(c4)C(C)(C)c4ccccc4-5)nc(N(c4cccc5c4sc4ccccc45)c4cccc5sc6ccccc6c45)n3)ccc21 ; CC1(C)c2ccccc2-c2cc(-c3nc(-c4cccc5c6ccccc6n(-c6ccccc6)c45)nc(N(c4cccc5sc6ccccc6c45)c4cccc5sc6ccccc6c45)n3)ccc21] 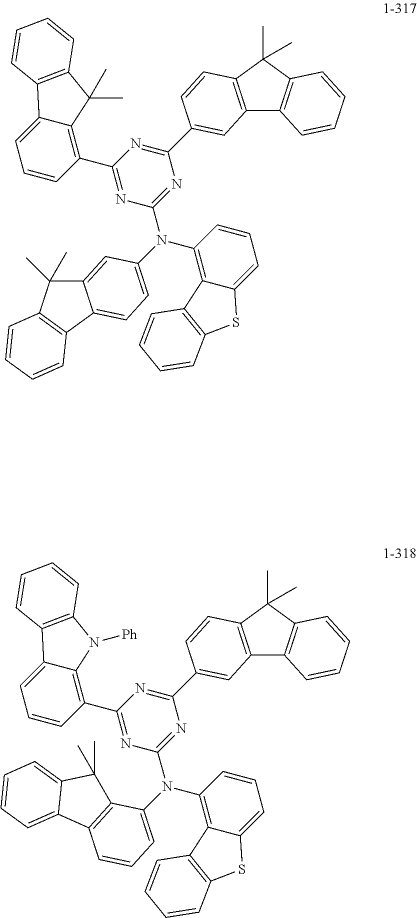 [SMILES: CC1(C)c2ccccc2-c2cc(-c3nc(-c4cccc5c4C(C)(C)c4ccccc4-5)nc(N(c4ccc5c(c4)C(C)(C)c4ccccc4-5)c4cccc5sc6ccccc6c45)n3)ccc21 ; CC1(C)c2ccccc2-c2cc(-c3nc(-c4cccc5c6ccccc6n(-c6ccccc6)c45)nc(N(c4cccc5c4C(C)(C)c4ccccc4-5)c4cccc5sc6ccccc6c45)n3)ccc21] 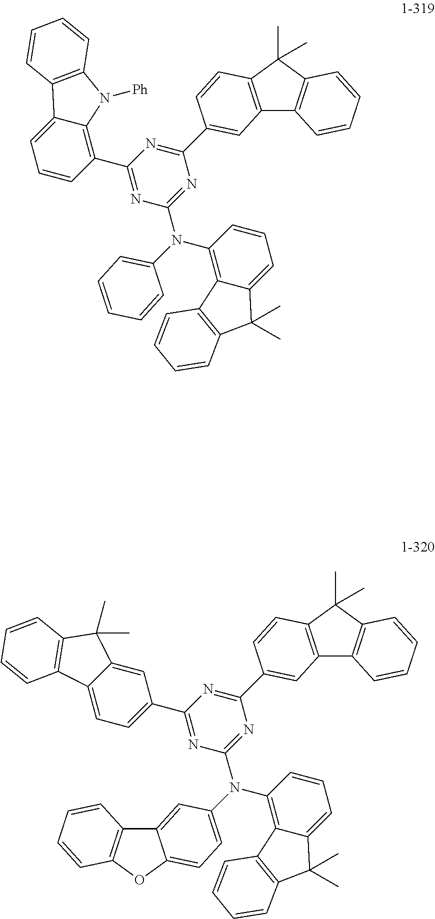 [SMILES: CC1(C)c2ccccc2-c2cc(-c3nc(-c4cccc5c6ccccc6n(-c6ccccc6)c45)nc(N(c4ccccc4)c4cccc5c4-c4ccccc4C5(C)C)n3)ccc21 ; CC1(C)c2ccccc2-c2cc(-c3nc(-c4ccc5c(c4)C(C)(C)c4ccccc4-5)nc(N(c4ccc5oc6ccccc6c5c4)c4cccc5c4-c4ccccc4C5(C)C)n3)ccc21] 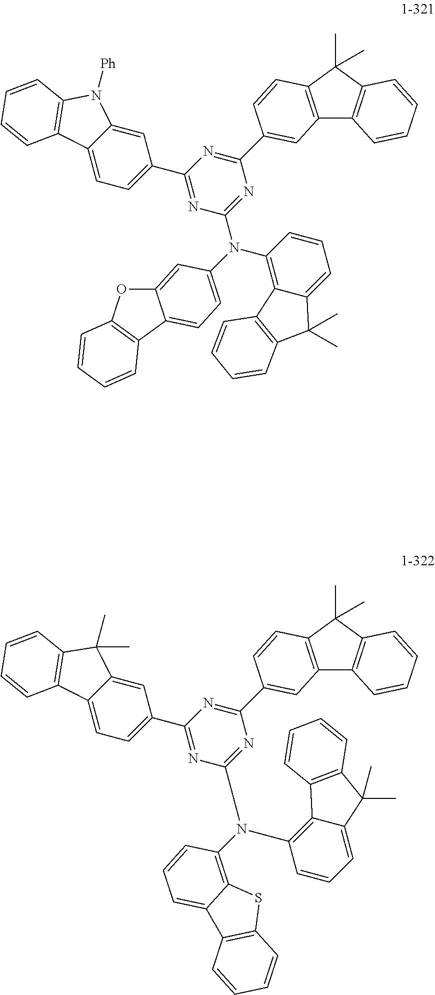 [SMILES: CC1(C)c2ccccc2-c2cc(-c3nc(-c4ccc5c6ccccc6n(-c6ccccc6)c5c4)nc(N(c4ccc5c(c4)oc4ccccc45)c4cccc5c4-c4ccccc4C5(C)C)n3)ccc21 ; CC1(C)c2ccccc2-c2cc(-c3nc(-c4ccc5c(c4)C(C)(C)c4ccccc4-5)nc(N(c4cccc5c4-c4ccccc4C5(C)C)c4cccc5c4sc4ccccc45)n3)ccc21] 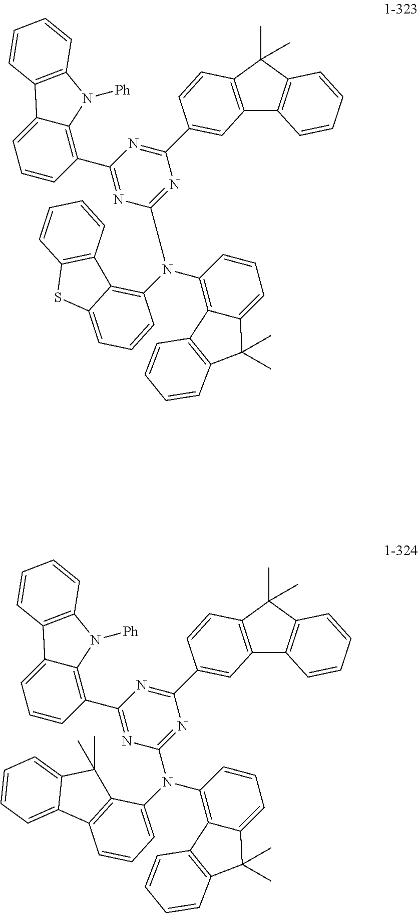 [SMILES: CC1(C)c2ccccc2-c2cc(-c3nc(-c4cccc5c6ccccc6n(-c6ccccc6)c45)nc(N(c4cccc5c4-c4ccccc4C5(C)C)c4cccc5sc6ccccc6c45)n3)ccc21 ; CC1(C)c2ccccc2-c2cc(-c3nc(-c4cccc5c6ccccc6n(-c6ccccc6)c45)nc(N(c4cccc5c4-c4ccccc4C5(C)C)c4cccc5c4C(C)(C)c4ccccc4-5)n3)ccc21] 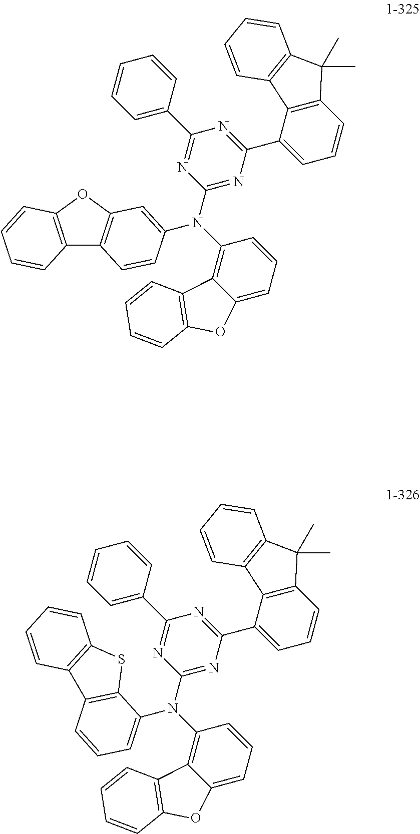 [SMILES: CC1(C)c2ccccc2-c2c(-c3nc(-c4ccccc4)nc(N(c4ccc5c(c4)oc4ccccc45)c4cccc5oc6ccccc6c45)n3)cccc21 ; CC1(C)c2ccccc2-c2c(-c3nc(-c4ccccc4)nc(N(c4cccc5c4sc4ccccc45)c4cccc5oc6ccccc6c45)n3)cccc21] 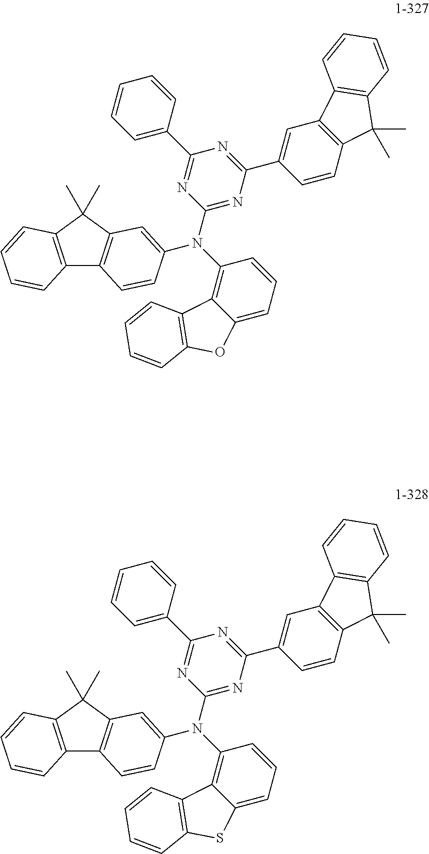 [SMILES: CC1(C)c2ccccc2-c2cc(-c3nc(-c4ccccc4)nc(N(c4ccc5c(c4)C(C)(C)c4ccccc4-5)c4cccc5oc6ccccc6c45)n3)ccc21 ; CC1(C)c2ccccc2-c2cc(-c3nc(-c4ccccc4)nc(N(c4ccc5c(c4)C(C)(C)c4ccccc4-5)c4cccc5sc6ccccc6c45)n3)ccc21] 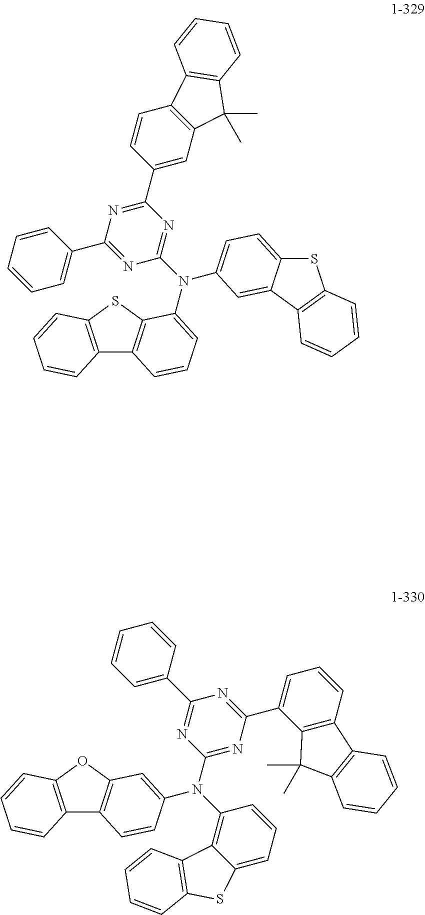 [SMILES: CC1(C)c2ccccc2-c2ccc(-c3nc(-c4ccccc4)nc(N(c4ccc5sc6ccccc6c5c4)c4cccc5c4sc4ccccc45)n3)cc21 ; CC1(C)c2ccccc2-c2cccc(-c3nc(-c4ccccc4)nc(N(c4ccc5c(c4)oc4ccccc45)c4cccc5sc6ccccc6c45)n3)c21] 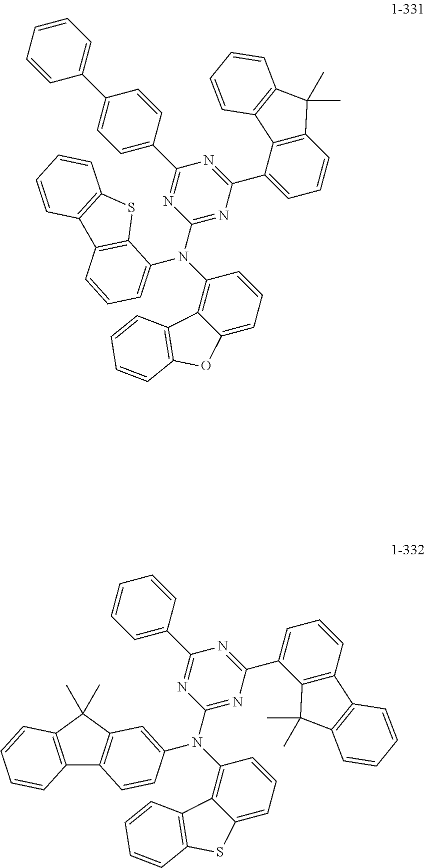 [SMILES: CC1(C)c2ccccc2-c2c(-c3nc(-c4ccc(-c5ccccc5)cc4)nc(N(c4cccc5c4sc4ccccc45)c4cccc5oc6ccccc6c45)n3)cccc21 ; CC1(C)c2ccccc2-c2ccc(N(c3nc(-c4ccccc4)nc(-c4cccc5c4C(C)(C)c4ccccc4-5)n3)c3cccc4sc5ccccc5c34)cc21] 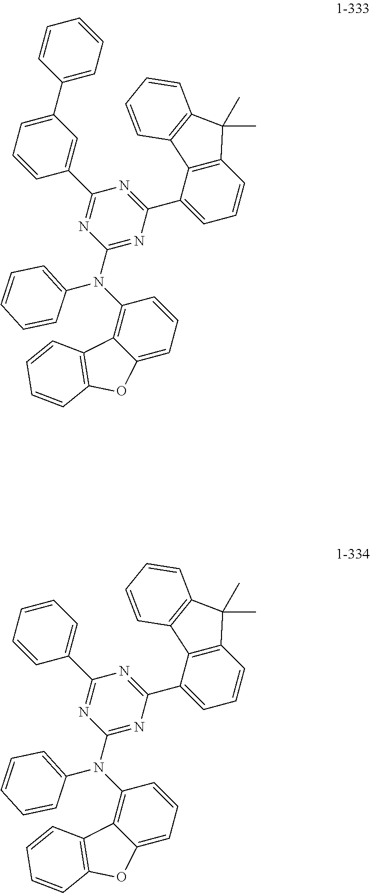 [SMILES: CC1(C)c2ccccc2-c2c(-c3nc(-c4cccc(-c5ccccc5)c4)nc(N(c4ccccc4)c4cccc5oc6ccccc6c45)n3)cccc21 ; CC1(C)c2ccccc2-c2c(-c3nc(-c4ccccc4)nc(N(c4ccccc4)c4cccc5oc6ccccc6c45)n3)cccc21] 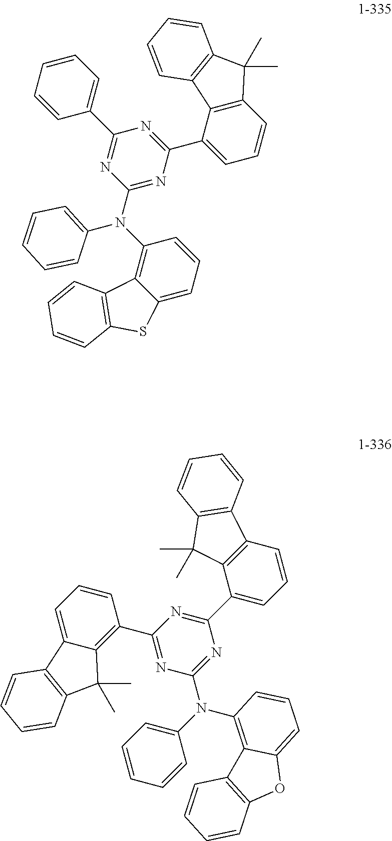 [SMILES: CC1(C)c2ccccc2-c2c(-c3nc(-c4ccccc4)nc(N(c4ccccc4)c4cccc5sc6ccccc6c45)n3)cccc21 ; CC1(C)c2ccccc2-c2cccc(-c3nc(-c4cccc5c4C(C)(C)c4ccccc4-5)nc(N(c4ccccc4)c4cccc5oc6ccccc6c45)n3)c21] 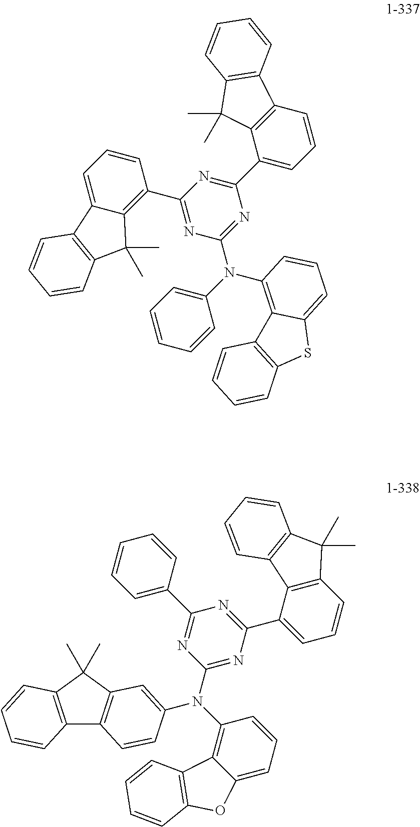 [SMILES: CC1(C)c2ccccc2-c2cccc(-c3nc(-c4cccc5c4C(C)(C)c4ccccc4-5)nc(N(c4ccccc4)c4cccc5sc6ccccc6c45)n3)c21 ; CC1(C)c2ccccc2-c2ccc(N(c3nc(-c4ccccc4)nc(-c4cccc5c4-c4ccccc4C5(C)C)n3)c3cccc4oc5ccccc5c34)cc21] 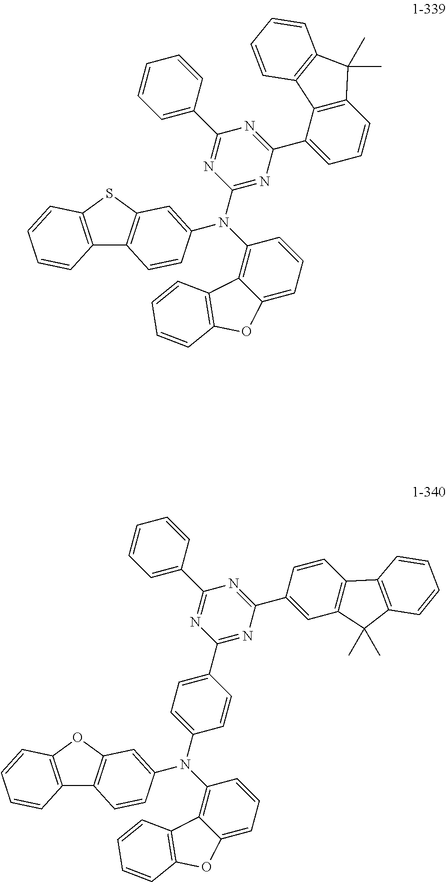 [SMILES: CC1(C)c2ccccc2-c2c(-c3nc(-c4ccccc4)nc(N(c4ccc5c(c4)sc4ccccc45)c4cccc5oc6ccccc6c45)n3)cccc21 ; CC1(C)c2ccccc2-c2ccc(-c3nc(-c4ccccc4)nc(-c4ccc(N(c5ccc6c(c5)oc5ccccc56)c5cccc6oc7ccccc7c56)cc4)n3)cc21] 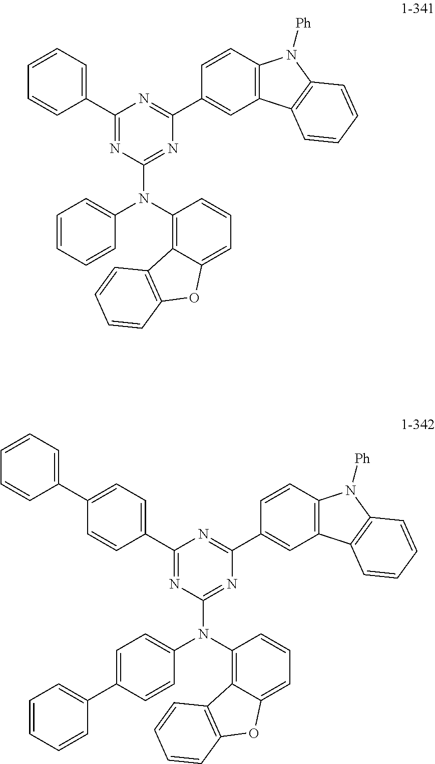 [SMILES: c1ccc(-c2nc(-c3ccc4c(c3)c3ccccc3n4-c3ccccc3)nc(N(c3ccccc3)c3cccc4oc5ccccc5c34)n2)cc1 ; c1ccc(-c2ccc(-c3nc(-c4ccc5c(c4)c4ccccc4n5-c4ccccc4)nc(N(c4ccc(-c5ccccc5)cc4)c4cccc5oc6ccccc6c45)n3)cc2)cc1] 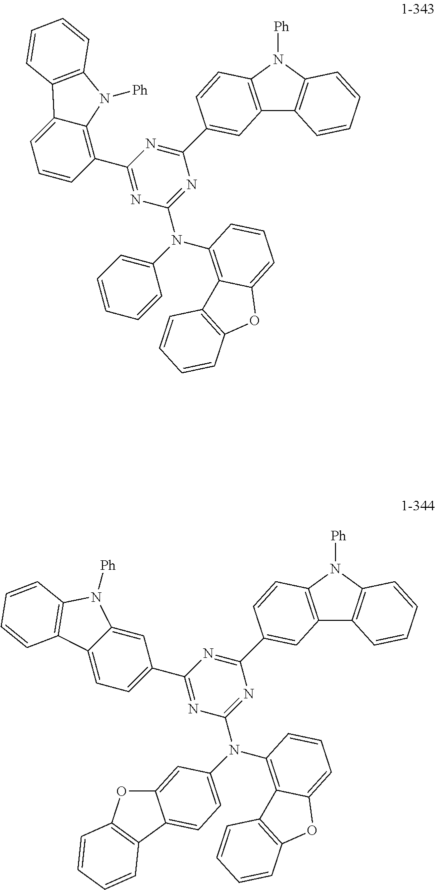 [SMILES: c1ccc(N(c2nc(-c3ccc4c(c3)c3ccccc3n4-c3ccccc3)nc(-c3cccc4c5ccccc5n(-c5ccccc5)c34)n2)c2cccc3oc4ccccc4c23)cc1 ; c1ccc(-n2c3ccccc3c3cc(-c4nc(-c5ccc6c7ccccc7n(-c7ccccc7)c6c5)nc(N(c5ccc6c(c5)oc5ccccc56)c5cccc6oc7ccccc7c56)n4)ccc32)cc1] 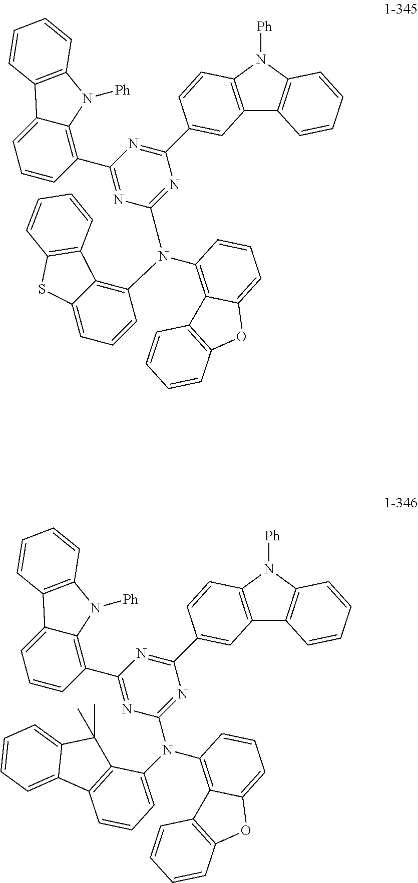 [SMILES: c1ccc(-n2c3ccccc3c3cc(-c4nc(-c5cccc6c7ccccc7n(-c7ccccc7)c56)nc(N(c5cccc6oc7ccccc7c56)c5cccc6sc7ccccc7c56)n4)ccc32)cc1 ; CC1(C)c2ccccc2-c2cccc(N(c3nc(-c4ccc5c(c4)c4ccccc4n5-c4ccccc4)nc(-c4cccc5c6ccccc6n(-c6ccccc6)c45)n3)c3cccc4oc5ccccc5c34)c21] 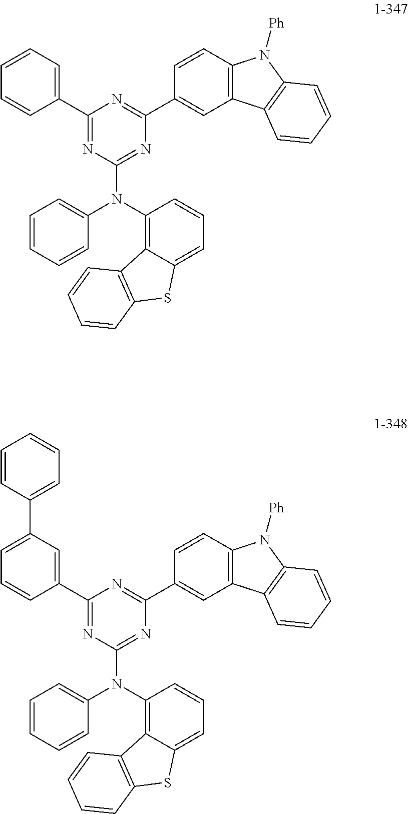 [SMILES: c1ccc(-c2nc(-c3ccc4c(c3)c3ccccc3n4-c3ccccc3)nc(N(c3ccccc3)c3cccc4sc5ccccc5c34)n2)cc1 ; c1ccc(-c2cccc(-c3nc(-c4ccc5c(c4)c4ccccc4n5-c4ccccc4)nc(N(c4ccccc4)c4cccc5sc6ccccc6c45)n3)c2)cc1] 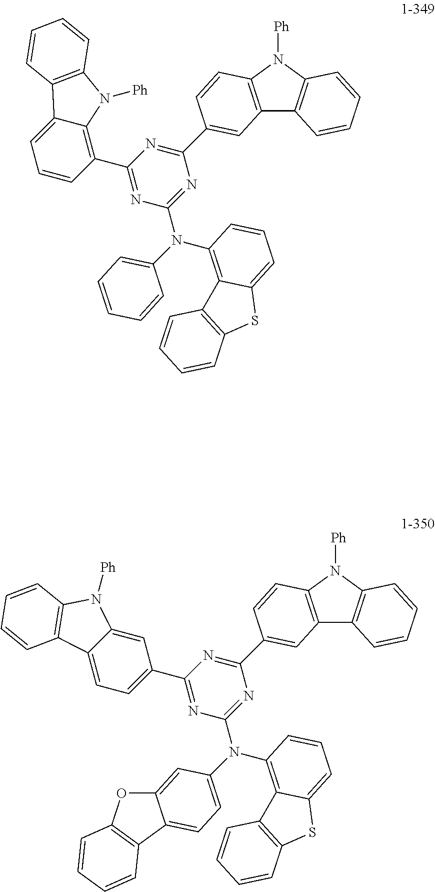 [SMILES: c1ccc(N(c2nc(-c3ccc4c(c3)c3ccccc3n4-c3ccccc3)nc(-c3cccc4c5ccccc5n(-c5ccccc5)c34)n2)c2cccc3sc4ccccc4c23)cc1 ; c1ccc(-n2c3ccccc3c3cc(-c4nc(-c5ccc6c7ccccc7n(-c7ccccc7)c6c5)nc(N(c5ccc6c(c5)oc5ccccc56)c5cccc6sc7ccccc7c56)n4)ccc32)cc1] 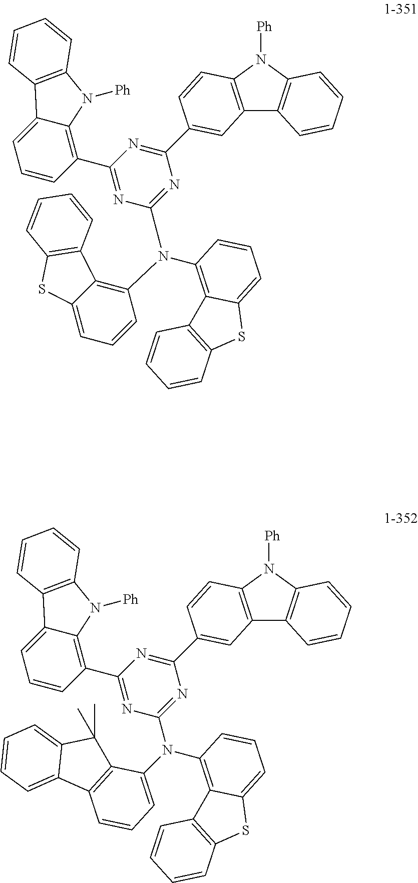 [SMILES: c1ccc(-n2c3ccccc3c3cc(-c4nc(-c5cccc6c7ccccc7n(-c7ccccc7)c56)nc(N(c5cccc6sc7ccccc7c56)c5cccc6sc7ccccc7c56)n4)ccc32)cc1 ; CC1(C)c2ccccc2-c2cccc(N(c3nc(-c4ccc5c(c4)c4ccccc4n5-c4ccccc4)nc(-c4cccc5c6ccccc6n(-c6ccccc6)c45)n3)c3cccc4sc5ccccc5c34)c21] 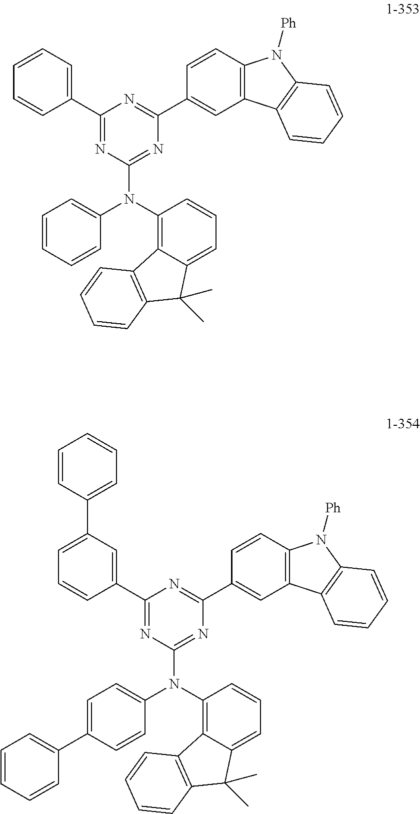 [SMILES: CC1(C)c2ccccc2-c2c(N(c3ccccc3)c3nc(-c4ccccc4)nc(-c4ccc5c(c4)c4ccccc4n5-c4ccccc4)n3)cccc21 ; CC1(C)c2ccccc2-c2c(N(c3ccc(-c4ccccc4)cc3)c3nc(-c4cccc(-c5ccccc5)c4)nc(-c4ccc5c(c4)c4ccccc4n5-c4ccccc4)n3)cccc21] 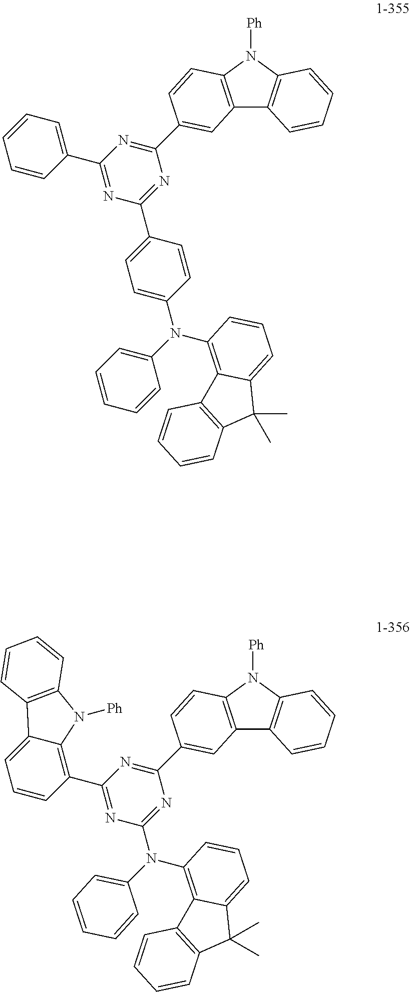 [SMILES: CC1(C)c2ccccc2-c2c(N(c3ccccc3)c3ccc(-c4nc(-c5ccccc5)nc(-c5ccc6c(c5)c5ccccc5n6-c5ccccc5)n4)cc3)cccc21 ; CC1(C)c2ccccc2-c2c(N(c3ccccc3)c3nc(-c4ccc5c(c4)c4ccccc4n5-c4ccccc4)nc(-c4cccc5c6ccccc6n(-c6ccccc6)c45)n3)cccc21] 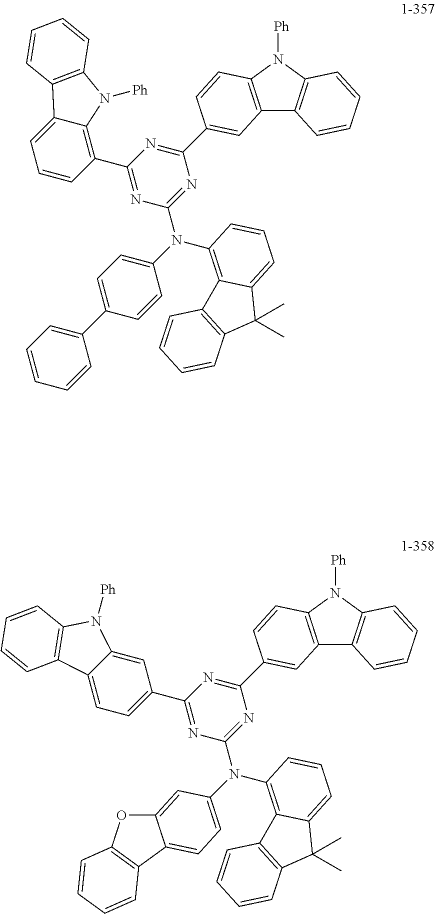 [SMILES: CC1(C)c2ccccc2-c2c(N(c3ccc(-c4ccccc4)cc3)c3nc(-c4ccc5c(c4)c4ccccc4n5-c4ccccc4)nc(-c4cccc5c6ccccc6n(-c6ccccc6)c45)n3)cccc21 ; CC1(C)c2ccccc2-c2c(N(c3ccc4c(c3)oc3ccccc34)c3nc(-c4ccc5c(c4)c4ccccc4n5-c4ccccc4)nc(-c4ccc5c6ccccc6n(-c6ccccc6)c5c4)n3)cccc21] 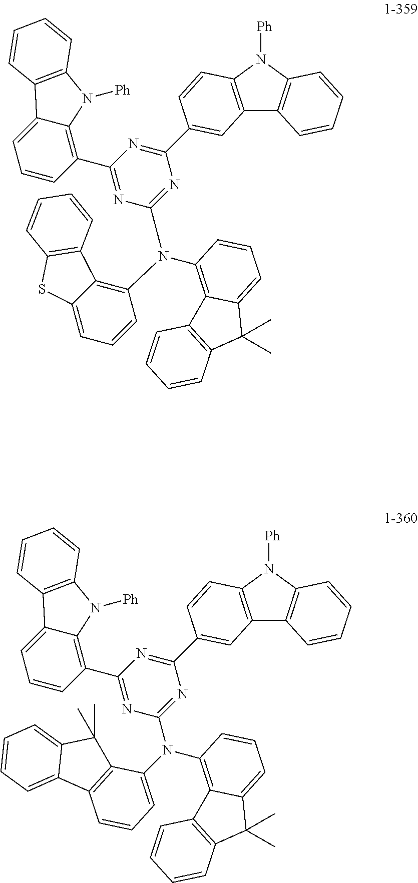 [SMILES: CC1(C)c2ccccc2-c2c(N(c3nc(-c4ccc5c(c4)c4ccccc4n5-c4ccccc4)nc(-c4cccc5c6ccccc6n(-c6ccccc6)c45)n3)c3cccc4sc5ccccc5c34)cccc21 ; CC1(C)c2ccccc2-c2c(N(c3nc(-c4ccc5c(c4)c4ccccc4n5-c4ccccc4)nc(-c4cccc5c6ccccc6n(-c6ccccc6)c45)n3)c3cccc4c3C(C)(C)c3ccccc3-4)cccc21] 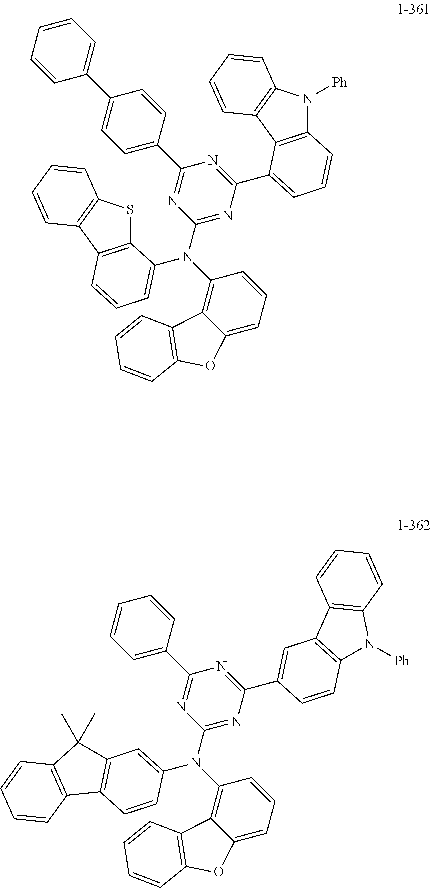 [SMILES: c1ccc(-c2ccc(-c3nc(-c4cccc5c4c4ccccc4n5-c4ccccc4)nc(N(c4cccc5c4sc4ccccc45)c4cccc5oc6ccccc6c45)n3)cc2)cc1 ; CC1(C)c2ccccc2-c2ccc(N(c3nc(-c4ccccc4)nc(-c4ccc5c(c4)c4ccccc4n5-c4ccccc4)n3)c3cccc4oc5ccccc5c34)cc21] 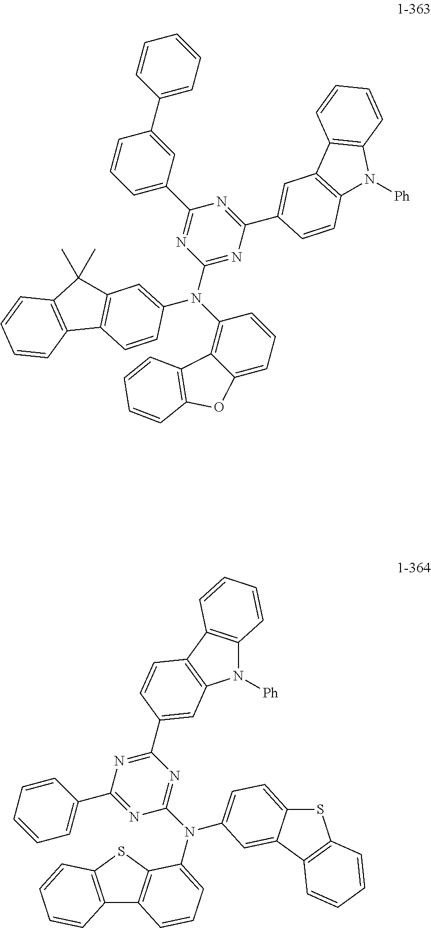 [SMILES: CC1(C)c2ccccc2-c2ccc(N(c3nc(-c4cccc(-c5ccccc5)c4)nc(-c4ccc5c(c4)c4ccccc4n5-c4ccccc4)n3)c3cccc4oc5ccccc5c34)cc21 ; c1ccc(-c2nc(-c3ccc4c5ccccc5n(-c5ccccc5)c4c3)nc(N(c3ccc4sc5ccccc5c4c3)c3cccc4c3sc3ccccc34)n2)cc1] 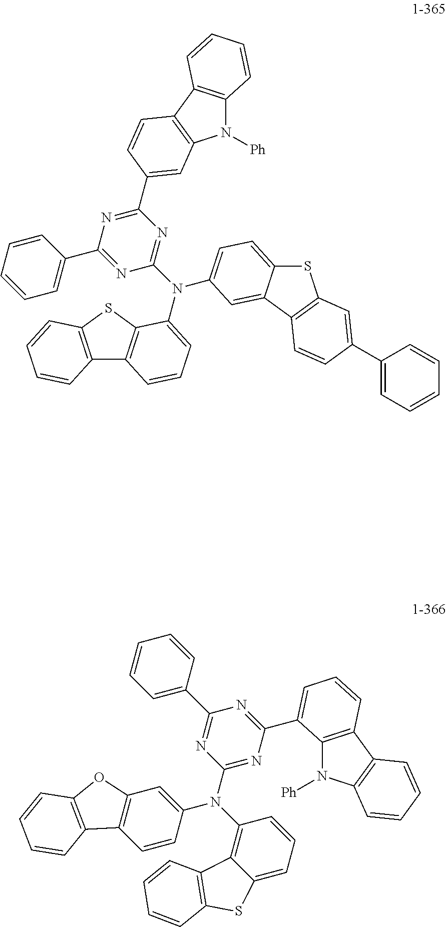 [SMILES: c1ccc(-c2ccc3c(c2)sc2ccc(N(c4nc(-c5ccccc5)nc(-c5ccc6c7ccccc7n(-c7ccccc7)c6c5)n4)c4cccc5c4sc4ccccc45)cc23)cc1 ; c1ccc(-c2nc(-c3cccc4c5ccccc5n(-c5ccccc5)c34)nc(N(c3ccc4c(c3)oc3ccccc34)c3cccc4sc5ccccc5c34)n2)cc1] 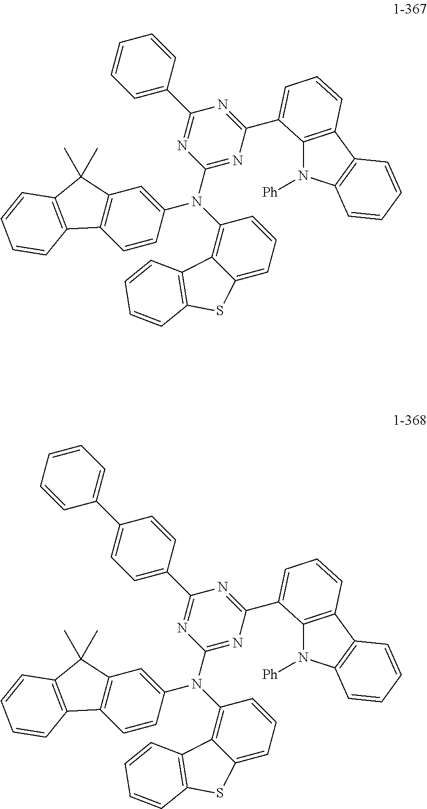 [SMILES: CC1(C)c2ccccc2-c2ccc(N(c3nc(-c4ccccc4)nc(-c4cccc5c6ccccc6n(-c6ccccc6)c45)n3)c3cccc4sc5ccccc5c34)cc21 ; CC1(C)c2ccccc2-c2ccc(N(c3nc(-c4ccc(-c5ccccc5)cc4)nc(-c4cccc5c6ccccc6n(-c6ccccc6)c45)n3)c3cccc4sc5ccccc5c34)cc21] 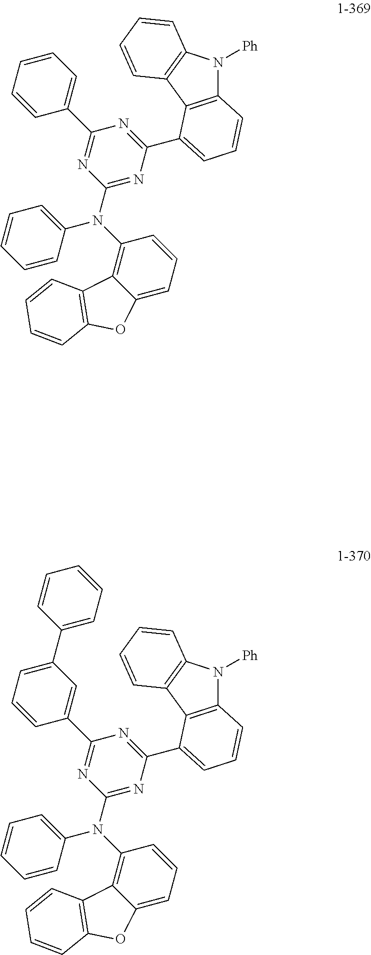 [SMILES: c1ccc(-c2nc(-c3cccc4c3c3ccccc3n4-c3ccccc3)nc(N(c3ccccc3)c3cccc4oc5ccccc5c34)n2)cc1 ; c1ccc(-c2cccc(-c3nc(-c4cccc5c4c4ccccc4n5-c4ccccc4)nc(N(c4ccccc4)c4cccc5oc6ccccc6c45)n3)c2)cc1] 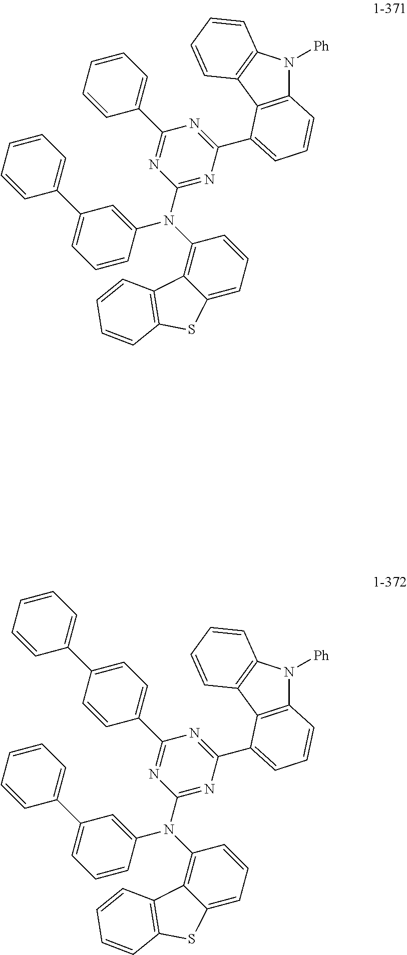 [SMILES: c1ccc(-c2cccc(N(c3nc(-c4ccccc4)nc(-c4cccc5c4c4ccccc4n5-c4ccccc4)n3)c3cccc4sc5ccccc5c34)c2)cc1 ; c1ccc(-c2ccc(-c3nc(-c4cccc5c4c4ccccc4n5-c4ccccc4)nc(N(c4cccc(-c5ccccc5)c4)c4cccc5sc6ccccc6c45)n3)cc2)cc1] 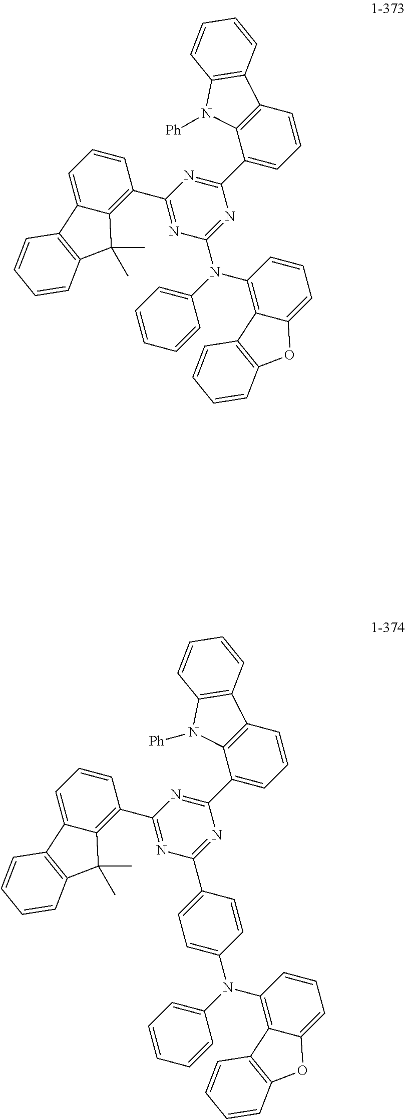 [SMILES: CC1(C)c2ccccc2-c2cccc(-c3nc(-c4cccc5c6ccccc6n(-c6ccccc6)c45)nc(N(c4ccccc4)c4cccc5oc6ccccc6c45)n3)c21 ; CC1(C)c2ccccc2-c2cccc(-c3nc(-c4ccc(N(c5ccccc5)c5cccc6oc7ccccc7c56)cc4)nc(-c4cccc5c6ccccc6n(-c6ccccc6)c45)n3)c21] 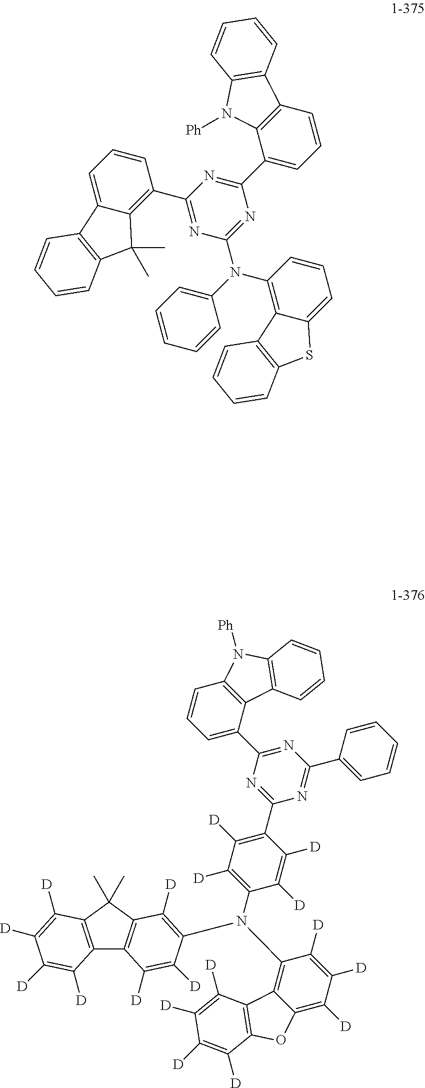 [SMILES: CC1(C)c2ccccc2-c2cccc(-c3nc(-c4cccc5c6ccccc6n(-c6ccccc6)c45)nc(N(c4ccccc4)c4cccc5sc6ccccc6c45)n3)c21 ; [2H]c1c([2H])c([2H])c2c(c1[2H])-c1c([2H])c([2H])c(N(c3c([2H])c([2H])c(-c4nc(-c5ccccc5)nc(-c5cccc6c5c5ccccc5n6-c5ccccc5)n4)c([2H])c3[2H])c3c([2H])c([2H])c([2H])c4oc5c([2H])c([2H])c([2H])c([2H])c5c34)c([2H])c1C2(C)C] 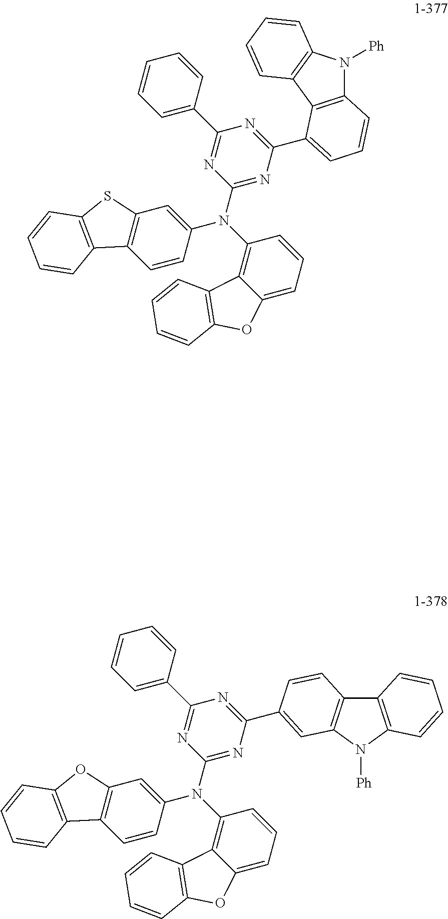 [SMILES: c1ccc(-c2nc(-c3cccc4c3c3ccccc3n4-c3ccccc3)nc(N(c3ccc4c(c3)sc3ccccc34)c3cccc4oc5ccccc5c34)n2)cc1 ; c1ccc(-c2nc(-c3ccc4c5ccccc5n(-c5ccccc5)c4c3)nc(N(c3ccc4c(c3)oc3ccccc34)c3cccc4oc5ccccc5c34)n2)cc1] 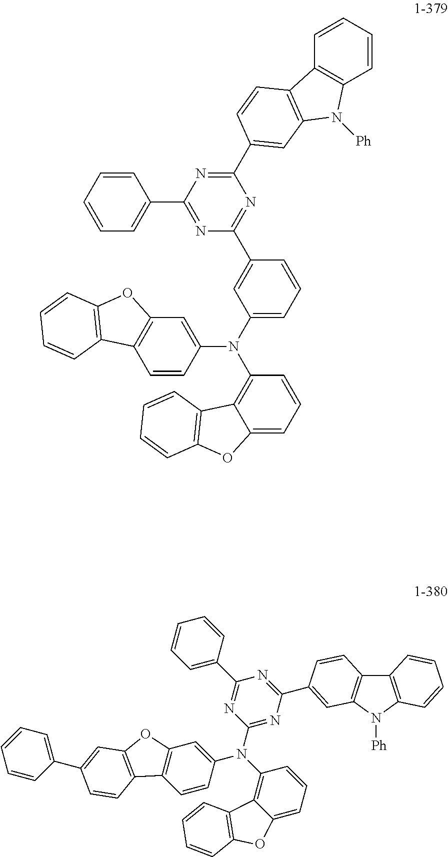 [SMILES: c1ccc(-c2nc(-c3cccc(N(c4ccc5c(c4)oc4ccccc45)c4cccc5oc6ccccc6c45)c3)nc(-c3ccc4c5ccccc5n(-c5ccccc5)c4c3)n2)cc1 ; c1ccc(-c2ccc3c(c2)oc2cc(N(c4nc(-c5ccccc5)nc(-c5ccc6c7ccccc7n(-c7ccccc7)c6c5)n4)c4cccc5oc6ccccc6c45)ccc23)cc1] 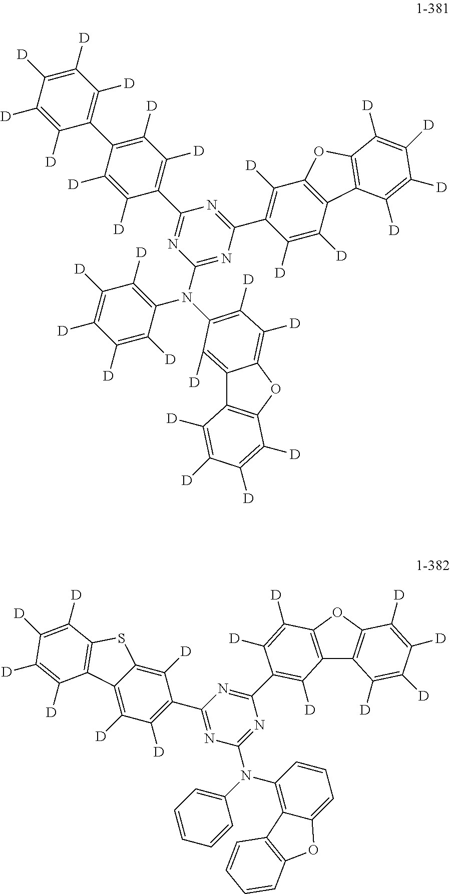 [SMILES: [2H]c1c([2H])c([2H])c(-c2c([2H])c([2H])c(-c3nc(-c4c([2H])c([2H])c5c(oc6c([2H])c([2H])c([2H])c([2H])c65)c4[2H])nc(N(c4c([2H])c([2H])c([2H])c([2H])c4[2H])c4c([2H])c([2H])c5oc6c([2H])c([2H])c([2H])c([2H])c6c5c4[2H])n3)c([2H])c2[2H])c([2H])c1[2H] ; [2H]c1c([2H])c([2H])c2c(oc3c([2H])c([2H])c(-c4nc(-c5c([2H])c([2H])c6c(sc7c([2H])c([2H])c([2H])c([2H])c76)c5[2H])nc(N(c5ccccc5)c5cccc6oc7ccccc7c56)n4)c([2H])c32)c1[2H]] 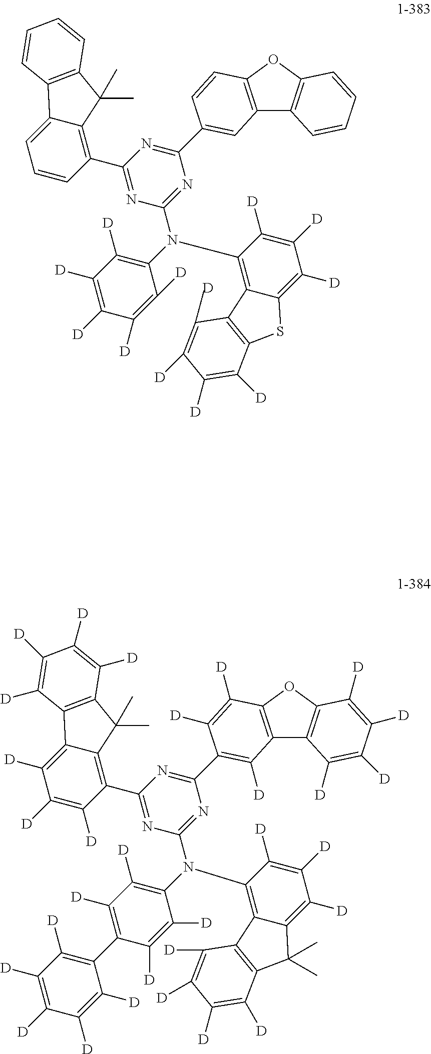 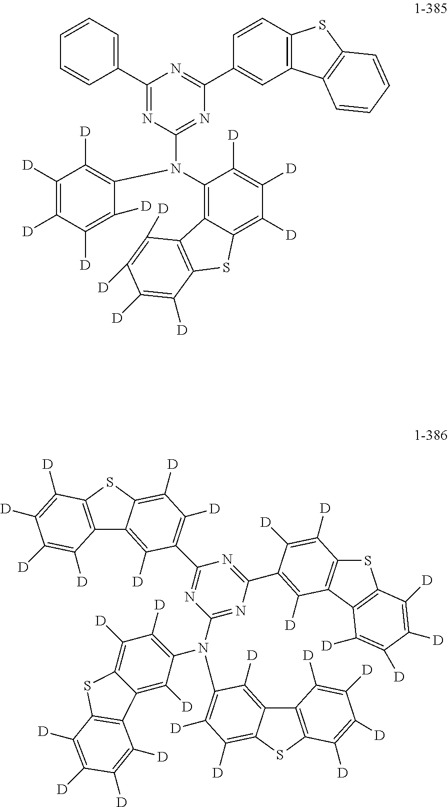 [SMILES: [2H]c1c([2H])c([2H])c(N(c2nc(-c3ccccc3)nc(-c3ccc4sc5ccccc5c4c3)n2)c2c([2H])c([2H])c([2H])c3sc4c([2H])c([2H])c([2H])c([2H])c4c23)c([2H])c1[2H] ; [2H]c1c([2H])c([2H])c2c(sc3c([2H])c([2H])c(-c4nc(-c5c([2H])c([2H])c6sc7c([2H])c([2H])c([2H])c([2H])c7c6c5[2H])nc(N(c5c([2H])c([2H])c6sc7c([2H])c([2H])c([2H])c([2H])c7c6c5[2H])c5c([2H])c([2H])c6sc7c([2H])c([2H])c([2H])c([2H])c7c6c5[2H])n4)c([2H])c32)c1[2H]] 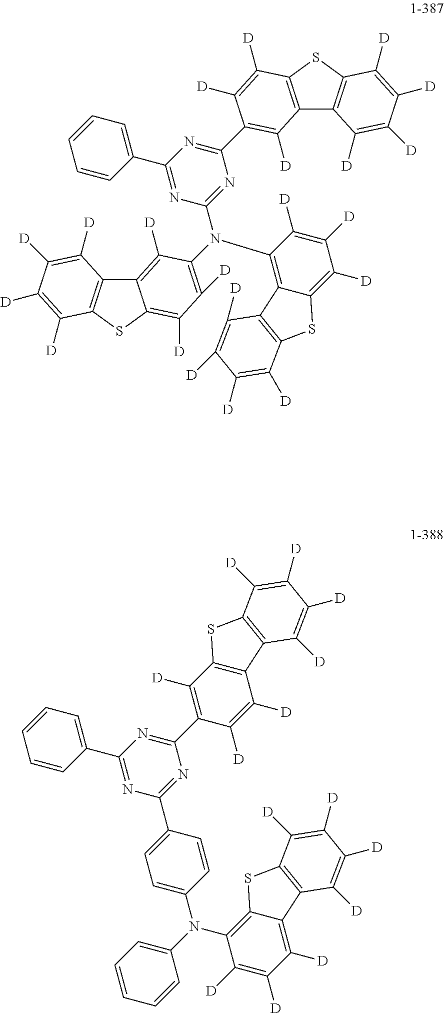 [SMILES: [2H]c1c([2H])c([2H])c2c(sc3c([2H])c([2H])c(-c4nc(-c5ccccc5)nc(N(c5c([2H])c([2H])c6sc7c([2H])c([2H])c([2H])c([2H])c7c6c5[2H])c5c([2H])c([2H])c([2H])c6sc7c([2H])c([2H])c([2H])c([2H])c7c56)n4)c([2H])c32)c1[2H] ; [2H]c1c([2H])c([2H])c2c(sc3c([2H])c(-c4nc(-c5ccccc5)nc(-c5ccc(N(c6ccccc6)c6c([2H])c([2H])c([2H])c7c6sc6c([2H])c([2H])c([2H])c([2H])c67)cc5)n4)c([2H])c([2H])c32)c1[2H]] 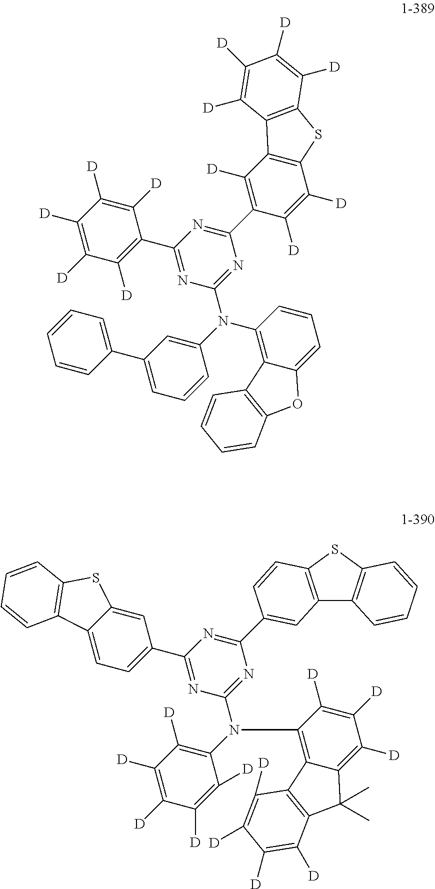 [SMILES: [2H]c1c([2H])c([2H])c(-c2nc(-c3c([2H])c([2H])c4sc5c([2H])c([2H])c([2H])c([2H])c5c4c3[2H])nc(N(c3cccc(-c4ccccc4)c3)c3cccc4oc5ccccc5c34)n2)c([2H])c1[2H] ; [2H]c1c([2H])c([2H])c(N(c2nc(-c3ccc4c(c3)sc3ccccc34)nc(-c3ccc4sc5ccccc5c4c3)n2)c2c([2H])c([2H])c([2H])c3c2-c2c([2H])c([2H])c([2H])c([2H])c2C3(C)C)c([2H])c1[2H]] 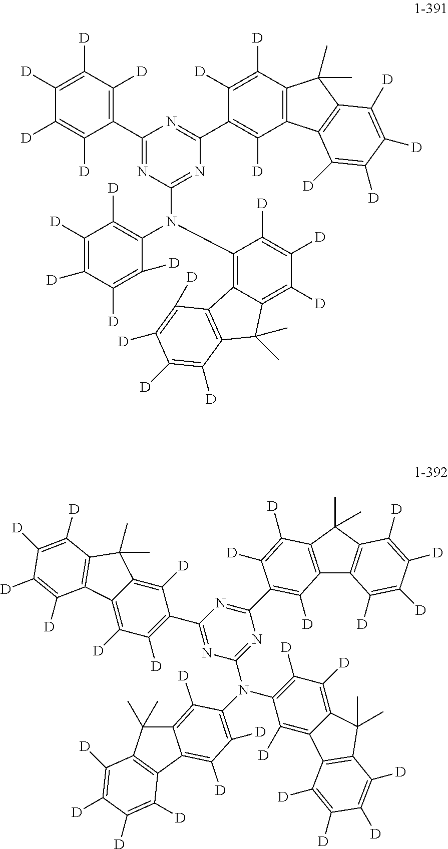 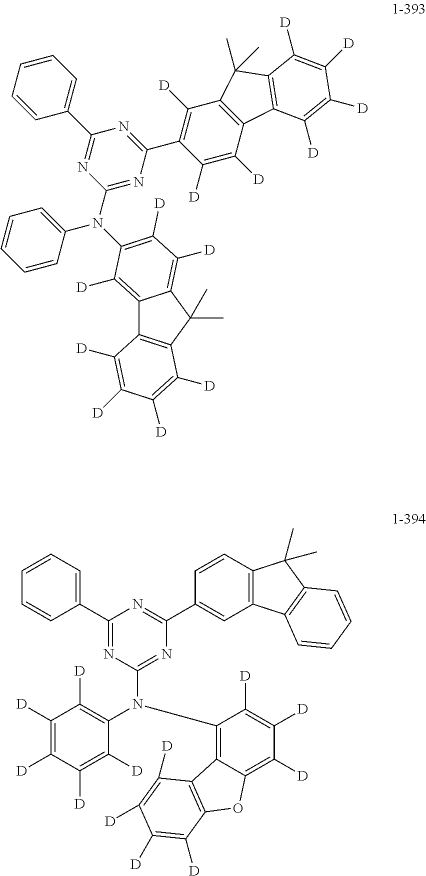 [SMILES: [2H]c1c([2H])c([2H])c2c(c1[2H])-c1c([2H])c(N(c3ccccc3)c3nc(-c4ccccc4)nc(-c4c([2H])c([2H])c5c(c4[2H])C(C)(C)c4c([2H])c([2H])c([2H])c([2H])c4-5)n3)c([2H])c([2H])c1C2(C)C ; [2H]c1c([2H])c([2H])c(N(c2nc(-c3ccccc3)nc(-c3ccc4c(c3)-c3ccccc3C4(C)C)n2)c2c([2H])c([2H])c([2H])c3oc4c([2H])c([2H])c([2H])c([2H])c4c23)c([2H])c1[2H]] 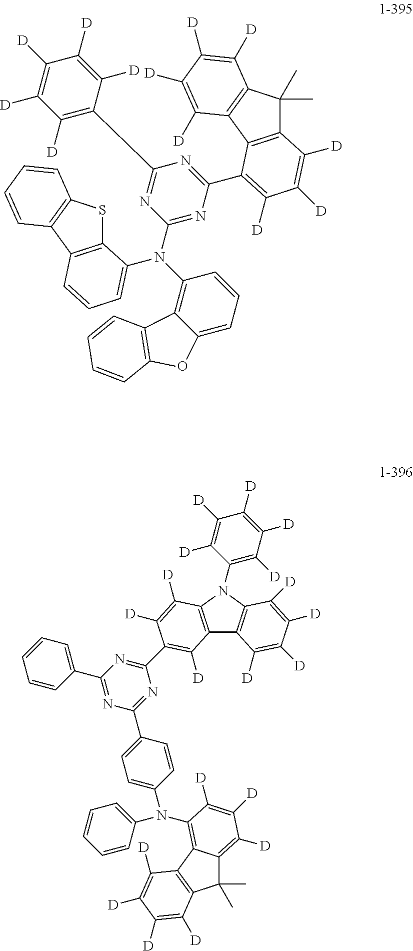 [SMILES: [2H]c1c([2H])c([2H])c(-c2nc(-c3c([2H])c([2H])c([2H])c4c3-c3c([2H])c([2H])c([2H])c([2H])c3C4(C)C)nc(N(c3cccc4c3sc3ccccc34)c3cccc4oc5ccccc5c34)n2)c([2H])c1[2H] ; [2H]c1c([2H])c([2H])c(-n2c3c([2H])c([2H])c([2H])c([2H])c3c3c([2H])c(-c4nc(-c5ccccc5)nc(-c5ccc(N(c6ccccc6)c6c([2H])c([2H])c([2H])c7c6-c6c([2H])c([2H])c([2H])c([2H])c6C7(C)C)cc5)n4)c([2H])c([2H])c32)c([2H])c1[2H]] 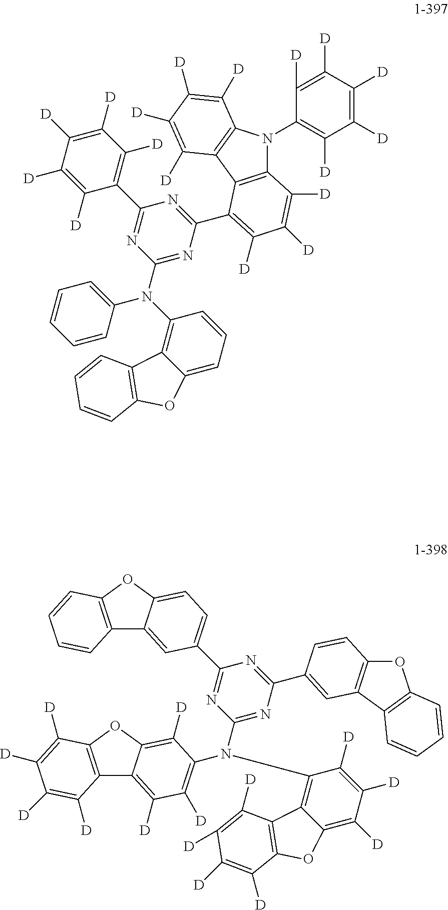 [SMILES: [2H]c1c([2H])c([2H])c(-c2nc(-c3c([2H])c([2H])c([2H])c4c3c3c([2H])c([2H])c([2H])c([2H])c3n4-c3c([2H])c([2H])c([2H])c([2H])c3[2H])nc(N(c3ccccc3)c3cccc4oc5ccccc5c34)n2)c([2H])c1[2H] ; [2H]c1c([2H])c([2H])c2c(oc3c([2H])c(N(c4nc(-c5ccc6oc7ccccc7c6c5)nc(-c5ccc6oc7ccccc7c6c5)n4)c4c([2H])c([2H])c([2H])c5oc6c([2H])c([2H])c([2H])c([2H])c6c45)c([2H])c([2H])c32)c1[2H]] 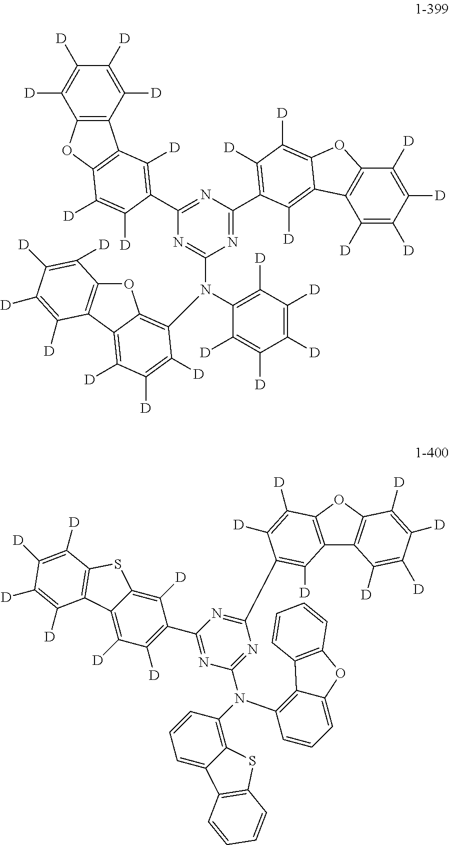 [SMILES: [2H]c1c([2H])c([2H])c(N(c2nc(-c3c([2H])c([2H])c4oc5c([2H])c([2H])c([2H])c([2H])c5c4c3[2H])nc(-c3c([2H])c([2H])c4oc5c([2H])c([2H])c([2H])c([2H])c5c4c3[2H])n2)c2c([2H])c([2H])c([2H])c3c2oc2c([2H])c([2H])c([2H])c([2H])c23)c([2H])c1[2H] ; [2H]c1c([2H])c([2H])c2c(oc3c([2H])c([2H])c(-c4nc(-c5c([2H])c([2H])c6c(sc7c([2H])c([2H])c([2H])c([2H])c76)c5[2H])nc(N(c5cccc6c5sc5ccccc56)c5cccc6oc7ccccc7c56)n4)c([2H])c32)c1[2H]]
in the compounds, Ph is a phenyl group.

8. An organic light emitting device comprising:
a first electrode;
a second electrode; and
one or more organic material layers provided between the first electrode and the second electrode,
wherein one or more layers of the organic material layers include the heterocyclic compound of claim 1.

9. The organic light emitting device of claim 8, wherein the organic material layer includes a light emitting layer, and the light emitting layer includes the heterocyclic compound.

10. The organic light emitting device of claim 8, wherein the organic material layer includes a light emitting layer, the light emitting layer includes a host, and the host includes the heterocyclic compound.

11. The organic light emitting device of claim 8, wherein the organic material layer including the heterocyclic compound further includes a compound of the following Chemical Formula 2: 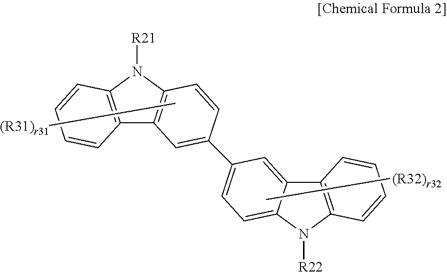
in Chemical Formula 2,
R21 and R22 are the same as or different from each other, and each independently deuterium; a halogen group; a cyano group; a substituted or unsubstituted C1 to C60 alkyl group; a substituted or unsubstituted C3 to C60 cycloalkyl group; or a substituted or unsubstituted C6 to C60 aryl group,
R31 and R32 are the same as or different from each other, and each independently hydrogen; deuterium; a halogen group; a cyano group; a substituted or unsubstituted C1 to C60 alkyl group; a substituted or unsubstituted C3 to C60 cycloalkyl group; a substituted or unsubstituted C6 to C60 aryl group; or a substituted or unsubstituted C2 to C60 heteroaryl group,
r31 is an integer of 0 to 7, and when 2 or greater, R31s are the same as or different from each other, and
r32 is an integer of 0 to 7, and when 2 or greater, R32s are the same as or different from each other.

12. The organic light emitting device of claim 11, wherein the compound of Chemical Formula 2 is represented by any one of the following compounds:

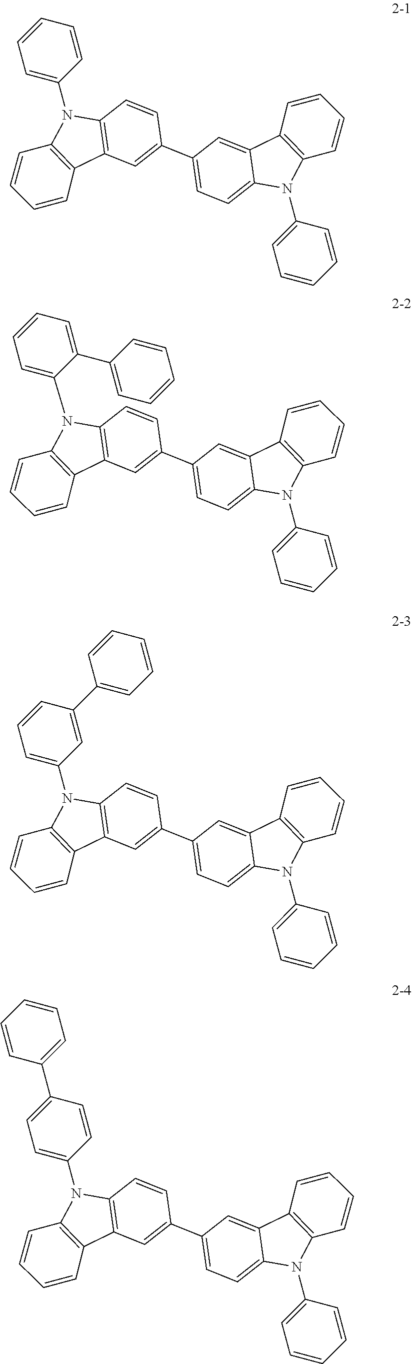 [SMILES: c1ccc(-n2c3ccccc3c3cc(-c4ccc5c(c4)c4ccccc4n5-c4ccccc4)ccc32)cc1 ; c1ccc(-c2ccccc2-n2c3ccccc3c3cc(-c4ccc5c(c4)c4ccccc4n5-c4ccccc4)ccc32)cc1 ; c1ccc(-c2cccc(-n3c4ccccc4c4cc(-c5ccc6c(c5)c5ccccc5n6-c5ccccc5)ccc43)c2)cc1 ; c1ccc(-c2ccc(-n3c4ccccc4c4cc(-c5ccc6c(c5)c5ccccc5n6-c5ccccc5)ccc43)cc2)cc1]

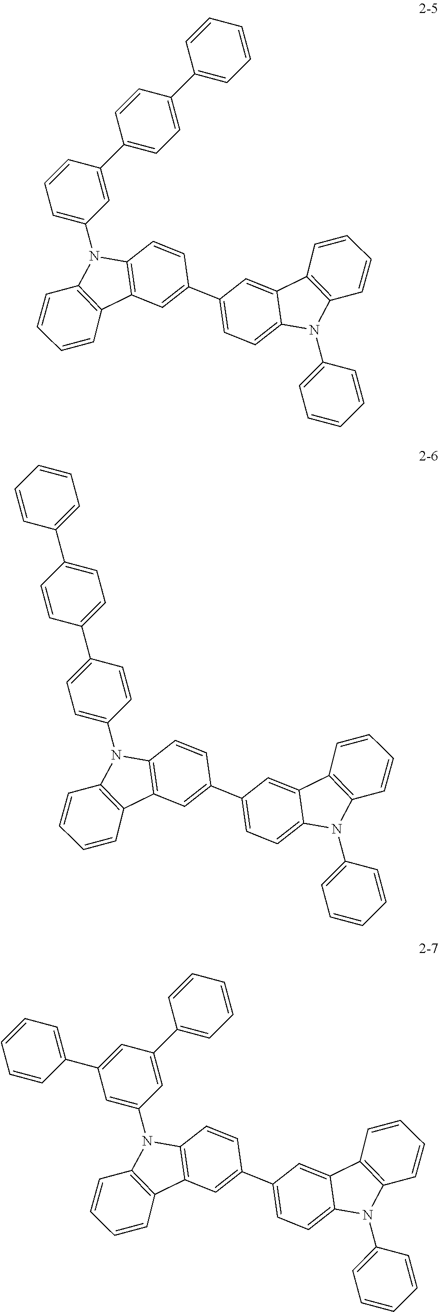 [SMILES: c1ccc(-c2ccc(-c3cccc(-n4c5ccccc5c5cc(-c6ccc7c(c6)c6ccccc6n7-c6ccccc6)ccc54)c3)cc2)cc1 ; c1ccc(-c2ccc(-c3ccc(-n4c5ccccc5c5cc(-c6ccc7c(c6)c6ccccc6n7-c6ccccc6)ccc54)cc3)cc2)cc1 ; c1ccc(-c2cc(-c3ccccc3)cc(-n3c4ccccc4c4cc(-c5ccc6c(c5)c5ccccc5n6-c5ccccc5)ccc43)c2)cc1]

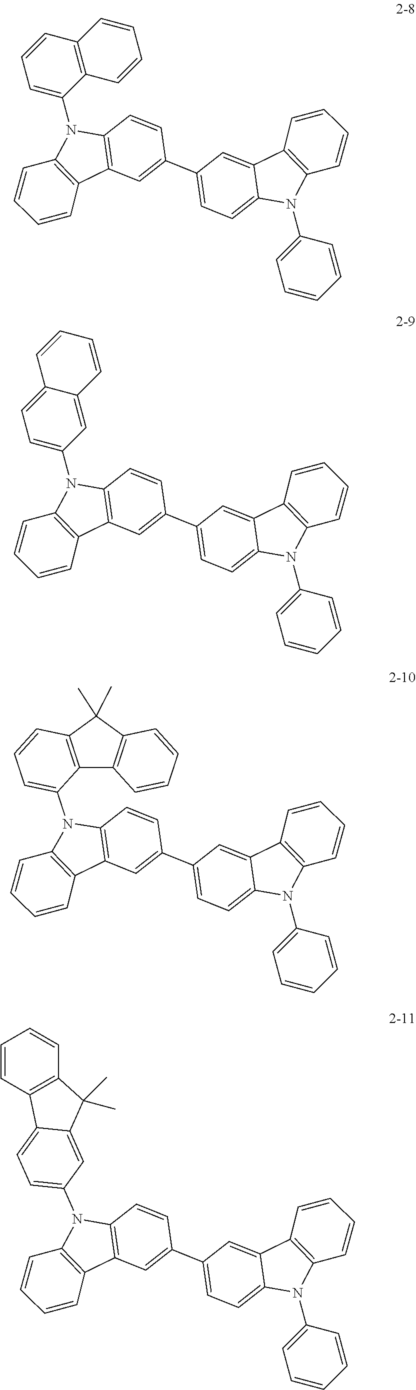 [SMILES: c1ccc(-n2c3ccccc3c3cc(-c4ccc5c(c4)c4ccccc4n5-c4cccc5ccccc45)ccc32)cc1 ; c1ccc(-n2c3ccccc3c3cc(-c4ccc5c(c4)c4ccccc4n5-c4ccc5ccccc5c4)ccc32)cc1 ; CC1(C)c2ccccc2-c2c(-n3c4ccccc4c4cc(-c5ccc6c(c5)c5ccccc5n6-c5ccccc5)ccc43)cccc21 ; CC1(C)c2ccccc2-c2ccc(-n3c4ccccc4c4cc(-c5ccc6c(c5)c5ccccc5n6-c5ccccc5)ccc43)cc21]

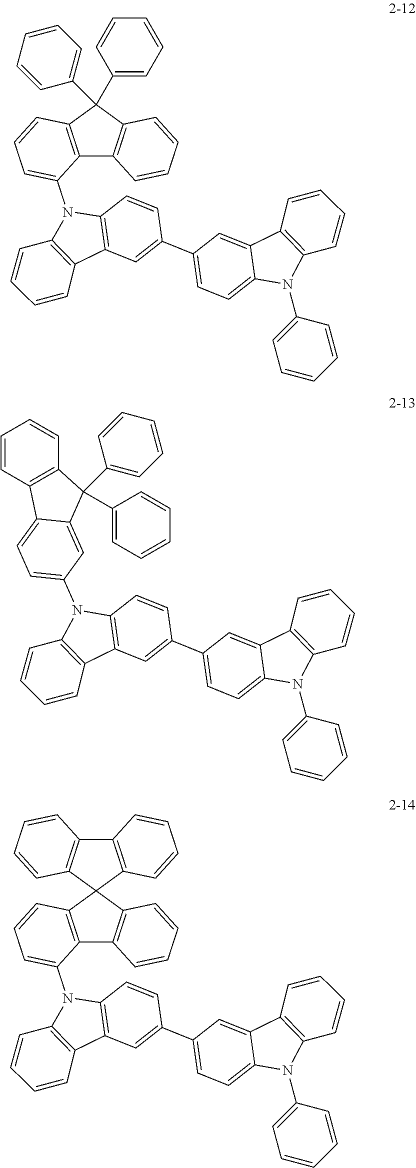 [SMILES: c1ccc(-n2c3ccccc3c3cc(-c4ccc5c(c4)c4ccccc4n5-c4cccc5c4-c4ccccc4C5(c4ccccc4)c4ccccc4)ccc32)cc1 ; c1ccc(-n2c3ccccc3c3cc(-c4ccc5c(c4)c4ccccc4n5-c4ccc5c(c4)C(c4ccccc4)(c4ccccc4)c4ccccc4-5)ccc32)cc1 ; c1ccc(-n2c3ccccc3c3cc(-c4ccc5c(c4)c4ccccc4n5-c4cccc5c4-c4ccccc4C54c5ccccc5-c5ccccc54)ccc32)cc1]

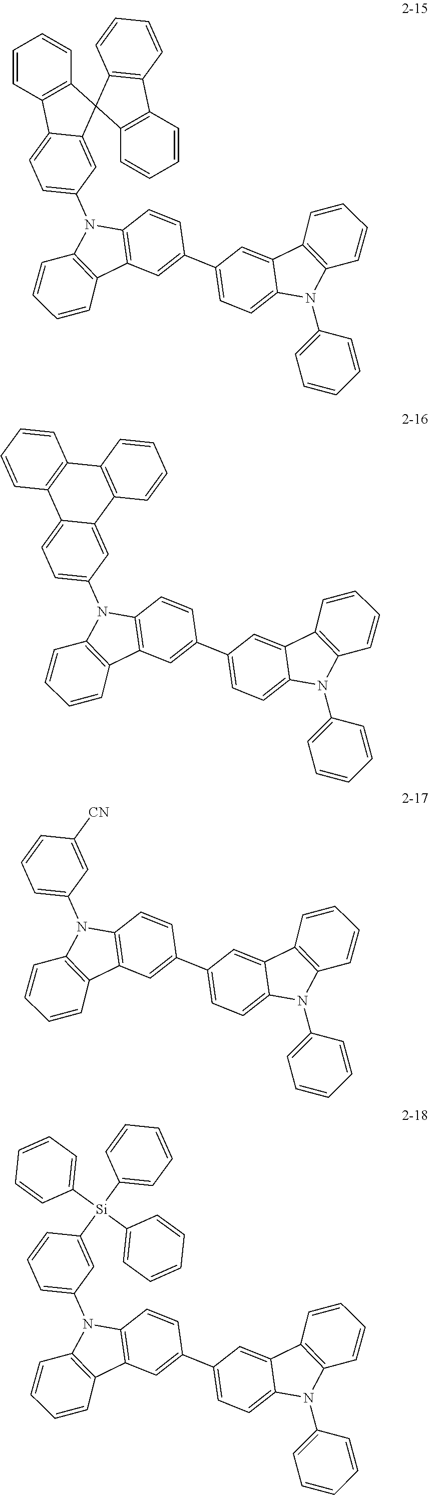 [SMILES: c1ccc(-n2c3ccccc3c3cc(-c4ccc5c(c4)c4ccccc4n5-c4ccc5c(c4)C4(c6ccccc6-c6ccccc64)c4ccccc4-5)ccc32)cc1 ; c1ccc(-n2c3ccccc3c3cc(-c4ccc5c(c4)c4ccccc4n5-c4ccc5c6ccccc6c6ccccc6c5c4)ccc32)cc1 ; N#Cc1cccc(-n2c3ccccc3c3cc(-c4ccc5c(c4)c4ccccc4n5-c4ccccc4)ccc32)c1 ; c1ccc(-n2c3ccccc3c3cc(-c4ccc5c(c4)c4ccccc4n5-c4cccc([Si](c5ccccc5)(c5ccccc5)c5ccccc5)c4)ccc32)cc1]

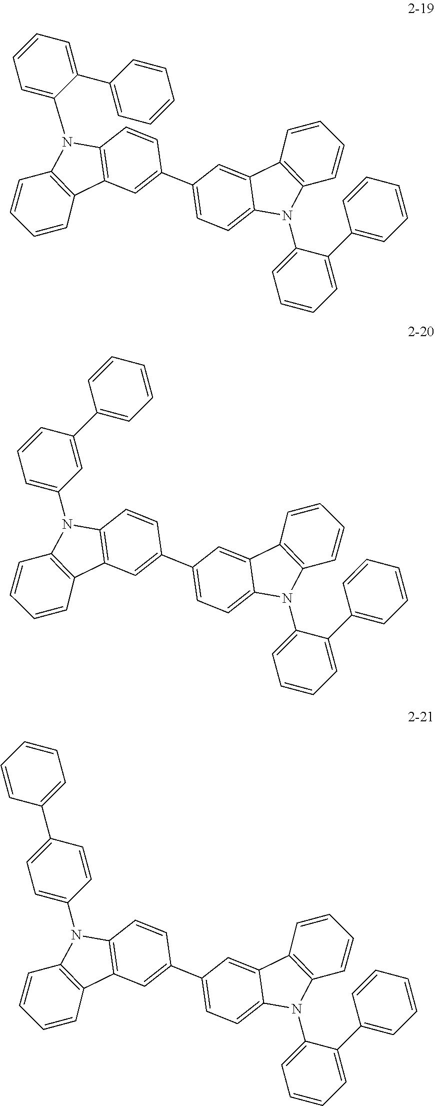 [SMILES: c1ccc(-c2ccccc2-n2c3ccccc3c3cc(-c4ccc5c(c4)c4ccccc4n5-c4ccccc4-c4ccccc4)ccc32)cc1 ; c1ccc(-c2cccc(-n3c4ccccc4c4cc(-c5ccc6c(c5)c5ccccc5n6-c5ccccc5-c5ccccc5)ccc43)c2)cc1 ; c1ccc(-c2ccc(-n3c4ccccc4c4cc(-c5ccc6c(c5)c5ccccc5n6-c5ccccc5-c5ccccc5)ccc43)cc2)cc1]

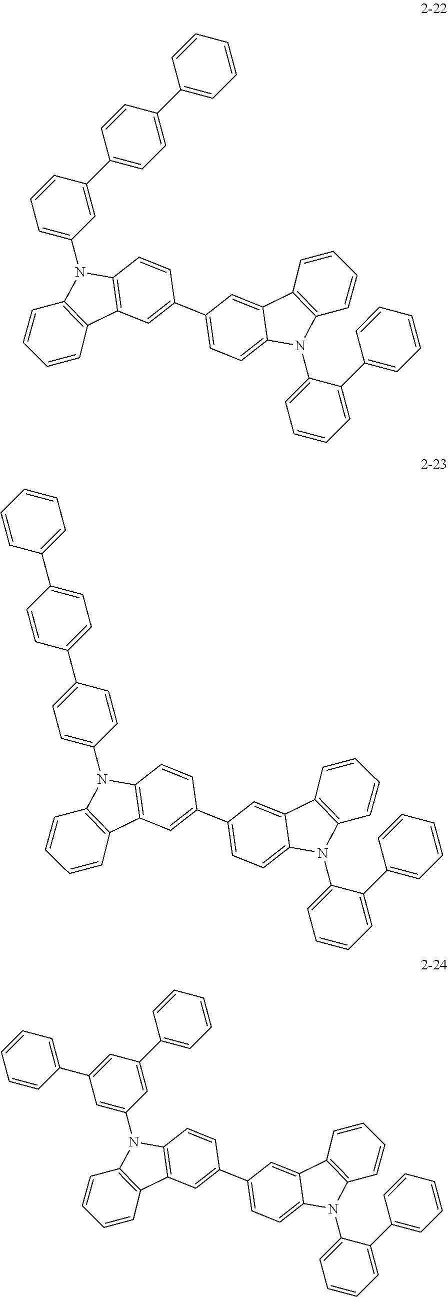 [SMILES: c1ccc(-c2ccc(-c3cccc(-n4c5ccccc5c5cc(-c6ccc7c(c6)c6ccccc6n7-c6ccccc6-c6ccccc6)ccc54)c3)cc2)cc1 ; c1ccc(-c2ccc(-c3ccc(-n4c5ccccc5c5cc(-c6ccc7c(c6)c6ccccc6n7-c6ccccc6-c6ccccc6)ccc54)cc3)cc2)cc1 ; c1ccc(-c2cc(-c3ccccc3)cc(-n3c4ccccc4c4cc(-c5ccc6c(c5)c5ccccc5n6-c5ccccc5-c5ccccc5)ccc43)c2)cc1]

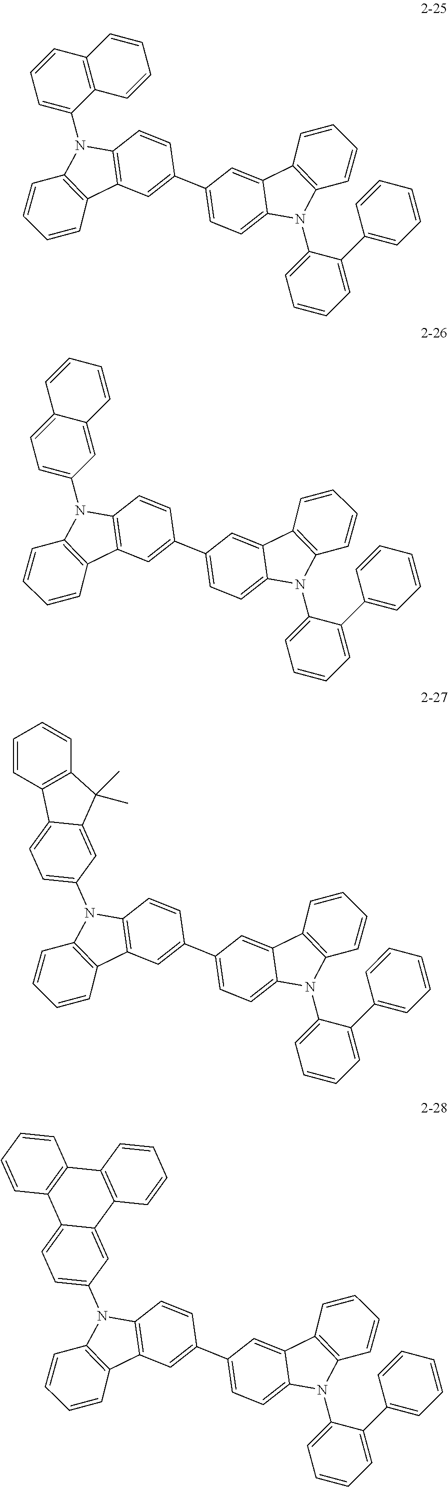 [SMILES: c1ccc(-c2ccccc2-n2c3ccccc3c3cc(-c4ccc5c(c4)c4ccccc4n5-c4cccc5ccccc45)ccc32)cc1 ; c1ccc(-c2ccccc2-n2c3ccccc3c3cc(-c4ccc5c(c4)c4ccccc4n5-c4ccc5ccccc5c4)ccc32)cc1 ; CC1(C)c2ccccc2-c2ccc(-n3c4ccccc4c4cc(-c5ccc6c(c5)c5ccccc5n6-c5ccccc5-c5ccccc5)ccc43)cc21 ; c1ccc(-c2ccccc2-n2c3ccccc3c3cc(-c4ccc5c(c4)c4ccccc4n5-c4ccc5c6ccccc6c6ccccc6c5c4)ccc32)cc1]

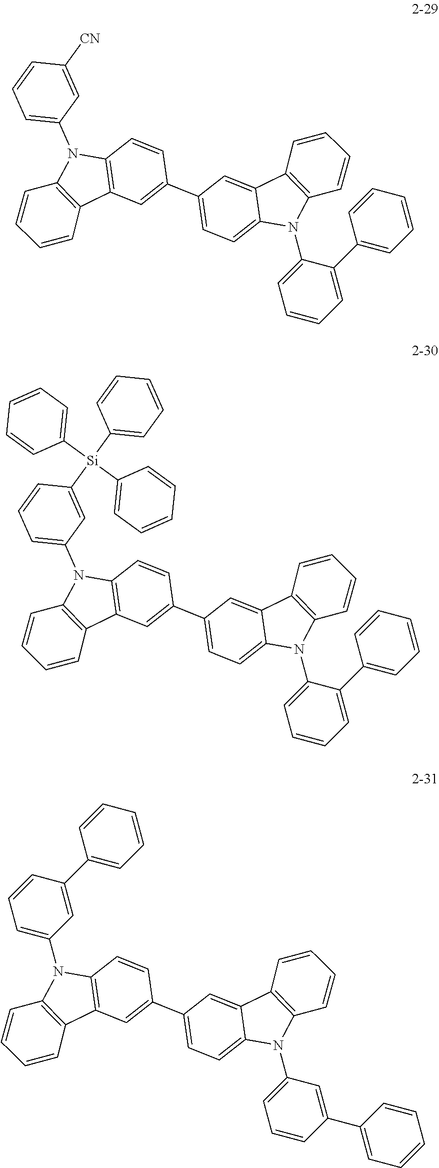 [SMILES: N#Cc1cccc(-n2c3ccccc3c3cc(-c4ccc5c(c4)c4ccccc4n5-c4ccccc4-c4ccccc4)ccc32)c1 ; c1ccc(-c2ccccc2-n2c3ccccc3c3cc(-c4ccc5c(c4)c4ccccc4n5-c4cccc([Si](c5ccccc5)(c5ccccc5)c5ccccc5)c4)ccc32)cc1 ; c1ccc(-c2cccc(-n3c4ccccc4c4cc(-c5ccc6c(c5)c5ccccc5n6-c5cccc(-c6ccccc6)c5)ccc43)c2)cc1]

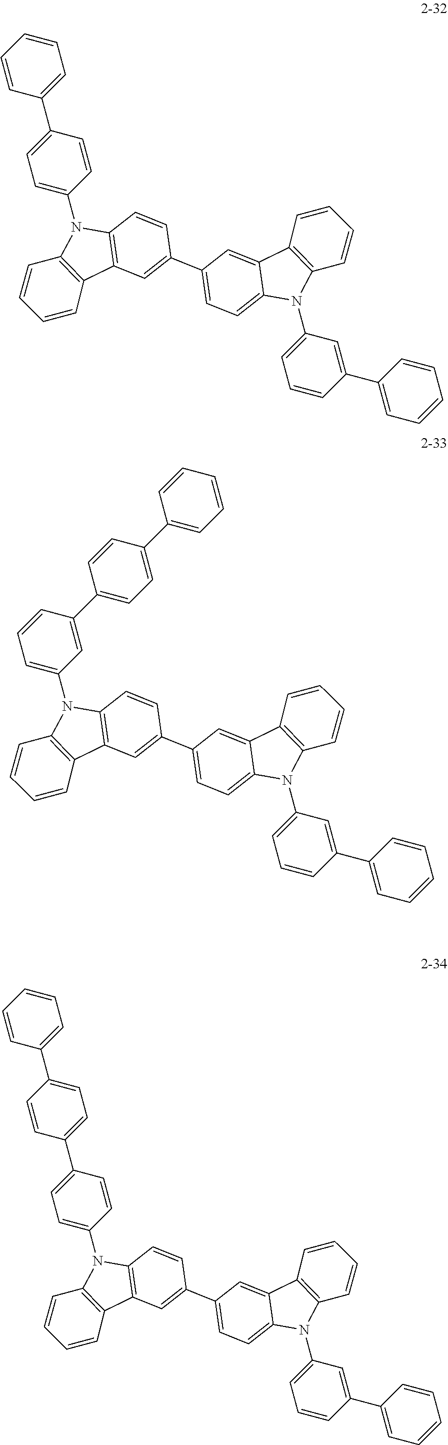 [SMILES: c1ccc(-c2ccc(-n3c4ccccc4c4cc(-c5ccc6c(c5)c5ccccc5n6-c5cccc(-c6ccccc6)c5)ccc43)cc2)cc1 ; c1ccc(-c2ccc(-c3cccc(-n4c5ccccc5c5cc(-c6ccc7c(c6)c6ccccc6n7-c6cccc(-c7ccccc7)c6)ccc54)c3)cc2)cc1 ; c1ccc(-c2ccc(-c3ccc(-n4c5ccccc5c5cc(-c6ccc7c(c6)c6ccccc6n7-c6cccc(-c7ccccc7)c6)ccc54)cc3)cc2)cc1]

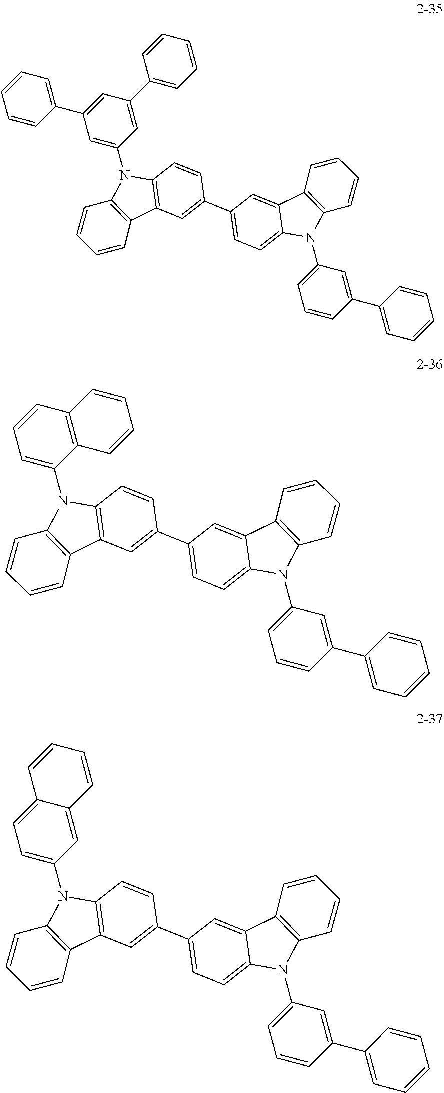 [SMILES: c1ccc(-c2cccc(-n3c4ccccc4c4cc(-c5ccc6c(c5)c5ccccc5n6-c5cc(-c6ccccc6)cc(-c6ccccc6)c5)ccc43)c2)cc1 ; c1ccc(-c2cccc(-n3c4ccccc4c4cc(-c5ccc6c(c5)c5ccccc5n6-c5cccc6ccccc56)ccc43)c2)cc1 ; c1ccc(-c2cccc(-n3c4ccccc4c4cc(-c5ccc6c(c5)c5ccccc5n6-c5ccc6ccccc6c5)ccc43)c2)cc1]

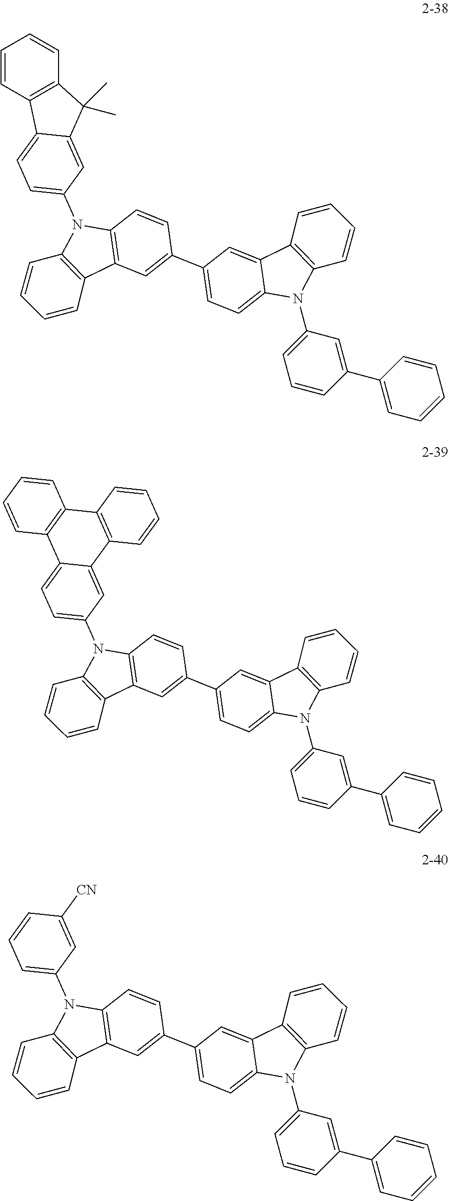 [SMILES: CC1(C)c2ccccc2-c2ccc(-n3c4ccccc4c4cc(-c5ccc6c(c5)c5ccccc5n6-c5cccc(-c6ccccc6)c5)ccc43)cc21 ; c1ccc(-c2cccc(-n3c4ccccc4c4cc(-c5ccc6c(c5)c5ccccc5n6-c5ccc6c7ccccc7c7ccccc7c6c5)ccc43)c2)cc1 ; N#Cc1cccc(-n2c3ccccc3c3cc(-c4ccc5c(c4)c4ccccc4n5-c4cccc(-c5ccccc5)c4)ccc32)c1]

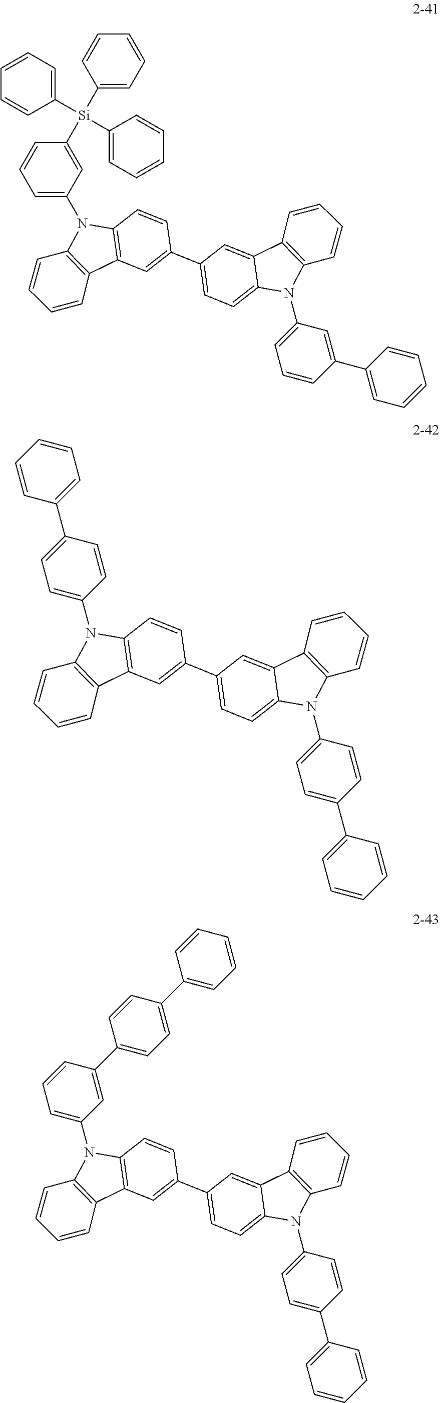 [SMILES: c1ccc(-c2cccc(-n3c4ccccc4c4cc(-c5ccc6c(c5)c5ccccc5n6-c5cccc([Si](c6ccccc6)(c6ccccc6)c6ccccc6)c5)ccc43)c2)cc1 ; c1ccc(-c2ccc(-n3c4ccccc4c4cc(-c5ccc6c(c5)c5ccccc5n6-c5ccc(-c6ccccc6)cc5)ccc43)cc2)cc1 ; c1ccc(-c2ccc(-c3cccc(-n4c5ccccc5c5cc(-c6ccc7c(c6)c6ccccc6n7-c6ccc(-c7ccccc7)cc6)ccc54)c3)cc2)cc1]

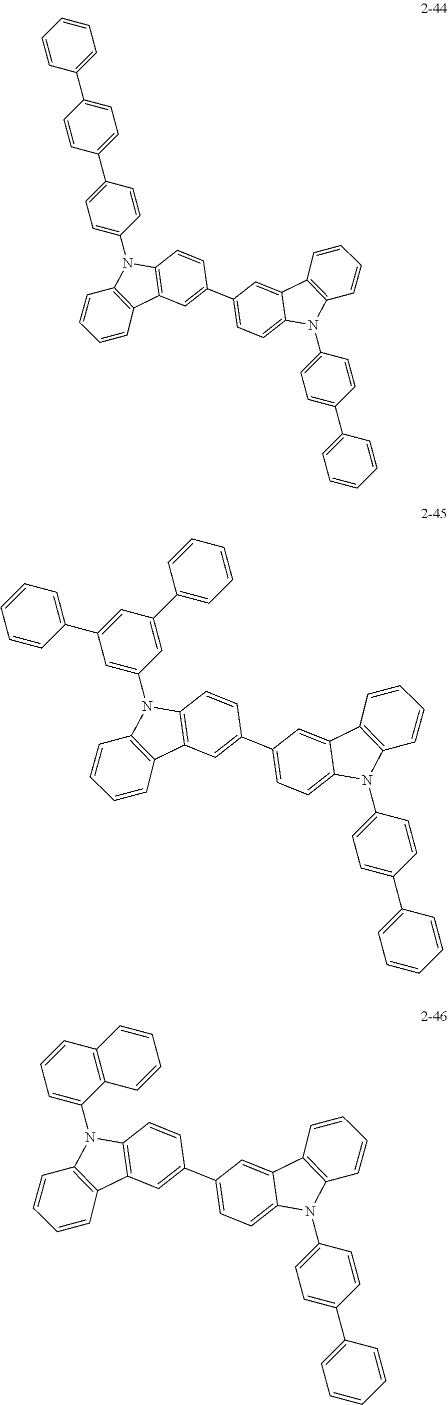 [SMILES: c1ccc(-c2ccc(-c3ccc(-n4c5ccccc5c5cc(-c6ccc7c(c6)c6ccccc6n7-c6ccc(-c7ccccc7)cc6)ccc54)cc3)cc2)cc1 ; c1ccc(-c2ccc(-n3c4ccccc4c4cc(-c5ccc6c(c5)c5ccccc5n6-c5cc(-c6ccccc6)cc(-c6ccccc6)c5)ccc43)cc2)cc1 ; c1ccc(-c2ccc(-n3c4ccccc4c4cc(-c5ccc6c(c5)c5ccccc5n6-c5cccc6ccccc56)ccc43)cc2)cc1]

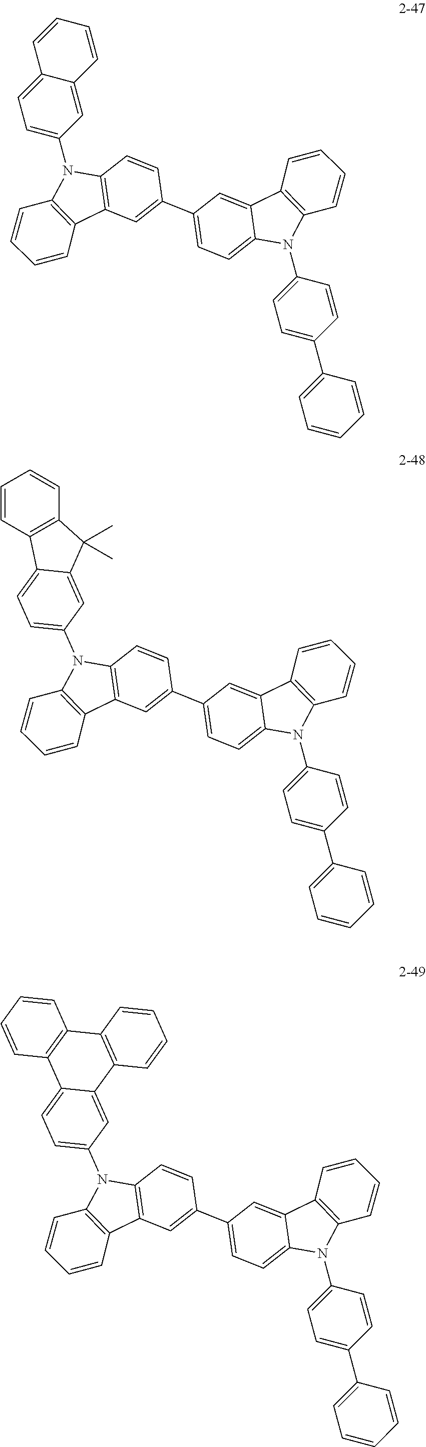 [SMILES: c1ccc(-c2ccc(-n3c4ccccc4c4cc(-c5ccc6c(c5)c5ccccc5n6-c5ccc6ccccc6c5)ccc43)cc2)cc1 ; CC1(C)c2ccccc2-c2ccc(-n3c4ccccc4c4cc(-c5ccc6c(c5)c5ccccc5n6-c5ccc(-c6ccccc6)cc5)ccc43)cc21 ; c1ccc(-c2ccc(-n3c4ccccc4c4cc(-c5ccc6c(c5)c5ccccc5n6-c5ccc6c7ccccc7c7ccccc7c6c5)ccc43)cc2)cc1]

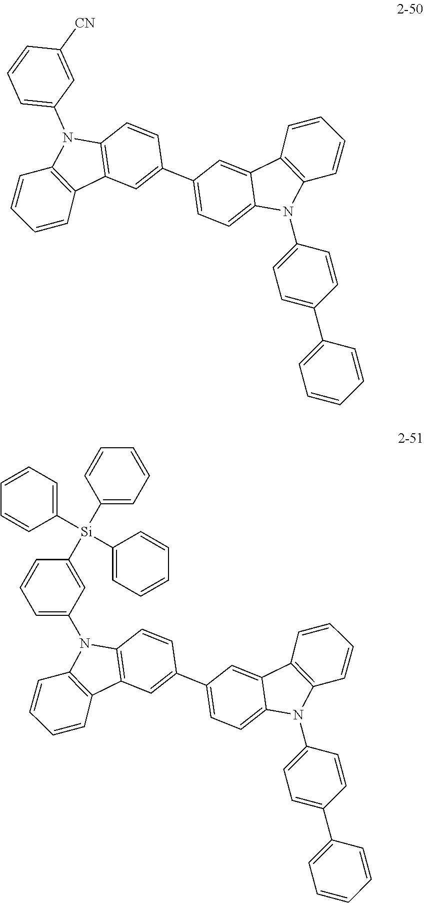 [SMILES: N#Cc1cccc(-n2c3ccccc3c3cc(-c4ccc5c(c4)c4ccccc4n5-c4ccc(-c5ccccc5)cc4)ccc32)c1 ; c1ccc(-c2ccc(-n3c4ccccc4c4cc(-c5ccc6c(c5)c5ccccc5n6-c5cccc([Si](c6ccccc6)(c6ccccc6)c6ccccc6)c5)ccc43)cc2)cc1]

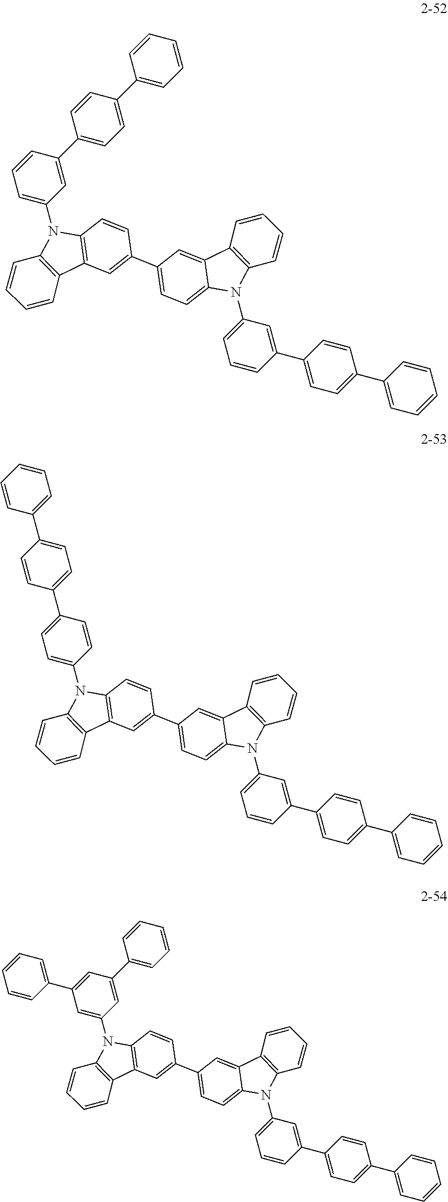 [SMILES: c1ccc(-c2ccc(-c3cccc(-n4c5ccccc5c5cc(-c6ccc7c(c6)c6ccccc6n7-c6cccc(-c7ccc(-c8ccccc8)cc7)c6)ccc54)c3)cc2)cc1 ; c1ccc(-c2ccc(-c3ccc(-n4c5ccccc5c5cc(-c6ccc7c(c6)c6ccccc6n7-c6cccc(-c7ccc(-c8ccccc8)cc7)c6)ccc54)cc3)cc2)cc1 ; c1ccc(-c2ccc(-c3cccc(-n4c5ccccc5c5cc(-c6ccc7c(c6)c6ccccc6n7-c6cc(-c7ccccc7)cc(-c7ccccc7)c6)ccc54)c3)cc2)cc1]

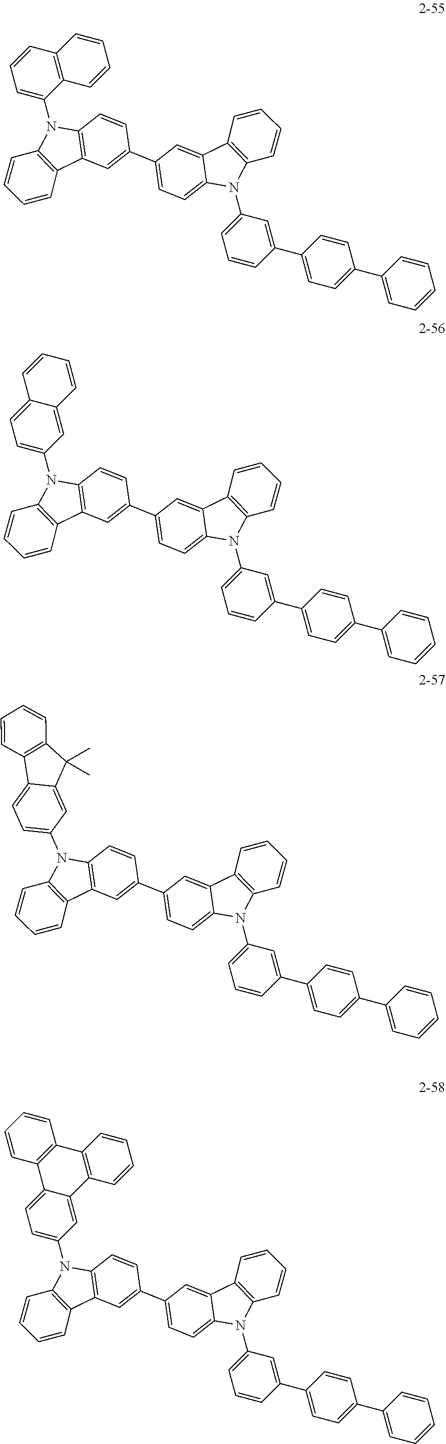 [SMILES: c1ccc(-c2ccc(-c3cccc(-n4c5ccccc5c5cc(-c6ccc7c(c6)c6ccccc6n7-c6cccc7ccccc67)ccc54)c3)cc2)cc1 ; c1ccc(-c2ccc(-c3cccc(-n4c5ccccc5c5cc(-c6ccc7c(c6)c6ccccc6n7-c6ccc7ccccc7c6)ccc54)c3)cc2)cc1 ; CC1(C)c2ccccc2-c2ccc(-n3c4ccccc4c4cc(-c5ccc6c(c5)c5ccccc5n6-c5cccc(-c6ccc(-c7ccccc7)cc6)c5)ccc43)cc21 ; c1ccc(-c2ccc(-c3cccc(-n4c5ccccc5c5cc(-c6ccc7c(c6)c6ccccc6n7-c6ccc7c8ccccc8c8ccccc8c7c6)ccc54)c3)cc2)cc1]

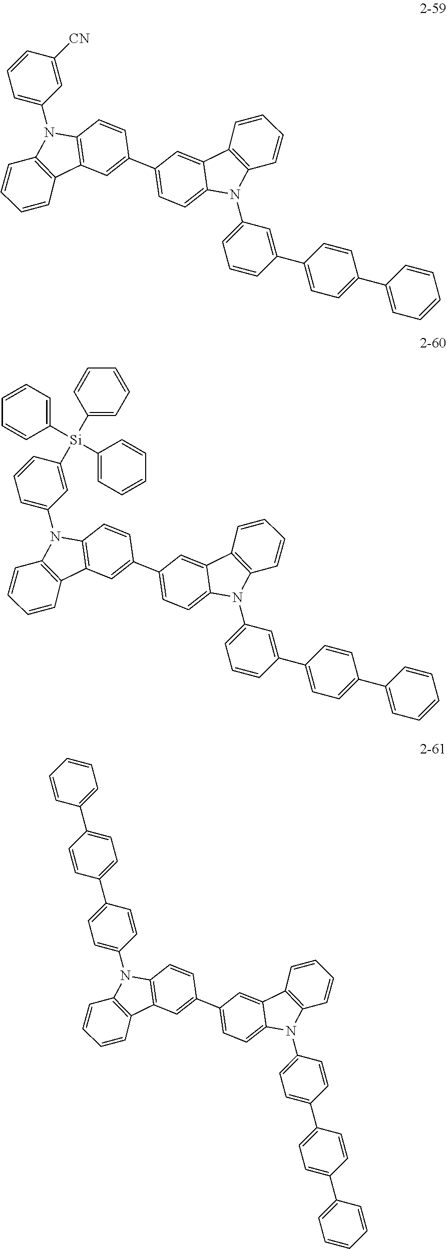 [SMILES: N#Cc1cccc(-n2c3ccccc3c3cc(-c4ccc5c(c4)c4ccccc4n5-c4cccc(-c5ccc(-c6ccccc6)cc5)c4)ccc32)c1 ; c1ccc(-c2ccc(-c3cccc(-n4c5ccccc5c5cc(-c6ccc7c(c6)c6ccccc6n7-c6cccc([Si](c7ccccc7)(c7ccccc7)c7ccccc7)c6)ccc54)c3)cc2)cc1 ; c1ccc(-c2ccc(-c3ccc(-n4c5ccccc5c5cc(-c6ccc7c(c6)c6ccccc6n7-c6ccc(-c7ccc(-c8ccccc8)cc7)cc6)ccc54)cc3)cc2)cc1]

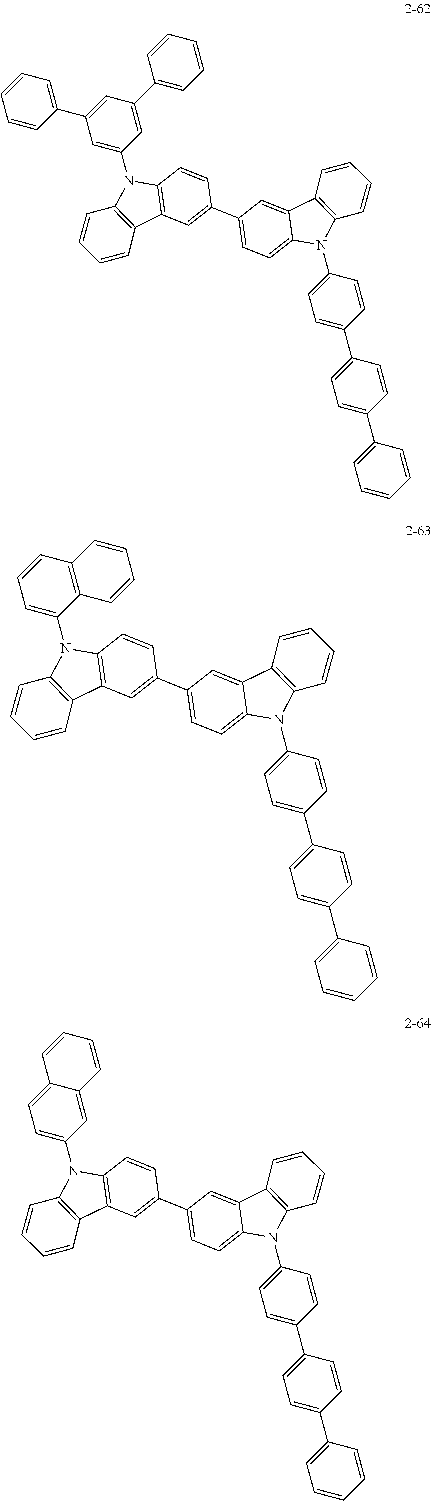 [SMILES: c1ccc(-c2ccc(-c3ccc(-n4c5ccccc5c5cc(-c6ccc7c(c6)c6ccccc6n7-c6cc(-c7ccccc7)cc(-c7ccccc7)c6)ccc54)cc3)cc2)cc1 ; c1ccc(-c2ccc(-c3ccc(-n4c5ccccc5c5cc(-c6ccc7c(c6)c6ccccc6n7-c6cccc7ccccc67)ccc54)cc3)cc2)cc1 ; c1ccc(-c2ccc(-c3ccc(-n4c5ccccc5c5cc(-c6ccc7c(c6)c6ccccc6n7-c6ccc7ccccc7c6)ccc54)cc3)cc2)cc1]

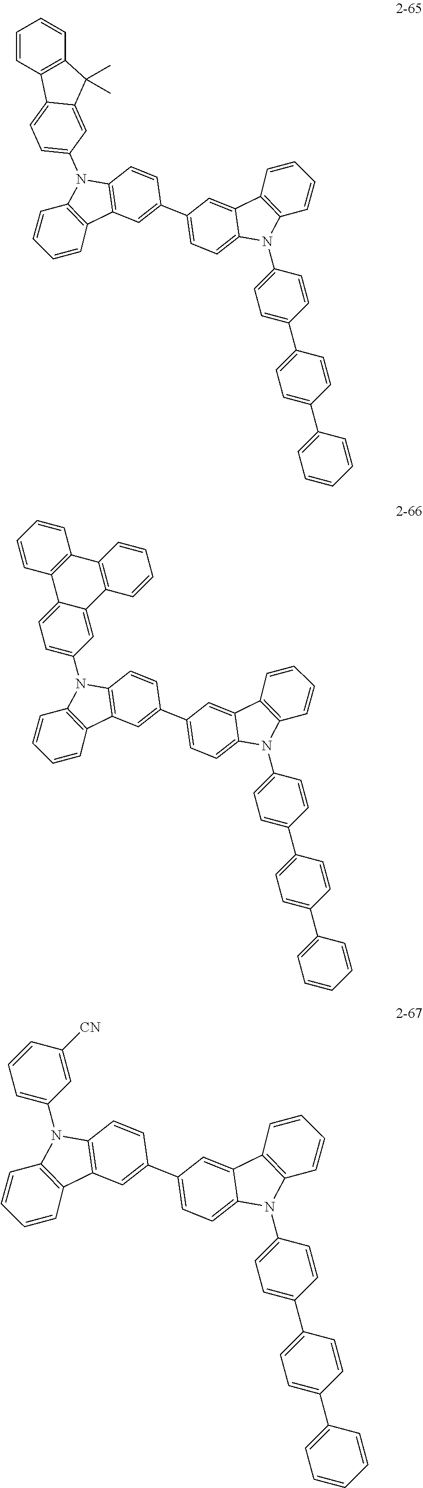 [SMILES: CC1(C)c2ccccc2-c2ccc(-n3c4ccccc4c4cc(-c5ccc6c(c5)c5ccccc5n6-c5ccc(-c6ccc(-c7ccccc7)cc6)cc5)ccc43)cc21 ; c1ccc(-c2ccc(-c3ccc(-n4c5ccccc5c5cc(-c6ccc7c(c6)c6ccccc6n7-c6ccc7c8ccccc8c8ccccc8c7c6)ccc54)cc3)cc2)cc1 ; N#Cc1cccc(-n2c3ccccc3c3cc(-c4ccc5c(c4)c4ccccc4n5-c4ccc(-c5ccc(-c6ccccc6)cc5)cc4)ccc32)c1]

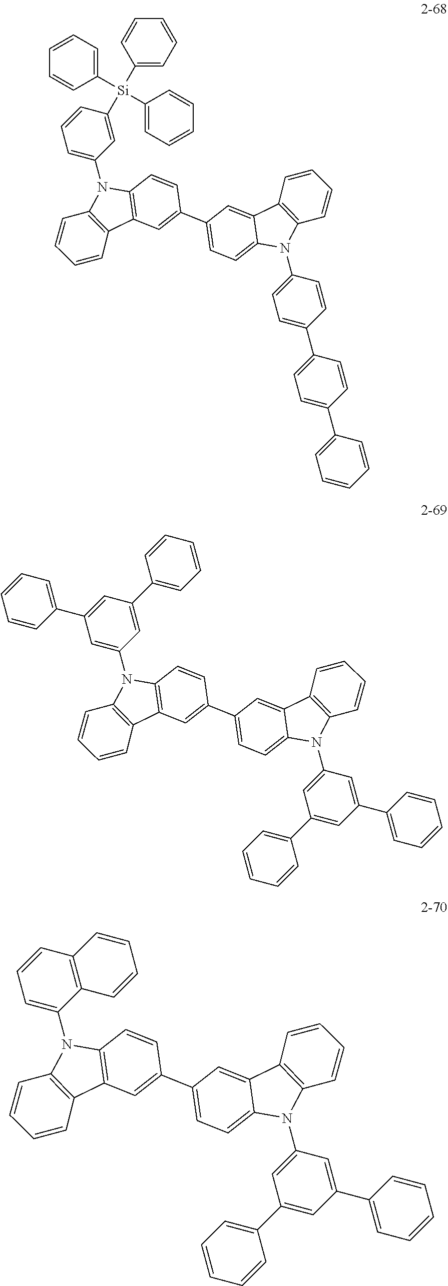 [SMILES: c1ccc(-c2ccc(-c3ccc(-n4c5ccccc5c5cc(-c6ccc7c(c6)c6ccccc6n7-c6cccc([Si](c7ccccc7)(c7ccccc7)c7ccccc7)c6)ccc54)cc3)cc2)cc1 ; c1ccc(-c2cc(-c3ccccc3)cc(-n3c4ccccc4c4cc(-c5ccc6c(c5)c5ccccc5n6-c5cc(-c6ccccc6)cc(-c6ccccc6)c5)ccc43)c2)cc1 ; c1ccc(-c2cc(-c3ccccc3)cc(-n3c4ccccc4c4cc(-c5ccc6c(c5)c5ccccc5n6-c5cccc6ccccc56)ccc43)c2)cc1]

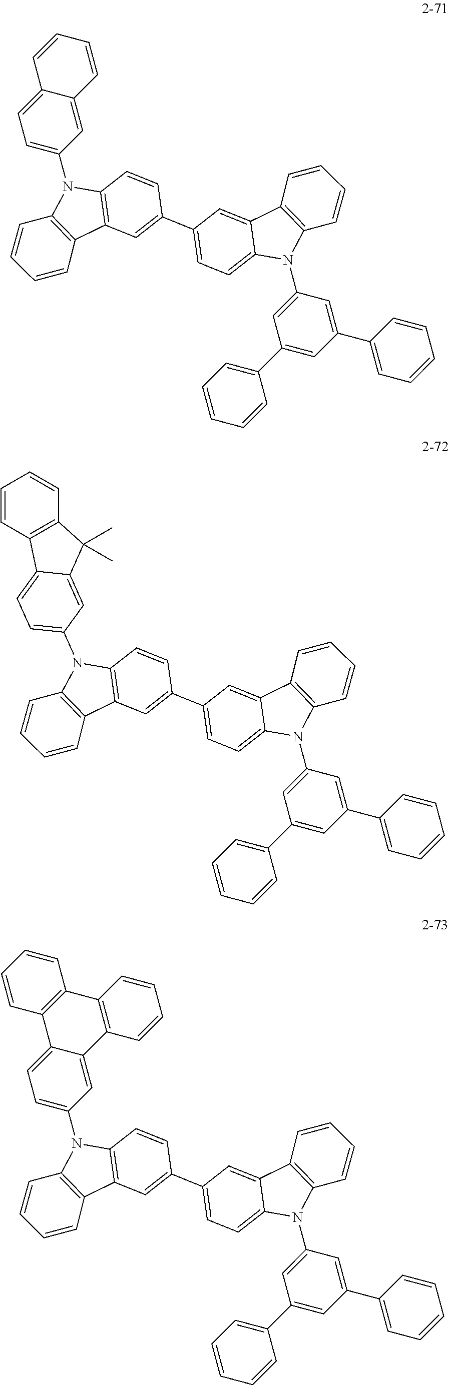 [SMILES: c1ccc(-c2cc(-c3ccccc3)cc(-n3c4ccccc4c4cc(-c5ccc6c(c5)c5ccccc5n6-c5ccc6ccccc6c5)ccc43)c2)cc1 ; CC1(C)c2ccccc2-c2ccc(-n3c4ccccc4c4cc(-c5ccc6c(c5)c5ccccc5n6-c5cc(-c6ccccc6)cc(-c6ccccc6)c5)ccc43)cc21 ; c1ccc(-c2cc(-c3ccccc3)cc(-n3c4ccccc4c4cc(-c5ccc6c(c5)c5ccccc5n6-c5ccc6c7ccccc7c7ccccc7c6c5)ccc43)c2)cc1]

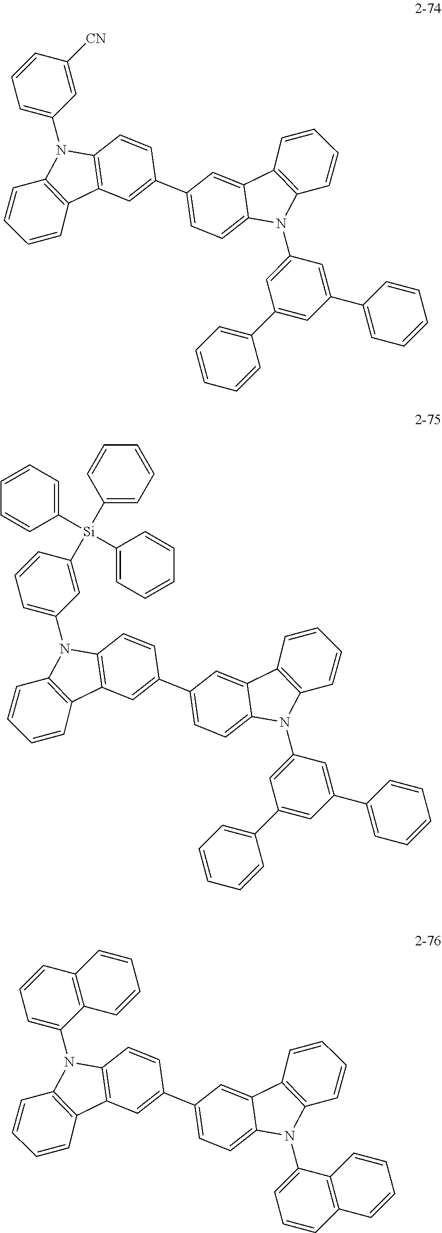 [SMILES: N#Cc1cccc(-n2c3ccccc3c3cc(-c4ccc5c(c4)c4ccccc4n5-c4cc(-c5ccccc5)cc(-c5ccccc5)c4)ccc32)c1 ; c1ccc(-c2cc(-c3ccccc3)cc(-n3c4ccccc4c4cc(-c5ccc6c(c5)c5ccccc5n6-c5cccc([Si](c6ccccc6)(c6ccccc6)c6ccccc6)c5)ccc43)c2)cc1 ; c1ccc2c(-n3c4ccccc4c4cc(-c5ccc6c(c5)c5ccccc5n6-c5cccc6ccccc56)ccc43)cccc2c1]

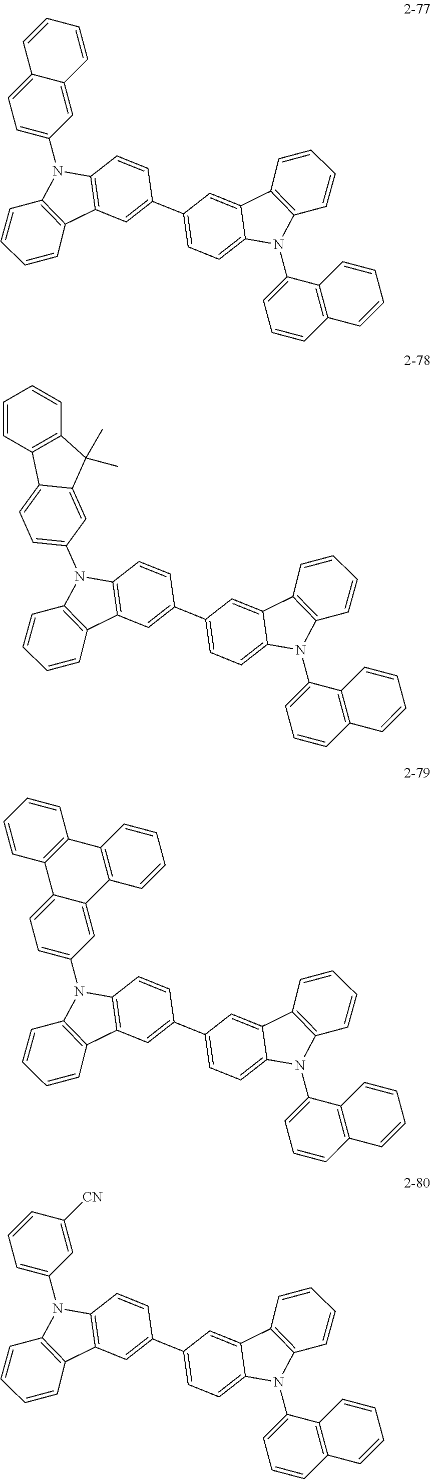 [SMILES: c1ccc2cc(-n3c4ccccc4c4cc(-c5ccc6c(c5)c5ccccc5n6-c5cccc6ccccc56)ccc43)ccc2c1 ; CC1(C)c2ccccc2-c2ccc(-n3c4ccccc4c4cc(-c5ccc6c(c5)c5ccccc5n6-c5cccc6ccccc56)ccc43)cc21 ; c1ccc2c(-n3c4ccccc4c4cc(-c5ccc6c(c5)c5ccccc5n6-c5ccc6c7ccccc7c7ccccc7c6c5)ccc43)cccc2c1 ; N#Cc1cccc(-n2c3ccccc3c3cc(-c4ccc5c(c4)c4ccccc4n5-c4cccc5ccccc45)ccc32)c1]

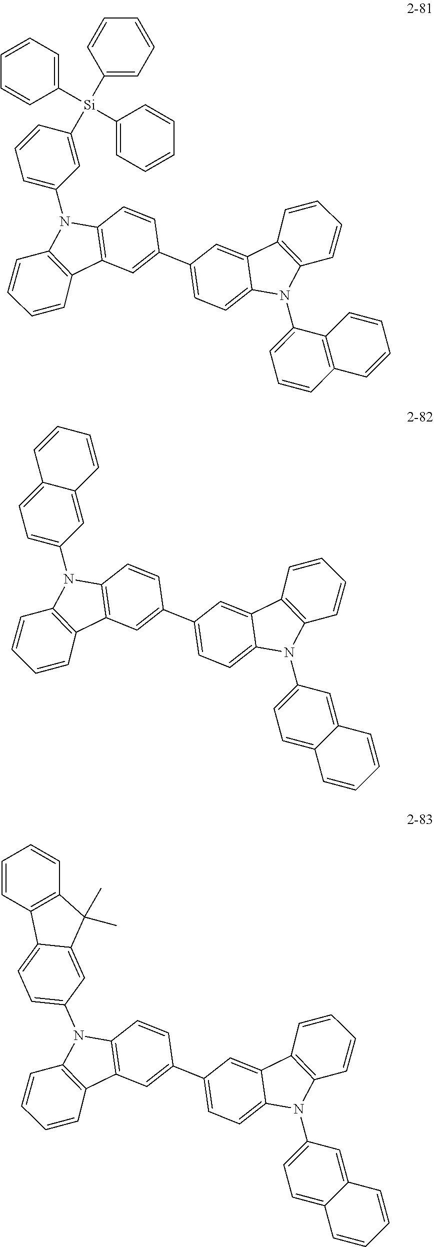 [SMILES: c1ccc([Si](c2ccccc2)(c2ccccc2)c2cccc(-n3c4ccccc4c4cc(-c5ccc6c(c5)c5ccccc5n6-c5cccc6ccccc56)ccc43)c2)cc1 ; c1ccc2cc(-n3c4ccccc4c4cc(-c5ccc6c(c5)c5ccccc5n6-c5ccc6ccccc6c5)ccc43)ccc2c1 ; CC1(C)c2ccccc2-c2ccc(-n3c4ccccc4c4cc(-c5ccc6c(c5)c5ccccc5n6-c5ccc6ccccc6c5)ccc43)cc21]

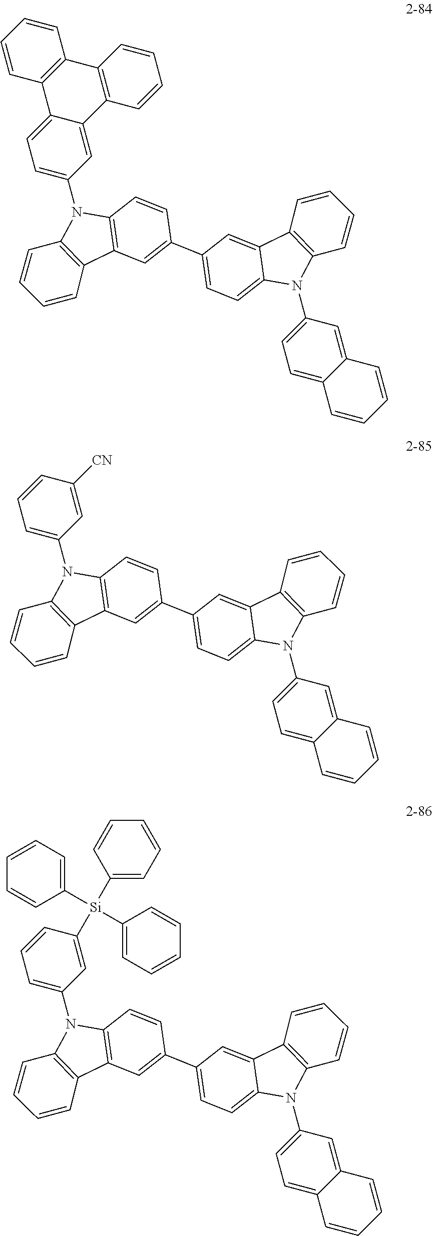 [SMILES: c1ccc2cc(-n3c4ccccc4c4cc(-c5ccc6c(c5)c5ccccc5n6-c5ccc6c7ccccc7c7ccccc7c6c5)ccc43)ccc2c1 ; N#Cc1cccc(-n2c3ccccc3c3cc(-c4ccc5c(c4)c4ccccc4n5-c4ccc5ccccc5c4)ccc32)c1 ; c1ccc([Si](c2ccccc2)(c2ccccc2)c2cccc(-n3c4ccccc4c4cc(-c5ccc6c(c5)c5ccccc5n6-c5ccc6ccccc6c5)ccc43)c2)cc1]

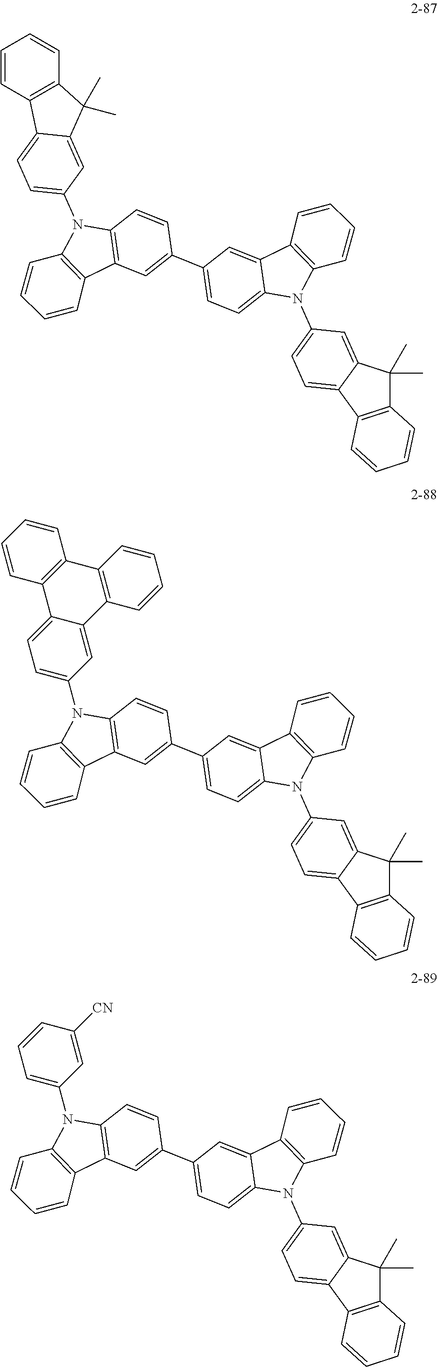 [SMILES: CC1(C)c2ccccc2-c2ccc(-n3c4ccccc4c4cc(-c5ccc6c(c5)c5ccccc5n6-c5ccc6c(c5)C(C)(C)c5ccccc5-6)ccc43)cc21 ; CC1(C)c2ccccc2-c2ccc(-n3c4ccccc4c4cc(-c5ccc6c(c5)c5ccccc5n6-c5ccc6c7ccccc7c7ccccc7c6c5)ccc43)cc21 ; CC1(C)c2ccccc2-c2ccc(-n3c4ccccc4c4cc(-c5ccc6c(c5)c5ccccc5n6-c5cccc(C#N)c5)ccc43)cc21]

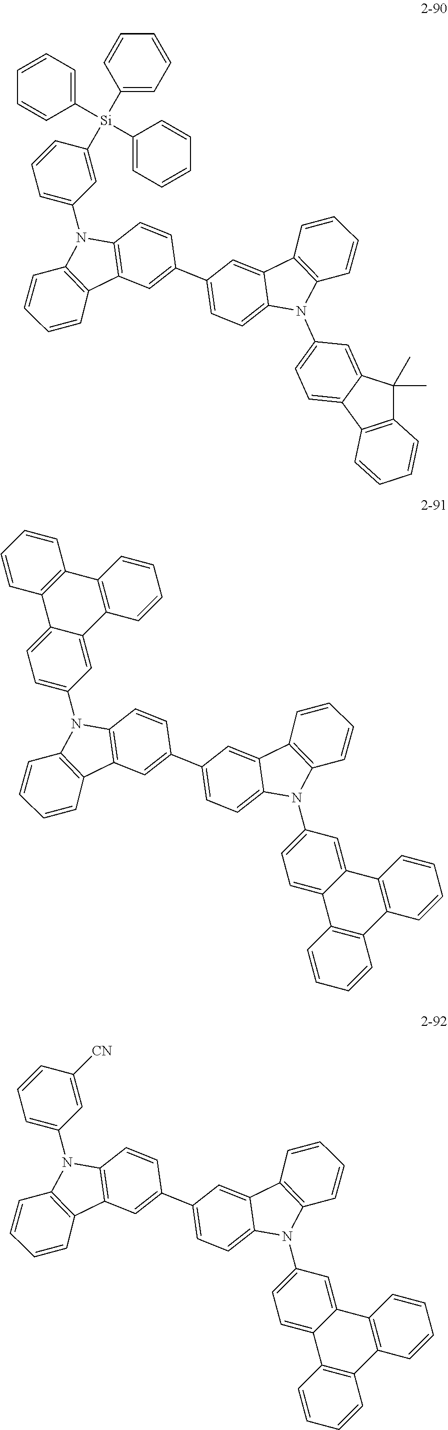 [SMILES: CC1(C)c2ccccc2-c2ccc(-n3c4ccccc4c4cc(-c5ccc6c(c5)c5ccccc5n6-c5cccc([Si](c6ccccc6)(c6ccccc6)c6ccccc6)c5)ccc43)cc21 ; c1ccc2c(c1)c1ccccc1c1cc(-n3c4ccccc4c4cc(-c5ccc6c(c5)c5ccccc5n6-c5ccc6c7ccccc7c7ccccc7c6c5)ccc43)ccc21 ; N#Cc1cccc(-n2c3ccccc3c3cc(-c4ccc5c(c4)c4ccccc4n5-c4ccc5c6ccccc6c6ccccc6c5c4)ccc32)c1]

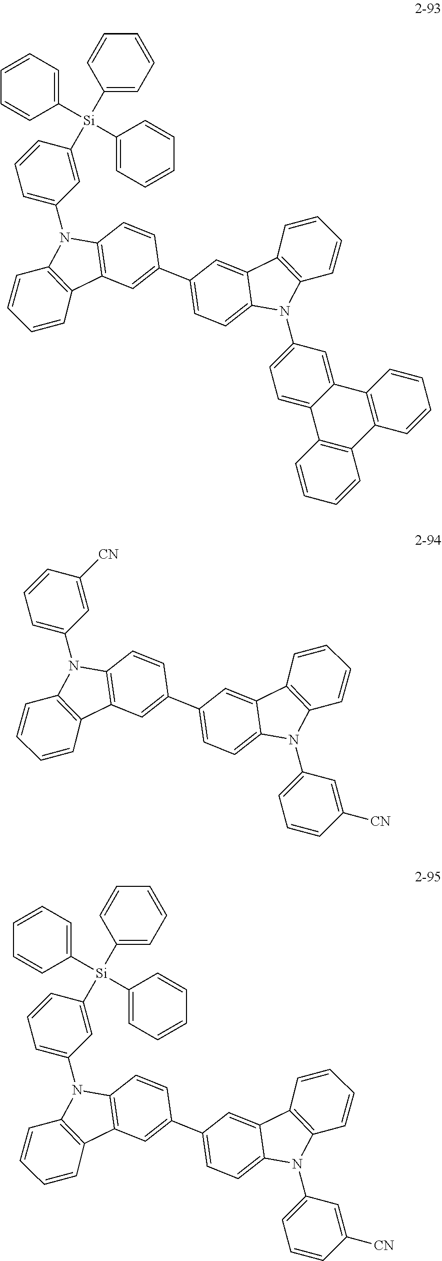 [SMILES: c1ccc([Si](c2ccccc2)(c2ccccc2)c2cccc(-n3c4ccccc4c4cc(-c5ccc6c(c5)c5ccccc5n6-c5ccc6c7ccccc7c7ccccc7c6c5)ccc43)c2)cc1 ; N#Cc1cccc(-n2c3ccccc3c3cc(-c4ccc5c(c4)c4ccccc4n5-c4cccc(C#N)c4)ccc32)c1 ; N#Cc1cccc(-n2c3ccccc3c3cc(-c4ccc5c(c4)c4ccccc4n5-c4cccc([Si](c5ccccc5)(c5ccccc5)c5ccccc5)c4)ccc32)c1]

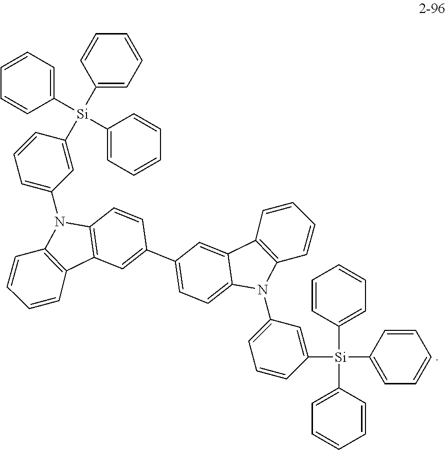 [SMILES: c1ccc([Si](c2ccccc2)(c2ccccc2)c2cccc(-n3c4ccccc4c4cc(-c5ccc6c(c5)c5ccccc5n6-c5cccc([Si](c6ccccc6)(c6ccccc6)c6ccccc6)c5)ccc43)c2)cc1]

13. The organic light emitting device of claim 8, further comprising one, two or more layers selected from the group consisting of a light emitting layer, a hole injection layer, a hole transfer layer, an electron injection layer, an electron transfer layer, an electron blocking layer and a hole blocking layer.

14. A composition for an organic material layer of an organic light emitting device, the composition comprising the heterocyclic compound of claim 1.

15. The composition for an organic material layer of an organic light emitting device of claim 14, further comprising a compound of the following Chemical Formula 2: 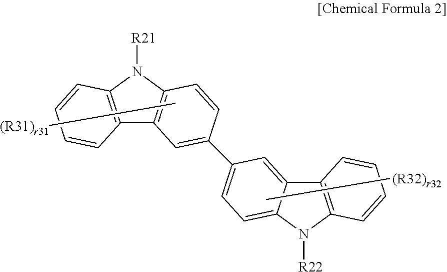
wherein, in Chemical Formula 2,
R21 and R22 are the same as or different from each other, and each independently deuterium; a halogen group; a cyano group; a substituted or unsubstituted C1 to C60 alkyl group; a substituted or unsubstituted C3 to C60 cycloalkyl group; or a substituted or unsubstituted C6 to C60 aryl group,
R31 and R32 are the same as or different from each other, and each independently hydrogen; deuterium; a halogen group; a cyano group; a substituted or unsubstituted C1 to C60 alkyl group; a substituted or unsubstituted C3 to C60 cycloalkyl group; a substituted or unsubstituted C6 to C60 aryl group; or a substituted or unsubstituted C2 to C60 heteroaryl group,
r31 is an integer of 0 to 7, and when 2 or greater, R31s are the same as or different from each other, and
r32 is an integer of 0 to 7, and when 2 or greater, R32s are the same as or different from each other.

16. The composition for an organic material layer of an organic light emitting device of claim 15, comprising the heterocyclic compound and the compound of Chemical Formula 2 in a weight ratio of 1:10 to 10:1.
